(19) United States
(12) Patent Application Publication
(10) Pub. No.: US 2025/0338770 A1
(43) Pub. Date: Oct. 30, 2025

(54) ORGANIC COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Xianbin XU, Xi'an (CN); Lei YANG, Xi'an (CN)

(21) Appl. No.: 18/870,792

(22) PCT Filed: Oct. 8, 2023
(86) PCT No.: PCT/CN2023/123453

§ 371 Date: Dec. 2, 2024

(30) Foreign Application Priority Data
May 23, 2023 (CN) 202310587788.X

(51) Int. Cl. H10K 85/60; C07B 59/00; C07D 498/04; C07D 513/04; C09K 11/02; H10K 50/11

(52) U.S. Cl. CPC H10K 85/636; C07B 59/002; C07D 498/04; C07D 513/04; C09K 11/02; H10K 85/633; H10K 50/11; H10K 85/615; H10K 85/626; H10K 85/657; H10K 85/6572; H10K 85/6574; H10K 85/6576

(57) ABSTRACT

The present disclosure belongs to the technical field of organic materials, and provides an organic compound, and the organic compound has a structure shown in a formula 1. The present disclosure also provides an organic electroluminescent device comprising the organic compound and an electronic apparatus. The organic compound, as a host material for an organic electroluminescent layer, can effectively reduce a driving voltage of an organic electroluminescent device, increase the luminous efficiency of the organic electroluminescent device, and prolong the service life of the organic electroluminescent device.

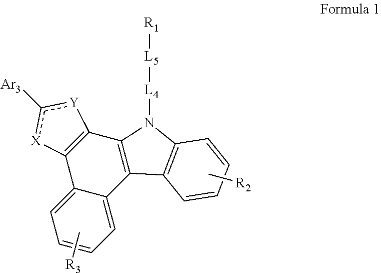

### CROSS REFERENCE TO RELATED APPLICATIONS

This disclosure claims the priority of Chinese patent application No. 202310587788.X, filed on May 23, 2023, the contents of which are incorporated here by reference in their entirety as part of the present disclosure.

### TECHNICAL FIELD

The present disclosure relates to the technical field of organic materials, in particular to an organic compound, an organic electroluminescent device and an electronic apparatus.

### BACKGROUND

An organic electroluminescent device, such as an organic light-emitting diode (OLED), typically comprises a cathode and an anode which are oppositely disposed, and a functional layer disposed between the cathode and the anode. The functional layer consists of a plurality of organic or inorganic film layers, and generally comprises an organic electroluminescent layer, a hole transport layer, an electron transport layer, and the like. When a voltage is applied to the cathode and the anode, an electric field is generated between the two electrodes, and electrons on a cathode side move toward an organic electroluminescent layer, and holes on an anode side also move toward the organic electroluminescent layer under the action of the electric field, and the electrons and the holes are combined in the organic electroluminescent layer to form excitons, the excitons are in an excited state and release energy outwards, and then the organic electroluminescent layer emits light outwards.

In the existing organic electroluminescent device, the most important problems are service life and efficiency, and as an area of a display increases, the driving voltage also increases, and the luminous efficiency and the current efficiency also need to be improved. Thus, it is necessary to continue to develop new materials to further improve the performance of the organic electroluminescent device.

### SUMMARY

An object of the present disclosure is to overcome the above defects in the prior art and provide an organic compound and an organic electroluminescent device comprising the same and an electronic apparatus. The organic compound can improve the luminous efficiency and prolong the service life of the device.

In order to achieve the above object, in a first aspect of the present disclosure, provided is an organic compound, having a structure shown in a Formula 1:

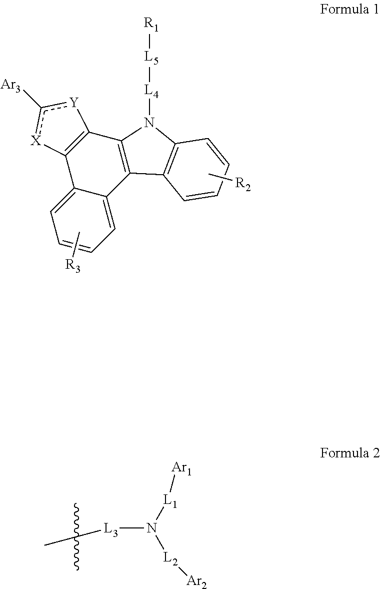

where X and Y are each independently selected from

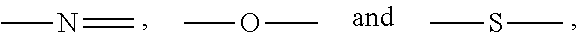

and one and only one of X and Y is

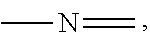

and the other is

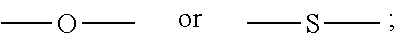

R₁ is selected from a hydrogen, a deuterium, or a structure represented by a Formula 2;
R₂ and R₃ are the same or different, and are each independently selected from a hydrogen, a deuterium, a cyano, a halogen group, an alkyl with 1 to 10 carbon atoms, a haloalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, a cycloalkyl with 3 to 10 carbon atoms, or the structure represented by the Formula 2;
and at least one of R₁, R₂, and R₃ is the group structure represented by the Formula 2;
L₁, L₂, L₃ and L₄ are the same or different, and are each independently selected from a single bond, a substituted or unsubstituted arylene with 6 to 30 carbon atoms, or a substituted or unsubstituted heteroarylene with 3 to 30 carbon atoms;
L₅ is selected from a substituted or unsubstituted arylene with 6 to 30 carbon atoms, or a substituted or unsubstituted heteroarylene with 3 to 30 carbon atoms;
Ar₁, Ar₂, and Ar₃ are the same or different, and are each independently selected from a substituted or unsubstituted aryl with 6 to 30 carbon atoms, or a substituted or unsubstituted heteroaryl with 3 to 30 carbon atoms;
substituent(s) in L₁, L₂, L₃, L₄, L₅, Ar₁, Ar₂, and Ar₃ are the same or different, and are each independently selected from a deuterium, a cyano, a halogen group, an alkyl with 1 to 10 carbon atoms, a haloalkyl with 1 to 10 carbon atoms, a trialkylsilyl with 3 to 12 carbon atoms, a triphenylsilyl, aryl with 6 to 20 carbon atoms, a heteroaryl with 3 to 20 carbon atoms, or a cycloalkyl with 3 to 10 carbon atoms; and optionally, in Ar₁ and Ar₂, any two adjacent substituents form a ring.

In a second aspect of the present disclosure, provided is an organic electroluminescent device, comprising an anode, a cathode, and at least one functional layer disposed between the anode and the cathode, where the functional layer comprises the organic compound according to the first aspect of the present disclosure.

In a third aspect of the present disclosure, provided is an electronic apparatus, comprising the organic electroluminescent device according to the second aspect of the present disclosure.

Through the above technical solutions, the structure of the organic compound of the present disclosure includes a parent core structure of oxazolo/thiazolobenzo[C]carbazole, and the parent core is linked to an arylamine compound, and thus the organic compound is used as a host material of an organic electroluminescent layer. On one hand, benzo[C]carbazole itself has a large conjugated system and a suitable first excited triplet energy level, and fusing benzo[C]carbazole with an oxazole/thiazole ring can further increase the conjugated system, after linking this parent core structure to an aromatic amine, the intermolecular force can be enhanced and the carrier mobility of the compound can be increased; on the other hand, after oxazole/thiazole is fused with benzo[C]carbazole, radical cations formed from the arylamine compound during hole transport can be stabilized and the electrochemical stability of the compound can be improved. When the organic compound of the present disclosure is used as the host material of the organic electroluminescent layer, the carrier balance in a light-emitting layer can be improved, the carrier recombination region can be broadened, the exciton generation and utilization efficiency can be increased, and the luminous efficiency and service life of the device can be increased.

Other features and advantages of the present disclosure will be described in detail in the subsequent detailed description.

### BRIEF DESCRIPTION OF THE DRAWINGS

The accompanying drawings here, which are incorporated into the specification and constitute a part of this specification, illustrate the examples consistent with the present disclosure and together with the specification, serve to explain the principles of the present disclosure.

FIG. 1 is a schematic structural diagram of an organic electroluminescent device according to one embodiment of the present disclosure.

FIG. 2 is a schematic structural diagram of an electronic apparatus according to one embodiment of the present disclosure.

### DESCRIPTION OF REFERENCE SIGNS

100, anode; 200, cathode; 300, functional layer; 310, hole injection layer; 321, hole transport layer; 322, hole auxiliary layer; 330, organic electroluminescent layer; 340, electron transport layer; 350, electron injection layer; and 400, electronic apparatus.

Through the above drawings, specific embodiments of the present disclosure have been illustrated and will be described in greater detail in the below. These drawings and written description are not intended to limit the scope of the concept of the present disclosure in any way, but rather to illustrate the concept of the present disclosure for those skilled in the art by reference to specific examples.

### DETAILED DESCRIPTION

Examples will now be described more fully with reference to the accompanying drawings. However, the examples may be implemented in various forms and should not be construed as limited to the instances set forth here; rather, these examples are provided so that the present disclosure will be thorough and complete, and the concept of the examples is fully conveyed to those skilled in the art. The described features, structures, or characteristics may be combined in any suitable manner in one or more examples. In the following description, many specific details are provided to provide a thorough understanding of the examples of the present disclosure.

In the drawings, thicknesses of regions and layers may be exaggerated for clarity. In the drawings, the same reference signs denote the same or similar structures, and thus their detailed descriptions will be omitted.

The described features, structures, or characteristics may be combined in any suitable manner in one or more examples. In the following description, many specific details are provided to provide a thorough understanding of the examples of the present disclosure.

In a first aspect of the present disclosure, provided is an organic compound, having a structure shown in a Formula 1:

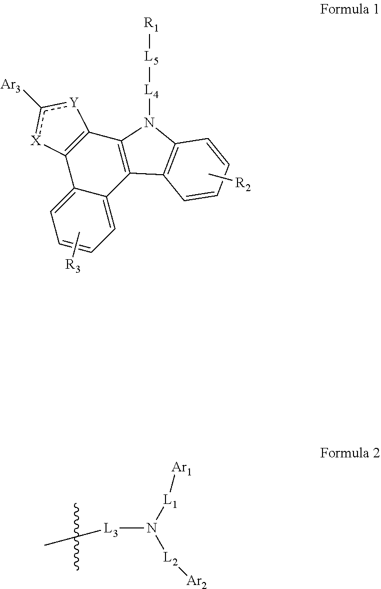

where X and Y are each independently selected from

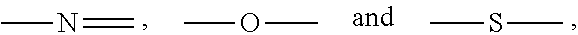

and one and only one of X and Y is

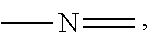

and the other is

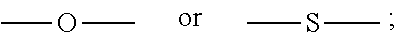

R₁ is selected from a hydrogen, a deuterium, or a structure represented by a Formula 2;
R₂ and R₃ are the same or different, and are each independently selected from a hydrogen, a deuterium, a cyano, a halogen group, an alkyl with 1 to 10 carbon atoms, a haloalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, a cycloalkyl with 3 to 10 carbon atoms, or the structure represented by the Formula 2;
and at least one of R₁, R₂, and R₃ is the group represented by the Formula 2;
L₁, L₂, L₃ and L₄ are the same or different, and are each independently selected from a single bond, a substituted or unsubstituted arylene with 6 to 30 carbon atoms, or a substituted or unsubstituted heteroarylene with 3 to 30 carbon atoms;
L₅ is selected from a substituted or unsubstituted arylene with 6 to 30 carbon atoms, or a substituted or unsubstituted heteroarylene with 3 to 30 carbon atoms;
Ar₁, Ar₂, and Ar₃ are the same or different, and are each independently selected from a substituted or unsubstituted aryl with 6 to 30 carbon atoms, or a substituted or unsubstituted heteroaryl with 3 to 30 carbon atoms;
substituent(s) in L₁, L₂, L₃, L₄, L₅, Ar₁, Ar₂, and Ar₃ are the same or different, and are each independently selected from a deuterium, a cyano, a halogen group, an alkyl with 1 to 10 carbon atoms, a haloalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, or a cycloalkyl with 3 to 10 carbon atoms; and
optionally, in Ar₁ and Ar₂, any two adjacent substituents form a ring.

In the present disclosure, the terms “optional” and “optionally” mean that the subsequently described event or circumstance may but need not occur, and that the description includes instances where the event or circumstance occurs and instances where the event or circumstance does not occur. For example, “optionally, two adjacent substituents form a ring”, which means that the two substituents can form a ring but do not necessarily form a ring, including the scenario where two adjacent substituents form a ring and the scenario where two adjacent substituents do not form a ring.

In the present disclosure, the adopted description modes “each . . . is independently”, “ . . . is respectively and independently” and “ . . . is independently selected from” can be interchanged, and should be understood in a broad sense, which means that in different groups, specific options expressed between the same symbols do not influence each other, or in a same group, specific options expressed between the same symbols do not influence each other. For example, the meaning of “

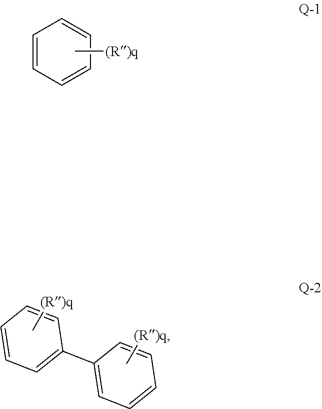

where each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, an unpositioned connecting bond refers to a single bond “ [symbol] ” extending from a ring system, which means that one end of the connecting bond can be connected with any position in the ring system through which the bond penetrates, and the other end of the connecting bond is connected with the remaining part of a compound molecule.

For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1)-(f-10).

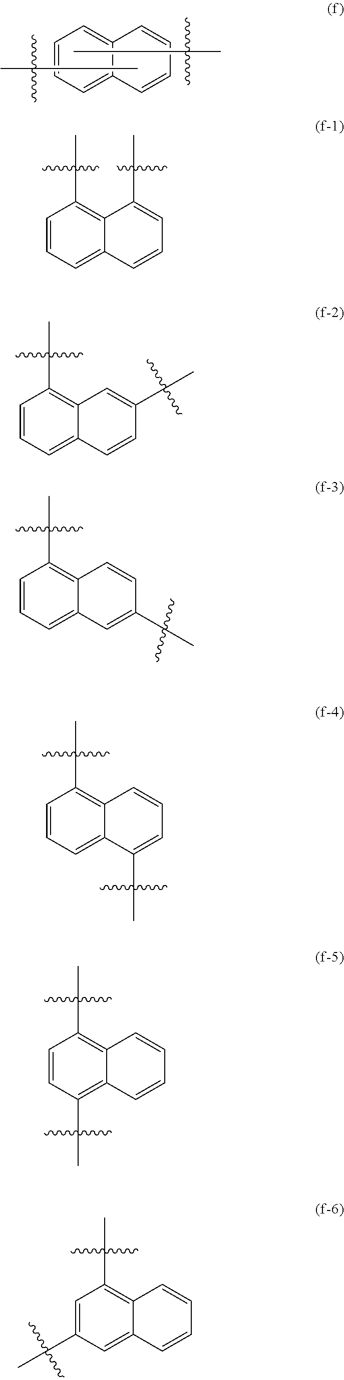

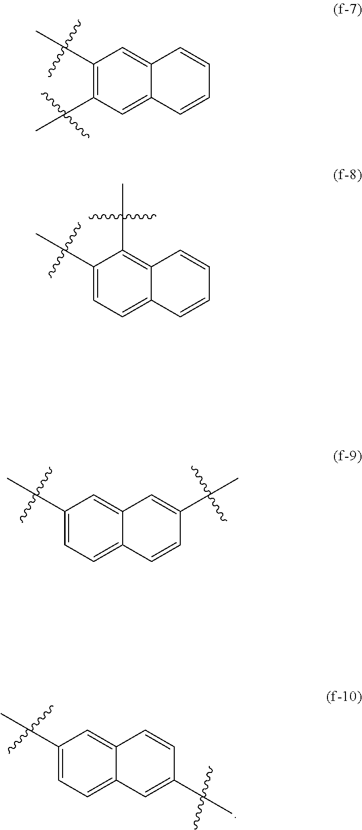 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in the following formula (X′), phenanthryl represented by the formula (X′) is connected with other positions of a molecule through one unpositioned connecting bond extending from the middle of a benzene ring on one side, and its meaning includes any one possible connecting mode represented by formulae (X′-1)-(X′-4).

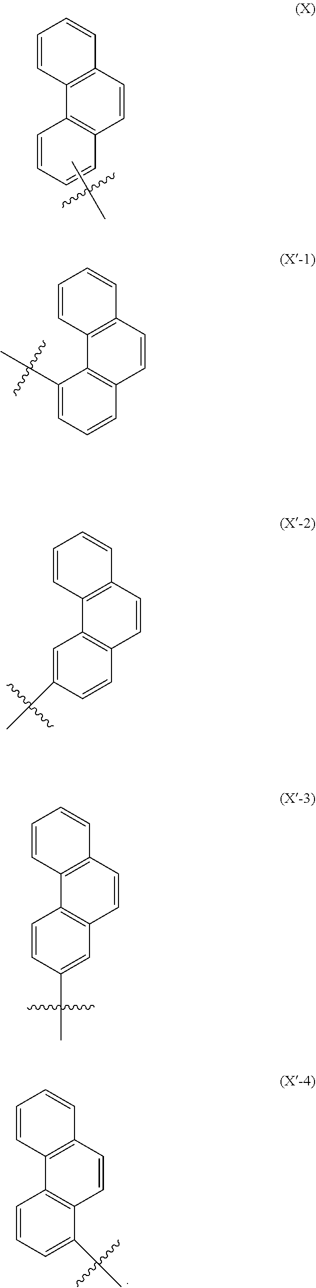

An unpositioned substituent in the present disclosure refers to a substituent connected by a single bond extending from the center of a ring system, which means that the substituent may be connected to any possible position in the ring system. For example, as shown in the following formula (Y), a substituent R′ represented by the formula (Y) is connected to a quinoline ring through an unpositioned connecting bond, and its meaning includes any one possible connecting mode represented by formulae (Y-1)-(Y-7).

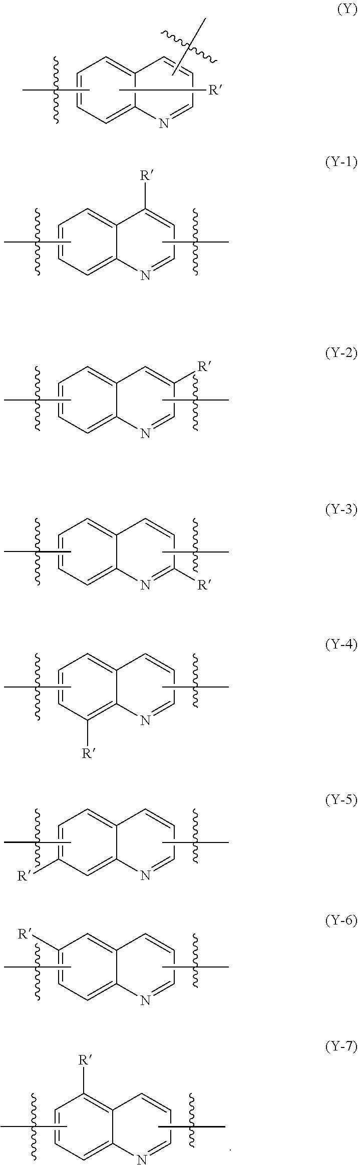

In the present disclosure, the number of carbon atoms of L₁, L₂, L₃, L₄, L₅, R₂, R₃, Ar₁, Ar₂ and Ar₃ refers to the number of all carbon atoms. For example, if L₁ is selected from a substituted arylene with 12 carbon atoms, then the number of all carbon atoms of the arylene and substituents on the arylene is 12. For example: if Ar₁ is

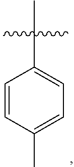 [SMILES: *c1ccc(C)cc1]

then the number of carbon atoms is 7; and if L₁ is

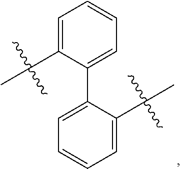 [SMILES: *c1ccccc1-c1ccccc1*]

then the number of carbon atoms is 12.

In the present disclosure, when no specific definition is otherwise provided, “hetero” refers to the inclusion of at least one heteroatom such as B, N, O, S, Se, Si, or P in one functional group with the remaining atoms being carbon and hydrogen. Unsubstituted alkyl may be a “saturated alkyl group” without any double or triple bonds.

In the present disclosure, “alkyl” may include linear alkyl or branched alkyl. The alkyl may have 1 to 10 carbon atoms, and in the present disclosure, numerical ranges such as “1 to 10” refer to integers in a given range; for example, “alkyl with 1 to 10 carbon atoms” refers to alkyl that may include 1, 2, 3, 4, 5, 6, 7, 8, 9, or 10 carbon atoms. Optionally, the alkyl is selected from alkyl with 1 to 5 carbon atoms, and specific examples include, but are not limited to, methyl, ethyl, n-propyl, isopropyl, n-butyl, isobutyl, sec-butyl, tert-butyl, and pentyl.

In the present disclosure, cycloalkyl refers to a group derived from a saturated cyclic carbon chain structure. The cycloalkyl may have 3 to 10 carbon atoms, and in the present disclosure, numerical ranges such as “3 to 10” refer to integers in a given range; for example, “5 to 10 carbon atoms” means that 5 carbon atoms, 6 carbon atoms, 7 carbon atoms, 8 carbon atoms, 9 carbon atoms, or 10 carbon atoms may be included. Optionally, specific examples of the cycloalkyl include, but are not limited to, cyclopentyl, cyclohexyl, adamantyl, norbornyl, and the like.

In the present disclosure, aryl refers to an optional functional group or substituent derived from an aromatic carbocyclic ring. The aryl can be monocyclic aryl (e.g., phenyl) or polycyclic aryl, in other words, the aryl can be monocyclic aryl, fused aryl, two or more monocyclic aryl linked by carbon-carbon bonds, monocyclic aryl and fused aryl which are linked by a carbon-carbon bond, or two or more fused aryl linked by carbon-carbon bonds. That is, unless otherwise indicated, two or more aromatic groups linked by carbon-carbon bonds may also be considered as the aryl in the present disclosure. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se, and Si. Examples of the aryl can include, but are not limited to, phenyl, naphthyl, fluorenyl, anthryl, phenanthryl, biphenyl, terphenyl, quaterphenyl, quinquephenyl, benzo[9,10]phenanthryl, triphenylene, pyrenyl, benzofluoranthenyl, chrysenyl, spirobifluorenyl, and the like. The “substituted or unsubstituted aryl” of the present disclosure may contain 6 to 30 carbon atoms, in some examples, the number of carbon atoms in the substituted or unsubstituted aryl may be 6 to 25, in other examples, the number of carbon atoms in the substituted or unsubstituted aryl may be 6 to 20, in other examples, the number of carbon atoms in the substituted or unsubstituted aryl may be 6 to 18, and in other examples, the number of carbon atoms in the substituted or unsubstituted aryl may be 6 to 15. For example, in the present disclosure, the number of carbon atoms of the substituted or unsubstituted aryl may also be 6, 10, 12, 13, 14, 15, 18, 20, 24, 25, or 30, and of course, the number of carbon atoms may also be other numbers, which will not be listed here. In the present disclosure, biphenyl can be understood as phenyl-substituted aryl or unsubstituted aryl.

In the present disclosure, the arylene involved refers to a divalent group formed by the further loss of one hydrogen atom of aryl.

In the present disclosure, the substituted aryl may be that one or two or more hydrogen atoms in the aryl are substituted by groups such as a deuterium atom, a halogen group, cyano, aryl, heteroaryl, trialkylsilyl, alkyl, cycloalkyl, alkoxy and alkylthio.

It should be understood that the number of carbon atoms of the substituted aryl refers to the total number of carbon atoms of the aryl and the substituents on the aryl, e.g., substituted aryl with 18 carbon atoms means that the total number of carbon atoms of the aryl and substituents is 18.

In the present disclosure, specific examples of aryl as a substituent include, but are not limited to, phenyl, naphthyl, anthryl, phenanthryl, dimethylfluorenyl, biphenyl, and the like.

In the present disclosure, fluorenyl may be substituted, two substituents may be bonded to each other to form a spiro structure, and specific examples include, but are not limited to, the following structures:

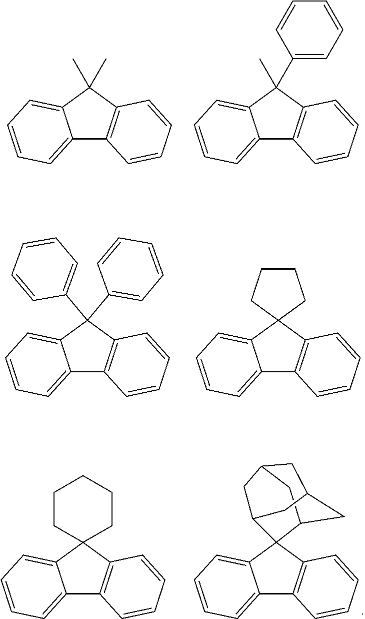

In the present disclosure, the heteroaryl refers to a monovalent aromatic ring containing 1, 2, 3, 4, 5 or 6 heteroatoms in the ring or its derivative, and the heteroatom may be at least one of B, O, N, P, Si, Se and S. The heteroaryl may be monocyclic heteroaryl or polycyclic heteroaryl, in other words, the heteroaryl may be a single aromatic ring system or a plurality of aromatic ring systems linked by carbon-carbon bonds, and any one aromatic ring system is a monocyclic aromatic ring or a fused aromatic ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silafluorenyl, dibenzofuranyl, and N-arylcarbazolyl (such as N-phenylcarbazolyl), N-heteroarylcarbazolyl (such as N-pyridylcarbazolyl), N-alkylcarbazolyl (such as N-methylcarbazolyl), and the like, but is not limited to this. Thienyl, furyl, phenanthrolinyl, etc. are heteroaryl of the single aromatic ring system, and N-arylcarbazolyl (e.g., N-phenylcarbazolyl), and N-heteroarylcarbazolyl are heteroaryl of polycyclic systems conjugatedly linked by carbon-carbon bonds. The “substituted or unsubstituted heteroaryl” of the present disclosure may contain 3 to 30 carbon atoms, in some examples, the number of carbon atoms in the substituted or unsubstituted heteroaryl can be 3 to 27, in other examples, the number of carbon atoms in the substituted or unsubstituted heteroaryl can be 12 to 24, and in other examples, the number of carbon atoms in the substituted or unsubstituted heteroaryl can be 12 to 20. For example, the number of carbon atoms may be 3, 4, 5, 7, 12, 13, 18 or 20, and of course, the number of carbon atoms may also be other numbers, which will not be listed here.

In the present disclosure, the heteroarylene involved refers to a divalent group formed by the further loss of one hydrogen atom of heteroaryl.

In the present disclosure, the substituted heteroaryl may be that one or two or more hydrogen atoms in the heteroaryl are substituted by groups such as a deuterium atom, a halogen group, cyano, aryl, heteroaryl, trialkylsilyl, alkyl, cycloalkyl, alkoxy, and alkylthio.

It should be understood that the number of carbon atoms of the substituted heteroaryl refers to the total number of carbon atoms of the heteroaryl and substituents on the heteroaryl.

In the present disclosure, specific examples of heteroaryl as a substituent include, but are not limited to, dibenzofuranyl, dibenzothienyl, carbazolyl, N-phenylcarbazolyl and the like.

In the present disclosure, the halogen group may include fluorine, iodine, bromine, chlorine, and the like.

In the present disclosure, deuterated aryl may be that one or two or more hydrogen atoms in the aryl are substituted by deuterium, and specific examples of the deuterated aryl include, but are not limited to, pentadeuterophenyl.

In the present disclosure, the haloaryl may be that one or two or more hydrogen atoms in the aryl are substituted by a halogen atom, and specific examples of the haloaryl include, but are not limited to, fluorophenyl and chlorophenyl.

In the present disclosure, the haloalkyl may be that one or two or more hydrogen atoms in the alkyl are substituted by a halogen atom, and specific examples of the haloalkyl include, but are not limited to, trifluoromethyl.

In the present disclosure, terphenyl includes

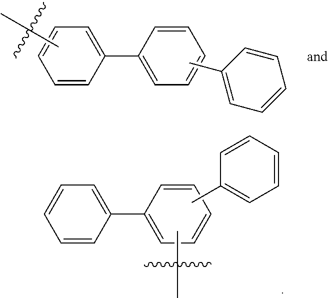

In the present disclosure, “X and Y are each independently selected from

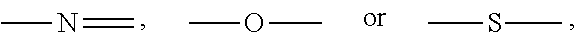

and one and only one of X and Y is

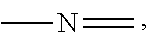

the other is

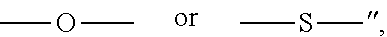

which means that one of X and Y is

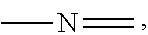

the other is

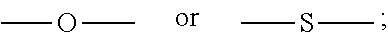

specifically, X is

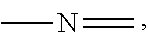

### and Y is

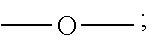

### or, X is

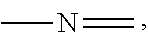

### and Y is

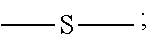

### or Y is

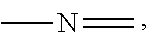

### and X is

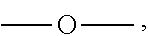

### or Y is

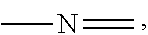

### and X is

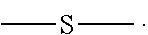

In some embodiments of the present disclosure, in the compound represented by the formula 1, one and only one of R₁, R₂, and R₃ is the group represented by the formula 2.

Specifically, the organic compound of the present disclosure is selected from structures shown in any one of Formulae 1-1 to 1-4:

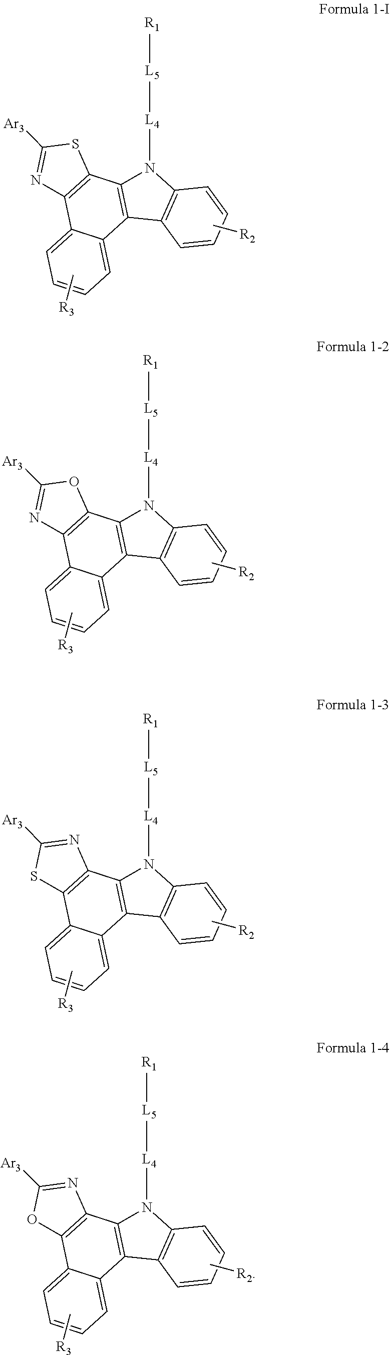

In some embodiments of the present disclosure, the organic compound is selected from structures shown as a Formula A, a Formula B, or a Formula C:

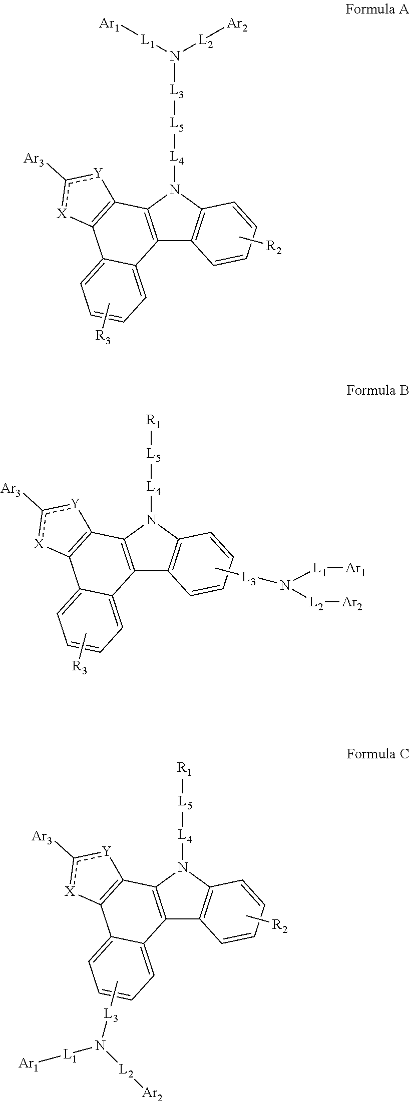

where in the formula B and the formula C, R₁ is a hydrogen or a deuterium.

In some specific embodiments of the present disclosure, the organic compound is selected from structure shown as a Formula AA or a Formula BB below:

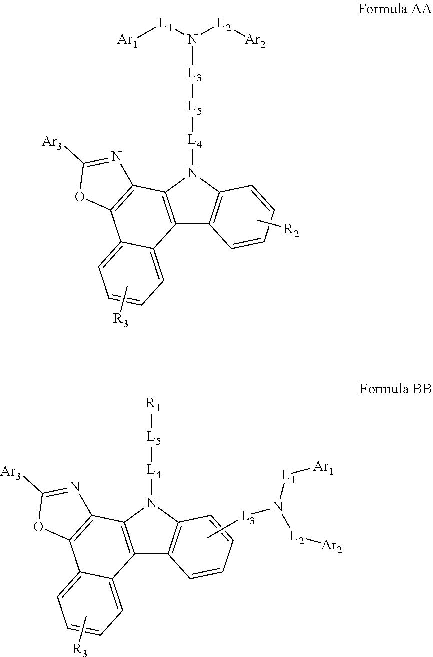

where in the formula BB, R₁ is a hydrogen or a deuterium.

In some embodiments of the present disclosure, L₁, L₂, L₃ and L₄ are the same or different, and are each independently selected from a single bond, a substituted or unsubstituted arylene with 6 to 15 carbon atoms, or a substituted or unsubstituted heteroarylene with 12 to 20 carbon atoms.

In some embodiments, L₁, L₂, L₃ and L₄ are the same or different, and are each independently selected from a single bond, a substituted or unsubstituted arylene with 6, 7, 8, 9, 10, 11, 12, 13, 14, or 15 carbon atoms, or a substituted or unsubstituted heteroarylene with 12, 13, 14, 15, 16, 17, 18, 19, or 20 carbon atoms.

Optionally, substituent(s) in L₁, L₂, L₃ and L₄ are the same or different, and are each independently selected from a deuterium, a halogen group, a cyano, an alkyl with 1 to 5 carbon atoms or phenyl.

In other embodiments of the present disclosure, L₁, L₂, L₃ and L₄ are the same or different, and are each independently selected from a single bond, a substituted or unsubstituted phenylene, a substituted or unsubstituted biphenylene, a substituted or unsubstituted naphthylene, a substituted or unsubstituted phenanthrylene, a substituted or unsubstituted fluorenylidene, a substituted or unsubstituted carbazolylene, a substituted or unsubstituted dibenzofurylene, or a substituted or unsubstituted dibenzothenylene.

Further optionally, L₃ and L₄ are the same or different, and are each independently selected from a single bond, a substituted or unsubstituted phenylene, a substituted or unsubstituted naphthylene, or a substituted or unsubstituted biphenylene.

Optionally, substituent(s) in L₁, L₂, L₃ and L₄ are the same or different, and are each independently selected from a deuterium, a fluorine, a cyano, a methyl, an ethyl, a n-propyl, an isopropyl, a tert-butyl or a phenyl.

In some embodiments of the present disclosure, L₁, L₂, L₃ and L₄ are the same or different, and are each independently selected from a single bond, and a substituted or unsubstituted group V, where the unsubstituted group V is selected from the group consisting of the following groups:

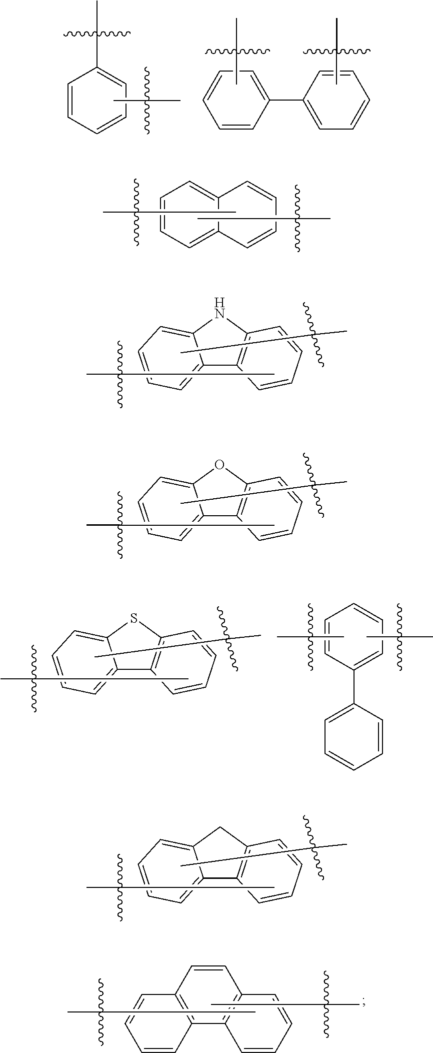

where [symbol] represents a chemical bond; the substituted group V contains one or more substituents selected from a deuterium, a fluorine, a cyano, a methyl, an ethyl, a n-propyl, an isopropyl, a tert-butyl or a phenyl; and when the substituted group V contains a plurality of substituents, the substituents are the same or different.

In some more specific embodiments of the present disclosure, L₁ and L₂ are the same or different, and are each independently selected from a single bond or the group consisting of the following groups:

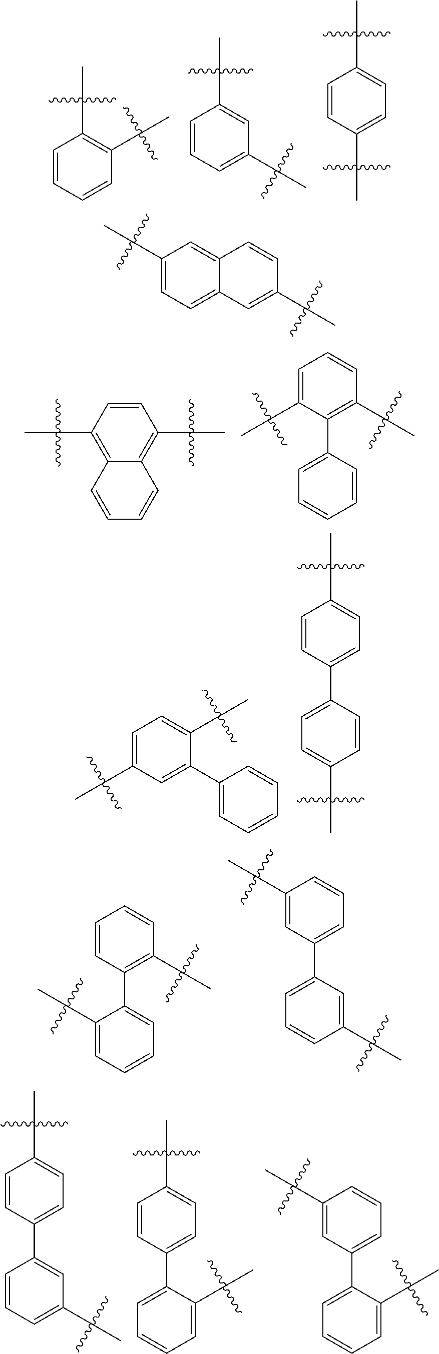

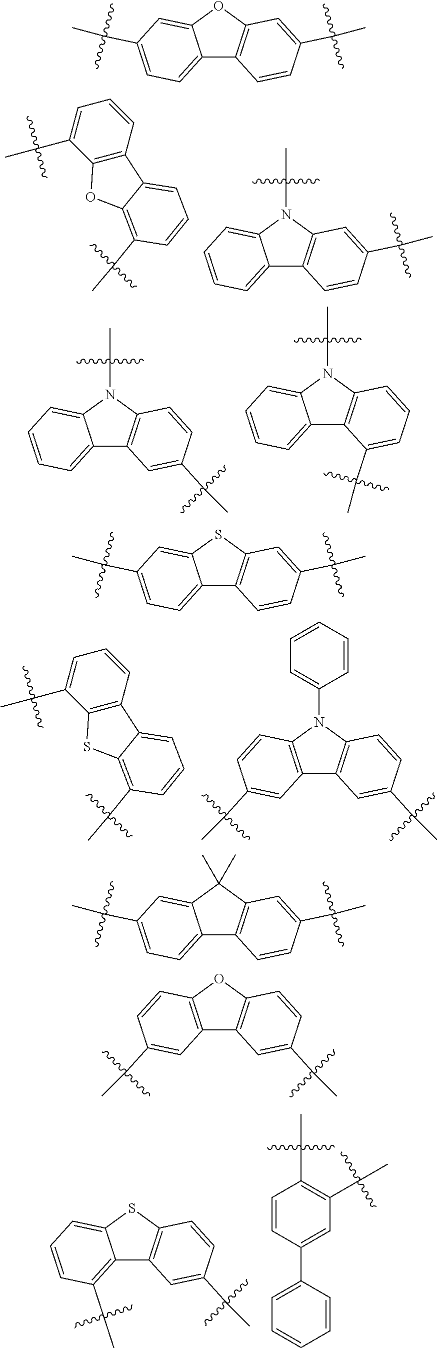

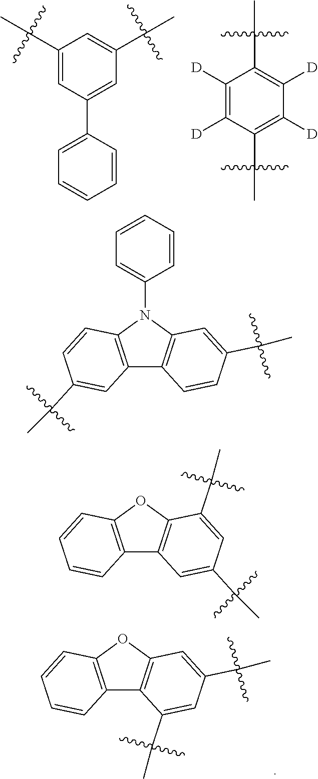

In some embodiments of the present disclosure, L₃ and L₄ are the same or different, and are each independently selected from a single bond or the group consisting of the following groups:

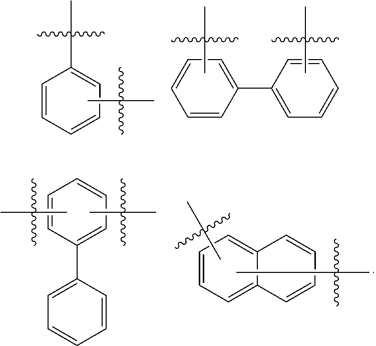

In particular, L₃ and L₄ are the same or different, and are each independently selected from a single bond or the group consisting of the following groups:

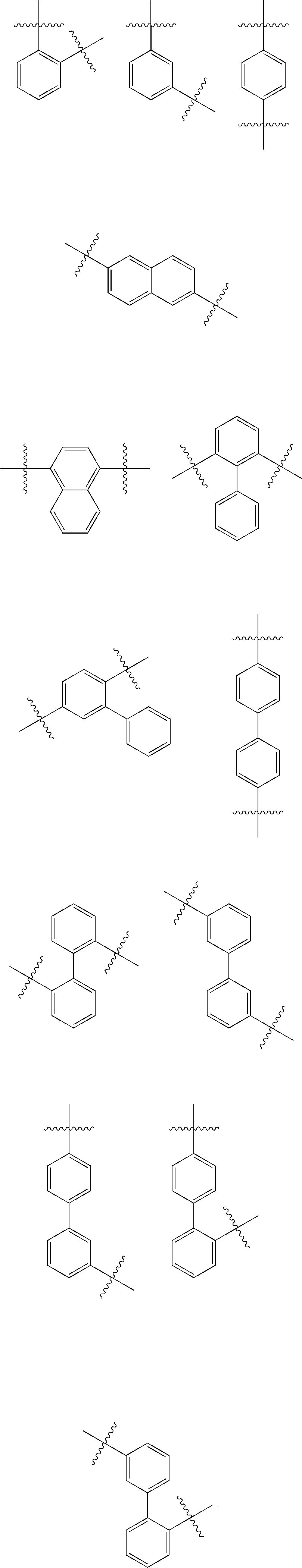

In some embodiments of the present disclosure, Ar₁ and Ar₂ are the same or different, and are each independently selected from a substituted or unsubstituted aryl with 6 to 25 carbon atoms, or a substituted or unsubstituted heteroaryl with 12 to 24 carbon atoms.

In some embodiments of the present disclosure, Ar₁ and Ar₂ are the same or different, and are each independently selected from a substituted or unsubstituted aryl with 6, 7, 8, 9, 10, 11, 12, 13, 14, 15, 16, 17, 18, 19, 20, 21, 22, 23, 24 or 25 carbon atoms, or a substituted or unsubstituted heteroaryl with 12, 13, 14, 15, 16, 17, 18, 19, 20, 21, 22, 23 or 24 carbon atoms.

Optionally, substituent(s) in Ar₁ and Ar₂ are the same or different, and are each independently selected from a deuterium, a halogen group, a cyano, an alkyl with 1 to 5 carbon atoms, an aryl with 6 to 12 carbon atoms, or a pentadeuterophenyl; and
optionally, in Ar₁ and Ar₂, any two adjacent substituents form a fluorene ring

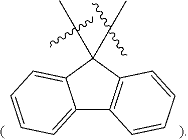 [SMILES: *C1(*)c2ccccc2-c2ccccc21]

In some embodiments of the present disclosure, Ar₁ and Ar₂ are the same or different, and are each independently selected from a substituted or unsubstituted phenyl, a substituted or unsubstituted naphthyl, a substituted or unsubstituted phenanthryl, a substituted or unsubstituted biphenyl, a substituted or unsubstituted terphenyl, a substituted or unsubstituted fluorenyl, a substituted or unsubstituted triphenylene, a substituted or unsubstituted 9,9′-spirobifluorenyl, a substituted or unsubstituted dibenzofuranyl, a substituted or unsubstituted dibenzothienyl, or a substituted or unsubstituted carbazolyl.

Optionally, substituent(s) in Ar₁ and Ar₂ are the same or different, and are each independently selected from a deuterium, a fluorine, a cyano, a methyl, an ethyl, a n-propyl, an isopropyl, a tert-butyl, a phenyl, a naphthyl or a pentadeuterophenyl.

Optionally, Ar₁ and Ar₂ are the same or different, and are each independently selected from the group consisting of the following groups:

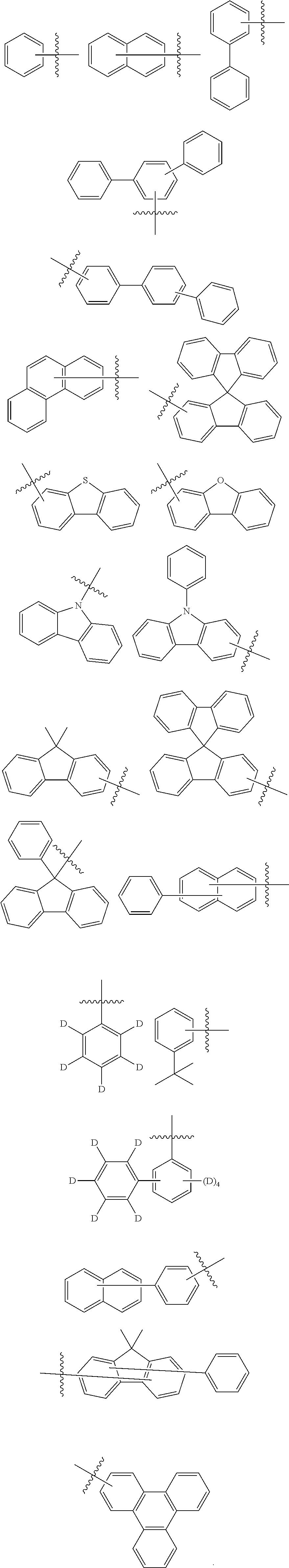

In particular, Ar₁ and Ar₂ are the same or different, and are each independently selected from the group consisting of the following groups:

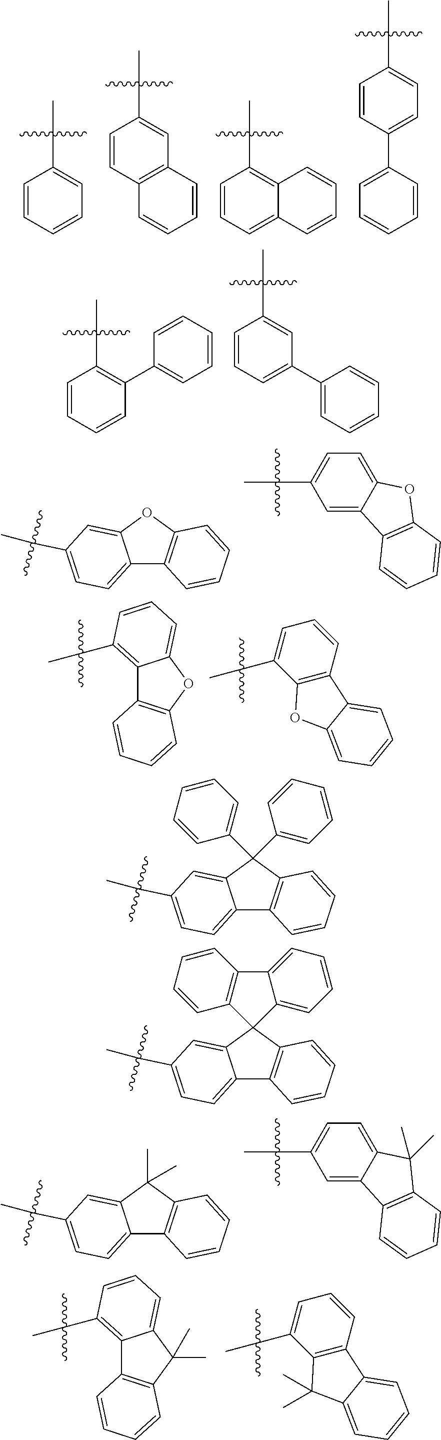

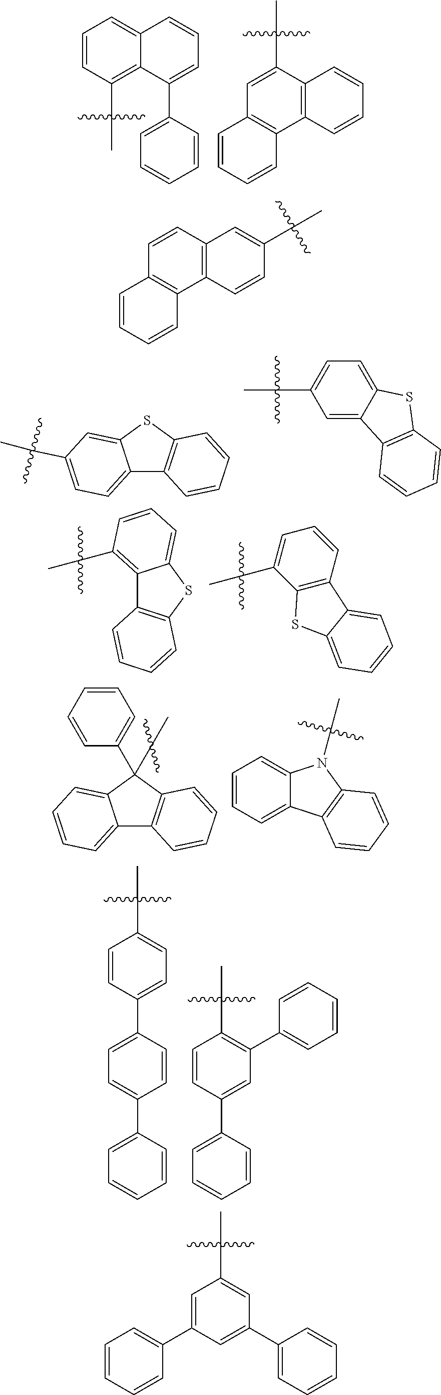

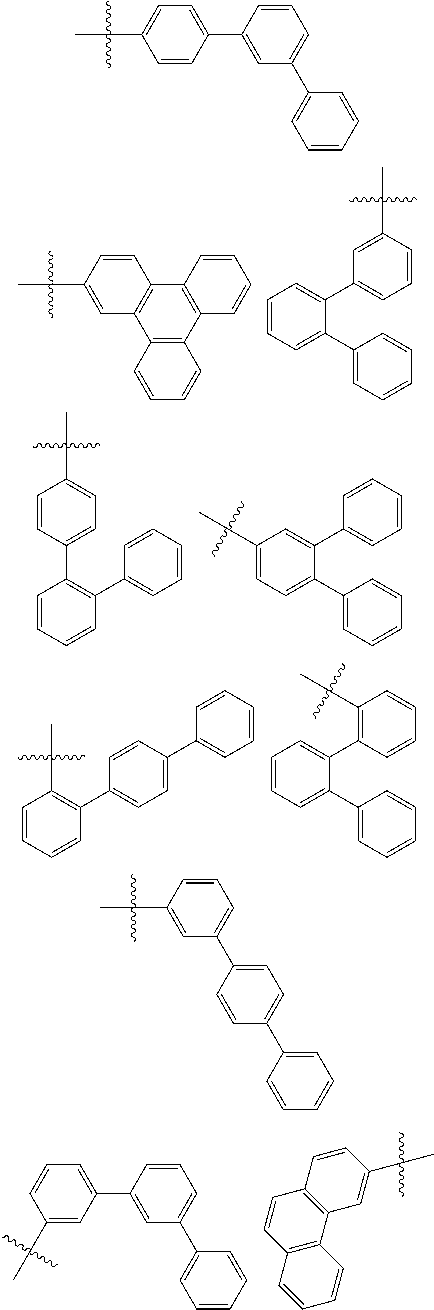

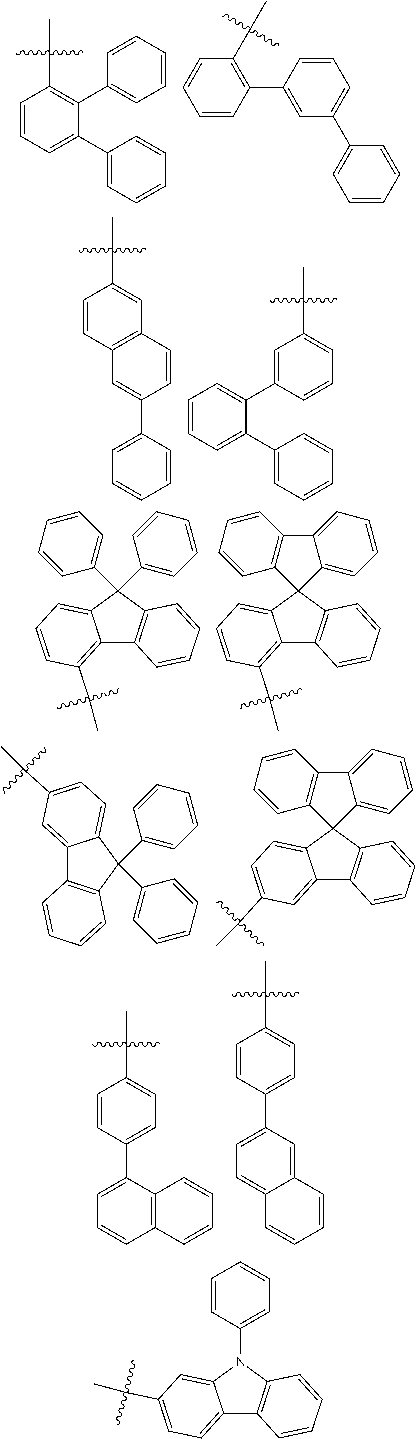

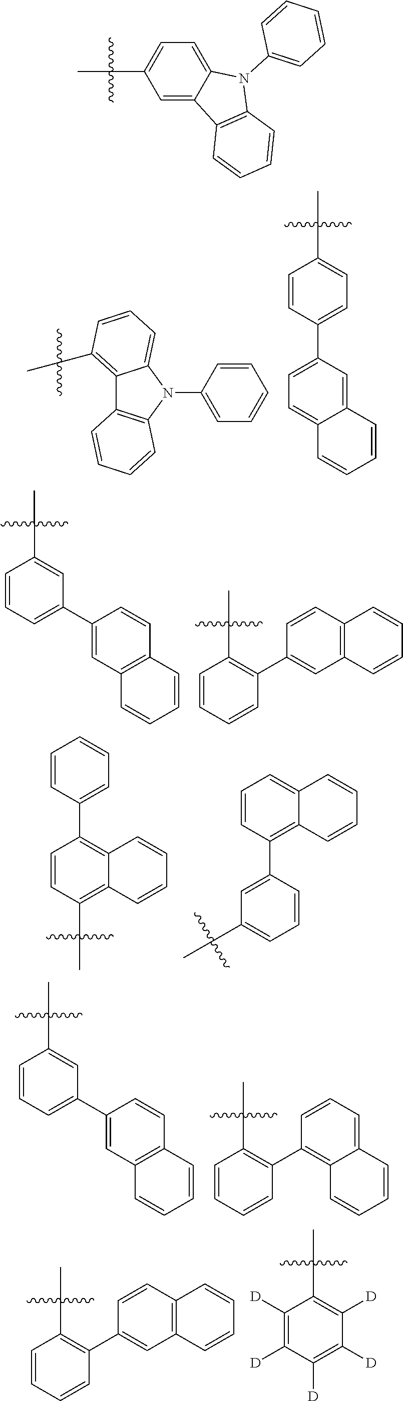

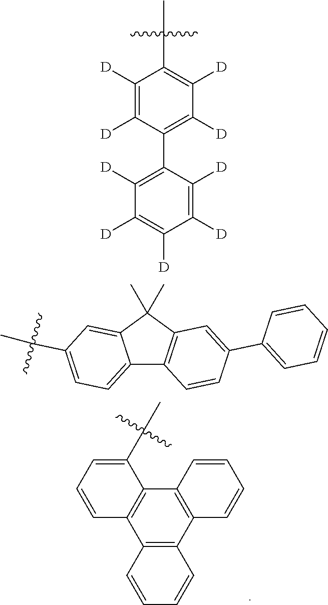

In some embodiments of the present disclosure,

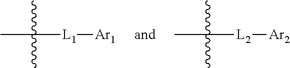

are the same or different, and are each independently selected from the group consisting of the following groups:

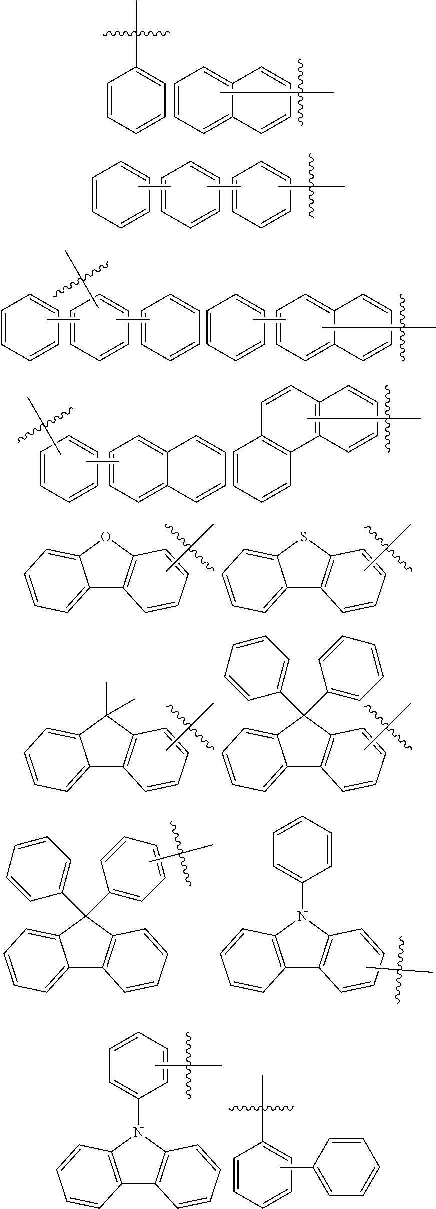

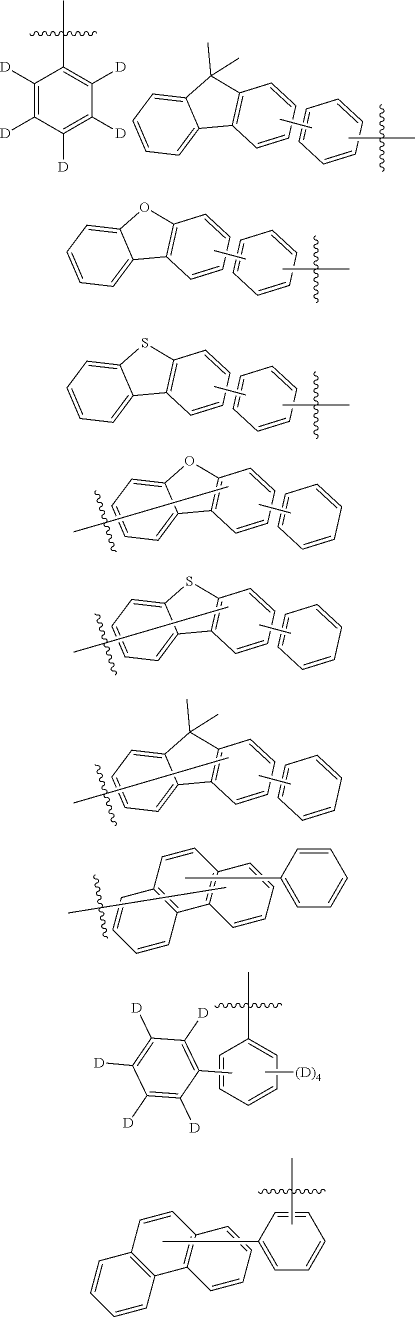

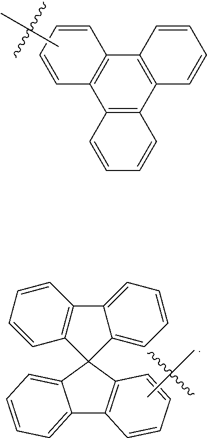

In particular,

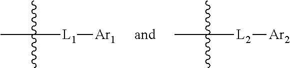

are the same or different, and are each independently selected from the group consisting of the following groups:

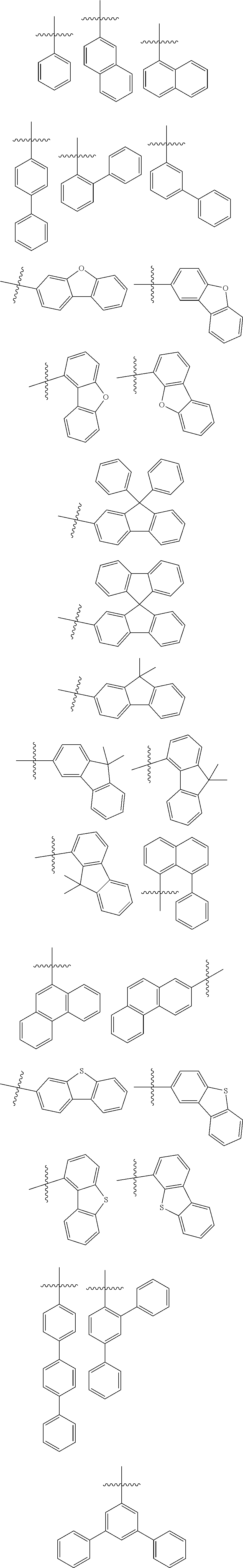

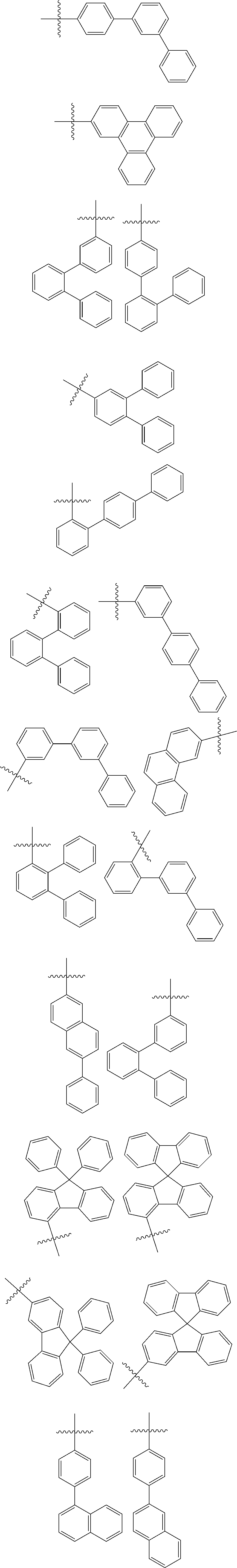

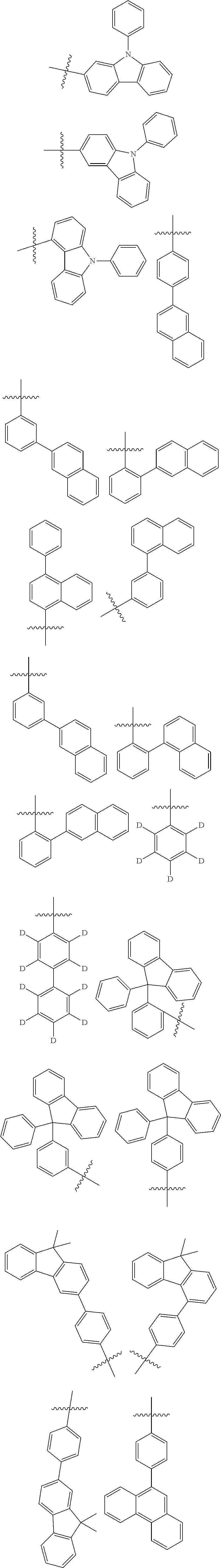

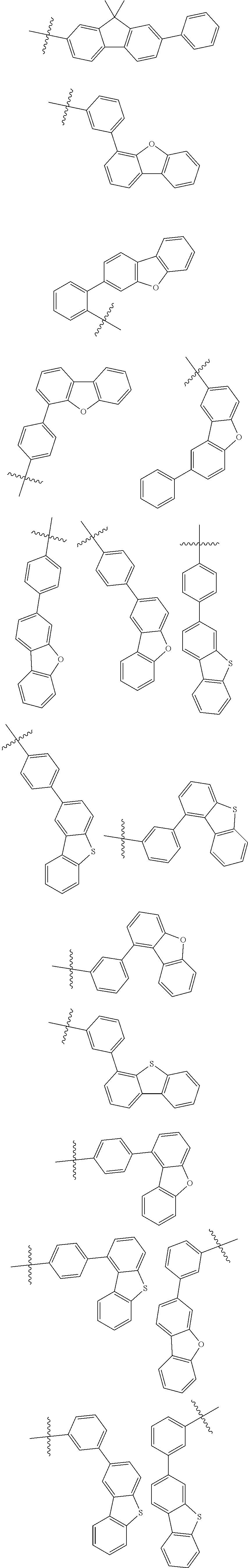

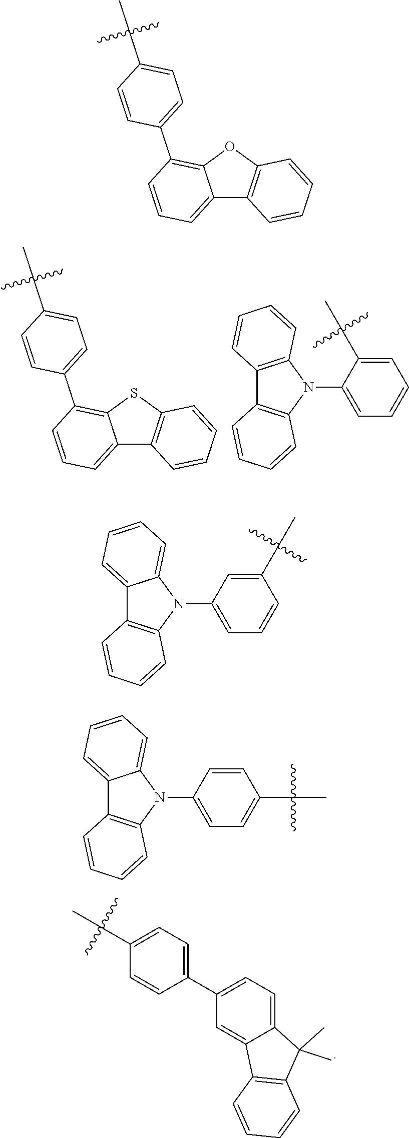

In some embodiments of the present disclosure, L₅ is selected from a substituted or unsubstituted phenylene, a substituted or unsubstituted naphthylene, a substituted or unsubstituted biphenylene, a substituted or unsubstituted dibenzofurylene, a substituted or unsubstituted dibenzothenylene, or a substituted or unsubstituted fluorenylidene.

Optionally, substituent(s) in L₅ are the same or different, and are each independently selected from a deuterium, a fluorine, a cyano, a methyl, an ethyl, a n-propyl, an isopropyl, a tert-butyl or a phenyl.

In other embodiments of the present disclosure, L₅ is selected from the group consisting of the following groups:

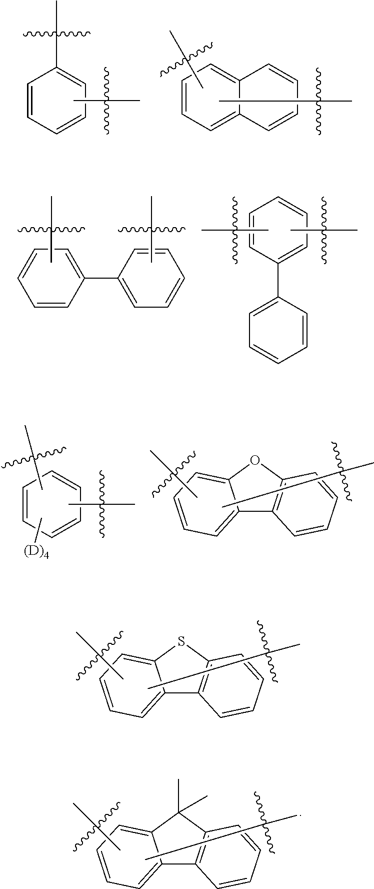

In particular, L₅ is selected from the group consisting of the following groups:

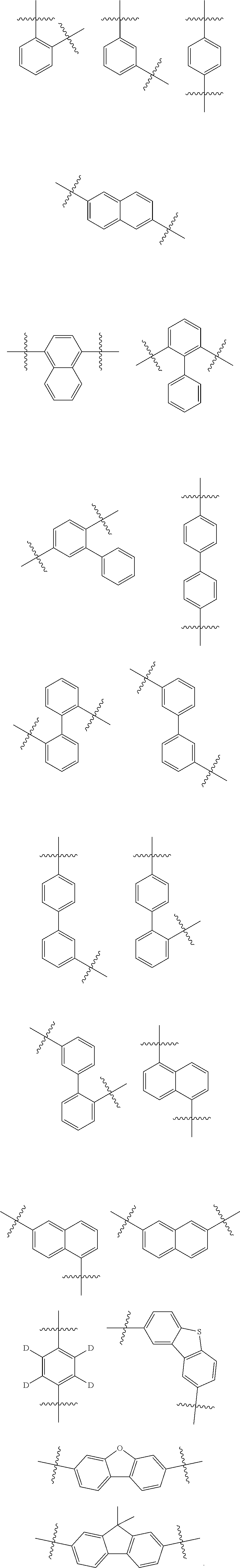

In some embodiments of the present disclosure, when R₁ is selected from a group represented by the Formula 2, L₅ is selected from a substituted or unsubstituted phenylene, a substituted or unsubstituted naphthylene, or a substituted or unsubstituted biphenylene.

Optionally, substituent(s) in L₅ are the same or different, and are each independently selected from a deuterium, a fluorine, a cyano, a methyl, an ethyl, a n-propyl, an isopropyl, a tert-butyl or a phenyl.

In other embodiments of the present disclosure, when R₁ is selected from the group represented by the Formula 2, L₅ is selected from the group consisting of the following groups:

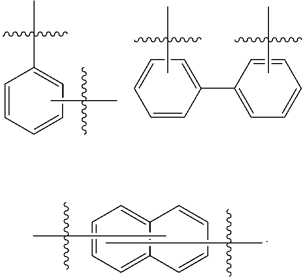

In particular, when R₁ is selected from the group represented by the Formula 2, L₅ is selected from the group consisting of the following groups:

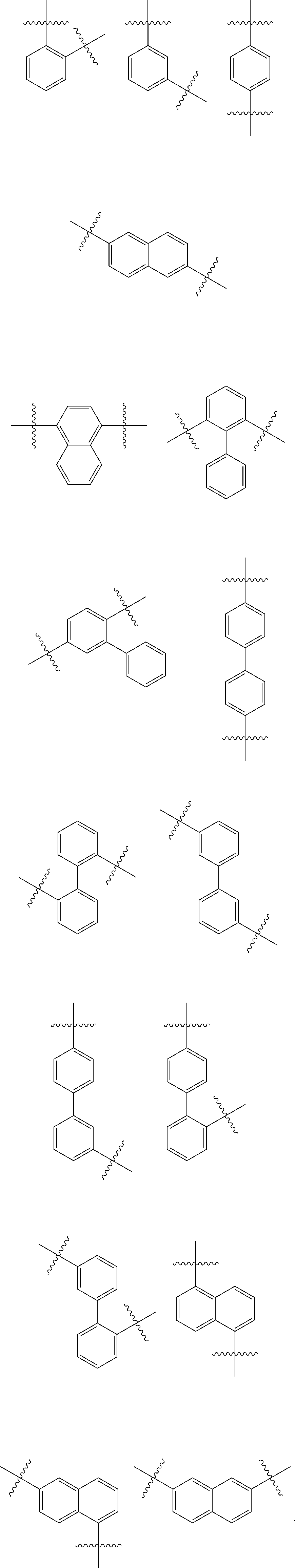

In some specific embodiments of the present disclosure, when R₁ is selected from a hydrogen or a deuterium, L₅ is selected from a substituted or unsubstituted phenylene, a substituted or unsubstituted naphthylene, a substituted or unsubstituted biphenylene, a substituted or unsubstituted dibenzofurylene, a substituted or unsubstituted dibenzothenylene or a substituted or unsubstituted fluorenylidene.

Optionally, substituent(s) in L₅ are the same or different, and are each independently selected from a deuterium, a fluorine, a cyano, a methyl, an ethyl, a n-propyl, an isopropyl, a tert-butyl or a phenyl.

Optionally, when R₁ is selected from hydrogen or deuterium,

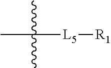

is selected from the group consisting of the following groups:

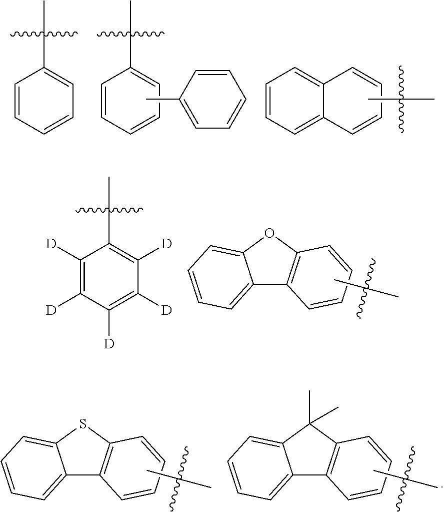

In particular, when R₁ is selected from a hydrogen or a deuterium,

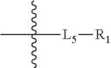

selected from the group consisting of the following groups:

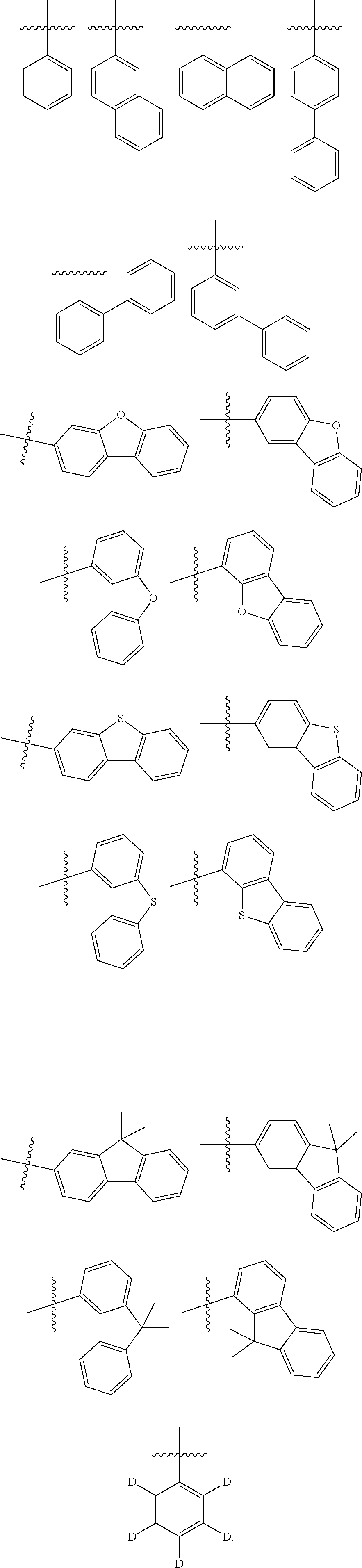

In some embodiments of the present disclosure,

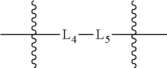

is selected from the group consisting of the following groups:

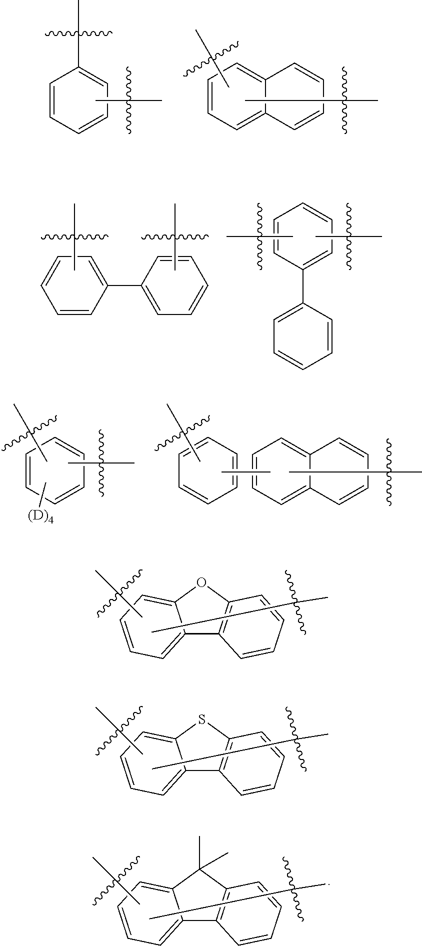

In particular,

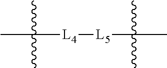

is selected from the group consisting of the following groups:

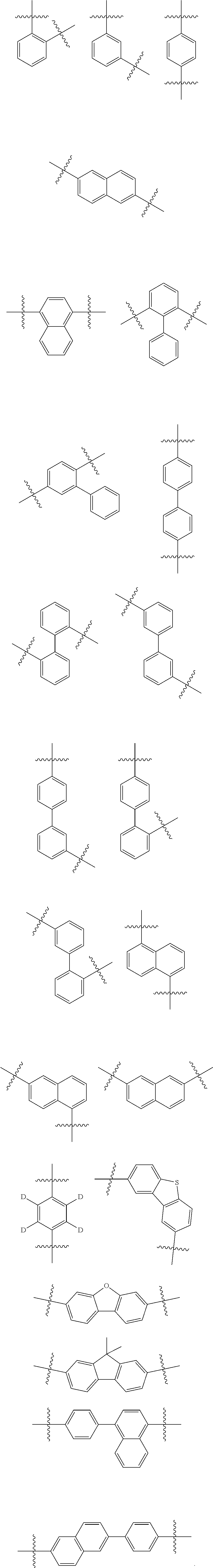

According to some embodiments of the present disclosure, L₃ is selected from a single bond or the group consisting of the following groups:

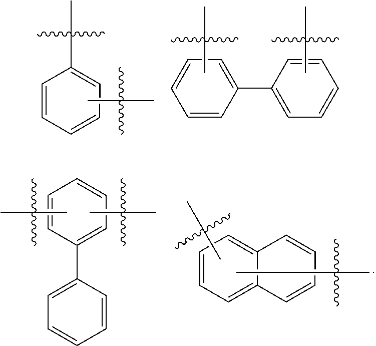

In particular, L₃ is selected from a single bond or the group consisting of the following groups:

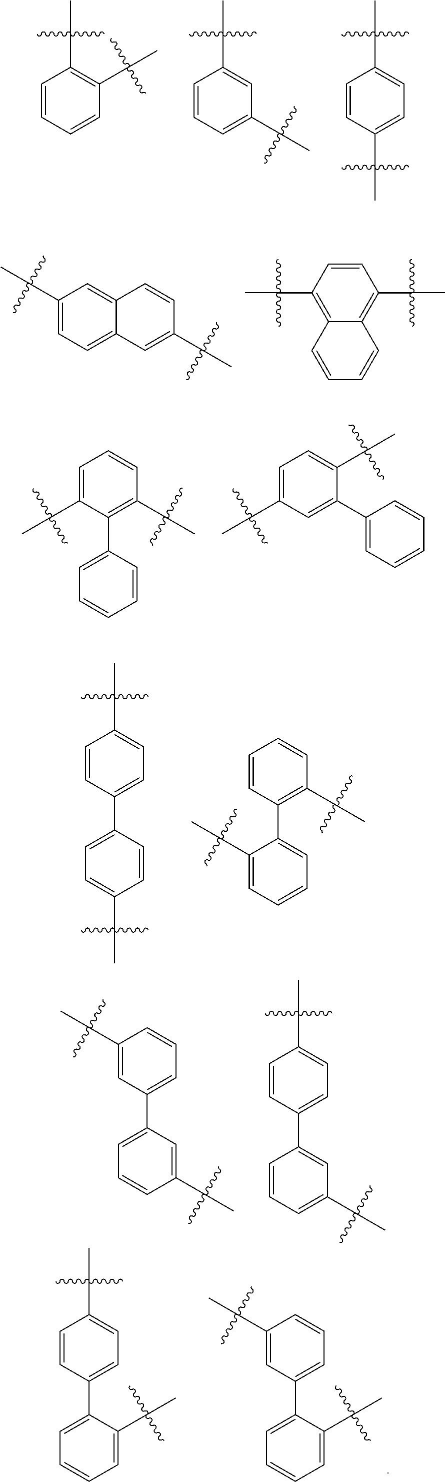

In some embodiments of the present disclosure, Ar₃ is selected from a substituted or unsubstituted aryl with 6 to 15 carbon atoms, or a substituted or unsubstituted heteroaryl with 12 to 18 carbon atoms.

Optionally, substituent(s) in Ar₃ are the same or different, and are each independently selected from a deuterium, a fluorine, a cyano, an alkyl with 1 to 5 carbon atoms or a phenyl.

In some embodiments of the present disclosure, Ar₃ is selected from a substituted or unsubstituted phenyl, a substituted or unsubstituted naphthyl, a substituted or unsubstituted biphenyl, a substituted or unsubstituted fluorenyl, a substituted or unsubstituted dibenzofuranyl, a substituted or unsubstituted dibenzothienyl, or a substituted or unsubstituted carbazolyl.

Optionally, substituent(s) in Ar₃ are the same or different, and are each independently selected from a deuterium, a fluorine, a cyano, a methyl, an ethyl, a n-propyl, an isopropyl, a tert-butyl or a phenyl.

In other embodiments of the present disclosure, Ar₃ is selected from the group consisting of the following groups:

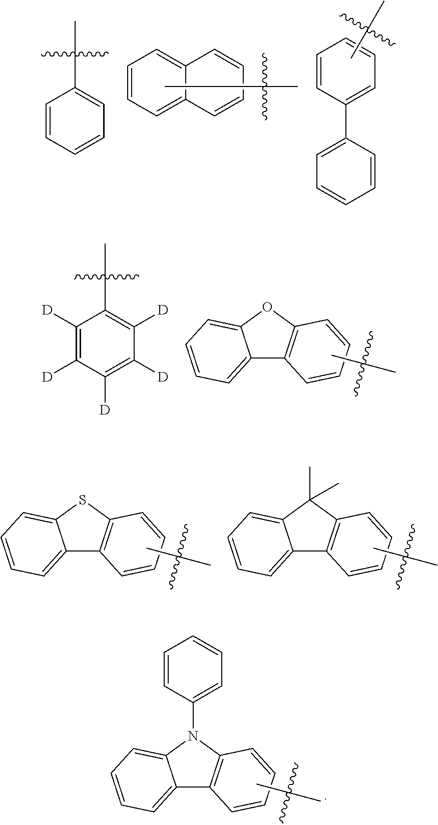

In particular, Ar₃ is selected from the group consisting of the following groups:

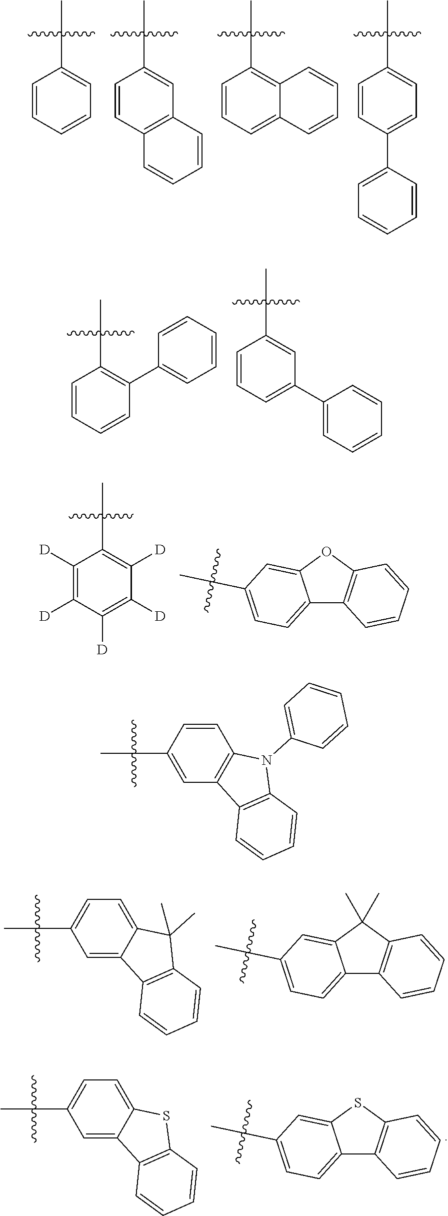

In the present disclosure, “at least one of R₁, R₂, and R₃ is the group represented by the formula 2”, which means that any one of R₁, R₂, and R₃ is the group represented by the Formula 2; or, any two of R₁, R₂, and R₃ are the group represented by the Formula 2; or, R₁, R₂, and R₃ are the group represented by the Formula 2.

In some embodiments of the present disclosure, R₁ is selected from a hydrogen, a deuterium or the group represented by the Formula 2; R₂ and R₃ are the same or different, and are each independently selected from a hydrogen or the group represented by the Formula 2; and one and only one of R₁, R₂, and R₃ is the group represented by the Formula 2.

In some specific embodiments of the present disclosure, R₂ and R₃ are both hydrogen, and R₁ is the group represented by the Formula 2.

In some specific embodiments of the present disclosure, R₁ is a hydrogen or a deuterium, R₃ is a hydrogen, and R₂ is the group represented by the Formula 2.

Optionally, the organic compound is selected from the group consisting of the following compounds:

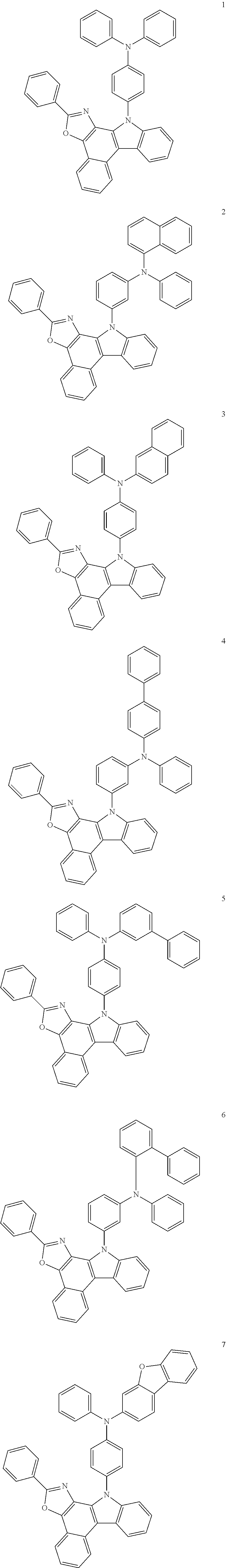

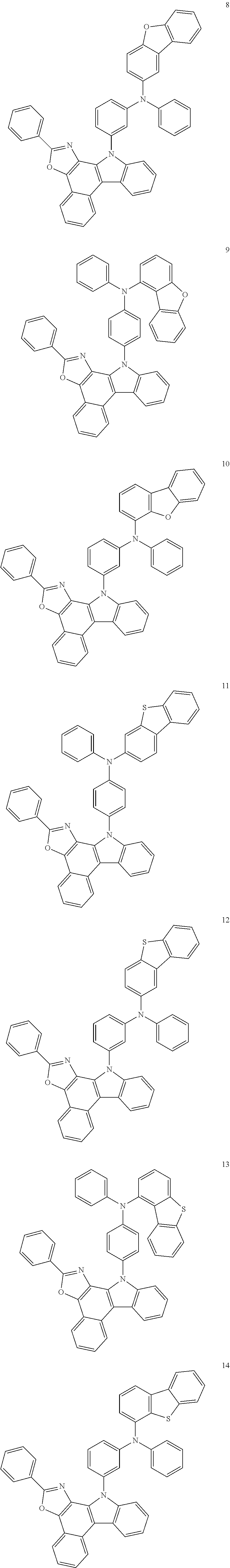

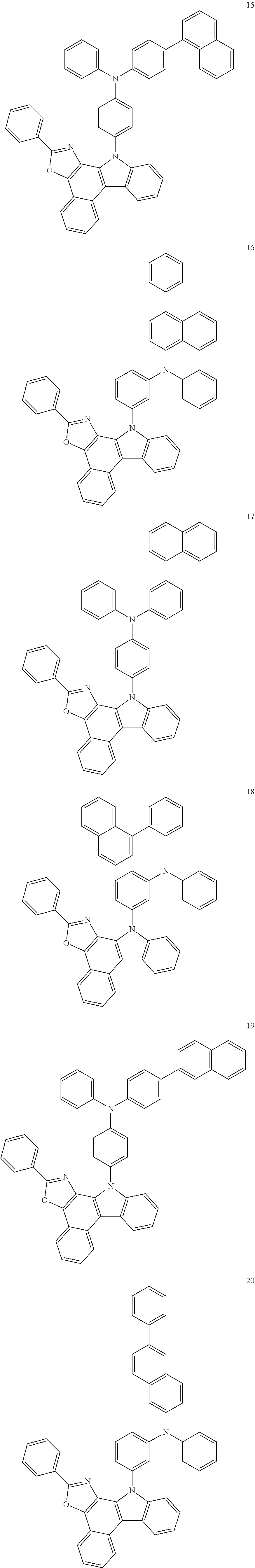

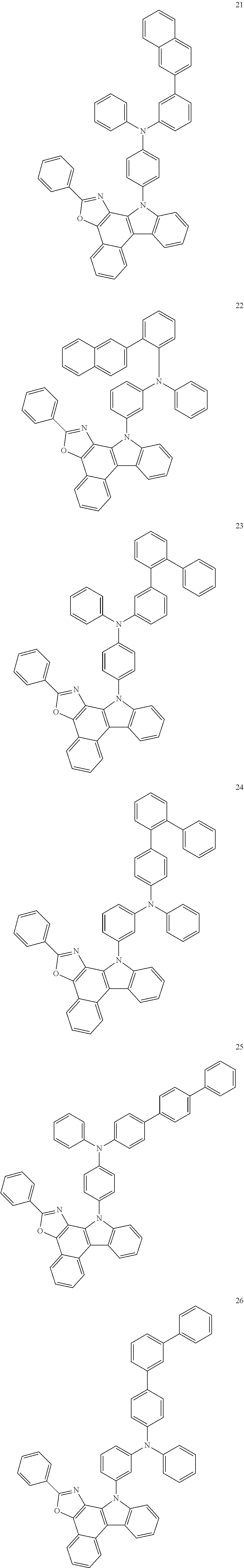

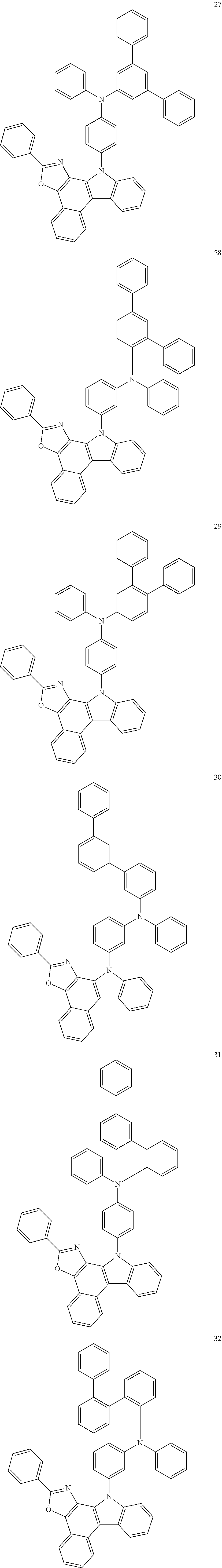

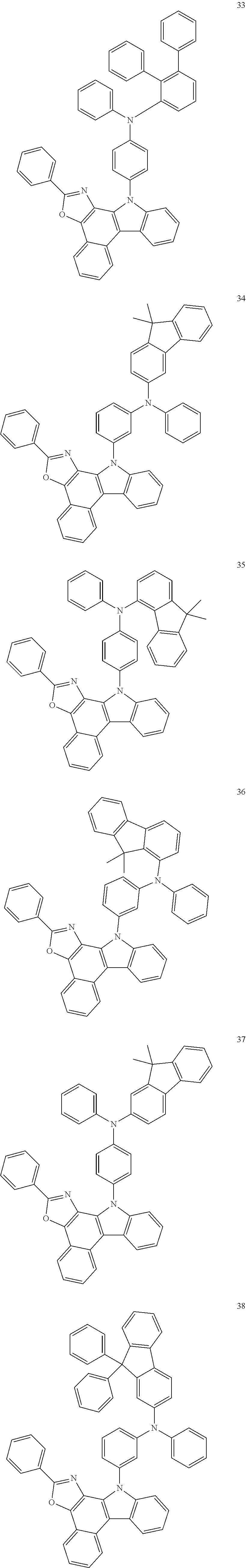

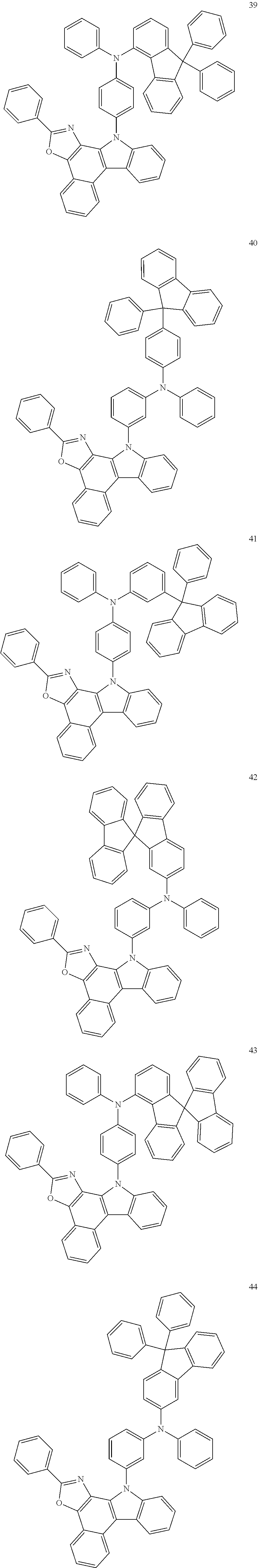

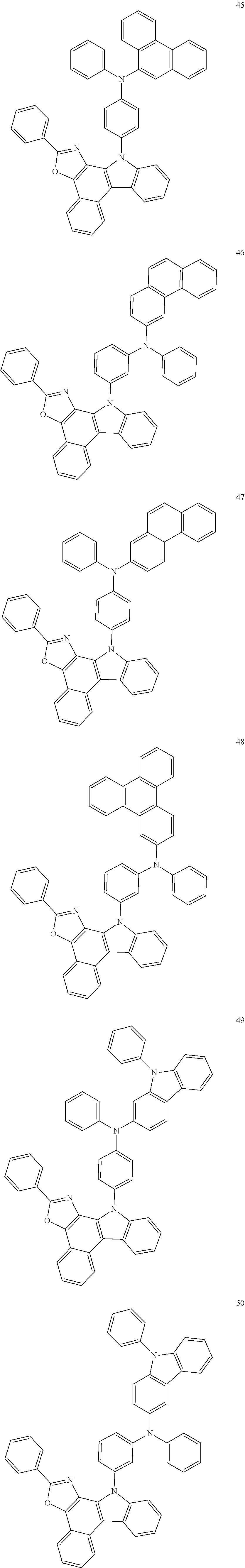

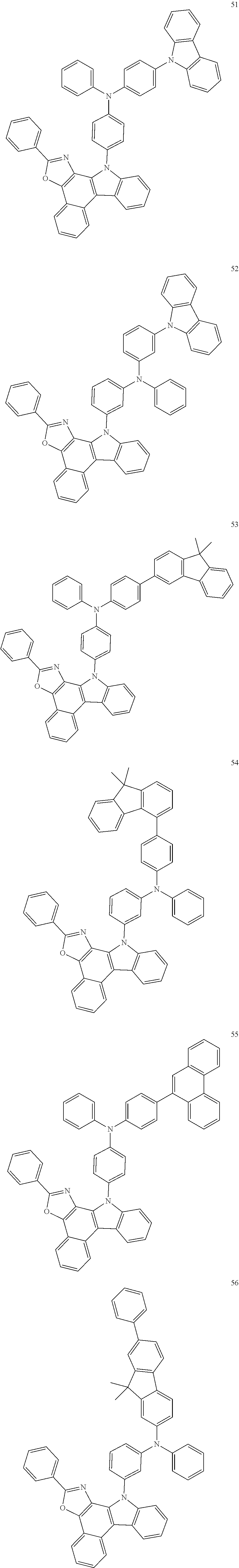

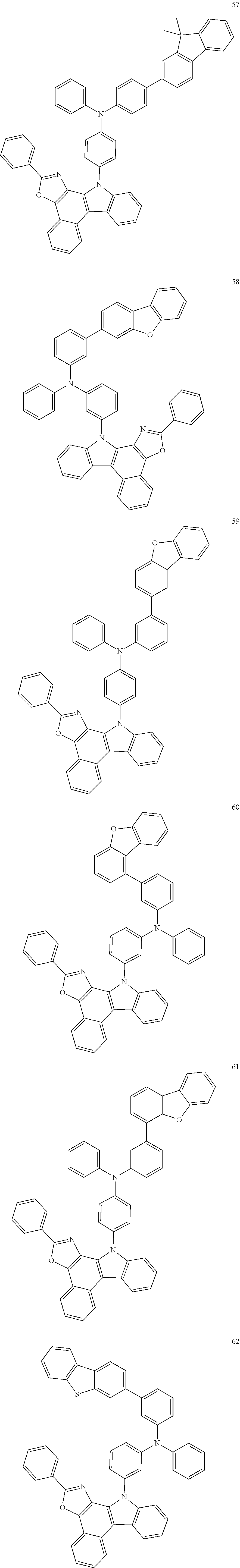

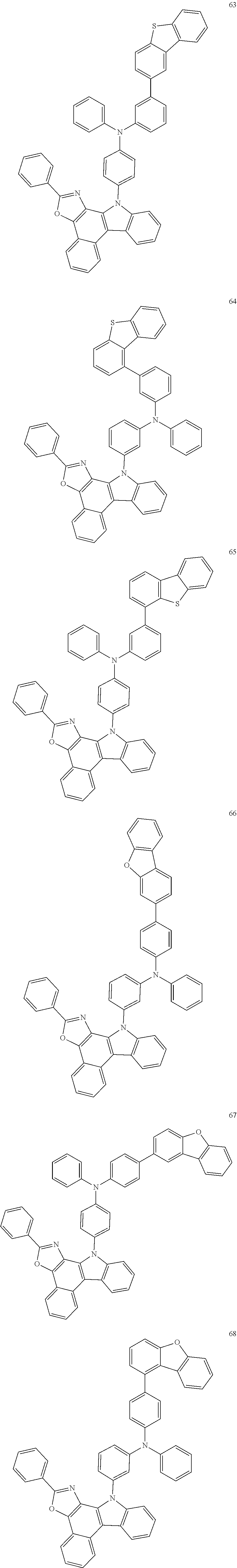

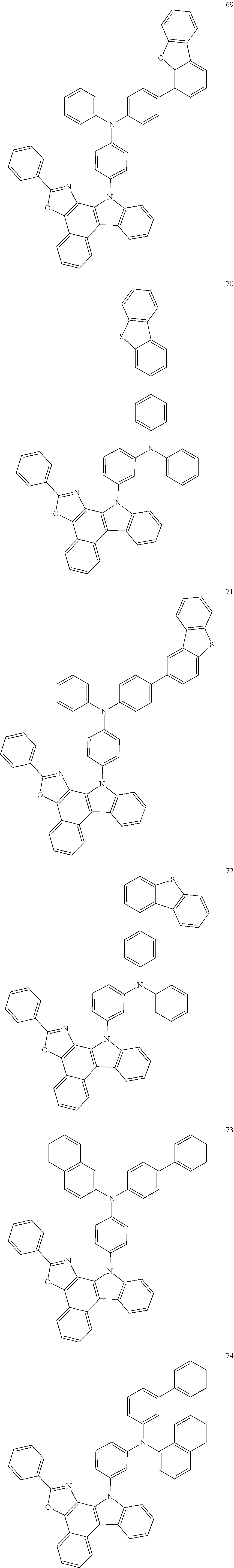

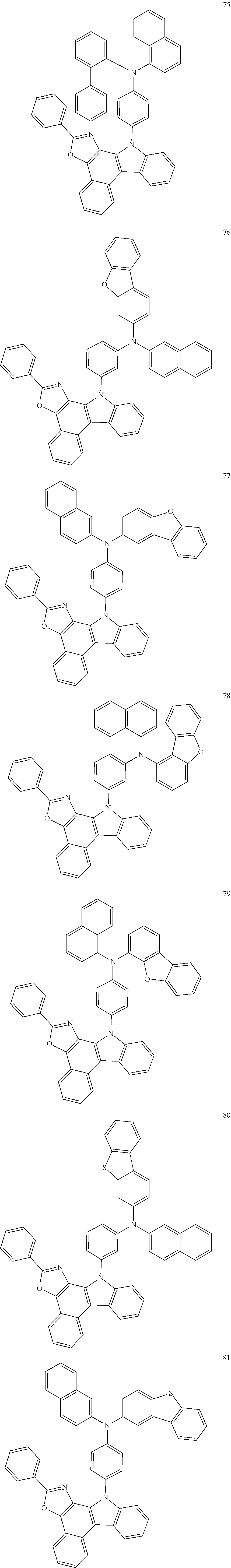

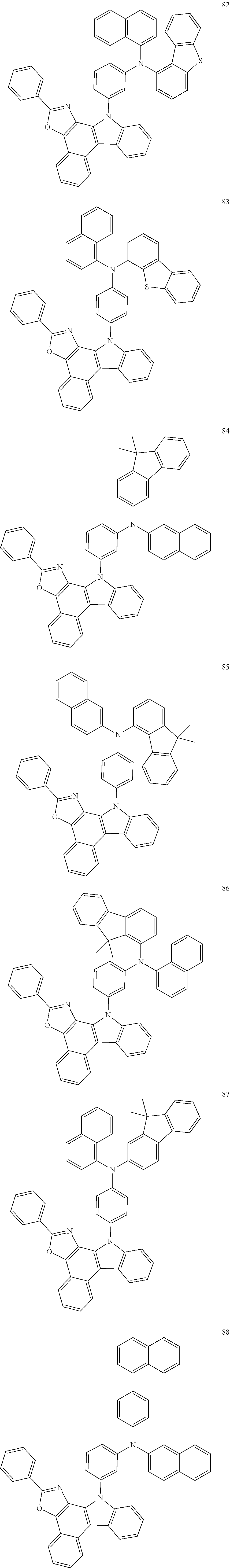

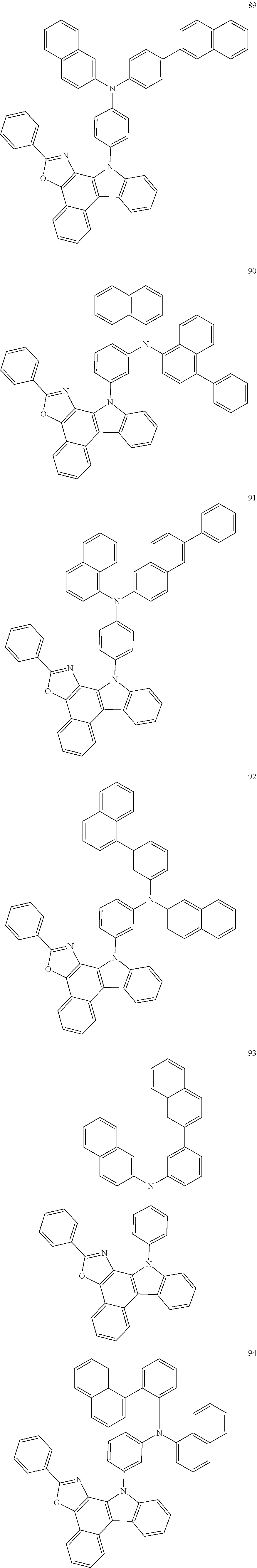

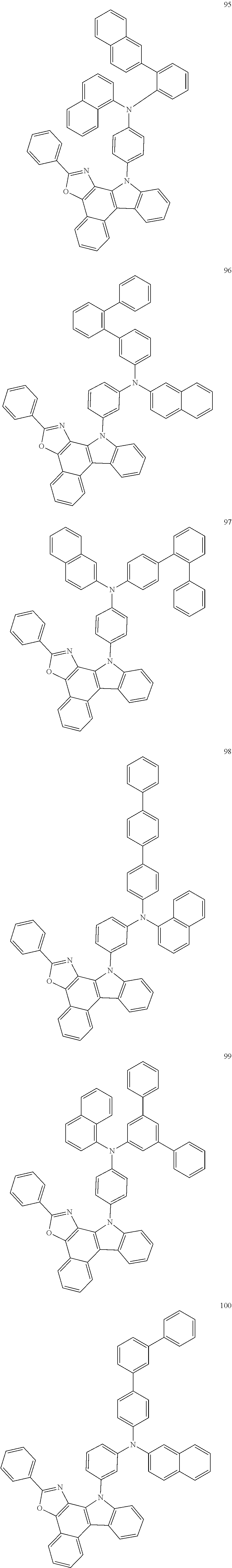

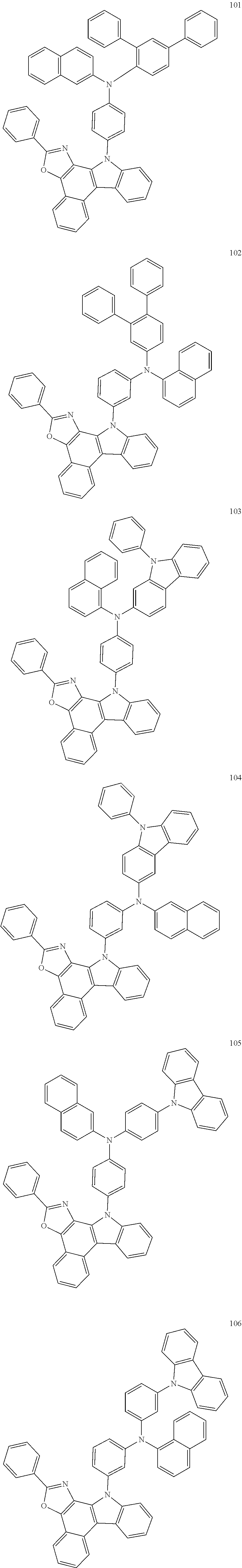

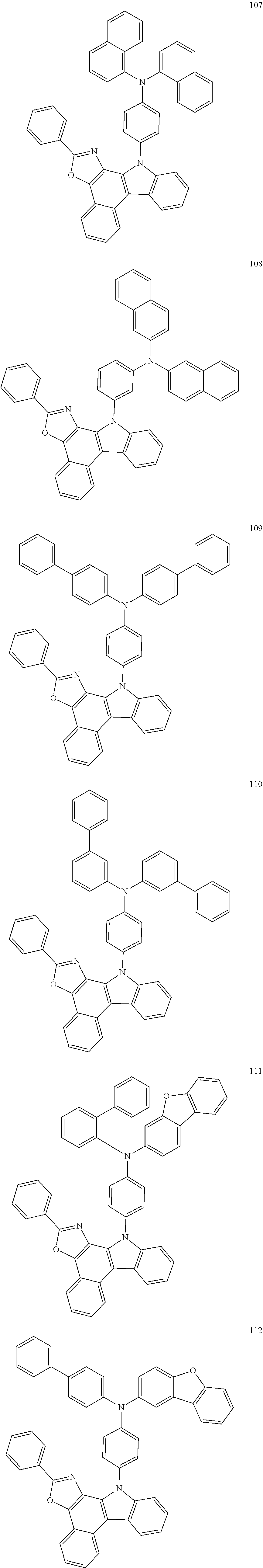

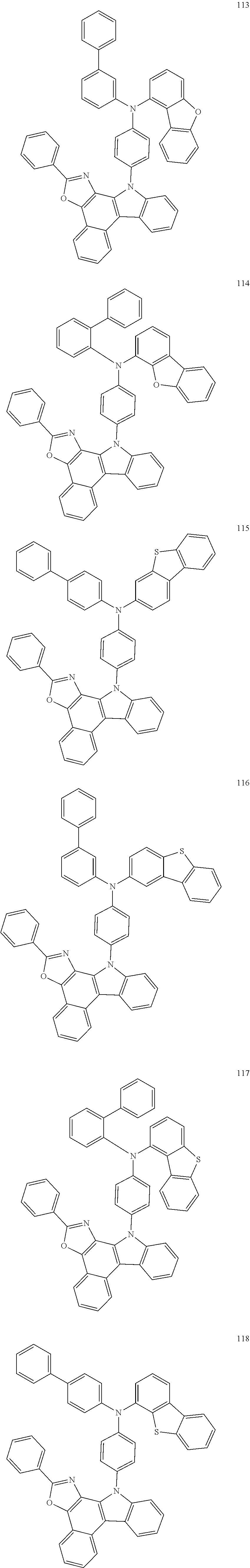

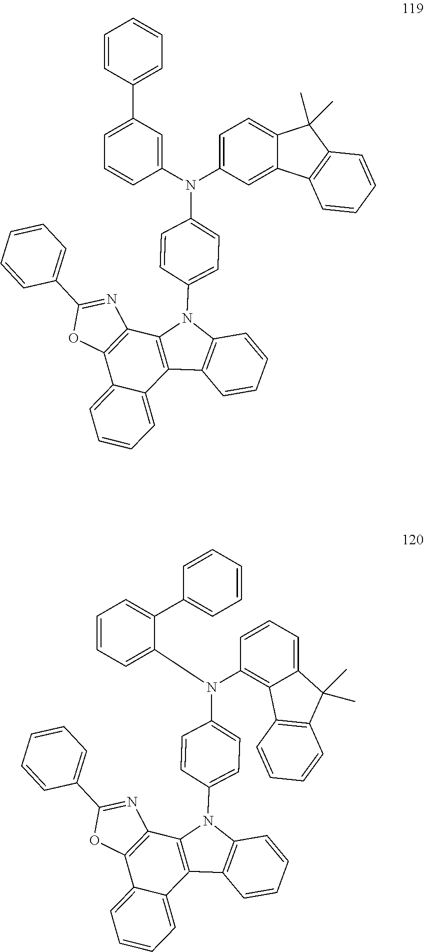 [SMILES: CC1(C)c2ccccc2-c2cc(N(c3ccc(-n4c5ccccc5c5c6ccccc6c6oc(-c7ccccc7)nc6c54)cc3)c3cccc(-c4ccccc4)c3)ccc21 ; CC1(C)c2ccccc2-c2c(N(c3ccc(-n4c5ccccc5c5c6ccccc6c6oc(-c7ccccc7)nc6c54)cc3)c3ccccc3-c3ccccc3)cccc21]

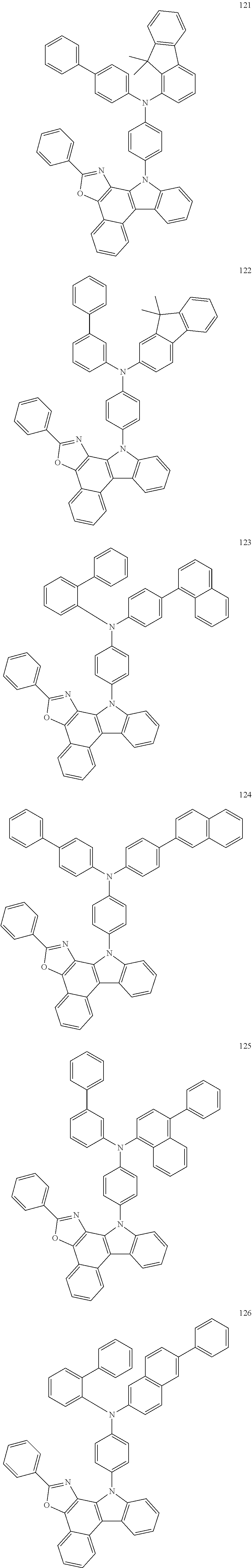

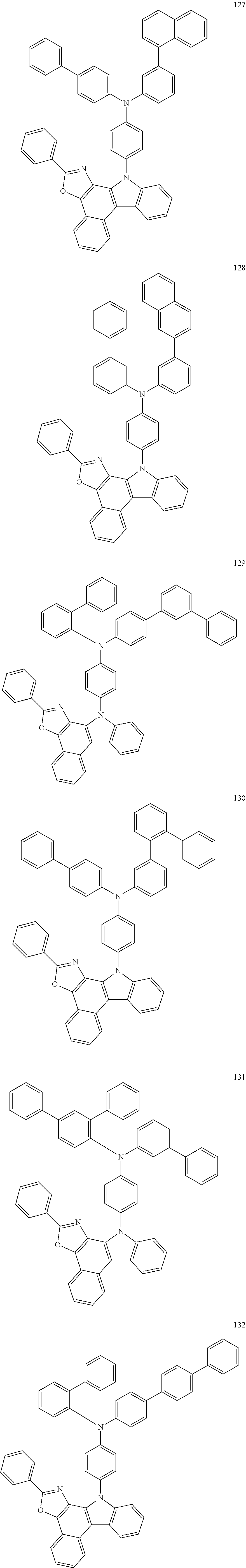

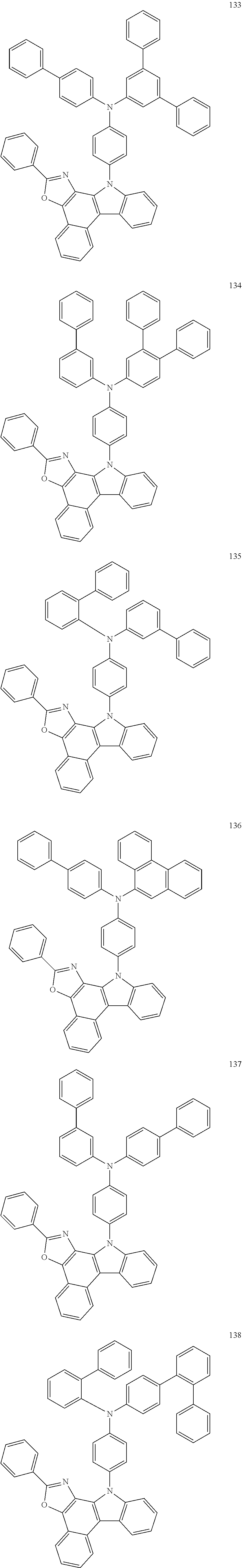

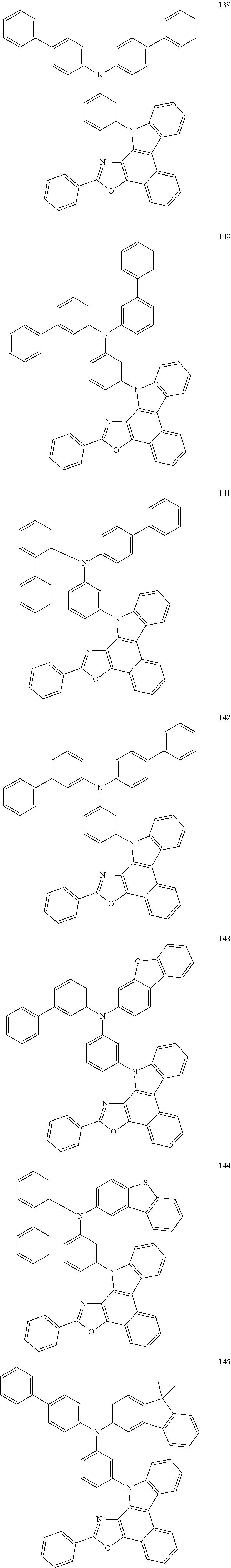

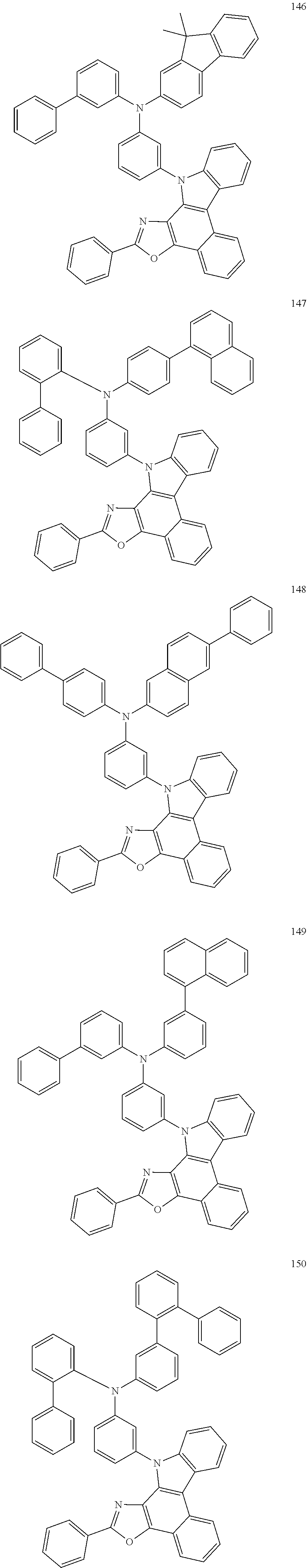

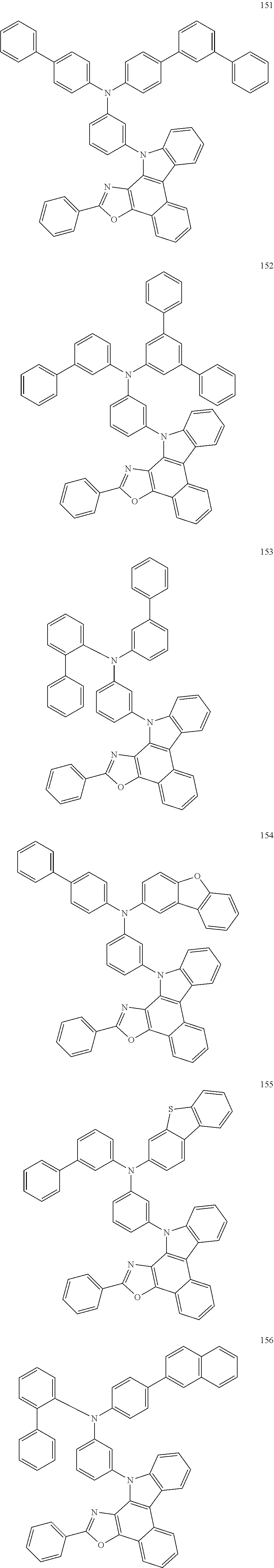

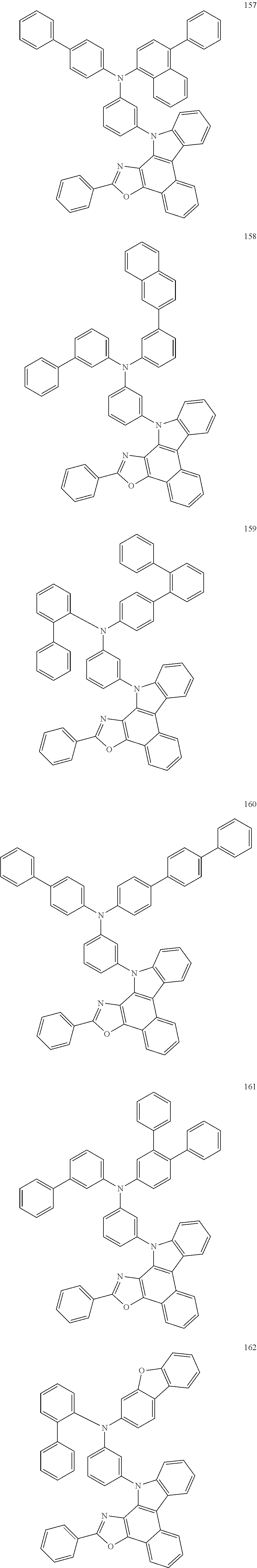

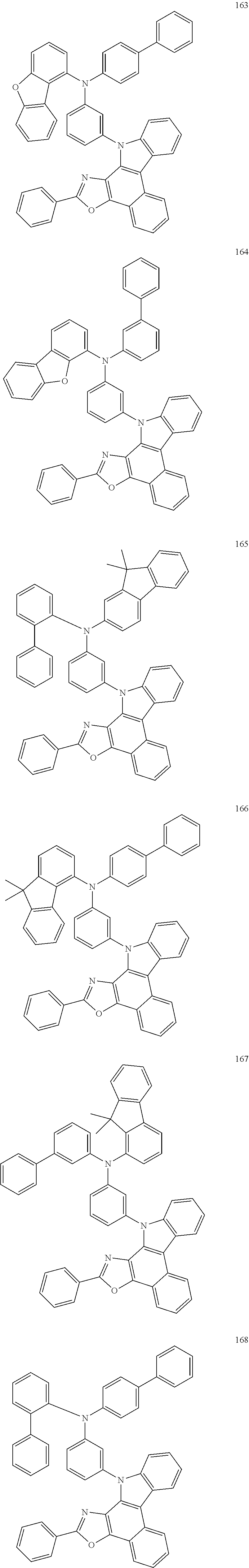

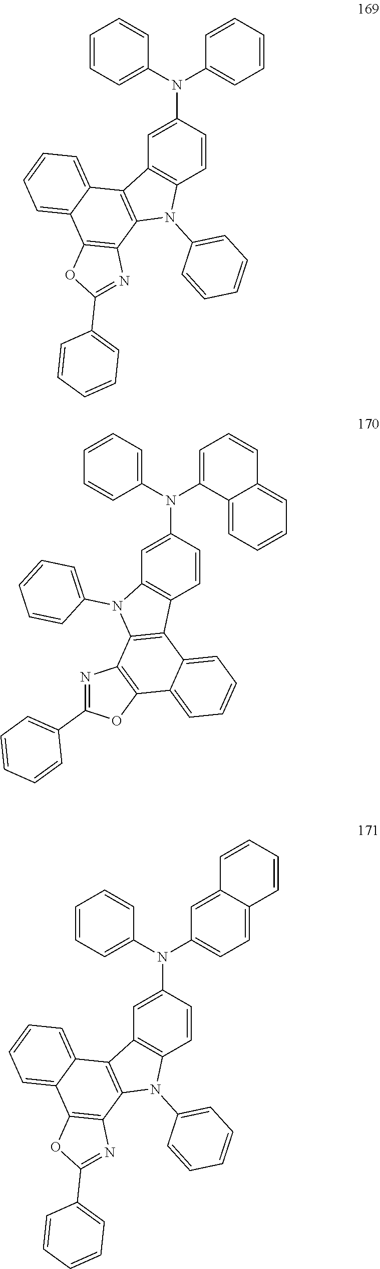

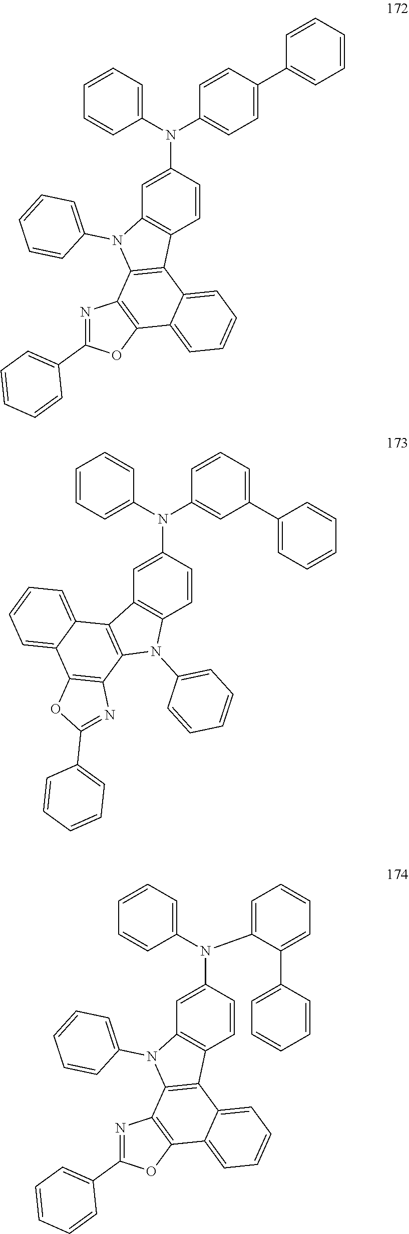 [SMILES: c1ccc(-c2ccc(N(c3ccccc3)c3ccc4c5c6ccccc6c6oc(-c7ccccc7)nc6c5n(-c5ccccc5)c4c3)cc2)cc1 ; c1ccc(-c2cccc(N(c3ccccc3)c3ccc4c(c3)c3c5ccccc5c5oc(-c6ccccc6)nc5c3n4-c3ccccc3)c2)cc1 ; c1ccc(-c2nc3c(o2)c2ccccc2c2c4ccc(N(c5ccccc5)c5ccccc5-c5ccccc5)cc4n(-c4ccccc4)c32)cc1]

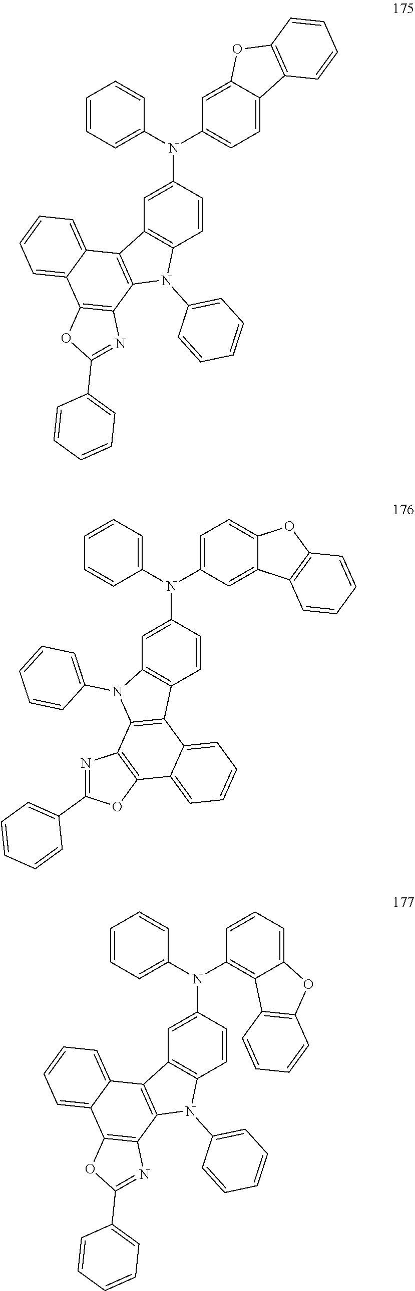 [SMILES: c1ccc(-c2nc3c(o2)c2ccccc2c2c4cc(N(c5ccccc5)c5ccc6c(c5)oc5ccccc56)ccc4n(-c4ccccc4)c32)cc1 ; c1ccc(-c2nc3c(o2)c2ccccc2c2c4ccc(N(c5ccccc5)c5ccc6oc7ccccc7c6c5)cc4n(-c4ccccc4)c32)cc1 ; c1ccc(-c2nc3c(o2)c2ccccc2c2c4cc(N(c5ccccc5)c5cccc6oc7ccccc7c56)ccc4n(-c4ccccc4)c32)cc1]

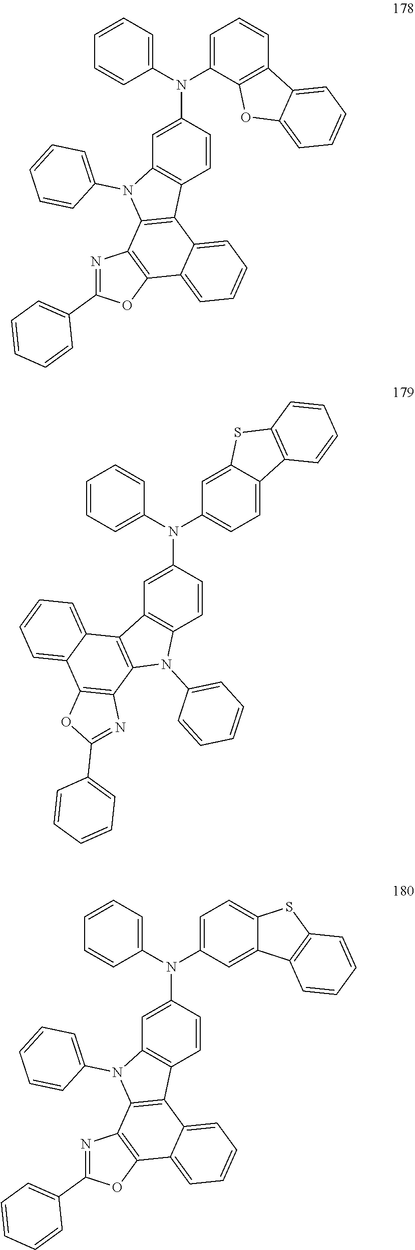 [SMILES: c1ccc(-c2nc3c(o2)c2ccccc2c2c4ccc(N(c5ccccc5)c5cccc6c5oc5ccccc56)cc4n(-c4ccccc4)c32)cc1 ; c1ccc(-c2nc3c(o2)c2ccccc2c2c4cc(N(c5ccccc5)c5ccc6c(c5)sc5ccccc56)ccc4n(-c4ccccc4)c32)cc1 ; c1ccc(-c2nc3c(o2)c2ccccc2c2c4ccc(N(c5ccccc5)c5ccc6sc7ccccc7c6c5)cc4n(-c4ccccc4)c32)cc1]

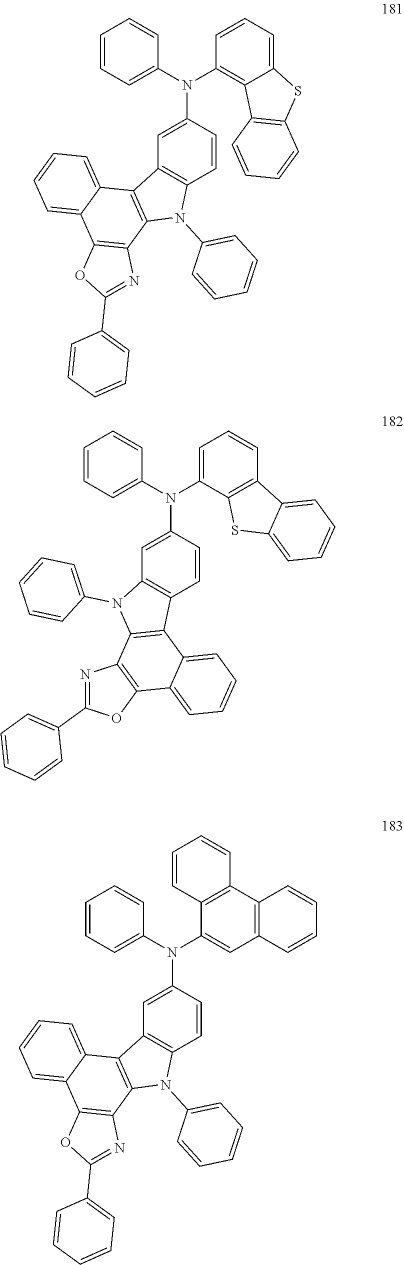

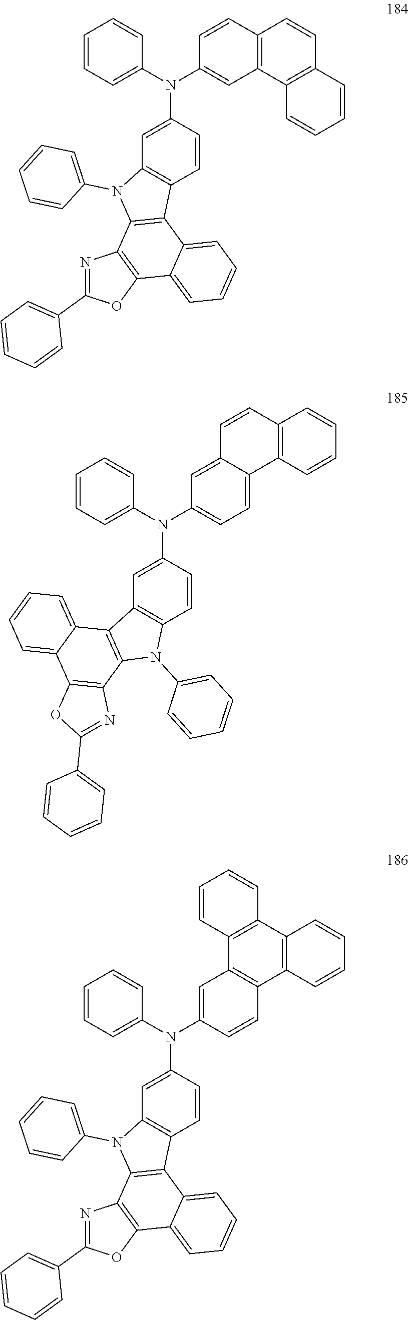 [SMILES: c1ccc(-c2nc3c(o2)c2ccccc2c2c4ccc(N(c5ccccc5)c5ccc6ccc7ccccc7c6c5)cc4n(-c4ccccc4)c32)cc1 ; c1ccc(-c2nc3c(o2)c2ccccc2c2c4cc(N(c5ccccc5)c5ccc6c(ccc7ccccc76)c5)ccc4n(-c4ccccc4)c32)cc1 ; c1ccc(-c2nc3c(o2)c2ccccc2c2c4ccc(N(c5ccccc5)c5ccc6c7ccccc7c7ccccc7c6c5)cc4n(-c4ccccc4)c32)cc1]

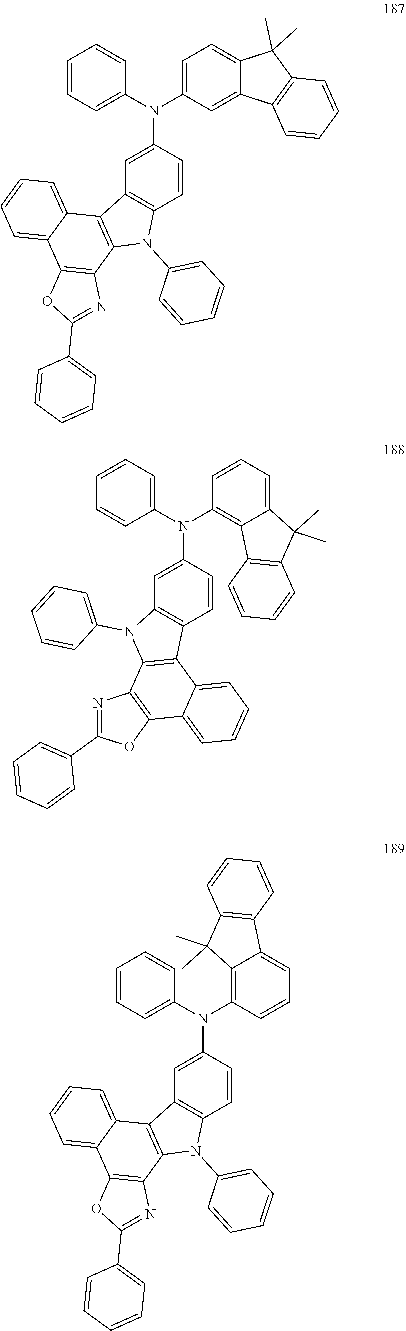 [SMILES: CC1(C)c2ccccc2-c2cc(N(c3ccccc3)c3ccc4c(c3)c3c5ccccc5c5oc(-c6ccccc6)nc5c3n4-c3ccccc3)ccc21 ; CC1(C)c2ccccc2-c2c(N(c3ccccc3)c3ccc4c5c6ccccc6c6oc(-c7ccccc7)nc6c5n(-c5ccccc5)c4c3)cccc21 ; CC1(C)c2ccccc2-c2cccc(N(c3ccccc3)c3ccc4c(c3)c3c5ccccc5c5oc(-c6ccccc6)nc5c3n4-c3ccccc3)c21]

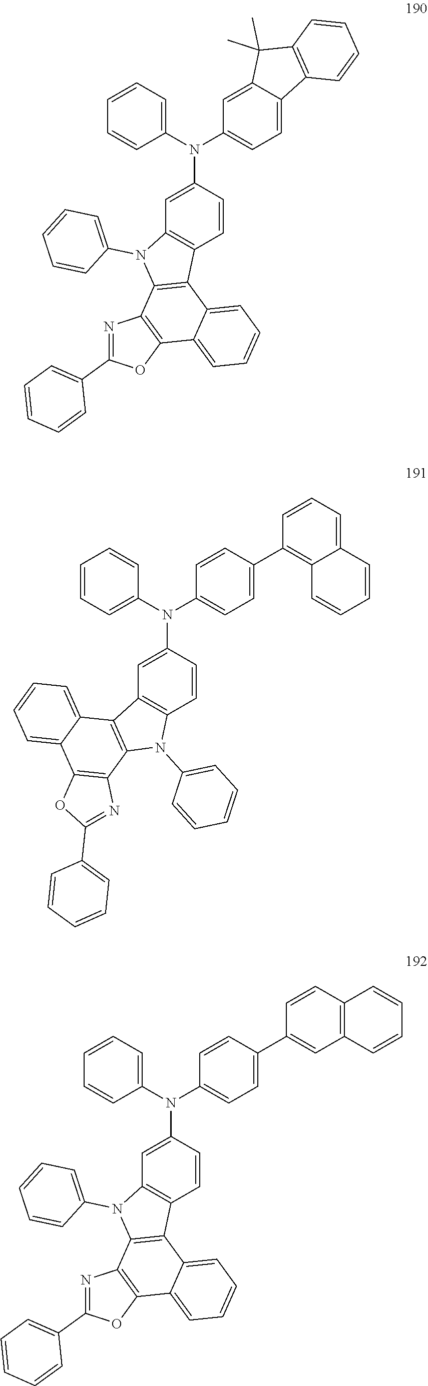 [SMILES: CC1(C)c2ccccc2-c2ccc(N(c3ccccc3)c3ccc4c5c6ccccc6c6oc(-c7ccccc7)nc6c5n(-c5ccccc5)c4c3)cc21 ; c1ccc(-c2nc3c(o2)c2ccccc2c2c4cc(N(c5ccccc5)c5ccc(-c6cccc7ccccc67)cc5)ccc4n(-c4ccccc4)c32)cc1 ; c1ccc(-c2nc3c(o2)c2ccccc2c2c4ccc(N(c5ccccc5)c5ccc(-c6ccc7ccccc7c6)cc5)cc4n(-c4ccccc4)c32)cc1]

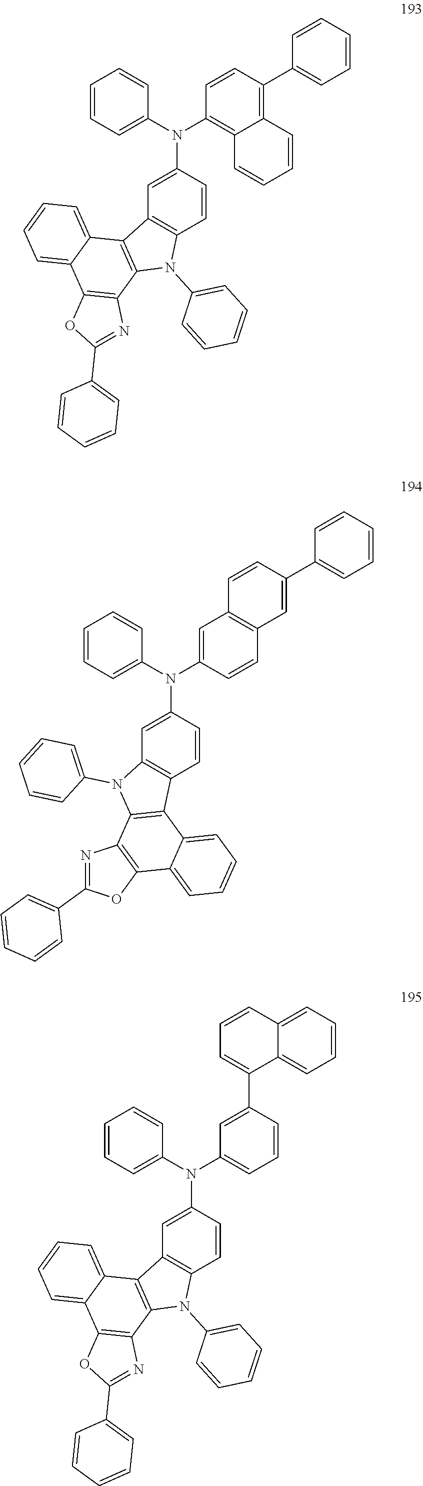 [SMILES: c1ccc(-c2nc3c(o2)c2ccccc2c2c4cc(N(c5ccccc5)c5ccc(-c6ccccc6)c6ccccc56)ccc4n(-c4ccccc4)c32)cc1 ; c1ccc(-c2ccc3cc(N(c4ccccc4)c4ccc5c6c7ccccc7c7oc(-c8ccccc8)nc7c6n(-c6ccccc6)c5c4)ccc3c2)cc1 ; c1ccc(-c2nc3c(o2)c2ccccc2c2c4cc(N(c5ccccc5)c5cccc(-c6cccc7ccccc67)c5)ccc4n(-c4ccccc4)c32)cc1]

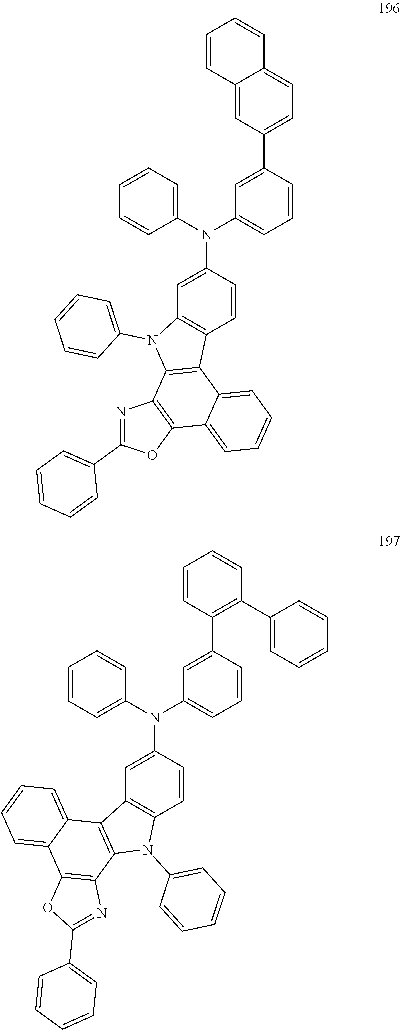 [SMILES: c1ccc(-c2nc3c(o2)c2ccccc2c2c4ccc(N(c5ccccc5)c5cccc(-c6ccc7ccccc7c6)c5)cc4n(-c4ccccc4)c32)cc1 ; c1ccc(-c2nc3c(o2)c2ccccc2c2c4cc(N(c5ccccc5)c5cccc(-c6ccccc6-c6ccccc6)c5)ccc4n(-c4ccccc4)c32)cc1]

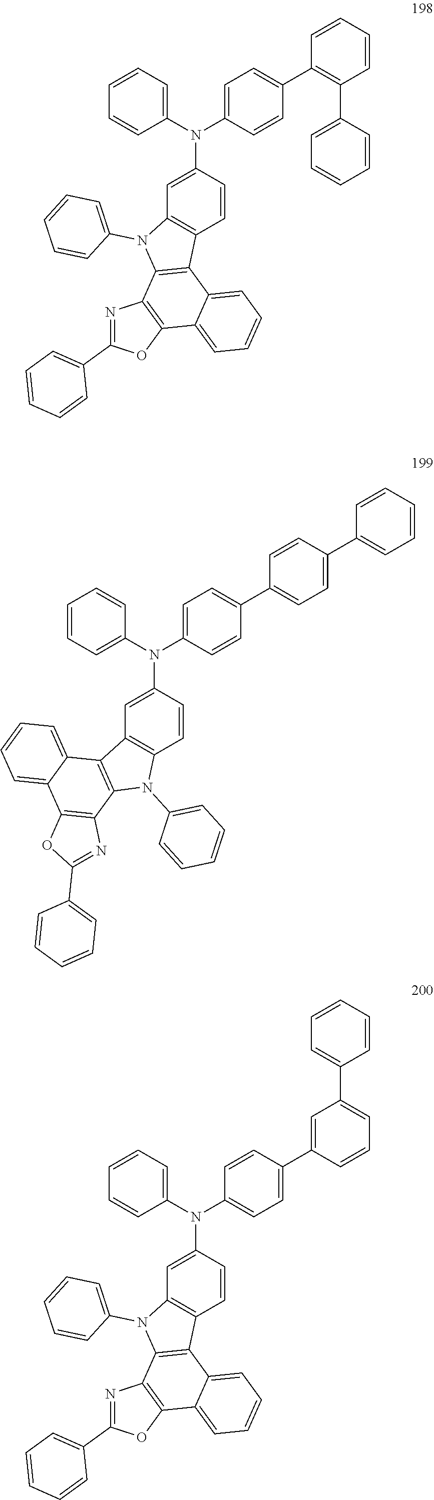 [SMILES: c1ccc(-c2nc3c(o2)c2ccccc2c2c4ccc(N(c5ccccc5)c5ccc(-c6ccccc6-c6ccccc6)cc5)cc4n(-c4ccccc4)c32)cc1 ; c1ccc(-c2ccc(-c3ccc(N(c4ccccc4)c4ccc5c(c4)c4c6ccccc6c6oc(-c7ccccc7)nc6c4n5-c4ccccc4)cc3)cc2)cc1 ; c1ccc(-c2cccc(-c3ccc(N(c4ccccc4)c4ccc5c6c7ccccc7c7oc(-c8ccccc8)nc7c6n(-c6ccccc6)c5c4)cc3)c2)cc1]

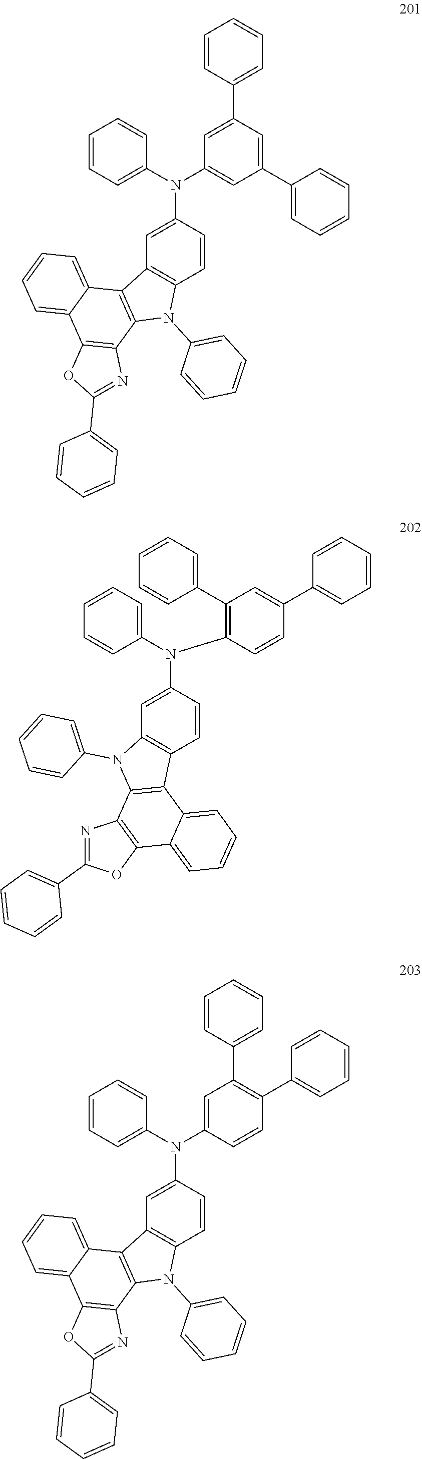 [SMILES: c1ccc(-c2cc(-c3ccccc3)cc(N(c3ccccc3)c3ccc4c(c3)c3c5ccccc5c5oc(-c6ccccc6)nc5c3n4-c3ccccc3)c2)cc1 ; c1ccc(-c2ccc(N(c3ccccc3)c3ccc4c5c6ccccc6c6oc(-c7ccccc7)nc6c5n(-c5ccccc5)c4c3)c(-c3ccccc3)c2)cc1 ; c1ccc(-c2nc3c(o2)c2ccccc2c2c4cc(N(c5ccccc5)c5ccc(-c6ccccc6)c(-c6ccccc6)c5)ccc4n(-c4ccccc4)c32)cc1]

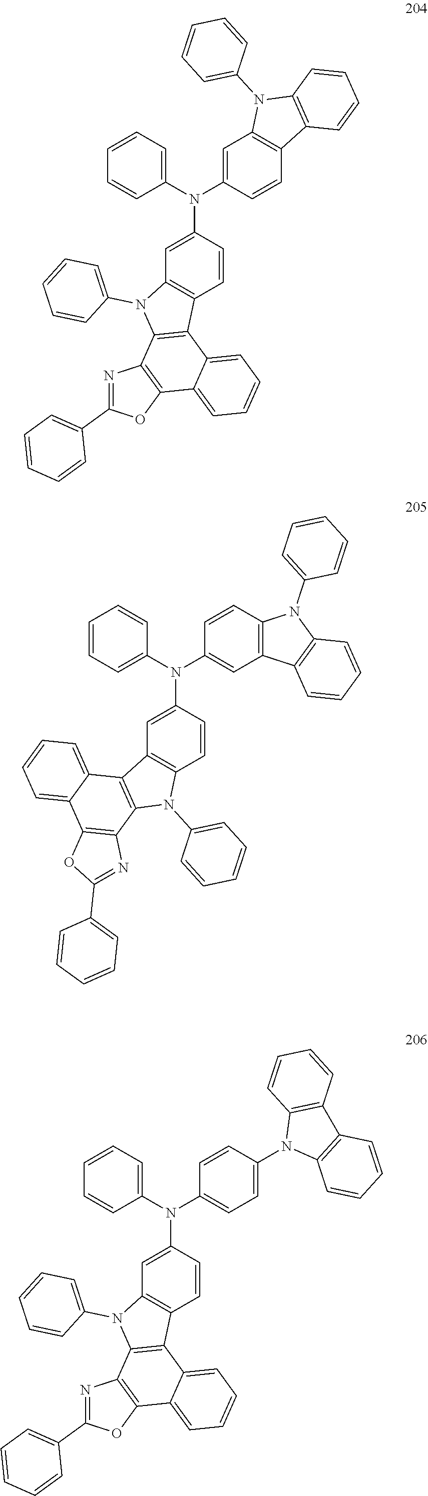 [SMILES: c1ccc(-c2nc3c(o2)c2ccccc2c2c4ccc(N(c5ccccc5)c5ccc6c7ccccc7n(-c7ccccc7)c6c5)cc4n(-c4ccccc4)c32)cc1 ; c1ccc(-c2nc3c(o2)c2ccccc2c2c4cc(N(c5ccccc5)c5ccc6c(c5)c5ccccc5n6-c5ccccc5)ccc4n(-c4ccccc4)c32)cc1 ; c1ccc(-c2nc3c(o2)c2ccccc2c2c4ccc(N(c5ccccc5)c5ccc(-n6c7ccccc7c7ccccc76)cc5)cc4n(-c4ccccc4)c32)cc1]

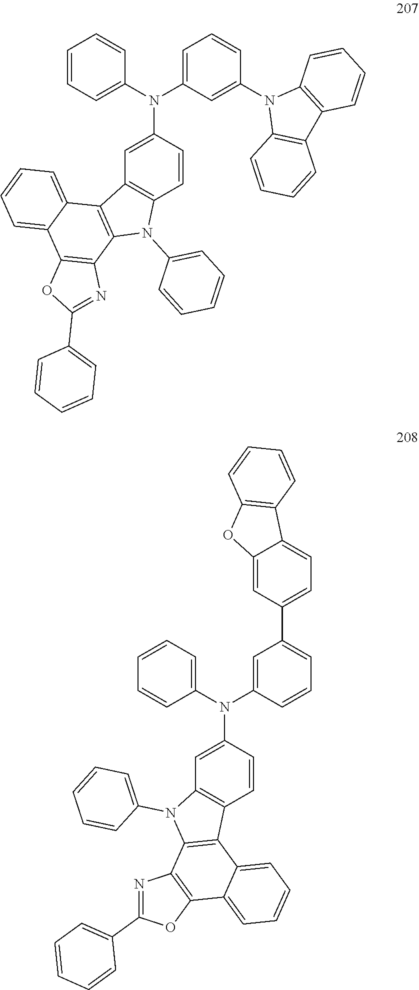 [SMILES: c1ccc(-c2nc3c(o2)c2ccccc2c2c4cc(N(c5ccccc5)c5cccc(-n6c7ccccc7c7ccccc76)c5)ccc4n(-c4ccccc4)c32)cc1 ; c1ccc(-c2nc3c(o2)c2ccccc2c2c4ccc(N(c5ccccc5)c5cccc(-c6ccc7c(c6)oc6ccccc67)c5)cc4n(-c4ccccc4)c32)cc1]

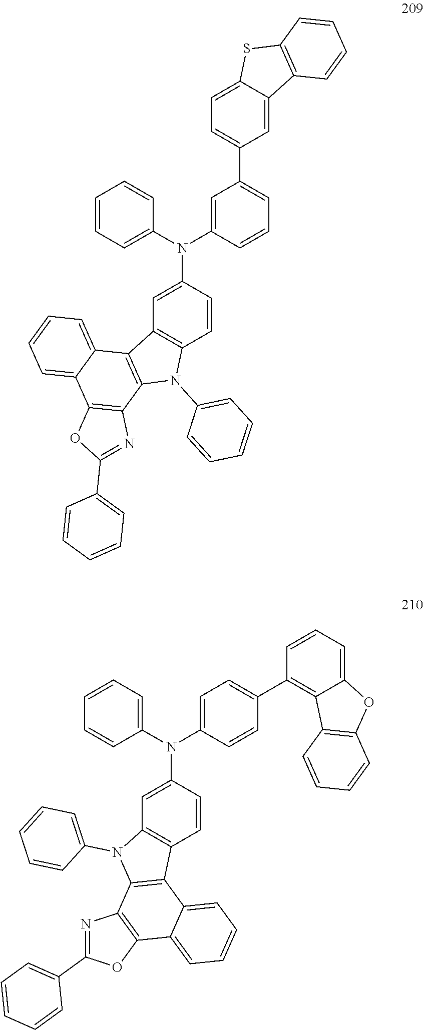 [SMILES: c1ccc(-c2nc3c(o2)c2ccccc2c2c4cc(N(c5ccccc5)c5cccc(-c6ccc7sc8ccccc8c7c6)c5)ccc4n(-c4ccccc4)c32)cc1 ; c1ccc(-c2nc3c(o2)c2ccccc2c2c4ccc(N(c5ccccc5)c5ccc(-c6cccc7oc8ccccc8c67)cc5)cc4n(-c4ccccc4)c32)cc1]

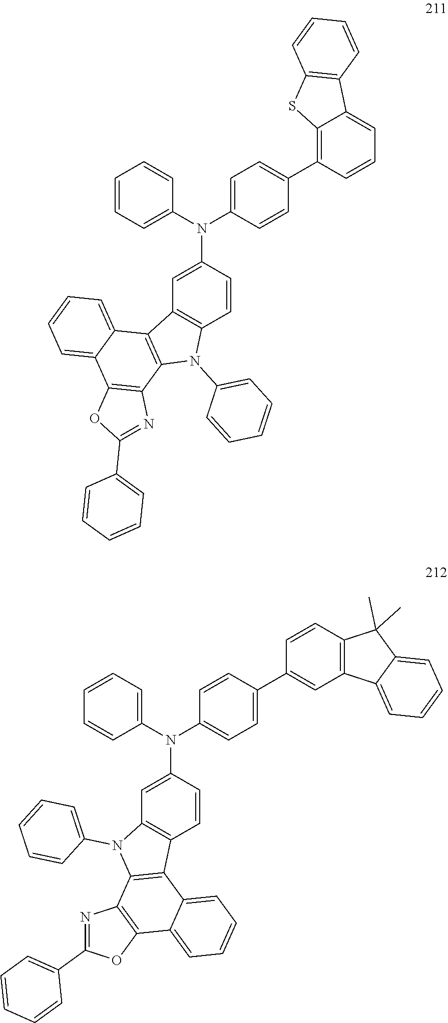 [SMILES: c1ccc(-c2nc3c(o2)c2ccccc2c2c4cc(N(c5ccccc5)c5ccc(-c6cccc7c6sc6ccccc67)cc5)ccc4n(-c4ccccc4)c32)cc1 ; CC1(C)c2ccccc2-c2cc(-c3ccc(N(c4ccccc4)c4ccc5c6c7ccccc7c7oc(-c8ccccc8)nc7c6n(-c6ccccc6)c5c4)cc3)ccc21]

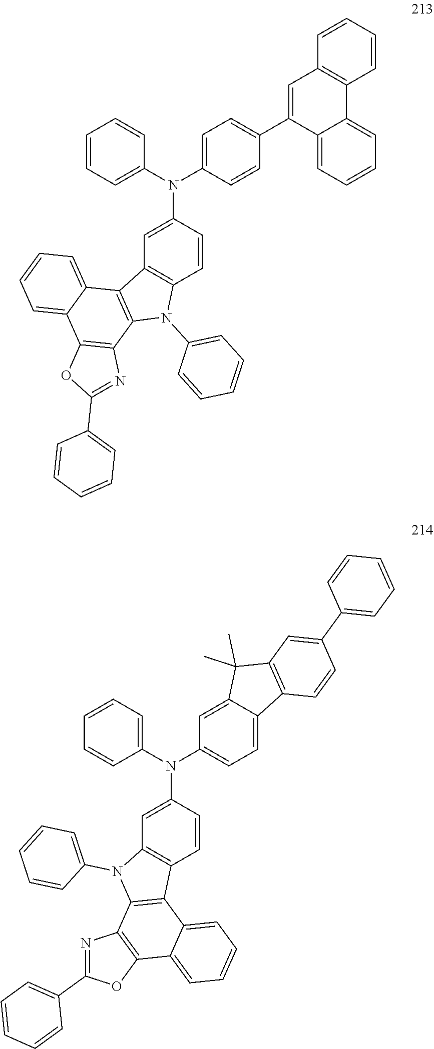 [SMILES: c1ccc(-c2nc3c(o2)c2ccccc2c2c4cc(N(c5ccccc5)c5ccc(-c6cc7ccccc7c7ccccc67)cc5)ccc4n(-c4ccccc4)c32)cc1 ; CC1(C)c2cc(-c3ccccc3)ccc2-c2ccc(N(c3ccccc3)c3ccc4c5c6ccccc6c6oc(-c7ccccc7)nc6c5n(-c5ccccc5)c4c3)cc21]

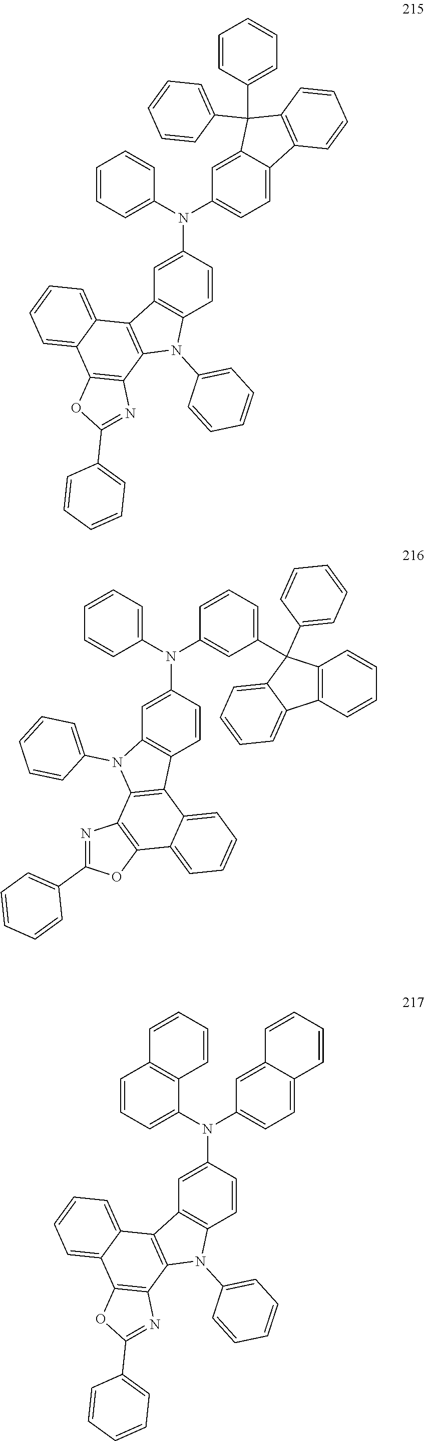 [SMILES: c1ccc(-c2nc3c(o2)c2ccccc2c2c4cc(N(c5ccccc5)c5ccc6c(c5)C(c5ccccc5)(c5ccccc5)c5ccccc5-6)ccc4n(-c4ccccc4)c32)cc1 ; c1ccc(-c2nc3c(o2)c2ccccc2c2c4ccc(N(c5ccccc5)c5cccc(C6(c7ccccc7)c7ccccc7-c7ccccc76)c5)cc4n(-c4ccccc4)c32)cc1 ; c1ccc(-c2nc3c(o2)c2ccccc2c2c4cc(N(c5ccc6ccccc6c5)c5cccc6ccccc56)ccc4n(-c4ccccc4)c32)cc1]

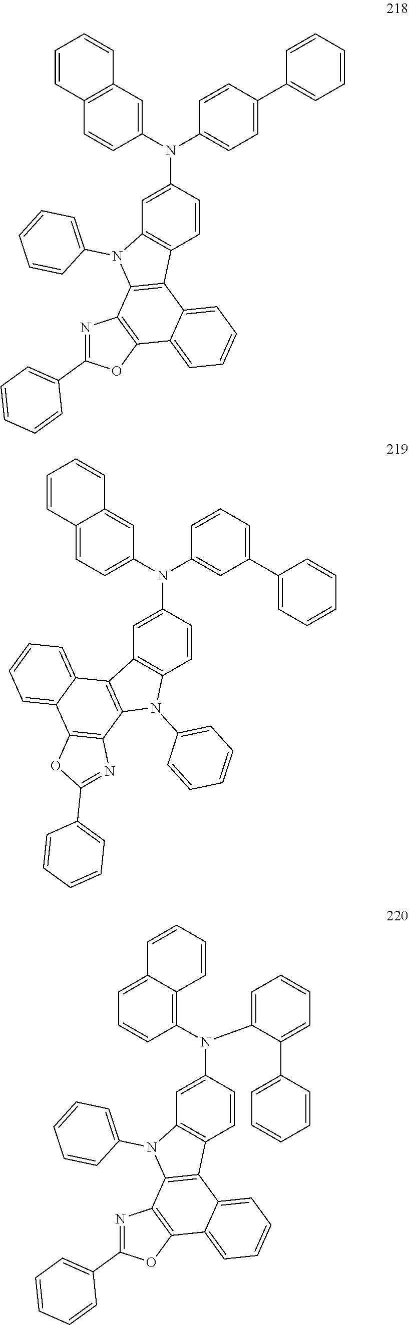

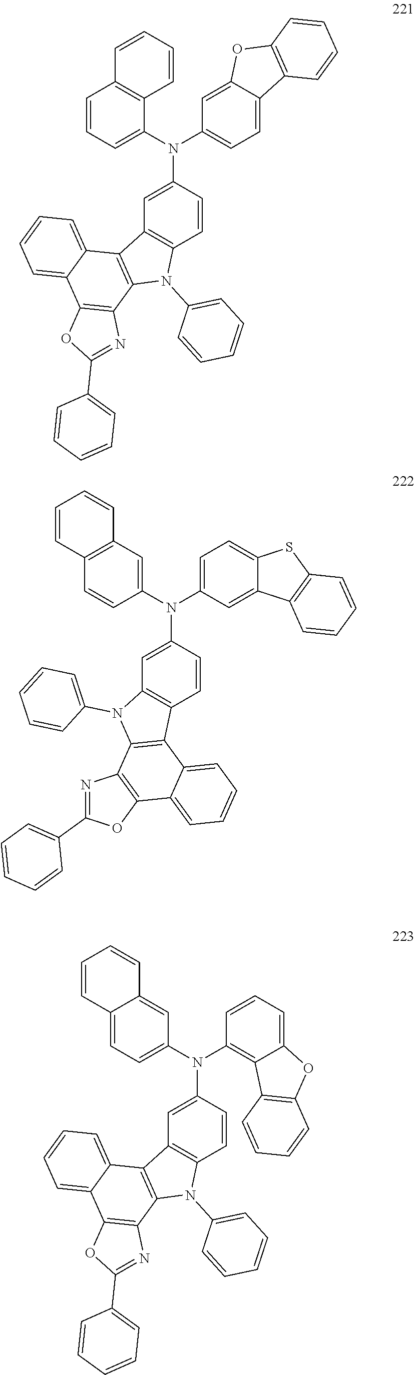

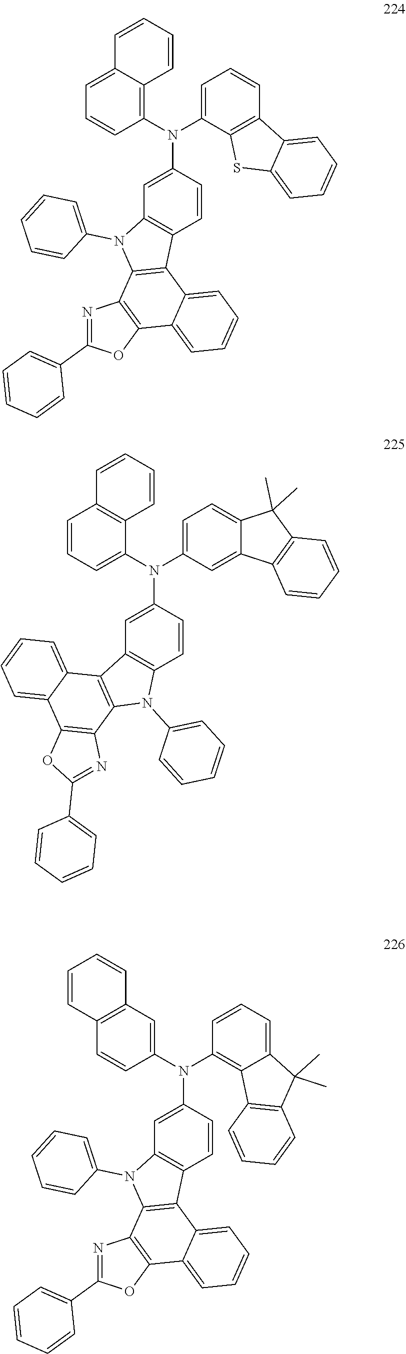 [SMILES: c1ccc(-c2nc3c(o2)c2ccccc2c2c4ccc(N(c5cccc6ccccc56)c5cccc6c5sc5ccccc56)cc4n(-c4ccccc4)c32)cc1 ; CC1(C)c2ccccc2-c2cc(N(c3ccc4c(c3)c3c5ccccc5c5oc(-c6ccccc6)nc5c3n4-c3ccccc3)c3cccc4ccccc34)ccc21 ; CC1(C)c2ccccc2-c2c(N(c3ccc4ccccc4c3)c3ccc4c5c6ccccc6c6oc(-c7ccccc7)nc6c5n(-c5ccccc5)c4c3)cccc21]

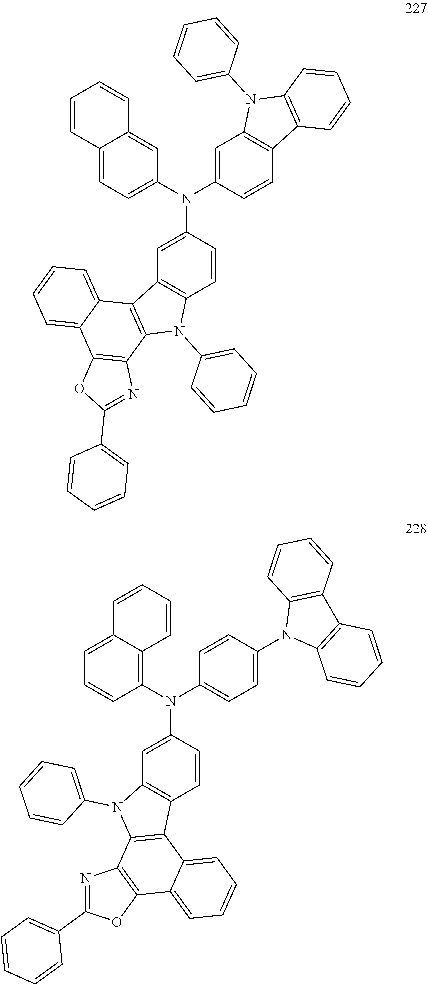 [SMILES: c1ccc(-c2nc3c(o2)c2ccccc2c2c4cc(N(c5ccc6ccccc6c5)c5ccc6c7ccccc7n(-c7ccccc7)c6c5)ccc4n(-c4ccccc4)c32)cc1 ; c1ccc(-c2nc3c(o2)c2ccccc2c2c4ccc(N(c5ccc(-n6c7ccccc7c7ccccc76)cc5)c5cccc6ccccc56)cc4n(-c4ccccc4)c32)cc1]

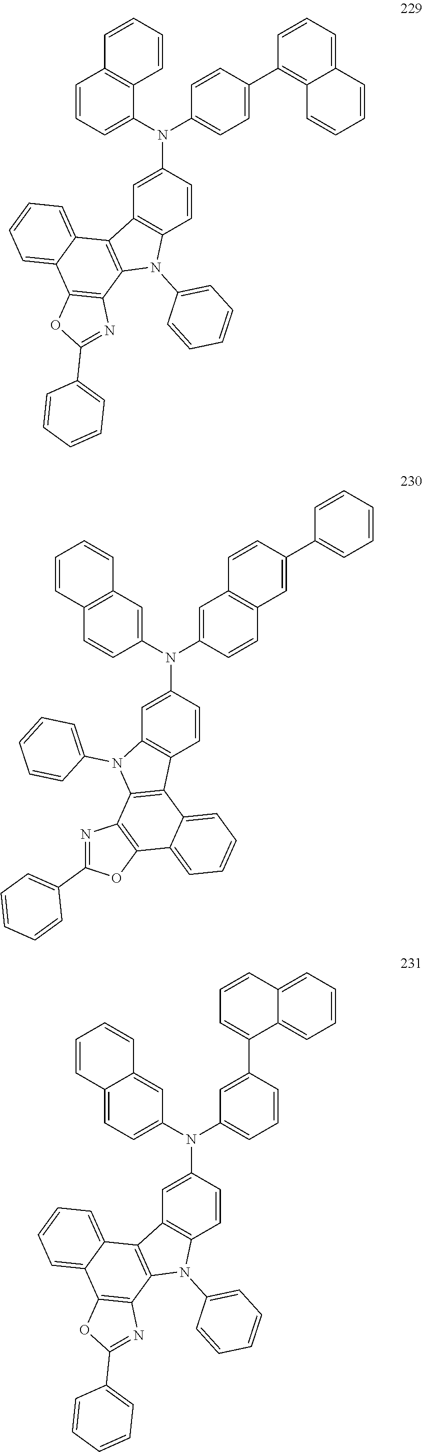 [SMILES: c1ccc(-c2nc3c(o2)c2ccccc2c2c4cc(N(c5ccc(-c6cccc7ccccc67)cc5)c5cccc6ccccc56)ccc4n(-c4ccccc4)c32)cc1 ; c1ccc(-c2ccc3cc(N(c4ccc5ccccc5c4)c4ccc5c6c7ccccc7c7oc(-c8ccccc8)nc7c6n(-c6ccccc6)c5c4)ccc3c2)cc1 ; c1ccc(-c2nc3c(o2)c2ccccc2c2c4cc(N(c5cccc(-c6cccc7ccccc67)c5)c5ccc6ccccc6c5)ccc4n(-c4ccccc4)c32)cc1]

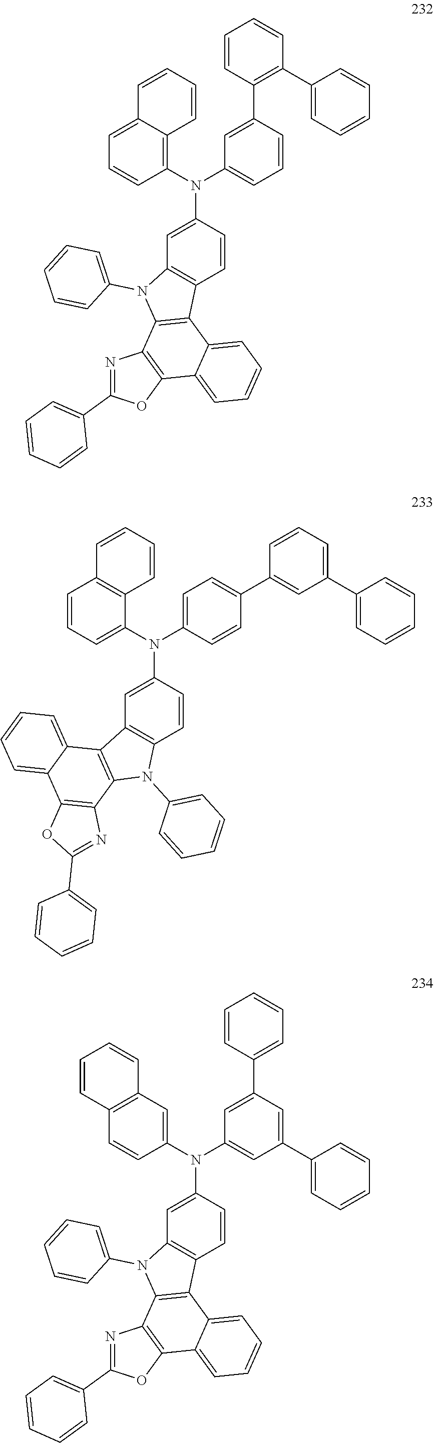 [SMILES: c1ccc(-c2nc3c(o2)c2ccccc2c2c4ccc(N(c5cccc(-c6ccccc6-c6ccccc6)c5)c5cccc6ccccc56)cc4n(-c4ccccc4)c32)cc1 ; c1ccc(-c2cccc(-c3ccc(N(c4ccc5c(c4)c4c6ccccc6c6oc(-c7ccccc7)nc6c4n5-c4ccccc4)c4cccc5ccccc45)cc3)c2)cc1 ; c1ccc(-c2cc(-c3ccccc3)cc(N(c3ccc4ccccc4c3)c3ccc4c5c6ccccc6c6oc(-c7ccccc7)nc6c5n(-c5ccccc5)c4c3)c2)cc1]

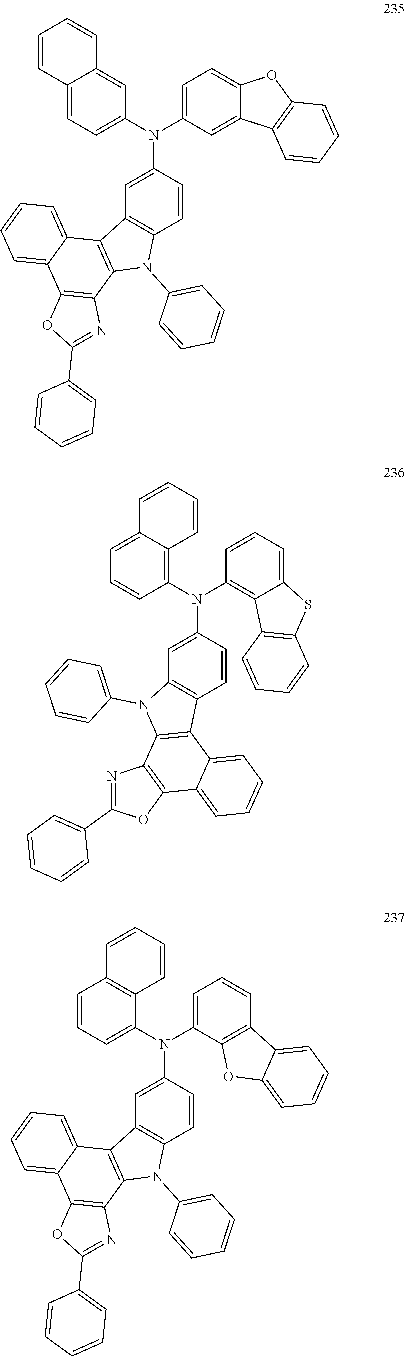 [SMILES: c1ccc(-c2nc3c(o2)c2ccccc2c2c4cc(N(c5ccc6ccccc6c5)c5ccc6oc7ccccc7c6c5)ccc4n(-c4ccccc4)c32)cc1 ; c1ccc(-c2nc3c(o2)c2ccccc2c2c4ccc(N(c5cccc6ccccc56)c5cccc6sc7ccccc7c56)cc4n(-c4ccccc4)c32)cc1 ; c1ccc(-c2nc3c(o2)c2ccccc2c2c4cc(N(c5cccc6ccccc56)c5cccc6c5oc5ccccc56)ccc4n(-c4ccccc4)c32)cc1]

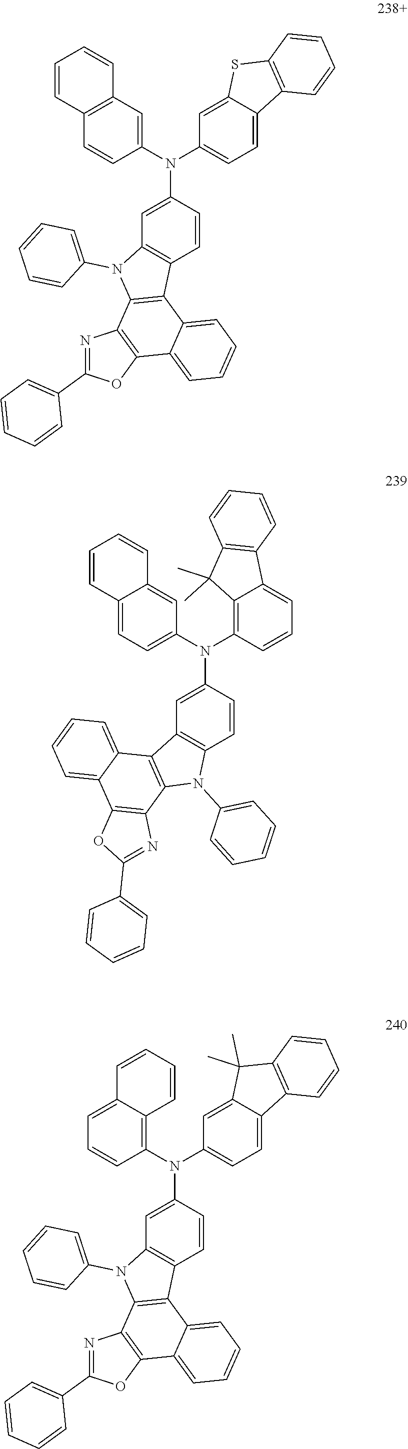 [SMILES: c1ccc(-c2nc3c(o2)c2ccccc2c2c4ccc(N(c5ccc6ccccc6c5)c5ccc6c(c5)sc5ccccc56)cc4n(-c4ccccc4)c32)cc1 ; CC1(C)c2ccccc2-c2cccc(N(c3ccc4ccccc4c3)c3ccc4c(c3)c3c5ccccc5c5oc(-c6ccccc6)nc5c3n4-c3ccccc3)c21 ; CC1(C)c2ccccc2-c2ccc(N(c3ccc4c5c6ccccc6c6oc(-c7ccccc7)nc6c5n(-c5ccccc5)c4c3)c3cccc4ccccc34)cc21]

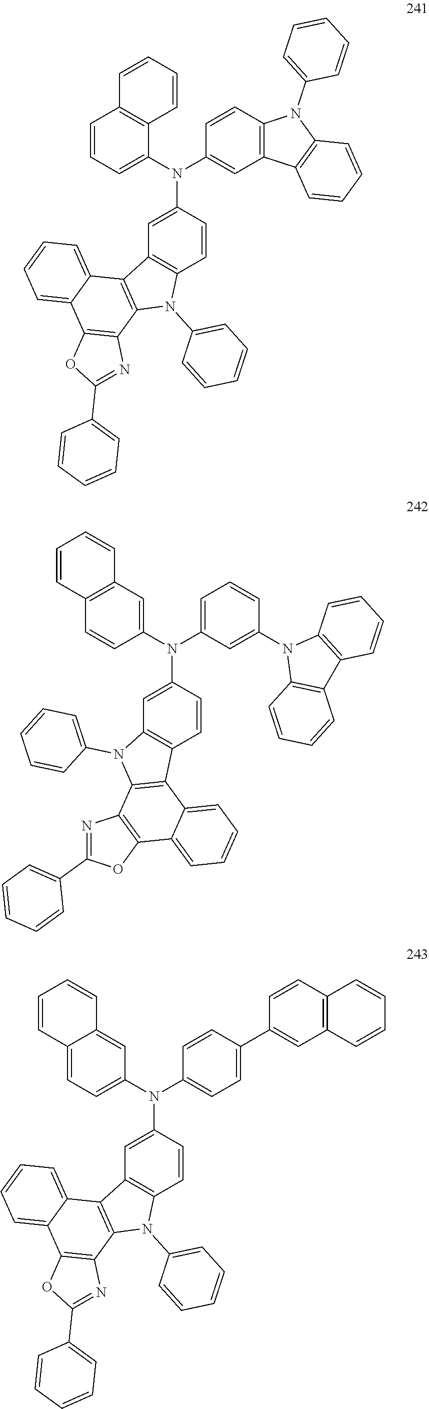

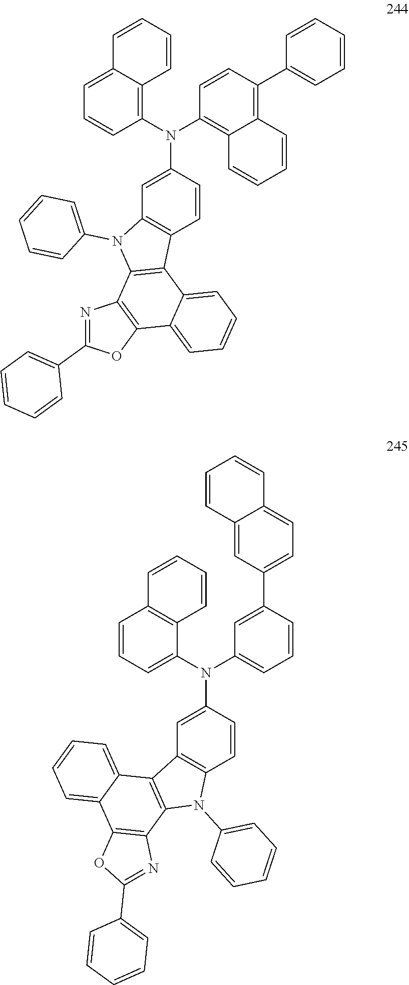 [SMILES: c1ccc(-c2nc3c(o2)c2ccccc2c2c4ccc(N(c5cccc6ccccc56)c5ccc(-c6ccccc6)c6ccccc56)cc4n(-c4ccccc4)c32)cc1 ; c1ccc(-c2nc3c(o2)c2ccccc2c2c4cc(N(c5cccc(-c6ccc7ccccc7c6)c5)c5cccc6ccccc56)ccc4n(-c4ccccc4)c32)cc1]

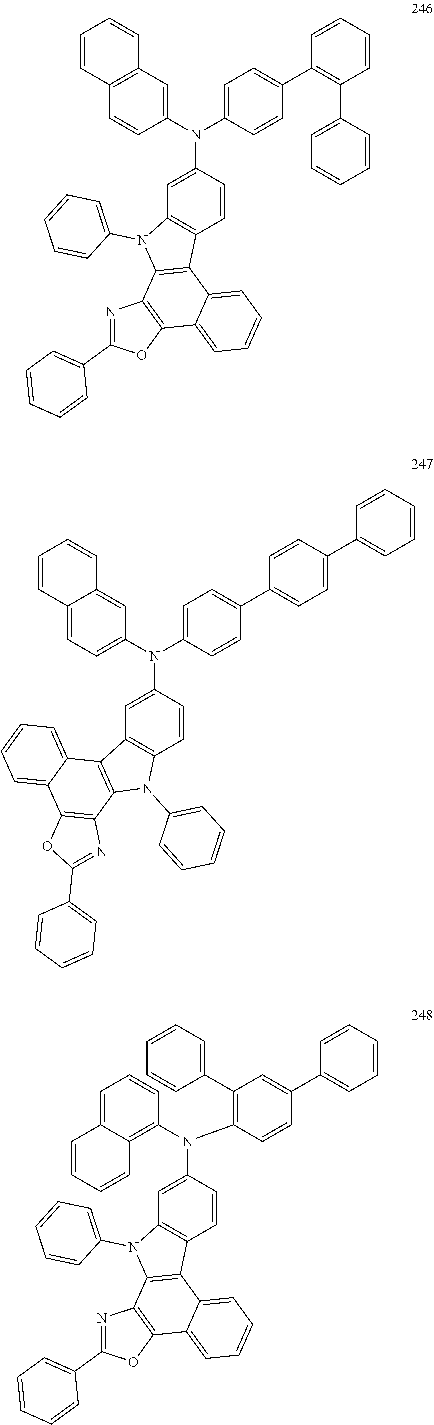 [SMILES: c1ccc(-c2nc3c(o2)c2ccccc2c2c4ccc(N(c5ccc(-c6ccccc6-c6ccccc6)cc5)c5ccc6ccccc6c5)cc4n(-c4ccccc4)c32)cc1 ; c1ccc(-c2ccc(-c3ccc(N(c4ccc5ccccc5c4)c4ccc5c(c4)c4c6ccccc6c6oc(-c7ccccc7)nc6c4n5-c4ccccc4)cc3)cc2)cc1 ; c1ccc(-c2ccc(N(c3ccc4c5c6ccccc6c6oc(-c7ccccc7)nc6c5n(-c5ccccc5)c4c3)c3cccc4ccccc34)c(-c3ccccc3)c2)cc1]

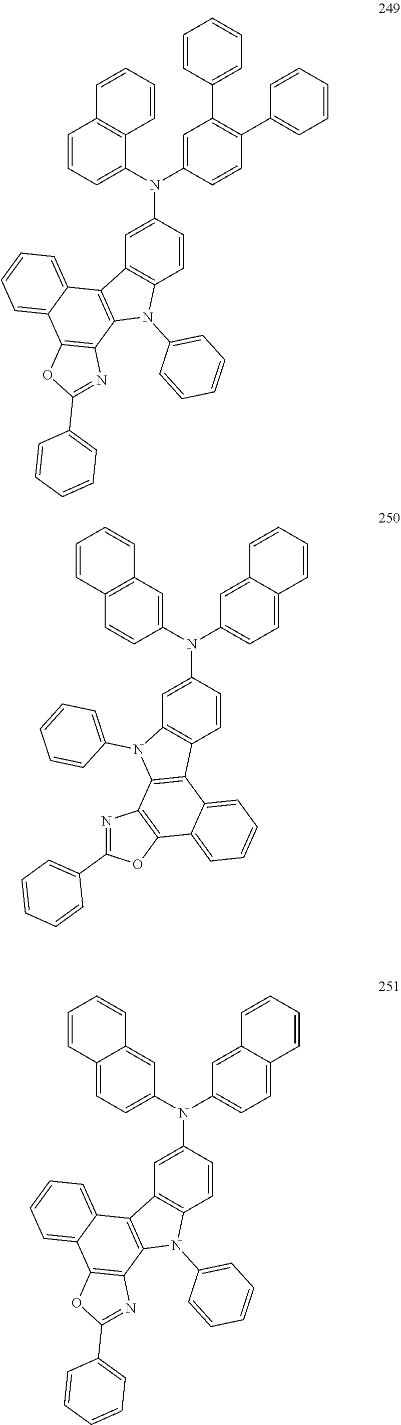

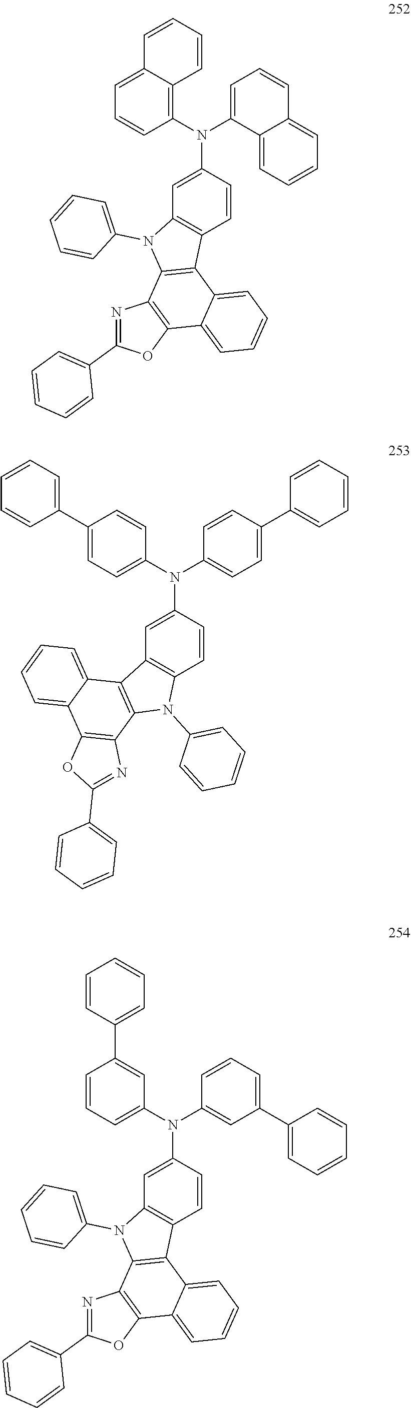 [SMILES: c1ccc(-c2nc3c(o2)c2ccccc2c2c4ccc(N(c5cccc6ccccc56)c5cccc6ccccc56)cc4n(-c4ccccc4)c32)cc1 ; c1ccc(-c2ccc(N(c3ccc(-c4ccccc4)cc3)c3ccc4c(c3)c3c5ccccc5c5oc(-c6ccccc6)nc5c3n4-c3ccccc3)cc2)cc1 ; c1ccc(-c2cccc(N(c3cccc(-c4ccccc4)c3)c3ccc4c5c6ccccc6c6oc(-c7ccccc7)nc6c5n(-c5ccccc5)c4c3)c2)cc1]

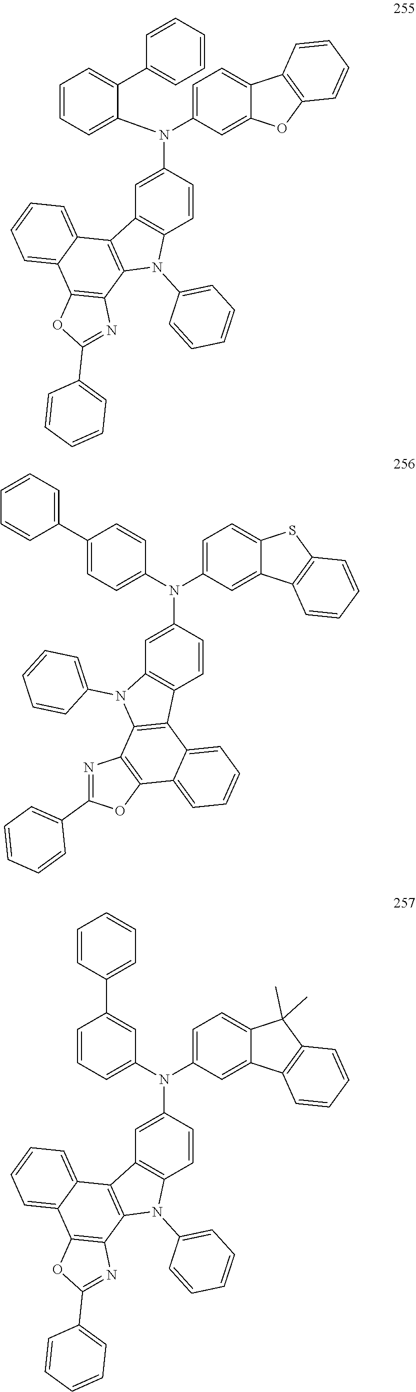

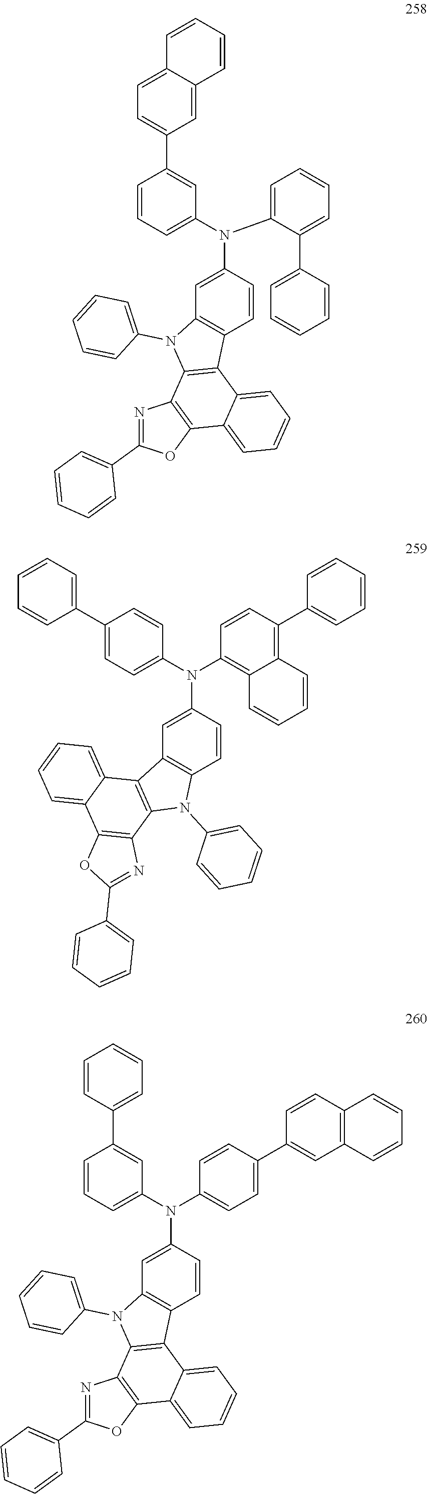 [SMILES: c1ccc(-c2nc3c(o2)c2ccccc2c2c4ccc(N(c5cccc(-c6ccc7ccccc7c6)c5)c5ccccc5-c5ccccc5)cc4n(-c4ccccc4)c32)cc1 ; c1ccc(-c2ccc(N(c3ccc4c(c3)c3c5ccccc5c5oc(-c6ccccc6)nc5c3n4-c3ccccc3)c3ccc(-c4ccccc4)c4ccccc34)cc2)cc1 ; c1ccc(-c2cccc(N(c3ccc(-c4ccc5ccccc5c4)cc3)c3ccc4c5c6ccccc6c6oc(-c7ccccc7)nc6c5n(-c5ccccc5)c4c3)c2)cc1]

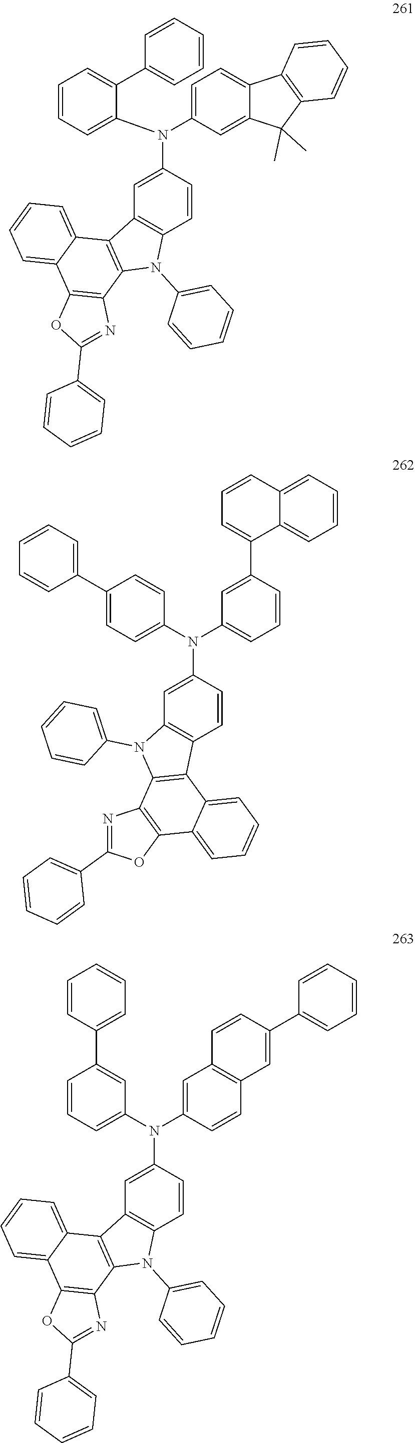 [SMILES: CC1(C)c2ccccc2-c2ccc(N(c3ccc4c(c3)c3c5ccccc5c5oc(-c6ccccc6)nc5c3n4-c3ccccc3)c3ccccc3-c3ccccc3)cc21 ; c1ccc(-c2ccc(N(c3cccc(-c4cccc5ccccc45)c3)c3ccc4c5c6ccccc6c6oc(-c7ccccc7)nc6c5n(-c5ccccc5)c4c3)cc2)cc1 ; c1ccc(-c2cccc(N(c3ccc4cc(-c5ccccc5)ccc4c3)c3ccc4c(c3)c3c5ccccc5c5oc(-c6ccccc6)nc5c3n4-c3ccccc3)c2)cc1]

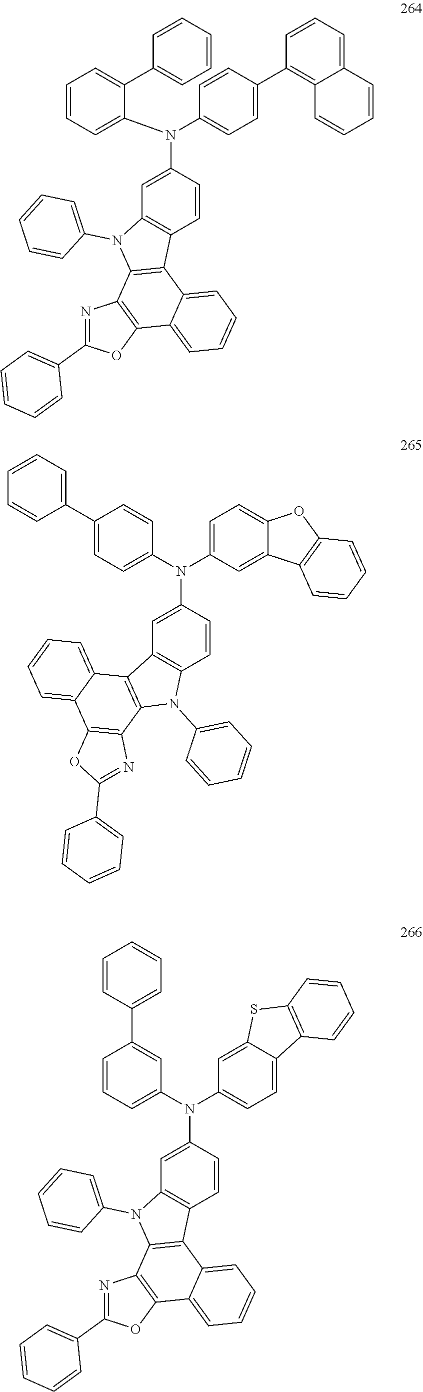

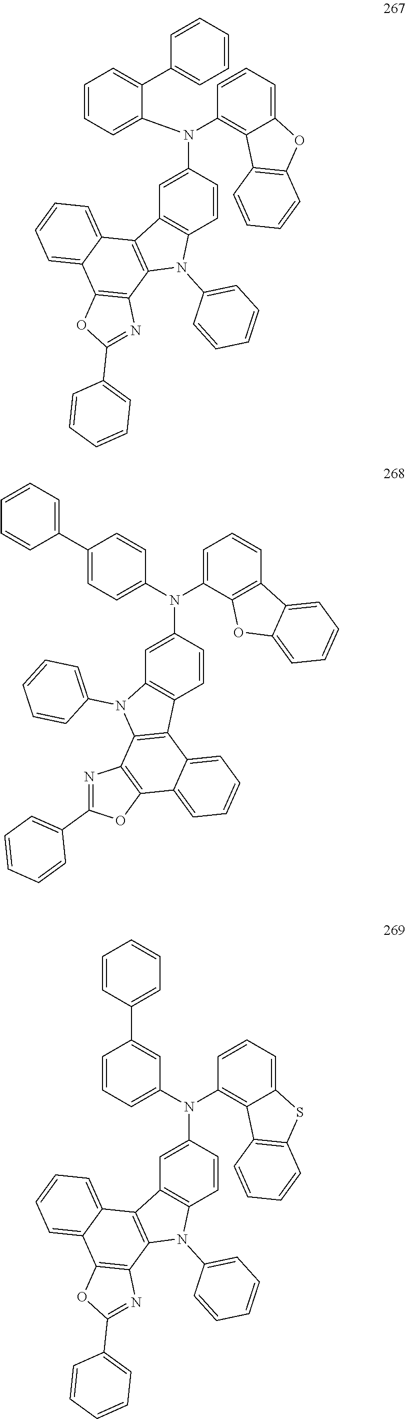 [SMILES: c1ccc(-c2nc3c(o2)c2ccccc2c2c4cc(N(c5ccccc5-c5ccccc5)c5cccc6oc7ccccc7c56)ccc4n(-c4ccccc4)c32)cc1 ; c1ccc(-c2ccc(N(c3ccc4c5c6ccccc6c6oc(-c7ccccc7)nc6c5n(-c5ccccc5)c4c3)c3cccc4c3oc3ccccc34)cc2)cc1 ; c1ccc(-c2cccc(N(c3ccc4c(c3)c3c5ccccc5c5oc(-c6ccccc6)nc5c3n4-c3ccccc3)c3cccc4sc5ccccc5c34)c2)cc1]

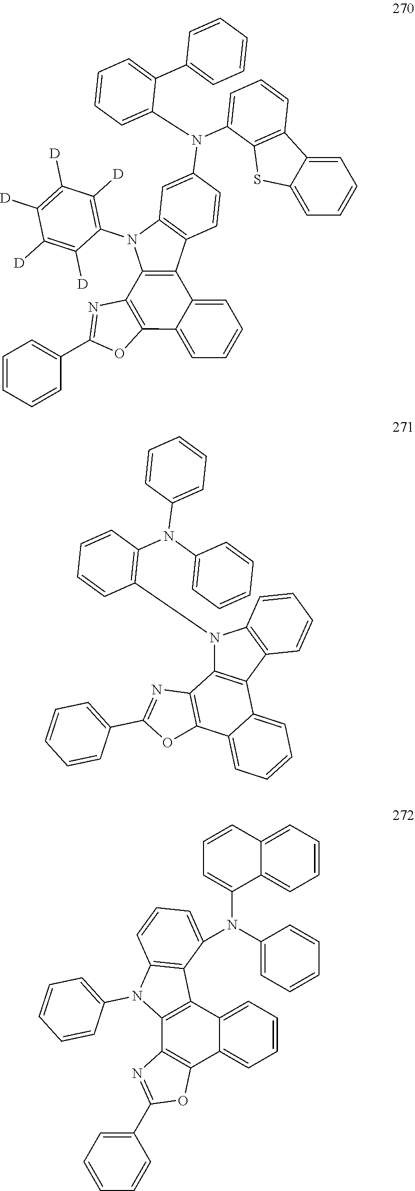 [SMILES: [2H]c1c([2H])c([2H])c(-n2c3cc(N(c4ccccc4-c4ccccc4)c4cccc5c4sc4ccccc45)ccc3c3c4ccccc4c4oc(-c5ccccc5)nc4c32)c([2H])c1[2H] ; c1ccc(-c2nc3c(o2)c2ccccc2c2c4ccccc4n(-c4ccccc4N(c4ccccc4)c4ccccc4)c32)cc1 ; c1ccc(-c2nc3c(o2)c2ccccc2c2c4c(N(c5ccccc5)c5cccc6ccccc56)cccc4n(-c4ccccc4)c32)cc1]

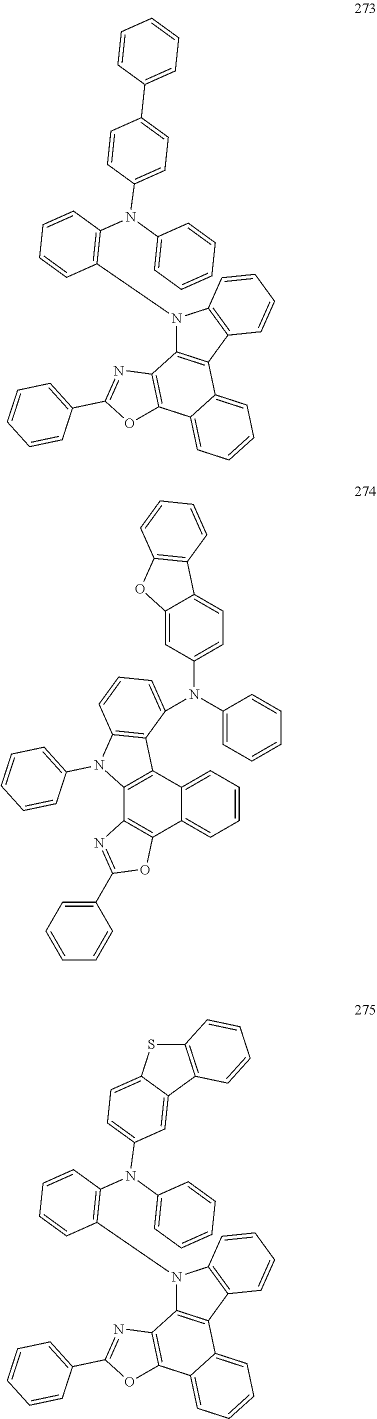 [SMILES: c1ccc(-c2ccc(N(c3ccccc3)c3ccccc3-n3c4ccccc4c4c5ccccc5c5oc(-c6ccccc6)nc5c43)cc2)cc1 ; c1ccc(-c2nc3c(o2)c2ccccc2c2c4c(N(c5ccccc5)c5ccc6c(c5)oc5ccccc56)cccc4n(-c4ccccc4)c32)cc1 ; c1ccc(-c2nc3c(o2)c2ccccc2c2c4ccccc4n(-c4ccccc4N(c4ccccc4)c4ccc5sc6ccccc6c5c4)c32)cc1]

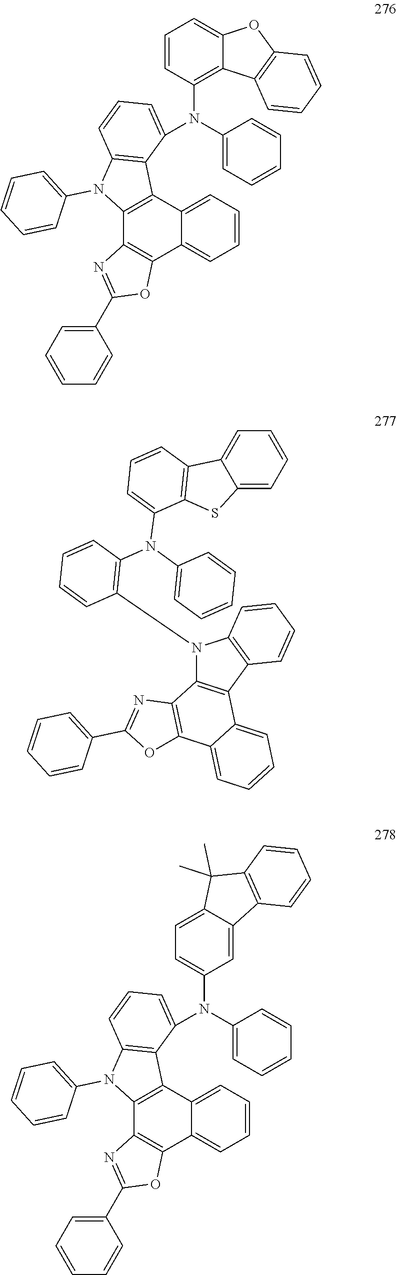 [SMILES: c1ccc(-c2nc3c(o2)c2ccccc2c2c4c(N(c5ccccc5)c5cccc6oc7ccccc7c56)cccc4n(-c4ccccc4)c32)cc1 ; c1ccc(-c2nc3c(o2)c2ccccc2c2c4ccccc4n(-c4ccccc4N(c4ccccc4)c4cccc5c4sc4ccccc45)c32)cc1 ; CC1(C)c2ccccc2-c2cc(N(c3ccccc3)c3cccc4c3c3c5ccccc5c5oc(-c6ccccc6)nc5c3n4-c3ccccc3)ccc21]

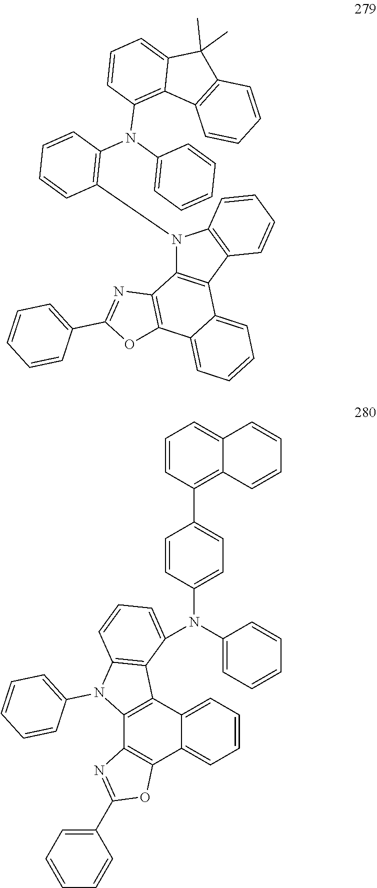 [SMILES: CC1(C)c2ccccc2-c2c(N(c3ccccc3)c3ccccc3-n3c4ccccc4c4c5ccccc5c5oc(-c6ccccc6)nc5c43)cccc21 ; c1ccc(-c2nc3c(o2)c2ccccc2c2c4c(N(c5ccccc5)c5ccc(-c6cccc7ccccc67)cc5)cccc4n(-c4ccccc4)c32)cc1]

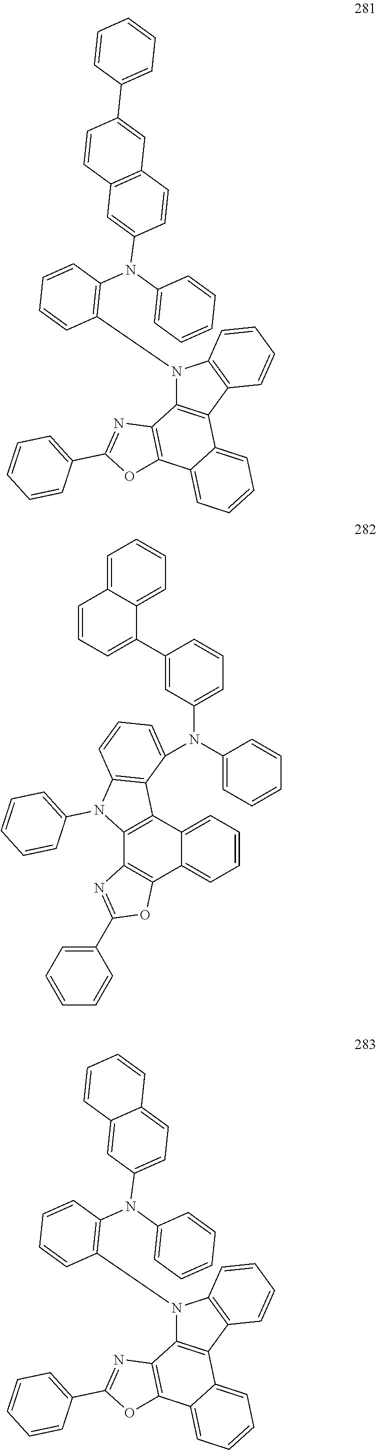 [SMILES: c1ccc(-c2ccc3cc(N(c4ccccc4)c4ccccc4-n4c5ccccc5c5c6ccccc6c6oc(-c7ccccc7)nc6c54)ccc3c2)cc1 ; c1ccc(-c2nc3c(o2)c2ccccc2c2c4c(N(c5ccccc5)c5cccc(-c6cccc7ccccc67)c5)cccc4n(-c4ccccc4)c32)cc1 ; c1ccc(-c2nc3c(o2)c2ccccc2c2c4ccccc4n(-c4ccccc4N(c4ccccc4)c4ccc5ccccc5c4)c32)cc1]

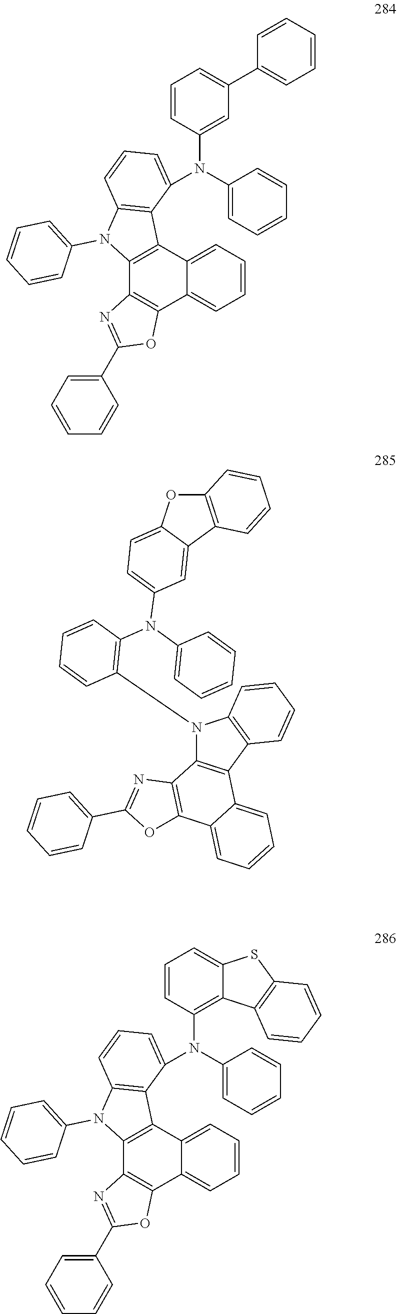 [SMILES: c1ccc(-c2cccc(N(c3ccccc3)c3cccc4c3c3c5ccccc5c5oc(-c6ccccc6)nc5c3n4-c3ccccc3)c2)cc1 ; c1ccc(-c2nc3c(o2)c2ccccc2c2c4ccccc4n(-c4ccccc4N(c4ccccc4)c4ccc5oc6ccccc6c5c4)c32)cc1 ; c1ccc(-c2nc3c(o2)c2ccccc2c2c4c(N(c5ccccc5)c5cccc6sc7ccccc7c56)cccc4n(-c4ccccc4)c32)cc1]

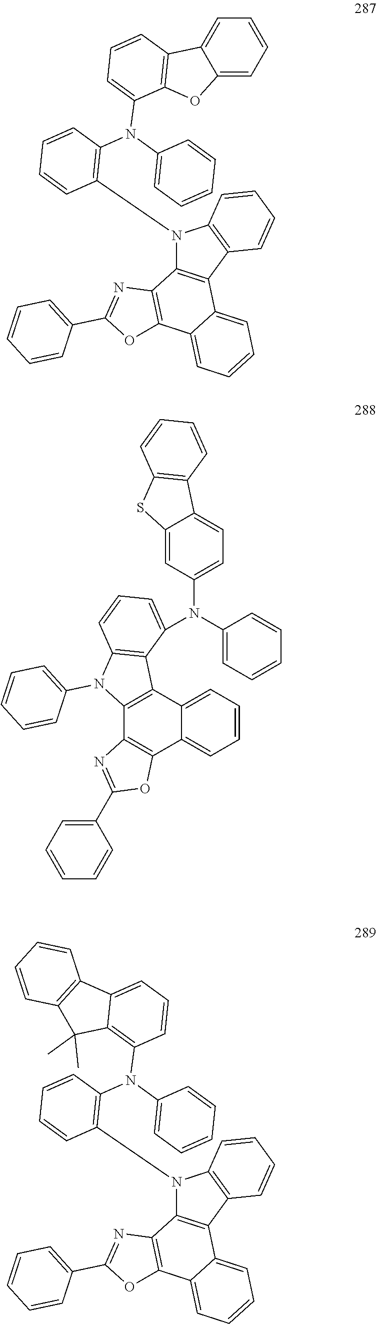 [SMILES: c1ccc(-c2nc3c(o2)c2ccccc2c2c4ccccc4n(-c4ccccc4N(c4ccccc4)c4cccc5c4oc4ccccc45)c32)cc1 ; c1ccc(-c2nc3c(o2)c2ccccc2c2c4c(N(c5ccccc5)c5ccc6c(c5)sc5ccccc56)cccc4n(-c4ccccc4)c32)cc1 ; CC1(C)c2ccccc2-c2cccc(N(c3ccccc3)c3ccccc3-n3c4ccccc4c4c5ccccc5c5oc(-c6ccccc6)nc5c43)c21]

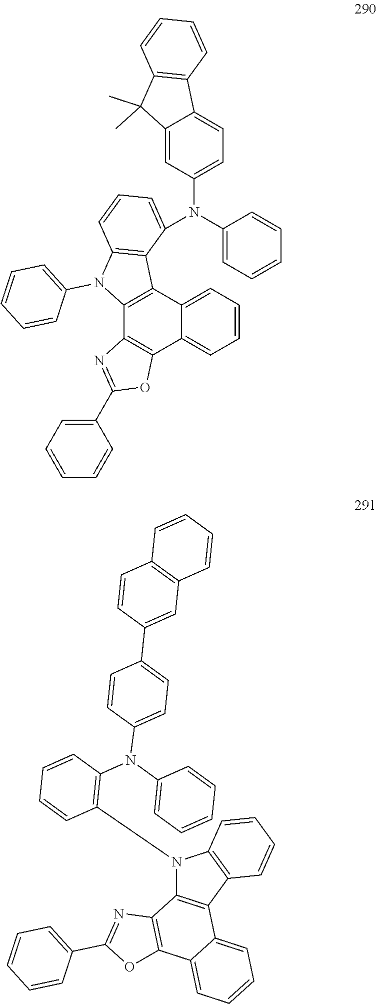 [SMILES: CC1(C)c2ccccc2-c2ccc(N(c3ccccc3)c3cccc4c3c3c5ccccc5c5oc(-c6ccccc6)nc5c3n4-c3ccccc3)cc21 ; c1ccc(-c2nc3c(o2)c2ccccc2c2c4ccccc4n(-c4ccccc4N(c4ccccc4)c4ccc(-c5ccc6ccccc6c5)cc4)c32)cc1]

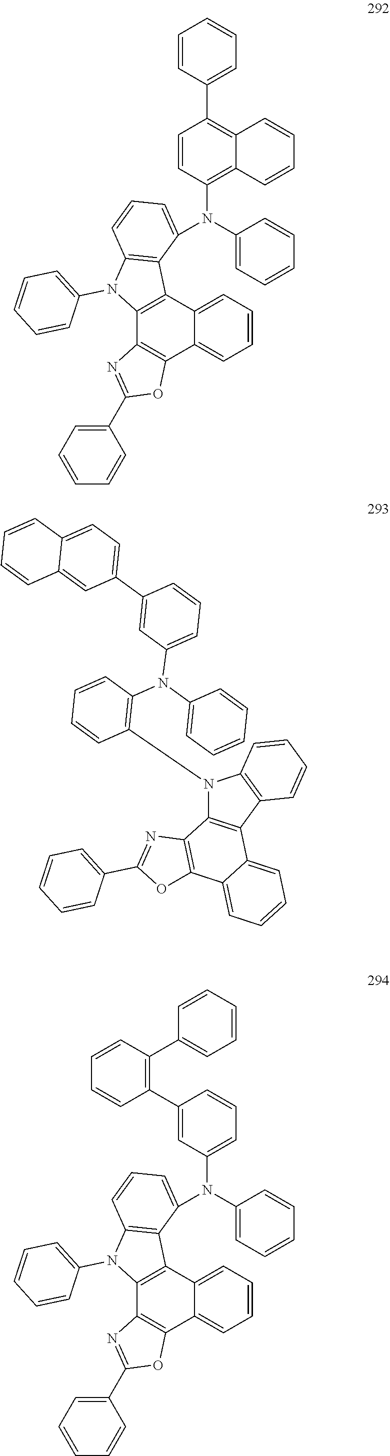

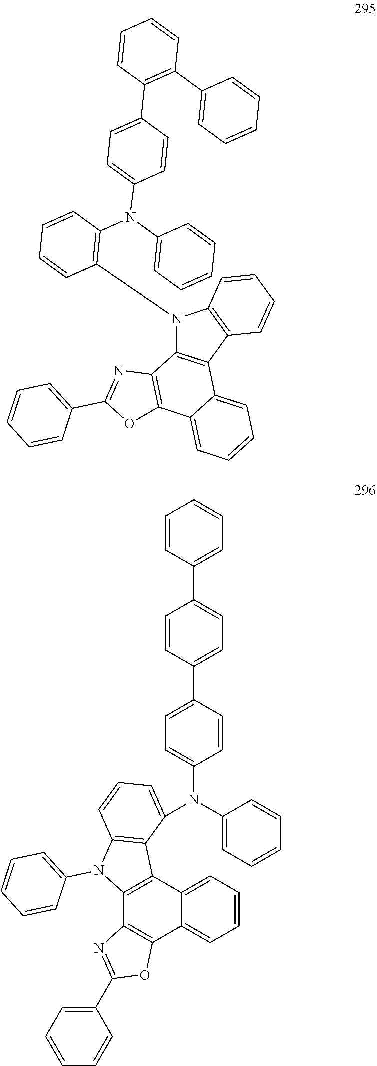 [SMILES: c1ccc(-c2nc3c(o2)c2ccccc2c2c4ccccc4n(-c4ccccc4N(c4ccccc4)c4ccc(-c5ccccc5-c5ccccc5)cc4)c32)cc1 ; c1ccc(-c2ccc(-c3ccc(N(c4ccccc4)c4cccc5c4c4c6ccccc6c6oc(-c7ccccc7)nc6c4n5-c4ccccc4)cc3)cc2)cc1]

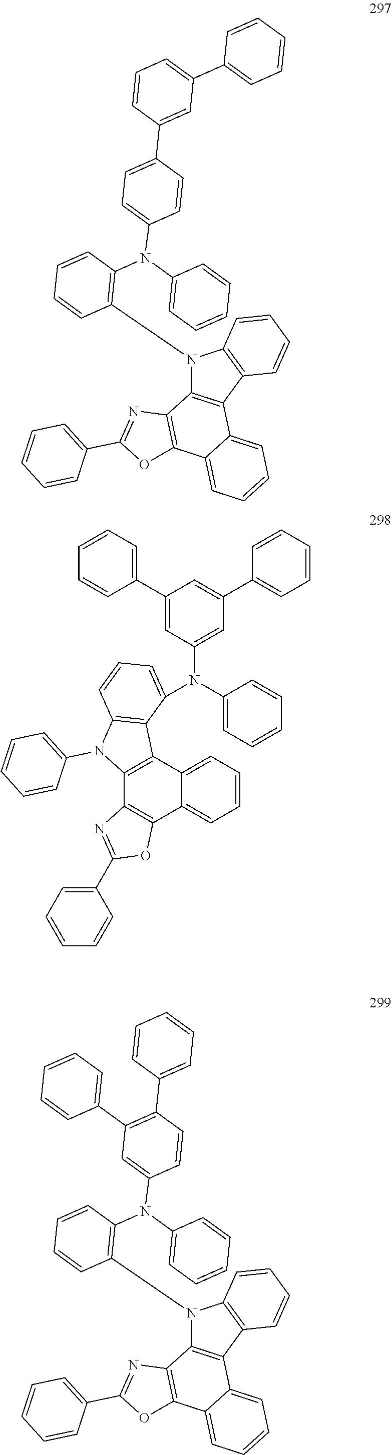

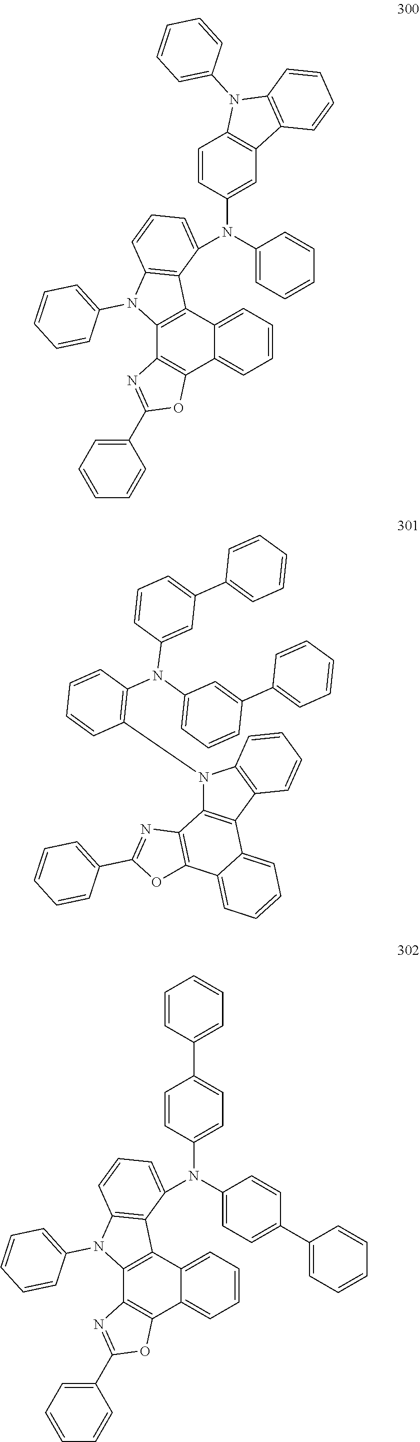 [SMILES: c1ccc(-c2nc3c(o2)c2ccccc2c2c4c(N(c5ccccc5)c5ccc6c(c5)c5ccccc5n6-c5ccccc5)cccc4n(-c4ccccc4)c32)cc1 ; c1ccc(-c2cccc(N(c3cccc(-c4ccccc4)c3)c3ccccc3-n3c4ccccc4c4c5ccccc5c5oc(-c6ccccc6)nc5c43)c2)cc1 ; c1ccc(-c2ccc(N(c3ccc(-c4ccccc4)cc3)c3cccc4c3c3c5ccccc5c5oc(-c6ccccc6)nc5c3n4-c3ccccc3)cc2)cc1]

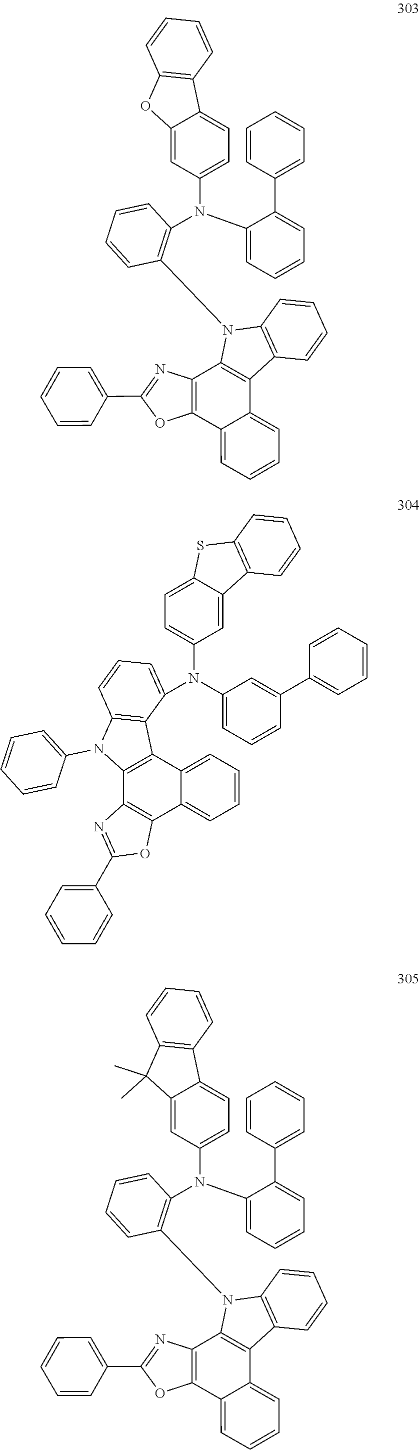

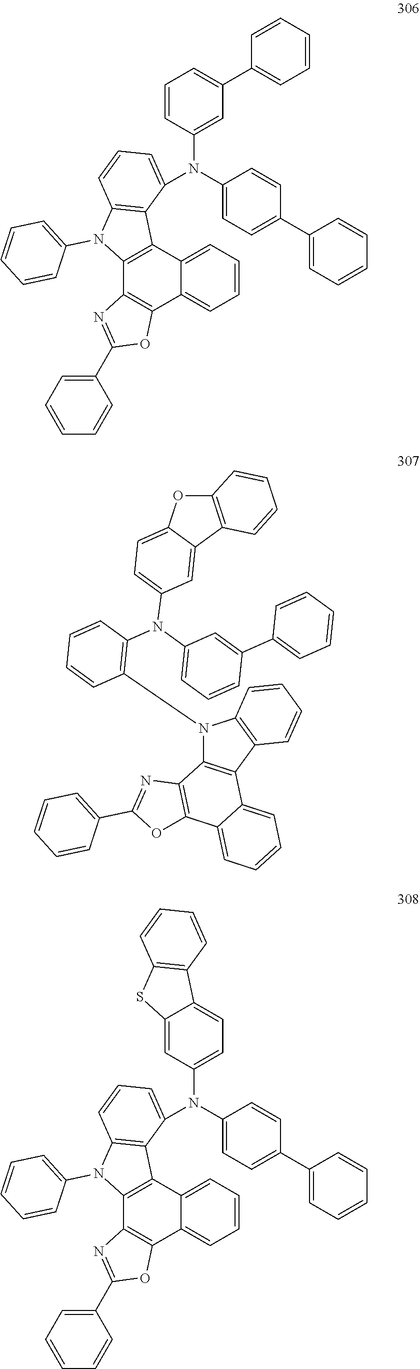 [SMILES: c1ccc(-c2ccc(N(c3cccc(-c4ccccc4)c3)c3cccc4c3c3c5ccccc5c5oc(-c6ccccc6)nc5c3n4-c3ccccc3)cc2)cc1 ; c1ccc(-c2cccc(N(c3ccc4oc5ccccc5c4c3)c3ccccc3-n3c4ccccc4c4c5ccccc5c5oc(-c6ccccc6)nc5c43)c2)cc1 ; c1ccc(-c2ccc(N(c3ccc4c(c3)sc3ccccc34)c3cccc4c3c3c5ccccc5c5oc(-c6ccccc6)nc5c3n4-c3ccccc3)cc2)cc1]

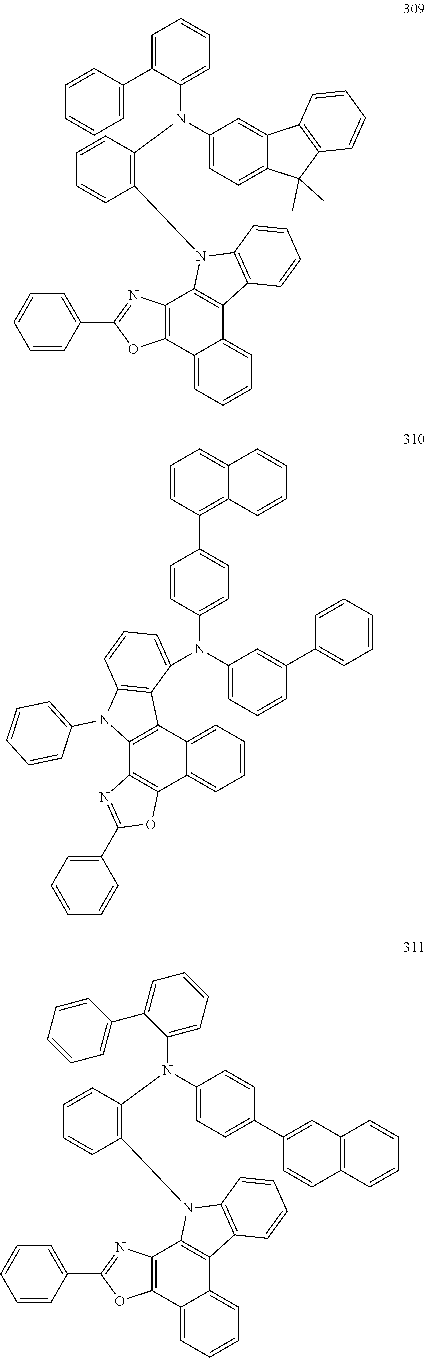 [SMILES: CC1(C)c2ccccc2-c2cc(N(c3ccccc3-c3ccccc3)c3ccccc3-n3c4ccccc4c4c5ccccc5c5oc(-c6ccccc6)nc5c43)ccc21 ; c1ccc(-c2cccc(N(c3ccc(-c4cccc5ccccc45)cc3)c3cccc4c3c3c5ccccc5c5oc(-c6ccccc6)nc5c3n4-c3ccccc3)c2)cc1 ; c1ccc(-c2nc3c(o2)c2ccccc2c2c4ccccc4n(-c4ccccc4N(c4ccc(-c5ccc6ccccc6c5)cc4)c4ccccc4-c4ccccc4)c32)cc1]

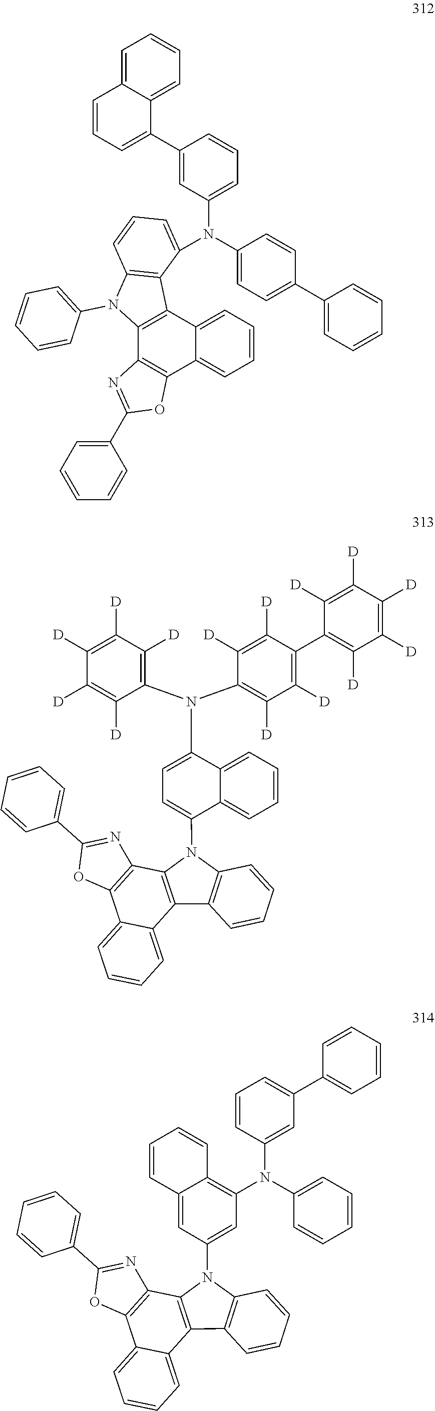 [SMILES: c1ccc(-c2ccc(N(c3cccc(-c4cccc5ccccc45)c3)c3cccc4c3c3c5ccccc5c5oc(-c6ccccc6)nc5c3n4-c3ccccc3)cc2)cc1 ; [2H]c1c([2H])c([2H])c(-c2c([2H])c([2H])c(N(c3c([2H])c([2H])c([2H])c([2H])c3[2H])c3ccc(-n4c5ccccc5c5c6ccccc6c6oc(-c7ccccc7)nc6c54)c4ccccc34)c([2H])c2[2H])c([2H])c1[2H] ; c1ccc(-c2cccc(N(c3ccccc3)c3cc(-n4c5ccccc5c5c6ccccc6c6oc(-c7ccccc7)nc6c54)cc4ccccc34)c2)cc1]

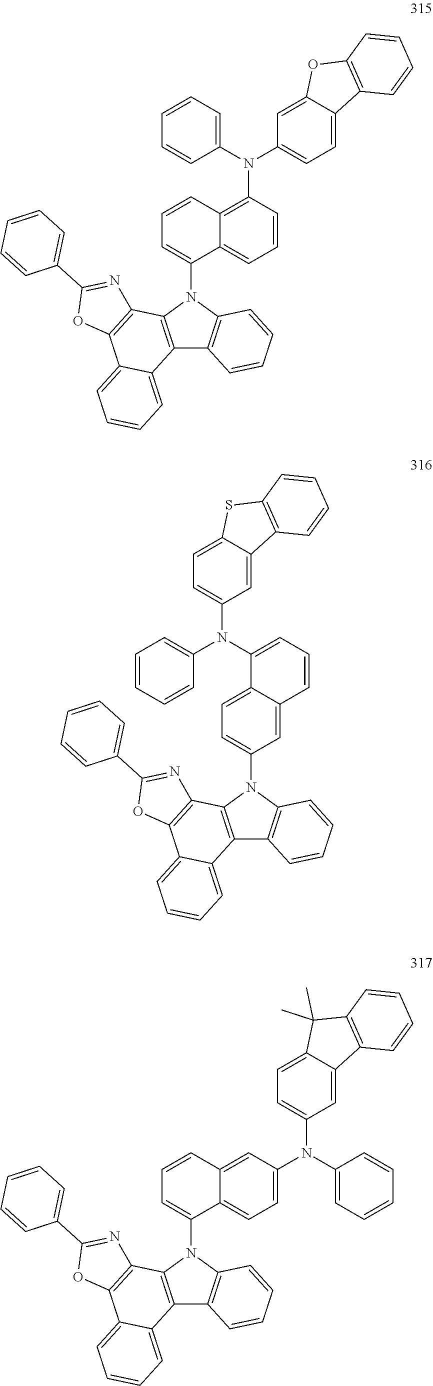 [SMILES: c1ccc(-c2nc3c(o2)c2ccccc2c2c4ccccc4n(-c4cccc5c(N(c6ccccc6)c6ccc7c(c6)oc6ccccc67)cccc45)c32)cc1 ; c1ccc(-c2nc3c(o2)c2ccccc2c2c4ccccc4n(-c4ccc5c(N(c6ccccc6)c6ccc7sc8ccccc8c7c6)cccc5c4)c32)cc1 ; CC1(C)c2ccccc2-c2cc(N(c3ccccc3)c3ccc4c(-n5c6ccccc6c6c7ccccc7c7oc(-c8ccccc8)nc7c65)cccc4c3)ccc21]

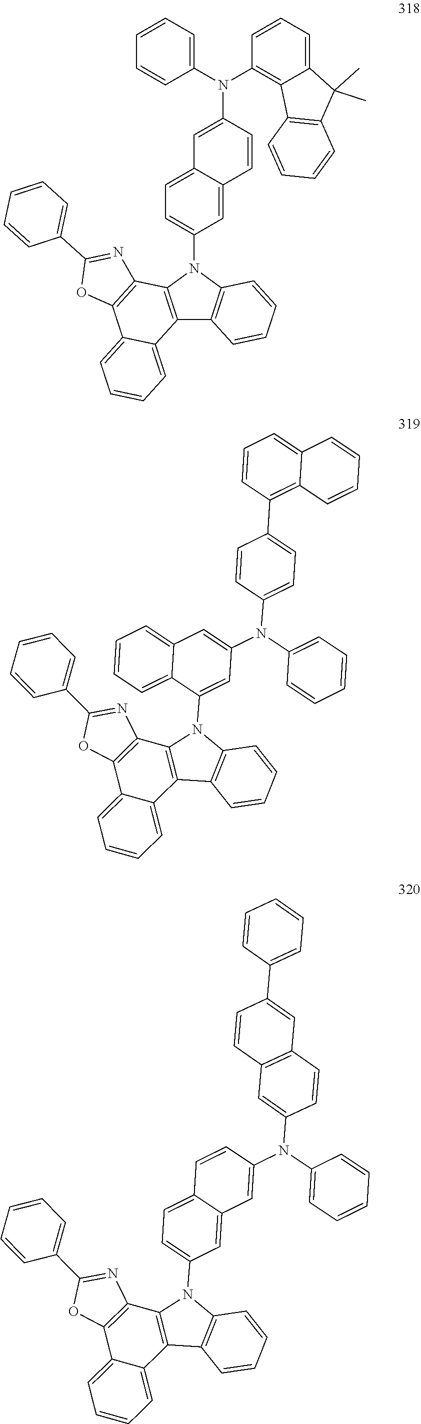 [SMILES: CC1(C)c2ccccc2-c2c(N(c3ccccc3)c3ccc4cc(-n5c6ccccc6c6c7ccccc7c7oc(-c8ccccc8)nc7c65)ccc4c3)cccc21 ; c1ccc(-c2nc3c(o2)c2ccccc2c2c4ccccc4n(-c4cc(N(c5ccccc5)c5ccc(-c6cccc7ccccc67)cc5)cc5ccccc45)c32)cc1 ; c1ccc(-c2ccc3cc(N(c4ccccc4)c4ccc5ccc(-n6c7ccccc7c7c8ccccc8c8oc(-c9ccccc9)nc8c76)cc5c4)ccc3c2)cc1]

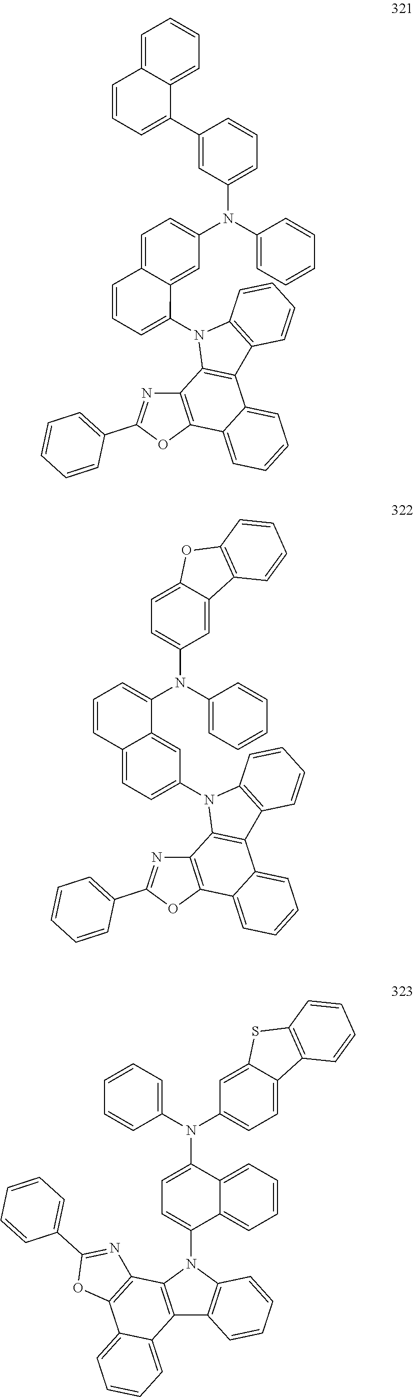 [SMILES: c1ccc(-c2nc3c(o2)c2ccccc2c2c4ccccc4n(-c4cccc5ccc(N(c6ccccc6)c6cccc(-c7cccc8ccccc78)c6)cc45)c32)cc1 ; c1ccc(-c2nc3c(o2)c2ccccc2c2c4ccccc4n(-c4ccc5cccc(N(c6ccccc6)c6ccc7oc8ccccc8c7c6)c5c4)c32)cc1 ; c1ccc(-c2nc3c(o2)c2ccccc2c2c4ccccc4n(-c4ccc(N(c5ccccc5)c5ccc6c(c5)sc5ccccc56)c5ccccc45)c32)cc1]

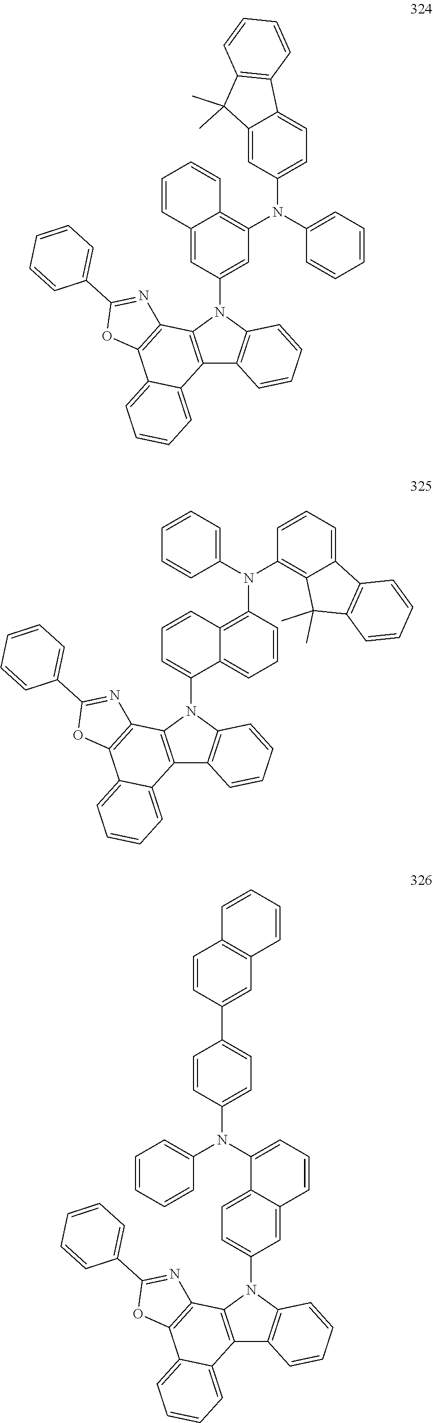 [SMILES: CC1(C)c2ccccc2-c2ccc(N(c3ccccc3)c3cc(-n4c5ccccc5c5c6ccccc6c6oc(-c7ccccc7)nc6c54)cc4ccccc34)cc21 ; CC1(C)c2ccccc2-c2cccc(N(c3ccccc3)c3cccc4c(-n5c6ccccc6c6c7ccccc7c7oc(-c8ccccc8)nc7c65)cccc34)c21 ; c1ccc(-c2nc3c(o2)c2ccccc2c2c4ccccc4n(-c4ccc5c(N(c6ccccc6)c6ccc(-c7ccc8ccccc8c7)cc6)cccc5c4)c32)cc1]

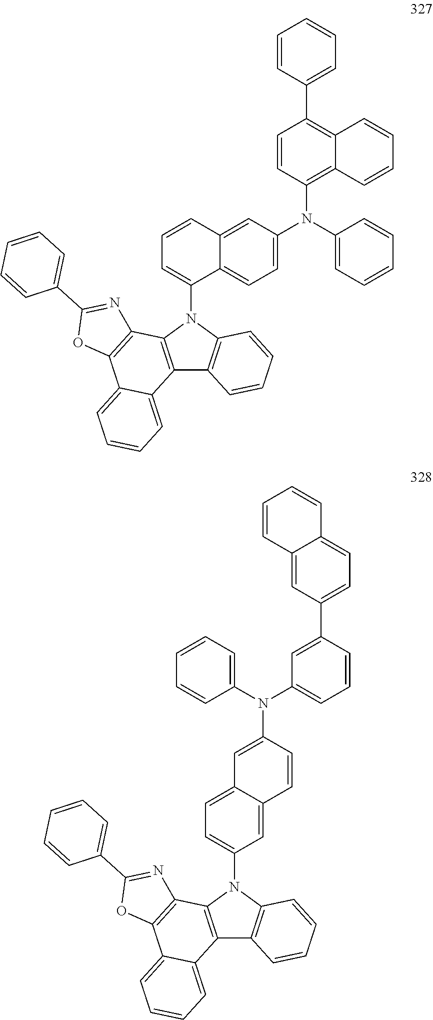 [SMILES: c1ccc(-c2nc3c(o2)c2ccccc2c2c4ccccc4n(-c4cccc5cc(N(c6ccccc6)c6ccc(-c7ccccc7)c7ccccc67)ccc45)c32)cc1 ; c1ccc(-c2nc3c(o2)c2ccccc2c2c4ccccc4n(-c4ccc5cc(N(c6ccccc6)c6cccc(-c7ccc8ccccc8c7)c6)ccc5c4)c32)cc1]

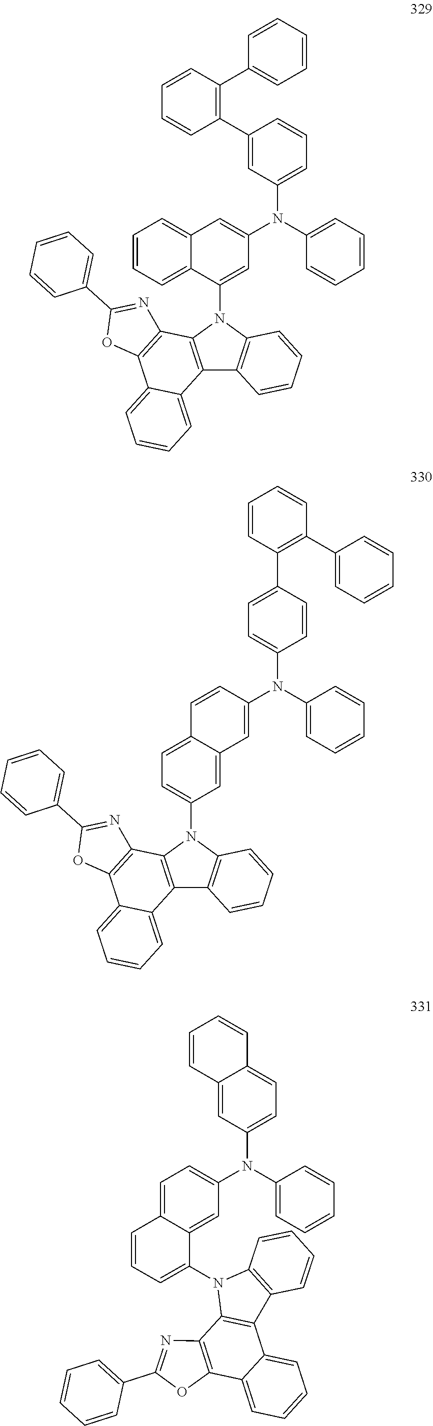 [SMILES: c1ccc(-c2nc3c(o2)c2ccccc2c2c4ccccc4n(-c4cc(N(c5ccccc5)c5cccc(-c6ccccc6-c6ccccc6)c5)cc5ccccc45)c32)cc1 ; c1ccc(-c2nc3c(o2)c2ccccc2c2c4ccccc4n(-c4ccc5ccc(N(c6ccccc6)c6ccc(-c7ccccc7-c7ccccc7)cc6)cc5c4)c32)cc1 ; c1ccc(-c2nc3c(o2)c2ccccc2c2c4ccccc4n(-c4cccc5ccc(N(c6ccccc6)c6ccc7ccccc7c6)cc45)c32)cc1]

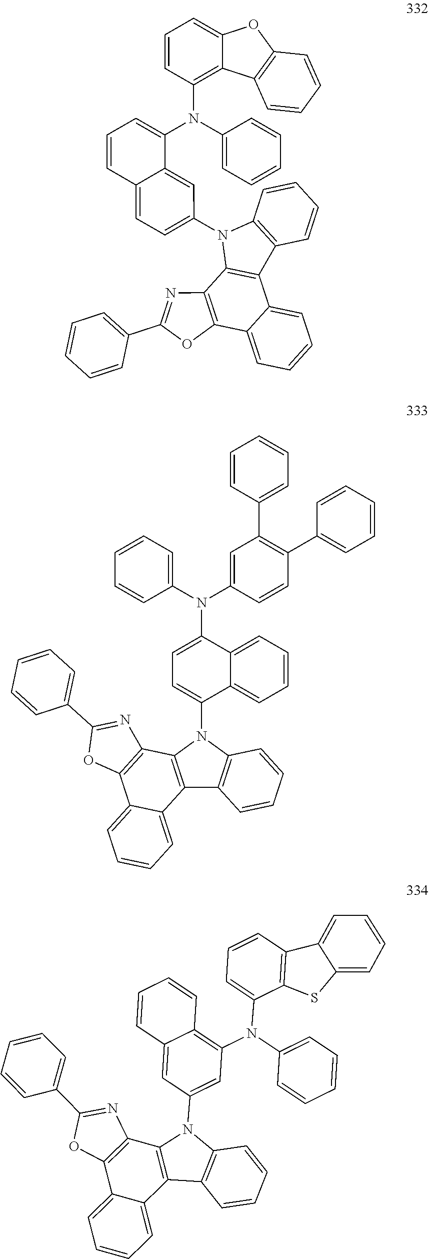 [SMILES: c1ccc(-c2nc3c(o2)c2ccccc2c2c4ccccc4n(-c4ccc5cccc(N(c6ccccc6)c6cccc7oc8ccccc8c67)c5c4)c32)cc1 ; c1ccc(-c2nc3c(o2)c2ccccc2c2c4ccccc4n(-c4ccc(N(c5ccccc5)c5ccc(-c6ccccc6)c(-c6ccccc6)c5)c5ccccc45)c32)cc1 ; c1ccc(-c2nc3c(o2)c2ccccc2c2c4ccccc4n(-c4cc(N(c5ccccc5)c5cccc6c5sc5ccccc56)c5ccccc5c4)c32)cc1]

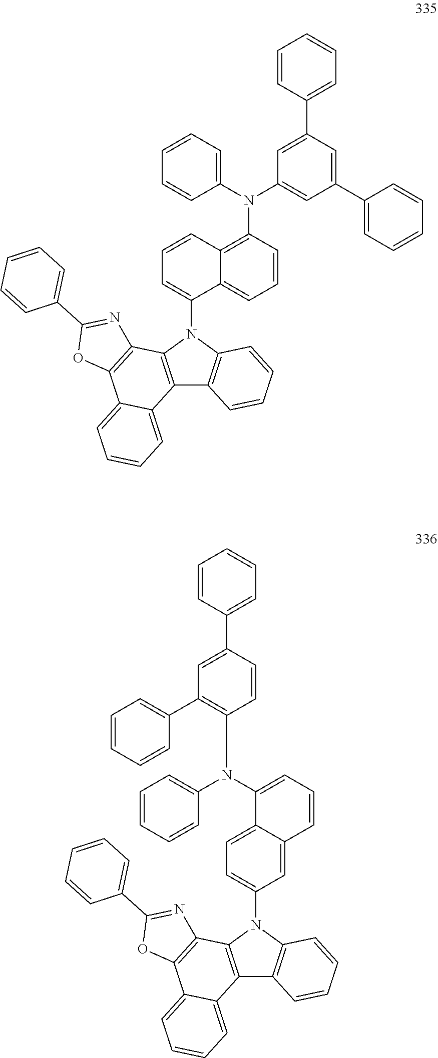 [SMILES: c1ccc(-c2cc(-c3ccccc3)cc(N(c3ccccc3)c3cccc4c(-n5c6ccccc6c6c7ccccc7c7oc(-c8ccccc8)nc7c65)cccc34)c2)cc1 ; c1ccc(-c2ccc(N(c3ccccc3)c3cccc4cc(-n5c6ccccc6c6c7ccccc7c7oc(-c8ccccc8)nc7c65)ccc34)c(-c3ccccc3)c2)cc1]

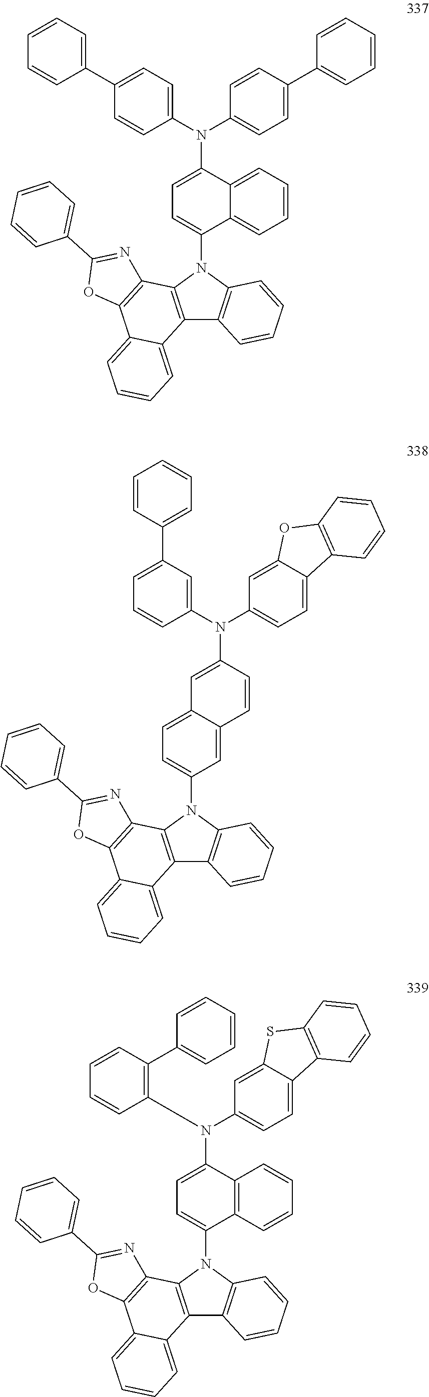 [SMILES: c1ccc(-c2ccc(N(c3ccc(-c4ccccc4)cc3)c3ccc(-n4c5ccccc5c5c6ccccc6c6oc(-c7ccccc7)nc6c54)c4ccccc34)cc2)cc1 ; c1ccc(-c2cccc(N(c3ccc4cc(-n5c6ccccc6c6c7ccccc7c7oc(-c8ccccc8)nc7c65)ccc4c3)c3ccc4c(c3)oc3ccccc34)c2)cc1 ; c1ccc(-c2nc3c(o2)c2ccccc2c2c4ccccc4n(-c4ccc(N(c5ccc6c(c5)sc5ccccc56)c5ccccc5-c5ccccc5)c5ccccc45)c32)cc1]

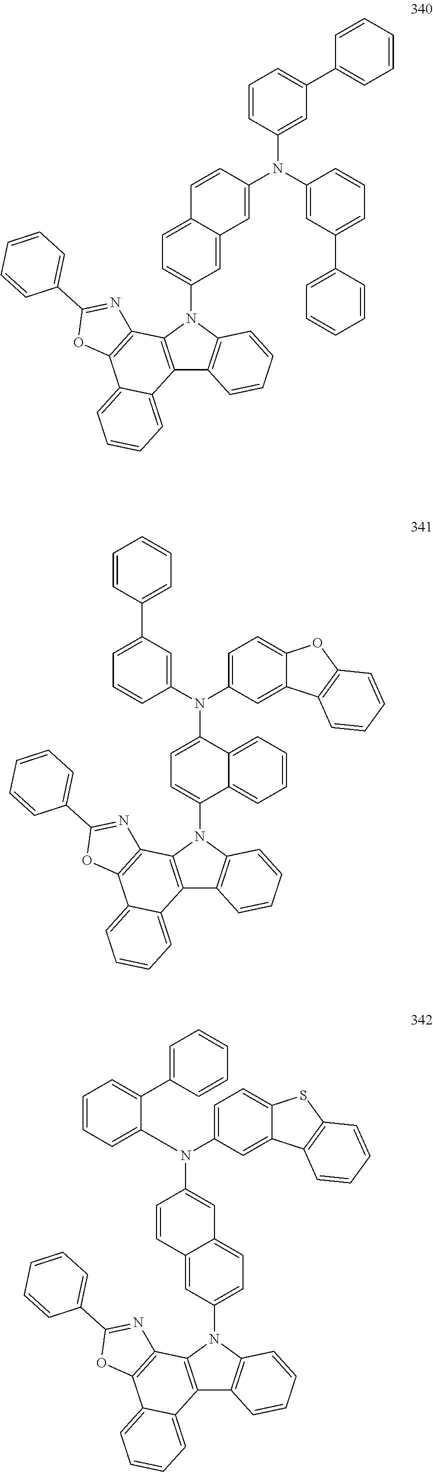 [SMILES: c1ccc(-c2cccc(N(c3cccc(-c4ccccc4)c3)c3ccc4ccc(-n5c6ccccc6c6c7ccccc7c7oc(-c8ccccc8)nc7c65)cc4c3)c2)cc1 ; c1ccc(-c2cccc(N(c3ccc4oc5ccccc5c4c3)c3ccc(-n4c5ccccc5c5c6ccccc6c6oc(-c7ccccc7)nc6c54)c4ccccc34)c2)cc1 ; c1ccc(-c2nc3c(o2)c2ccccc2c2c4ccccc4n(-c4ccc5cc(N(c6ccc7sc8ccccc8c7c6)c6ccccc6-c6ccccc6)ccc5c4)c32)cc1]

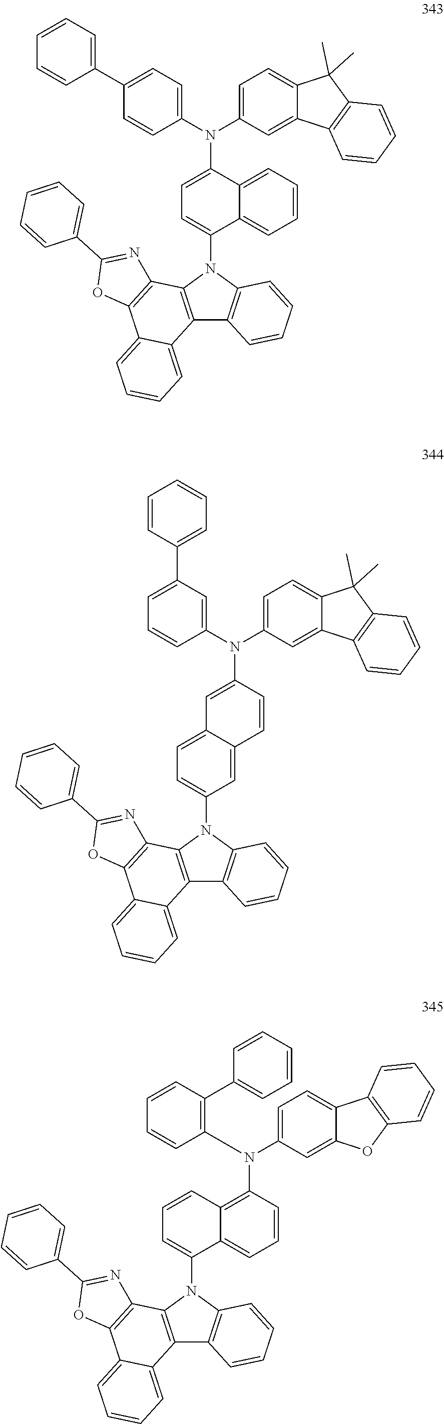 [SMILES: CC1(C)c2ccccc2-c2cc(N(c3ccc(-c4ccccc4)cc3)c3ccc(-n4c5ccccc5c5c6ccccc6c6oc(-c7ccccc7)nc6c54)c4ccccc34)ccc21 ; CC1(C)c2ccccc2-c2cc(N(c3cccc(-c4ccccc4)c3)c3ccc4cc(-n5c6ccccc6c6c7ccccc7c7oc(-c8ccccc8)nc7c65)ccc4c3)ccc21 ; c1ccc(-c2nc3c(o2)c2ccccc2c2c4ccccc4n(-c4cccc5c(N(c6ccc7c(c6)oc6ccccc67)c6ccccc6-c6ccccc6)cccc45)c32)cc1]

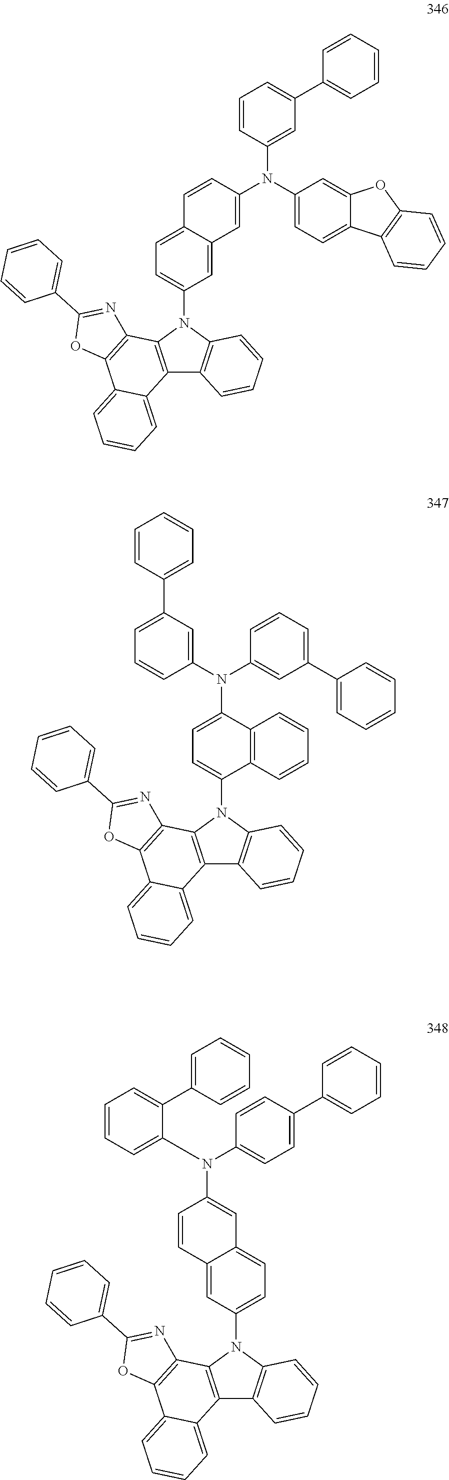 [SMILES: c1ccc(-c2cccc(N(c3ccc4ccc(-n5c6ccccc6c6c7ccccc7c7oc(-c8ccccc8)nc7c65)cc4c3)c3ccc4c(c3)oc3ccccc34)c2)cc1 ; c1ccc(-c2cccc(N(c3cccc(-c4ccccc4)c3)c3ccc(-n4c5ccccc5c5c6ccccc6c6oc(-c7ccccc7)nc6c54)c4ccccc34)c2)cc1 ; c1ccc(-c2ccc(N(c3ccc4cc(-n5c6ccccc6c6c7ccccc7c7oc(-c8ccccc8)nc7c65)ccc4c3)c3ccccc3-c3ccccc3)cc2)cc1]

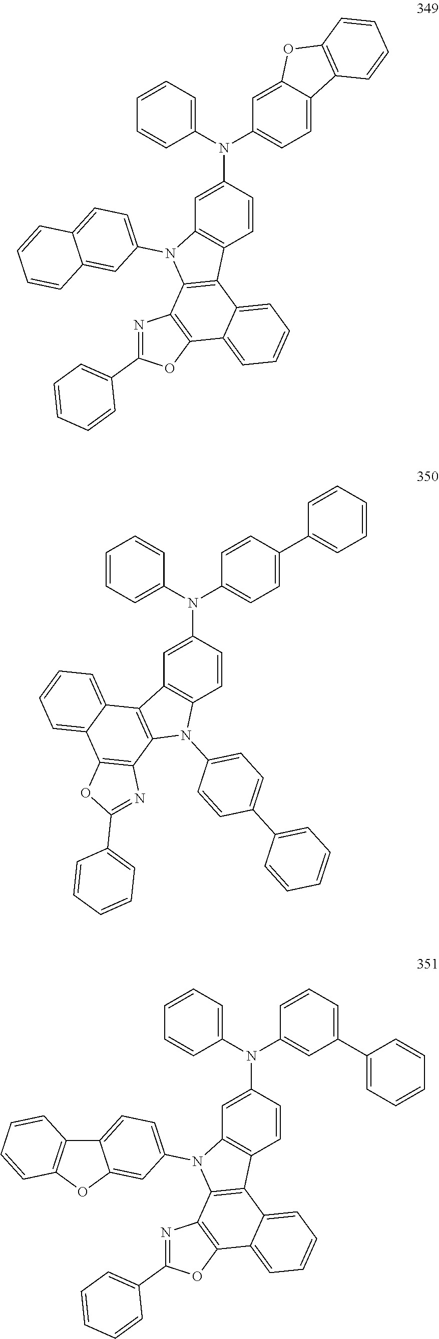 [SMILES: c1ccc(-c2nc3c(o2)c2ccccc2c2c4ccc(N(c5ccccc5)c5ccc6c(c5)oc5ccccc56)cc4n(-c4ccc5ccccc5c4)c32)cc1 ; c1ccc(-c2ccc(N(c3ccccc3)c3ccc4c(c3)c3c5ccccc5c5oc(-c6ccccc6)nc5c3n4-c3ccc(-c4ccccc4)cc3)cc2)cc1 ; c1ccc(-c2cccc(N(c3ccccc3)c3ccc4c5c6ccccc6c6oc(-c7ccccc7)nc6c5n(-c5ccc6c(c5)oc5ccccc56)c4c3)c2)cc1]

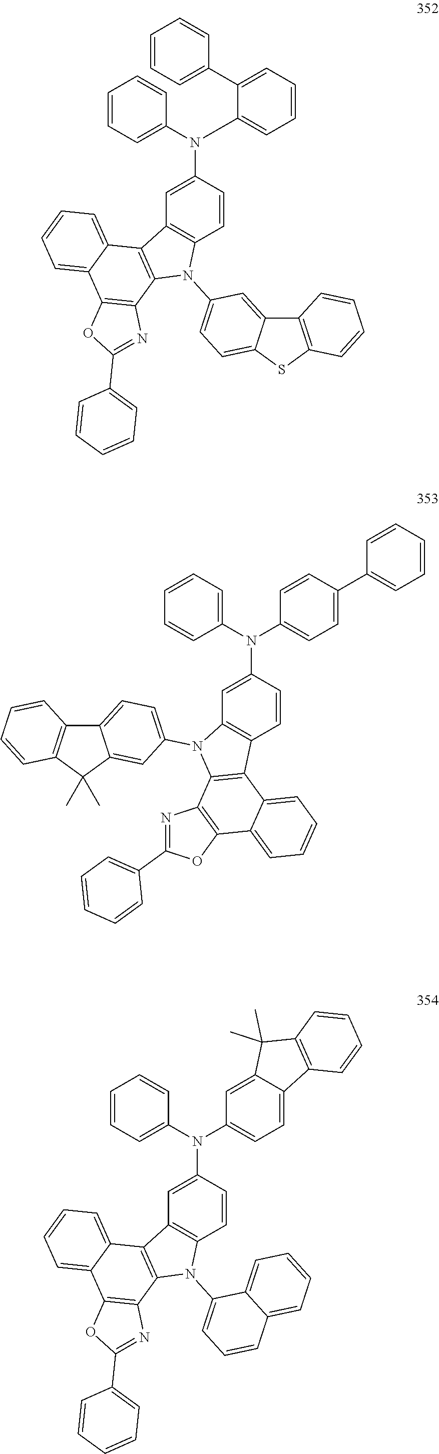 [SMILES: c1ccc(-c2nc3c(o2)c2ccccc2c2c4cc(N(c5ccccc5)c5ccccc5-c5ccccc5)ccc4n(-c4ccc5sc6ccccc6c5c4)c32)cc1 ; CC1(C)c2ccccc2-c2ccc(-n3c4cc(N(c5ccccc5)c5ccc(-c6ccccc6)cc5)ccc4c4c5ccccc5c5oc(-c6ccccc6)nc5c43)cc21 ; CC1(C)c2ccccc2-c2ccc(N(c3ccccc3)c3ccc4c(c3)c3c5ccccc5c5oc(-c6ccccc6)nc5c3n4-c3cccc4ccccc34)cc21]

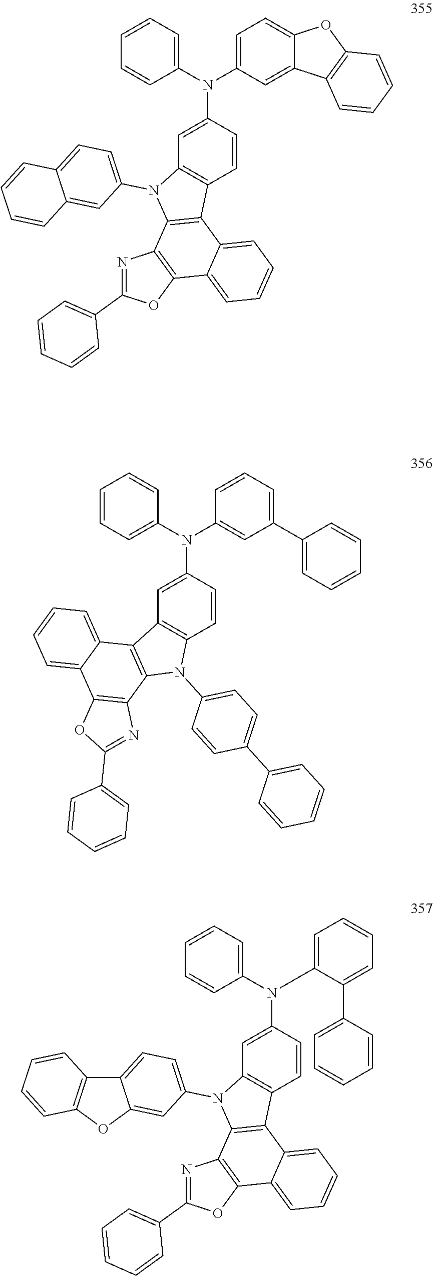 [SMILES: c1ccc(-c2nc3c(o2)c2ccccc2c2c4ccc(N(c5ccccc5)c5ccc6oc7ccccc7c6c5)cc4n(-c4ccc5ccccc5c4)c32)cc1 ; c1ccc(-c2ccc(-n3c4ccc(N(c5ccccc5)c5cccc(-c6ccccc6)c5)cc4c4c5ccccc5c5oc(-c6ccccc6)nc5c43)cc2)cc1 ; c1ccc(-c2nc3c(o2)c2ccccc2c2c4ccc(N(c5ccccc5)c5ccccc5-c5ccccc5)cc4n(-c4ccc5c(c4)oc4ccccc45)c32)cc1]

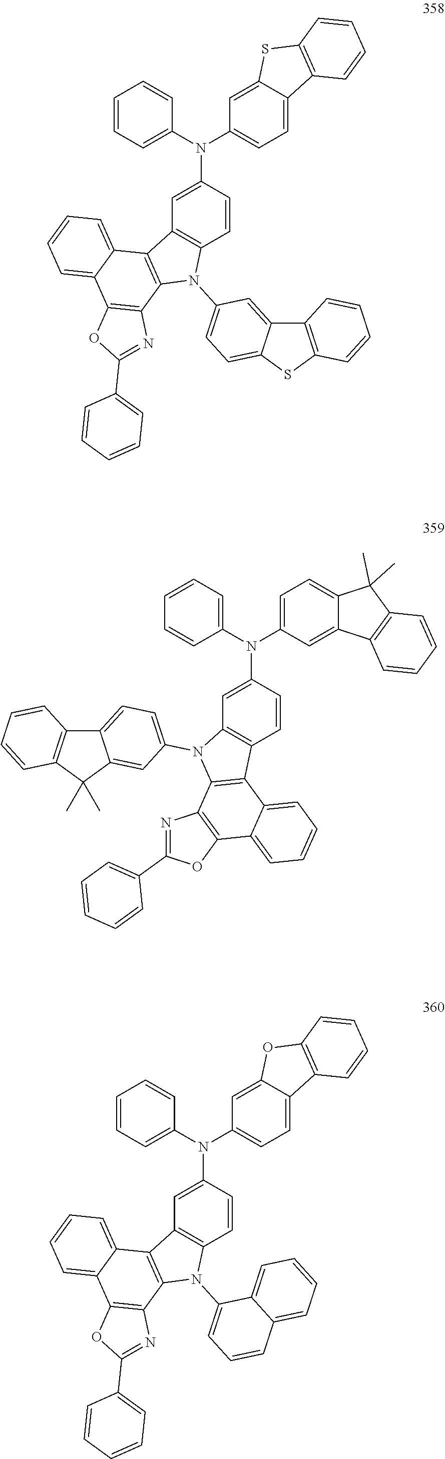 [SMILES: c1ccc(-c2nc3c(o2)c2ccccc2c2c4cc(N(c5ccccc5)c5ccc6c(c5)sc5ccccc56)ccc4n(-c4ccc5sc6ccccc6c5c4)c32)cc1 ; CC1(C)c2ccccc2-c2cc(N(c3ccccc3)c3ccc4c5c6ccccc6c6oc(-c7ccccc7)nc6c5n(-c5ccc6c(c5)C(C)(C)c5ccccc5-6)c4c3)ccc21 ; c1ccc(-c2nc3c(o2)c2ccccc2c2c4cc(N(c5ccccc5)c5ccc6c(c5)oc5ccccc56)ccc4n(-c4cccc5ccccc45)c32)cc1]

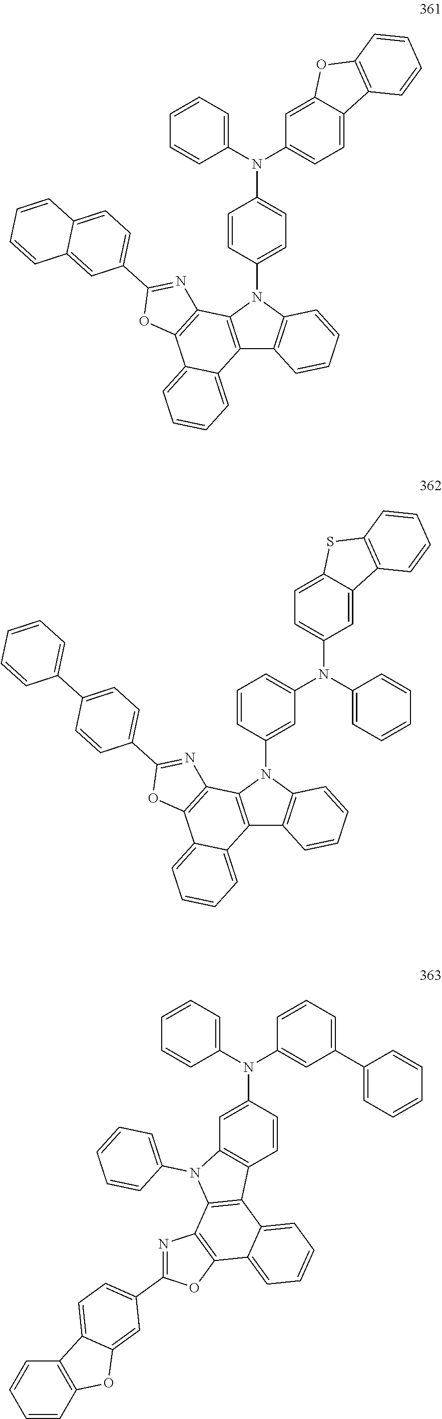 [SMILES: c1ccc(N(c2ccc(-n3c4ccccc4c4c5ccccc5c5oc(-c6ccc7ccccc7c6)nc5c43)cc2)c2ccc3c(c2)oc2ccccc23)cc1 ; c1ccc(-c2ccc(-c3nc4c(o3)c3ccccc3c3c5ccccc5n(-c5cccc(N(c6ccccc6)c6ccc7sc8ccccc8c7c6)c5)c43)cc2)cc1 ; c1ccc(-c2cccc(N(c3ccccc3)c3ccc4c5c6ccccc6c6oc(-c7ccc8c(c7)oc7ccccc78)nc6c5n(-c5ccccc5)c4c3)c2)cc1]

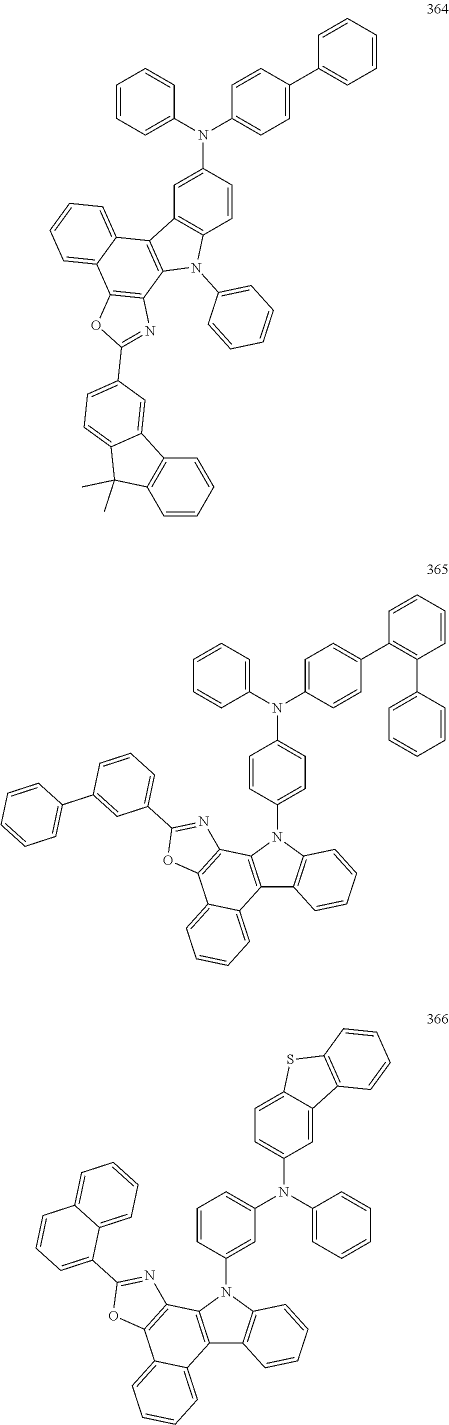 [SMILES: CC1(C)c2ccccc2-c2cc(-c3nc4c(o3)c3ccccc3c3c5cc(N(c6ccccc6)c6ccc(-c7ccccc7)cc6)ccc5n(-c5ccccc5)c43)ccc21 ; c1ccc(-c2cccc(-c3nc4c(o3)c3ccccc3c3c5ccccc5n(-c5ccc(N(c6ccccc6)c6ccc(-c7ccccc7-c7ccccc7)cc6)cc5)c43)c2)cc1 ; c1ccc(N(c2cccc(-n3c4ccccc4c4c5ccccc5c5oc(-c6cccc7ccccc67)nc5c43)c2)c2ccc3sc4ccccc4c3c2)cc1]

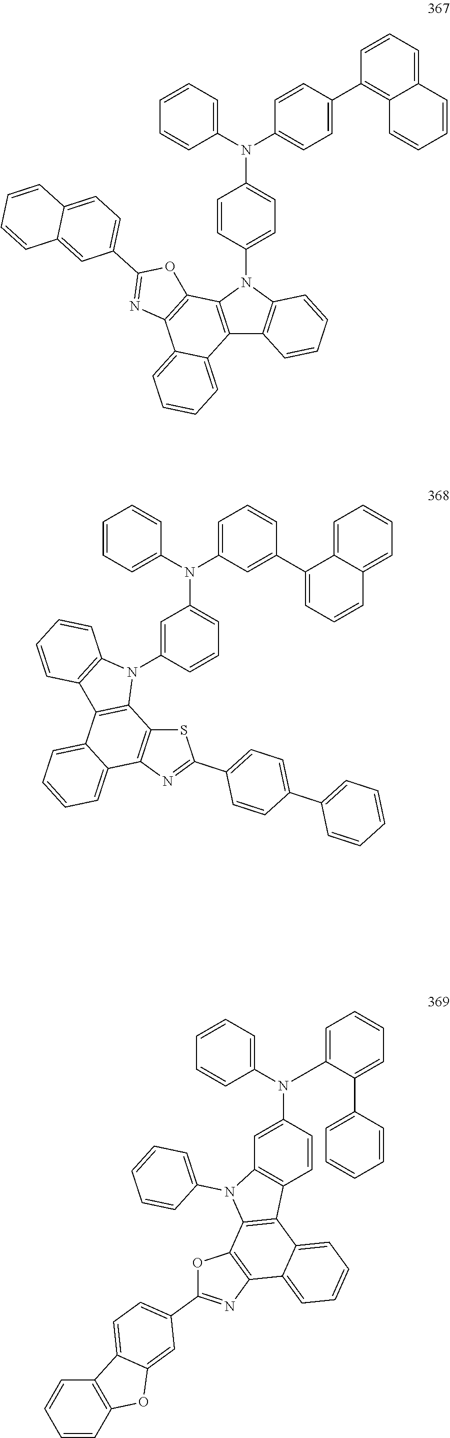 [SMILES: c1ccc(N(c2ccc(-c3cccc4ccccc34)cc2)c2ccc(-n3c4ccccc4c4c5ccccc5c5nc(-c6ccc7ccccc7c6)oc5c43)cc2)cc1 ; c1ccc(-c2ccc(-c3nc4c5ccccc5c5c6ccccc6n(-c6cccc(N(c7ccccc7)c7cccc(-c8cccc9ccccc89)c7)c6)c5c4s3)cc2)cc1 ; c1ccc(-c2ccccc2N(c2ccccc2)c2ccc3c4c5ccccc5c5nc(-c6ccc7c(c6)oc6ccccc67)oc5c4n(-c4ccccc4)c3c2)cc1]

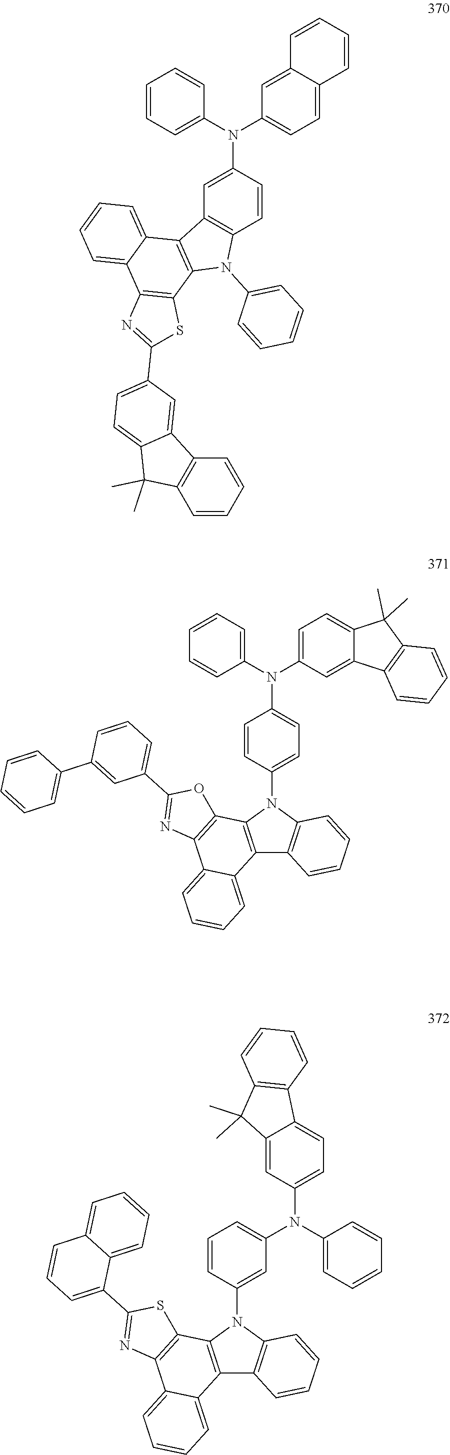 [SMILES: CC1(C)c2ccccc2-c2cc(-c3nc4c5ccccc5c5c6cc(N(c7ccccc7)c7ccc8ccccc8c7)ccc6n(-c6ccccc6)c5c4s3)ccc21 ; CC1(C)c2ccccc2-c2cc(N(c3ccccc3)c3ccc(-n4c5ccccc5c5c6ccccc6c6nc(-c7cccc(-c8ccccc8)c7)oc6c54)cc3)ccc21 ; CC1(C)c2ccccc2-c2ccc(N(c3ccccc3)c3cccc(-n4c5ccccc5c5c6ccccc6c6nc(-c7cccc8ccccc78)sc6c54)c3)cc21]

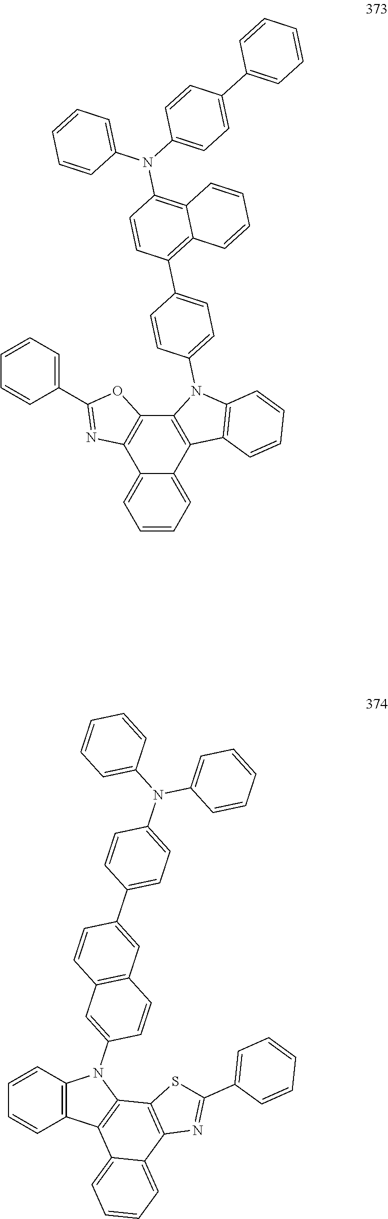 [SMILES: c1ccc(-c2ccc(N(c3ccccc3)c3ccc(-c4ccc(-n5c6ccccc6c6c7ccccc7c7nc(-c8ccccc8)oc7c65)cc4)c4ccccc34)cc2)cc1 ; c1ccc(-c2nc3c4ccccc4c4c5ccccc5n(-c5ccc6cc(-c7ccc(N(c8ccccc8)c8ccccc8)cc7)ccc6c5)c4c3s2)cc1]

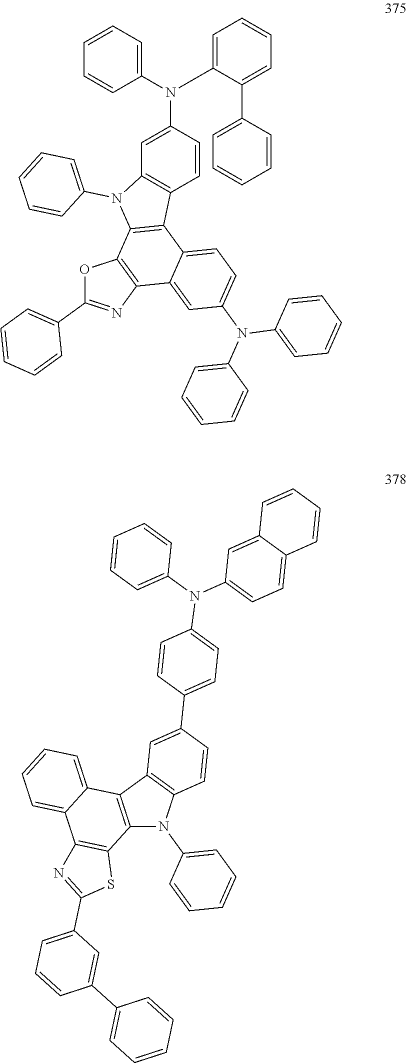 [SMILES: c1ccc(-c2nc3c4cc(N(c5ccccc5)c5ccccc5)ccc4c4c5ccc(N(c6ccccc6)c6ccccc6-c6ccccc6)cc5n(-c5ccccc5)c4c3o2)cc1 ; c1ccc(-c2cccc(-c3nc4c5ccccc5c5c6cc(-c7ccc(N(c8ccccc8)c8ccc9ccccc9c8)cc7)ccc6n(-c6ccccc6)c5c4s3)c2)cc1]

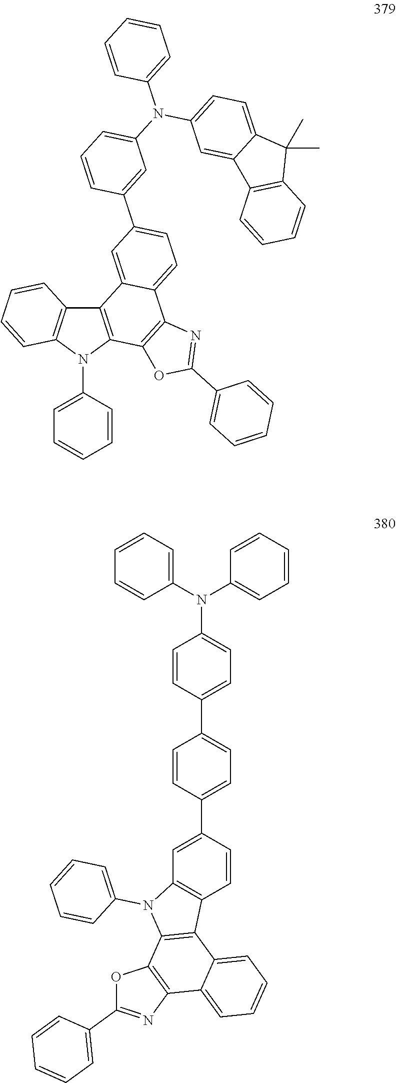 [SMILES: CC1(C)c2ccccc2-c2cc(N(c3ccccc3)c3cccc(-c4ccc5c(c4)c4c6ccccc6n(-c6ccccc6)c4c4oc(-c6ccccc6)nc54)c3)ccc21 ; c1ccc(-c2nc3c4ccccc4c4c5ccc(-c6ccc(-c7ccc(N(c8ccccc8)c8ccccc8)cc7)cc6)cc5n(-c5ccccc5)c4c3o2)cc1]

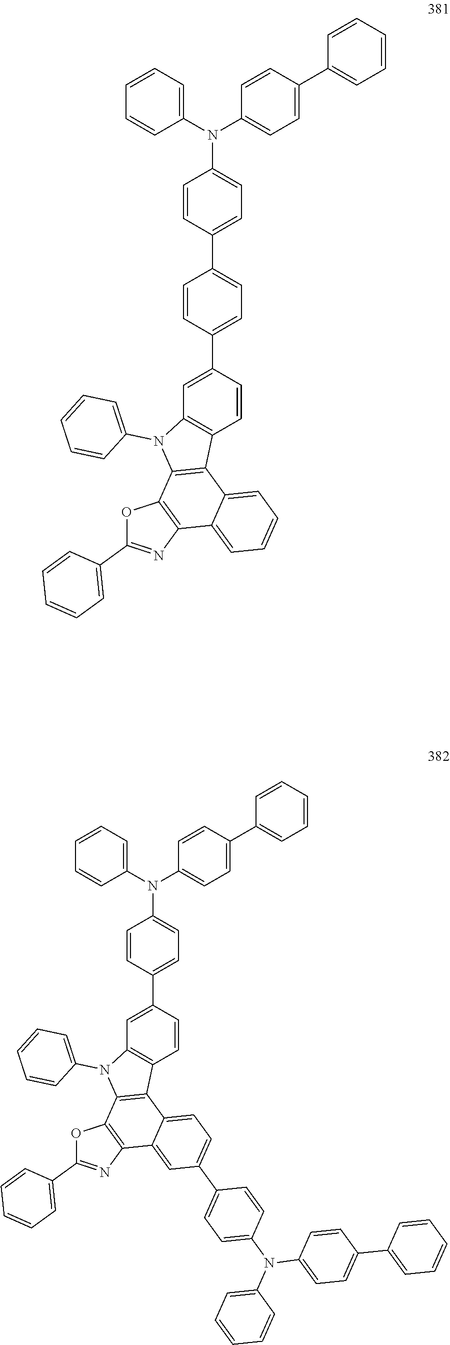 [SMILES: c1ccc(-c2ccc(N(c3ccccc3)c3ccc(-c4ccc(-c5ccc6c7c8ccccc8c8nc(-c9ccccc9)oc8c7n(-c7ccccc7)c6c5)cc4)cc3)cc2)cc1 ; c1ccc(-c2ccc(N(c3ccccc3)c3ccc(-c4ccc5c(c4)c4nc(-c6ccccc6)oc4c4c5c5ccc(-c6ccc(N(c7ccccc7)c7ccc(-c8ccccc8)cc7)cc6)cc5n4-c4ccccc4)cc3)cc2)cc1]

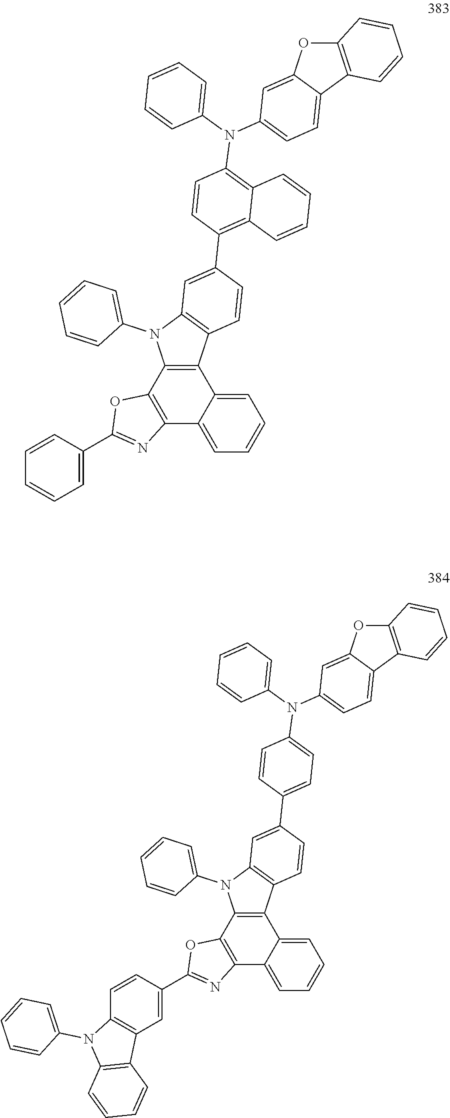 [SMILES: c1ccc(-c2nc3c4ccccc4c4c5ccc(-c6ccc(N(c7ccccc7)c7ccc8c(c7)oc7ccccc78)c7ccccc67)cc5n(-c5ccccc5)c4c3o2)cc1 ; c1ccc(N(c2ccc(-c3ccc4c5c6ccccc6c6nc(-c7ccc8c(c7)c7ccccc7n8-c7ccccc7)oc6c5n(-c5ccccc5)c4c3)cc2)c2ccc3c(c2)oc2ccccc23)cc1]

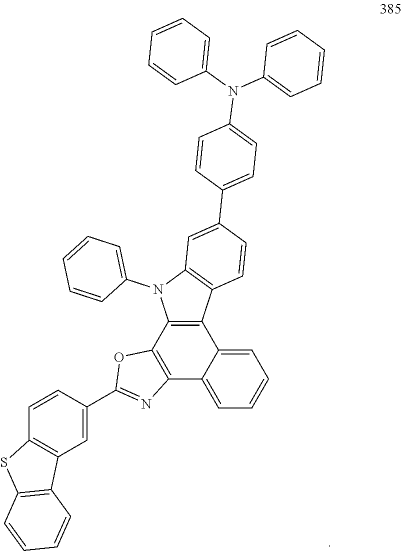 [SMILES: c1ccc(N(c2ccccc2)c2ccc(-c3ccc4c5c6ccccc6c6nc(-c7ccc8sc9ccccc9c8c7)oc6c5n(-c5ccccc5)c4c3)cc2)cc1]

A synthesis method for the organic compounds provided is not particularly limited in the present disclosure, and suitable synthesis methods can be determined by those skilled in the art according to the organic compounds of the present disclosure in combination with preparation methods provided in preparation examples. All of the organic compounds provided in the present disclosure can be obtained by those skilled in the art according to these exemplary preparation methods, and all the specific preparation methods for preparing the organic compounds are not detailed here, which should not be understand by those skilled in the art as limiting the present disclosure.

In a second aspect of the present disclosure, provided is an organic electroluminescent device, comprising an anode, a cathode, and a functional layer disposed between the cathode and the anode, where the functional layer comprises the organic compound according to the first aspect of the present disclosure.

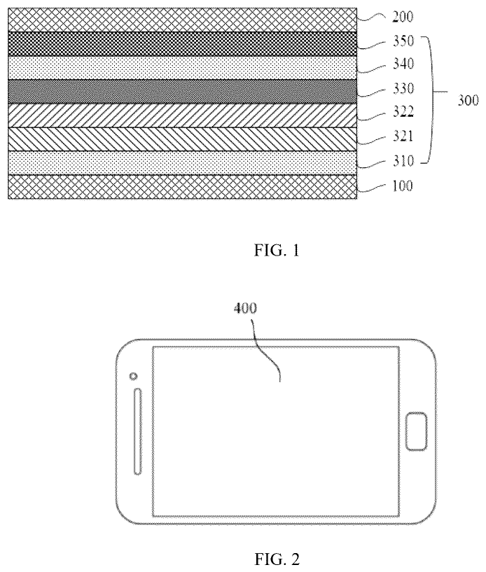

For example, as shown in FIG. 1, the organic electroluminescent device may comprise an anode 100 and a cathode 200 which are oppositely disposed, and a functional layer 300 disposed between the anode 100 and the cathode 200; and the functional layer 300 comprises the organic compound provided in the first aspect of the present disclosure.

In another specific embodiment of the present disclosure, the organic electroluminescent device may be, for example, a red organic electroluminescent device.

In another specific embodiment of the present disclosure, the functional layer comprises an organic electroluminescent layer, and the organic electroluminescent layer comprises the organic compound.

In one specific embodiment, the organic electroluminescent device may comprise an anode 100, a hole transport layer 321, a hole auxiliary layer 322, an organic electroluminescent layer 330 as an energy conversion layer, an electron transport layer 340, and a cathode 200 which are sequentially stacked.

In one specific embodiment, the anode 100 comprises the following anode materials which are optionally materials having a large work function that facilitate hole injection into the functional layer. The anode materials specifically include metals such as nickel, platinum, vanadium, chromium, copper, zinc, and gold, or their alloys; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene] (PEDT), polypyrrole, and polyaniline, but are not limited to this. Also preferably, indiumtinoxide (ITO) is comprised as a transparent electrode of the anode.

In one specific embodiment, the hole transport layer 321 may comprise one or more hole transport materials, and the hole transport materials may be selected from carbazole multimers, carbazole-linked triarylamine compounds, or other types of compounds. In one specific embodiment, the hole transport layer 321 is made of a compound HT-1.

In one specific embodiment, the hole auxiliary layer 322 may comprise one or more materials, which may be selected from carbazole multimers or other types of compounds, which are not particularly limited in the present disclosure. In one specific embodiment, the hole auxiliary layer 322 is made of a compound HT-2.

Optionally, the hole auxiliary layer is also referred to as a hole buffer layer, a hole adjustment layer, a second hole transport layer, or an electron blocking layer.

In the present disclosure, the electron transport layer 340 may have a single-layer structure or a multi-layer structure, and may comprise one or more electron transport materials, and the electron transport materials can also select from a benzimidazole derivative, an oxadiazole derivative, a quinoxaline derivative, or other electron transport materials, which are not particularly limited in the present disclosure. In one specific embodiment, the electron transport layer 340 is composed of a compound LiQ together with a compound ET.

In the present disclosure, the organic electroluminescent layer 330 may be composed of a single light-emitting material, or may be composed of a host material and a guest material. Preferably, the organic electroluminescent layer 330 is composed of the host material and the guest material, holes injected into the organic electroluminescent layer 330 and electrons injected into the organic electroluminescent layer 330 may be recombined in the organic electroluminescent layer 330 to form excitons, and the excitons transfer energy to the host material, and the host material transfers energy to the guest material, thus enabling the guest material to emit light.

The host material of the organic electroluminescent layer 330 may be a metal chelated compound, a bistyryl derivative, an aromatic amine derivative, a dibenzofuran derivative, or other types of materials, and in one specific embodiment, the host material of the organic electroluminescent layer is composed of the organic compound of the present disclosure together with a compound RH-N.

The guest material of the organic electroluminescent layer 330 may be a compound having a condensed aryl ring or its derivative, a compound having a heteroaryl ring or its derivative, an aromatic amine derivative, or other materials, which is not particularly limited in the present disclosure. In one specific embodiment, the guest material is composed of a compound RD.

In one specific embodiment, the cathode 200 comprises a cathode material that is a material having a small work function that facilitates electron injection into the functional layer. Specifically, specific examples of the cathode material include, but are not limited to: metals such as magnesium, calcium, sodium, potassium, titanium, indium, yttrium, lithium, gadolinium, aluminum, silver, tin, and lead, or their alloys; multilayer materials such as LiF/Al, Liq/Al, LiO₂/Al, LiF/Ca, LiF/Al and BaF₂/Ca, but are not limited to this. Preferably, a metal electrode comprising silver and magnesium is used as the cathode.

In the present disclosure, as shown in FIG. 1, a hole injection layer 310 may further be disposed between the anode 100 and the hole transport layer 321 to enhance the ability to inject holes to the hole transport layer 321. The hole injection layer 310 may be made of a benzidine derivative, a starburst arylamine compound, a phthalocyanine derivative, or other materials, which is not particularly limited in the present disclosure. In some embodiments of the present disclosure, the hole injection layer 310 may consist of a compound PD and a compound HT-1.

In one specific embodiment, as shown in FIG. 1, an electron injection layer 350 may further be disposed between the cathode 200 and the electron transport layer 340 to enhance the ability to inject electrons into the electron transport layer 350. In one specific embodiment, the electron injection layer 350 may comprise ytterbium (Yb).

In a third aspect of the present disclosure, provided is an electronic apparatus, comprising the organic electroluminescent device according to the second aspect of the present disclosure.

According to one embodiment, as shown in FIG. 2, the electronic apparatus is an electronic apparatus 400, comprising the organic electroluminescent device described above. The electronic apparatus 400 may be, for example, a display device, a lighting device, an optical communication device, or other type of electronic apparatus, and may include, for example, but is not limited to, a computer screen, a mobile phone screen, a television, electronic paper, an emergency light, a light module, and the like.

A synthesis method for the nitrogen compound of the present disclosure is specifically illustrated below with reference to synthesis examples, but the present disclosure is not limited in any way accordingly.

Compounds of which synthetic methods are not mentioned in the present disclosure are commercially available raw material products.

### Synthesis of Intermediate Sub-a1

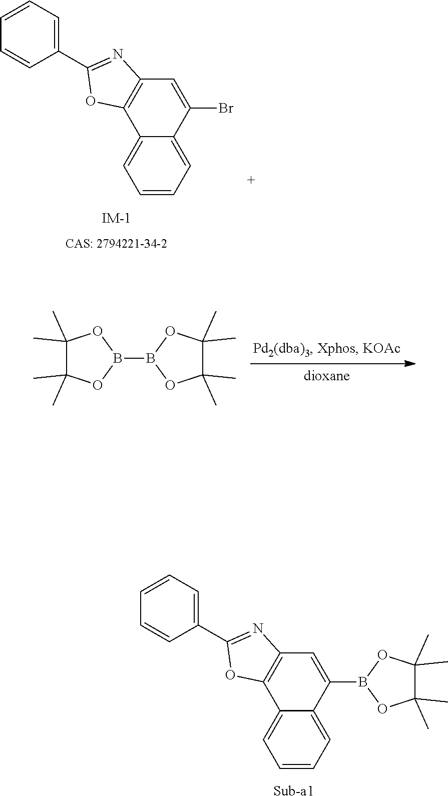

Under the protection of nitrogen, IM-1 (16.21 g, 50 mmol), bis(pinacolato)diboron (14.00 g, 55 mmol), potassium acetate (10.80 g, 110 mmol) and 1,4-dioxane (160 mL) were sequentially added into a 500 mL three-necked flask, stirring and heating were started, after the system was heated to 40° C., tris(dibenzylideneacetone)dipalladium (Pd₂(dba)₃, 0.46 g, 0.50 mmol) and 2-dicyclohexylphosphino-2′,4′,6′-triisopropylbiphenyl (XPhos, 0.48 g, 1.0 mmol) were added into the reaction solution rapidly, heating was continued to be performed to reflux, and a reaction was carried out under stirring overnight. After the system was cooled to room temperature, 200 mL of water was added into the system, well stirring was performed for 30 min, suction filtration was performed under reduced pressure, and a filter cake was washed with deionized water to be neutral, and rinsed with 100 mL of absolute ethanol to obtain a gray solid; and the obtained crude product was pupled once with n-heptane, the pupled material was dissolved with 200 mL of toluene to be clear, and the obtained solution was allowed to pass through a silica gel column, a catalyst was removed, and concentration was performed to obtain Sub-a1 (12.43 g, yield: 67%) as a white solid.

### Synthesis of Intermediate Sub-b1

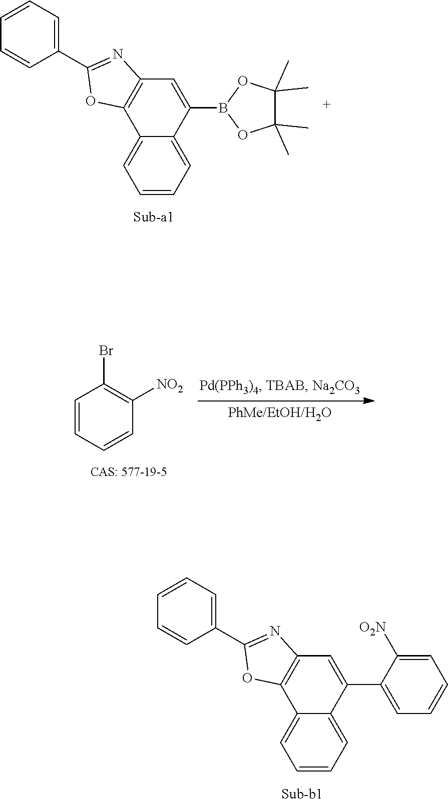

Under the protection of nitrogen, o-bromonitrobenzene (10.10 g, 50 mmol), Sub-a1 (20.42 g, 55 mmol), tetrakis(triphenylphosphine) palladium (0.58 g, 0.5 mmol), anhydrous sodium carbonate (10.60 g, 100 mmol), toluene (140 mL), anhydrous ethanol (35 mL), and deionized water (35 mL) were sequentially added into a 500 mL three-necked flask, stirring and heating were started, heating was performed to reflux, and a reaction was carried out for 8 h. After the system was cooled to room temperature, the resulting reaction solution was extracted with dichloromethane (100 mL×3), organic phases were mixed, dried over anhydrous magnesium sulfate, and filtered, and distillation was performed under reduced pressure to remove a solvent to obtain a crude product. The crude product was purified by silica gel column chromatography using a dichloromethane/n-heptane mixed solvent as a mobile phase to obtain Sub-b1 (13.18 g, yield 72%) as an orange-yellow solid.

Referring to the synthesis method for the Intermediate Sub-b1, by using Reactant A shown in Table 1 instead of o-bromonitrobenzene, Intermediates Sub-bX were synthesized, and the yields are listed in Table 1.

TABLE 1

| Sub-bX | Reactant A | Structure of Sub-bX | Yield (%) |
|---|---|---|---|
| Sub-b2 | 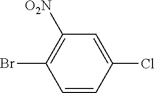 [SMILES: O=[N+]([O-])c1cc(Cl)ccc1Br] CAS: 41513-04-6 | 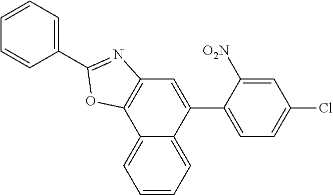 [SMILES: O=[N+]([O-])c1cc(Cl)ccc1-c1cc2nc(-c3ccccc3)oc2c2ccccc12] | 77 |
| Sub-b3 | 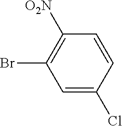 [SMILES: O=[N+]([O-])c1ccc(Cl)cc1Br] CAS: 63860-31-1 | 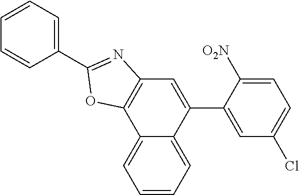 [SMILES: O=[N+]([O-])c1ccc(Cl)cc1-c1cc2nc(-c3ccccc3)oc2c2ccccc12] | 74 |
| Sub-b4 | 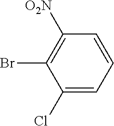 [SMILES: O=[N+]([O-])c1cccc(Cl)c1Br] CAS: 19128-48-4 | 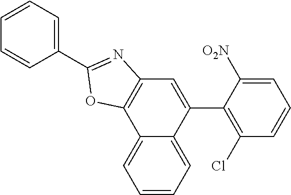 [SMILES: O=[N+]([O-])c1cccc(Cl)c1-c1cc2nc(-c3ccccc3)oc2c2ccccc12] | 58 |

### Synthesis of Intermediate Sub-c1

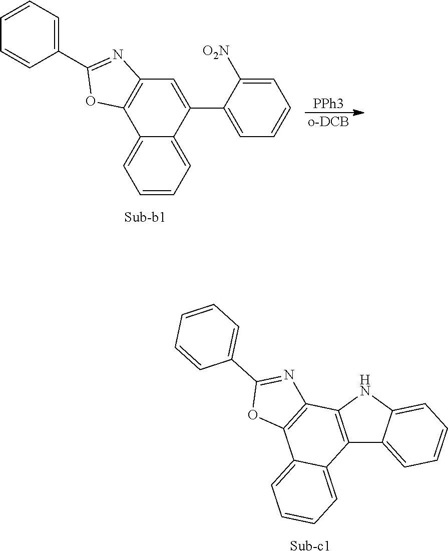

Under the protection of nitrogen, Sub-b1 (18.32 g, 50 mmol), triphenylphosphine (32.78 g, 125 mmol) and o-dichlorobenzene (180 mL) were added into a 500 mL three-necked flask, stirring and heating were started, heating was performed to reflux, and a reaction was carried out for 24 h. After the system was cooled to room temperature, and distillation was performed under reduced pressure to remove a solvent to obtain a crude product. The crude product was purified by silica gel column chromatography using a n-heptane/dichloromethane mixed solvent as a mobile phase to obtain Sub-c1 (9.70 g, yield 58%) as a white solid.

Referring to the synthesis method for the Intermediate Sub-c1, by using Reactant B shown in Table 2 instead of Sub-b1, Intermediates Sub-cX were synthesized, and the yields are listed in Table 2.

TABLE 2

| Sub-cX | Reactant B | Structure of Sub-cX | Yield (%) |
|---|---|---|---|
| Sub-c2 | 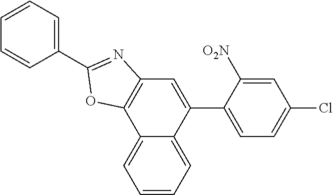 [SMILES: O=[N+]([O-])c1cc(Cl)ccc1-c1cc2nc(-c3ccccc3)oc2c2ccccc12] | 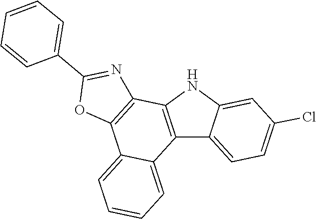 [SMILES: Clc1ccc2c(c1)[nH]c1c3nc(-c4ccccc4)oc3c3ccccc3c21] | 55 |
| Sub-c3 | 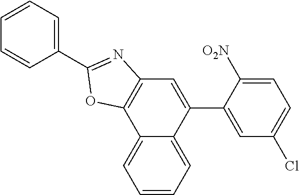 [SMILES: O=[N+]([O-])c1ccc(Cl)cc1-c1cc2nc(-c3ccccc3)oc2c2ccccc12] | 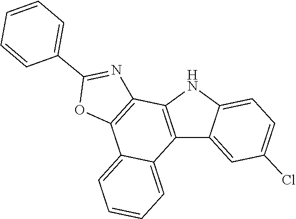 [SMILES: Clc1ccc2[nH]c3c4nc(-c5ccccc5)oc4c4ccccc4c3c2c1] | 57 |
| Sub-c4 | 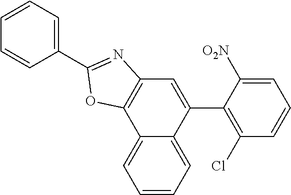 [SMILES: O=[N+]([O-])c1cccc(Cl)c1-c1cc2nc(-c3ccccc3)oc2c2ccccc12] | 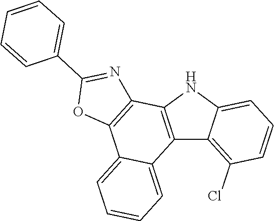 [SMILES: Clc1cccc2[nH]c3c4nc(-c5ccccc5)oc4c4ccccc4c3c12] | 50 |

### Synthesis of Intermediate Sub-d1

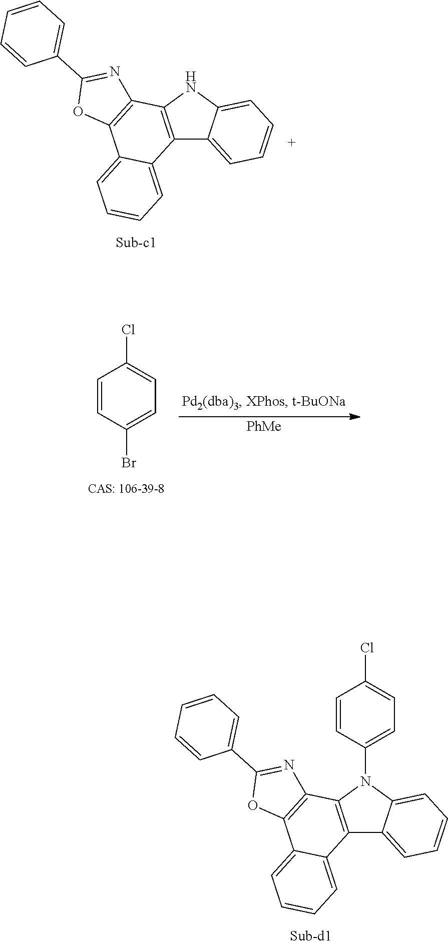

Under the protection of nitrogen, Sub-c1 (16.71 g, 50 mmol), 4-chlorobromobenzene (9.57 g, 50 mmol), tris(dibenzylideneacetone)dipalladium (Pd₂(dba)₃, 0.916 g, 1 mmol), (2-dicyclohexylphosphino-2′,4′,6′-triisopropylbiphenyl) (XPhos, 0.95 g, 2 mmol), sodium tert-butoxide (9.61 g, 100 mmol) and toluene (220 mL) were sequentially added into a 500 mL three-necked flask, heating was performed to reflux, and a reaction was carried out under stirring overnight; after the system was cooled to room temperature, the resulting reaction solution was extracted with dichloromethane (100 mL×3), organic phases were mixed, dried over anhydrous magnesium sulfate, and filtered, and distillation was performed under reduced pressure to remove a solvent to obtain a crude product. The crude product was purified by silica gel column chromatography using a dichloromethane/n-heptane mixed solvent as a mobile phase to obtain an orange-yellow solid (18.24 g, yield: 82%).

Referring to the synthesis method for the Intermediate Sub-d1, by using Reactant C shown in Table 3 instead of Sub-c1 and Reactant D instead of 4-chlorobromobenzene shown in Table 3, Intermediates Sub-dX were synthesized, and the yields are listed in Table 3.

TABLE 3

| Sub-dX No. | Reactant C | Reactant D | Structure of Sub-dX | Yield (%) |
|---|---|---|---|---|
| Sub-d2 | 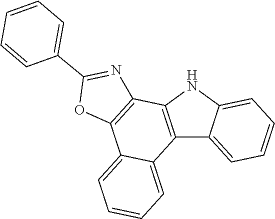 [SMILES: c1ccc(-c2nc3c4[nH]c5ccccc5c4c4ccccc4c3o2)cc1] | 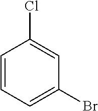 CAS: 108-37-2 | 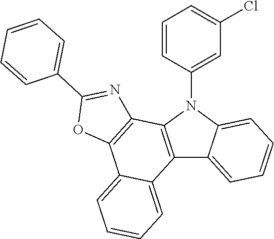 [SMILES: Clc1cccc(-n2c3ccccc3c3c4ccccc4c4oc(-c5ccccc5)nc4c32)c1] | 81 |
| Sub-d3 | 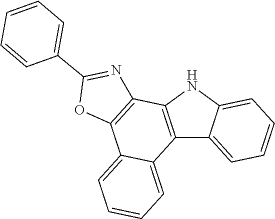 [SMILES: c1ccc(-c2nc3c4[nH]c5ccccc5c4c4ccccc4c3o2)cc1] | 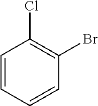 CAS: 694-80-4 | 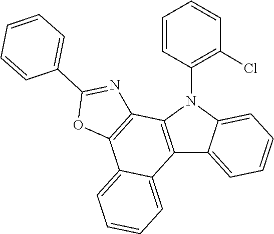 [SMILES: Clc1ccccc1-n1c2ccccc2c2c3ccccc3c3oc(-c4ccccc4)nc3c21] | 66 |
| Sub-d4 | 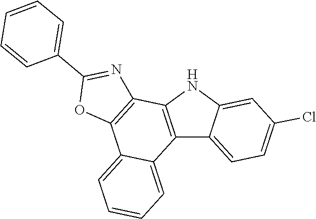 [SMILES: Clc1ccc2c(c1)[nH]c1c3nc(-c4ccccc4)oc3c3ccccc3c21] | 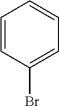 CAS: 108-86-1 | 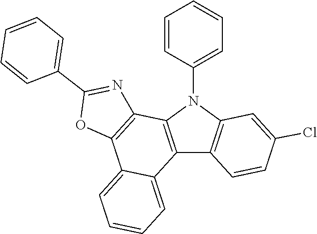 [SMILES: Clc1ccc2c3c4ccccc4c4oc(-c5ccccc5)nc4c3n(-c3ccccc3)c2c1] | 72 |
| Sub-d5 | 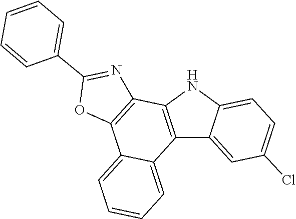 [SMILES: Clc1ccc2[nH]c3c4nc(-c5ccccc5)oc4c4ccccc4c3c2c1] | 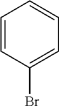 CAS: 108-86-1 | 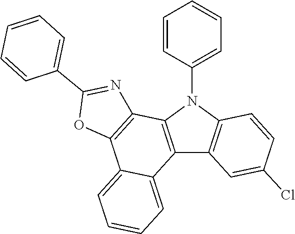 [SMILES: Clc1ccc2c(c1)c1c3ccccc3c3oc(-c4ccccc4)nc3c1n2-c1ccccc1] | 72 |
| Sub-d6 | 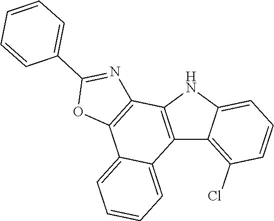 [SMILES: Clc1cccc2[nH]c3c4nc(-c5ccccc5)oc4c4ccccc4c3c12] | 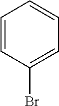 CAS: 108-86-1 | 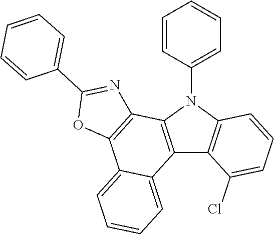 [SMILES: Clc1cccc2c1c1c3ccccc3c3oc(-c4ccccc4)nc3c1n2-c1ccccc1] | 83 |
| Sub-d7 | 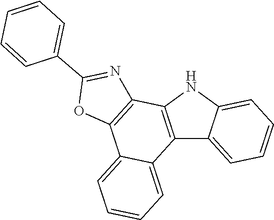 [SMILES: c1ccc(-c2nc3c4[nH]c5ccccc5c4c4ccccc4c3o2)cc1] | 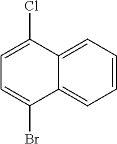 [SMILES: Clc1ccc(Br)c2ccccc12] CAS: 53220-82-9 | 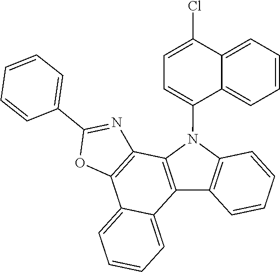 [SMILES: Clc1ccc(-n2c3ccccc3c3c4ccccc4c4oc(-c5ccccc5)nc4c32)c2ccccc12] | 78 |
| Sub-d8 | 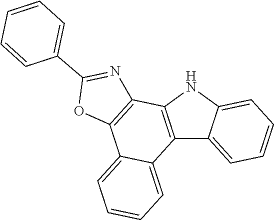 [SMILES: c1ccc(-c2nc3c4[nH]c5ccccc5c4c4ccccc4c3o2)cc1] | 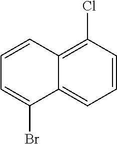 [SMILES: Clc1cccc2c(Br)cccc12] CAS: 77332-65-1 | 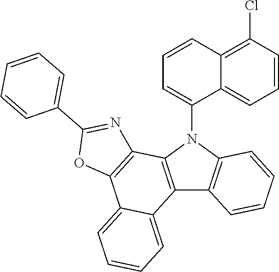 [SMILES: Clc1cccc2c(-n3c4ccccc4c4c5ccccc5c5oc(-c6ccccc6)nc5c43)cccc12] | 80 |
| Sub-d9 | 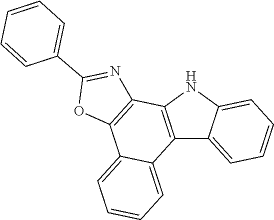 [SMILES: c1ccc(-c2nc3c4[nH]c5ccccc5c4c4ccccc4c3o2)cc1] | 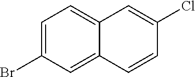 [SMILES: Clc1ccc2cc(Br)ccc2c1] CAS: 870822-84-7 | 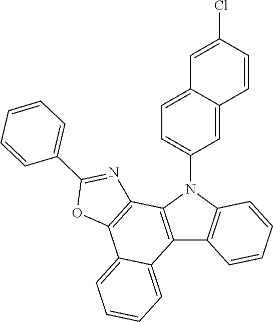 [SMILES: Clc1ccc2cc(-n3c4ccccc4c4c5ccccc5c5oc(-c6ccccc6)nc5c43)ccc2c1] | 81 |
| Sub-d10 | 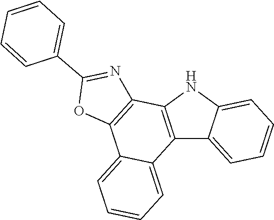 [SMILES: c1ccc(-c2nc3c4[nH]c5ccccc5c4c4ccccc4c3o2)cc1] | 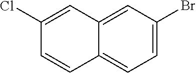 [SMILES: Clc1ccc2ccc(Br)cc2c1] CAS: 321939-67-7 | 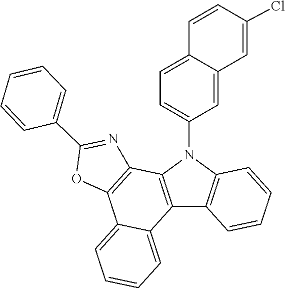 [SMILES: Clc1ccc2ccc(-n3c4ccccc4c4c5ccccc5c5oc(-c6ccccc6)nc5c43)cc2c1] | 81 |
| Sub-d 11 | 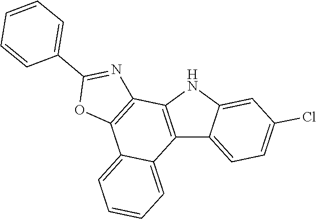 [SMILES: Clc1ccc2c(c1)[nH]c1c3nc(-c4ccccc4)oc3c3ccccc3c21] | 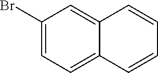 [SMILES: Brc1ccc2ccccc2c1] CAS: 580-13-2 | 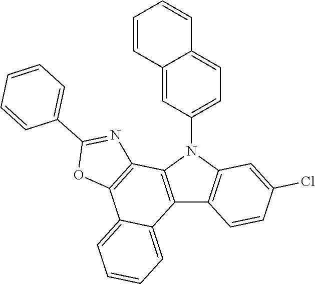 [SMILES: Clc1ccc2c3c4ccccc4c4oc(-c5ccccc5)nc4c3n(-c3ccc4ccccc4c3)c2c1] | 82 |
| Sub-d12 | 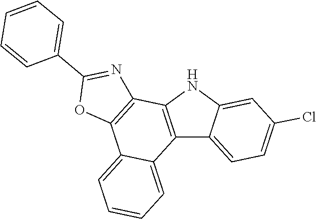 [SMILES: Clc1ccc2c(c1)[nH]c1c3nc(-c4ccccc4)oc3c3ccccc3c21] | 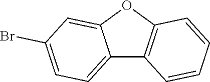 [SMILES: Brc1ccc2c(c1)oc1ccccc12] CAS: 26608-06-0 | 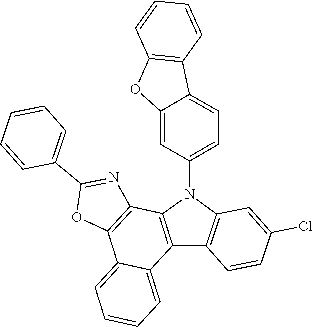 [SMILES: Clc1ccc2c3c4ccccc4c4oc(-c5ccccc5)nc4c3n(-c3ccc4c(c3)oc3ccccc34)c2c1] | 85 |
| Sub-d13 | 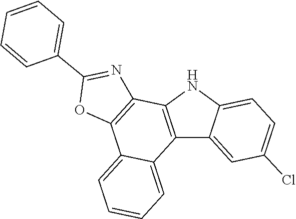 [SMILES: Clc1ccc2[nH]c3c4nc(-c5ccccc5)oc4c4ccccc4c3c2c1] | 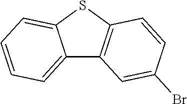 [SMILES: Brc1ccc2sc3ccccc3c2c1] CAS: 22439-61-8 | 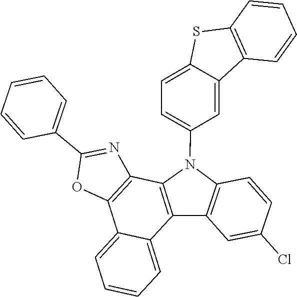 [SMILES: Clc1ccc2c(c1)c1c3ccccc3c3oc(-c4ccccc4)nc3c1n2-c1ccc2sc3ccccc3c2c1] | 80 |
| Sub-d14 | 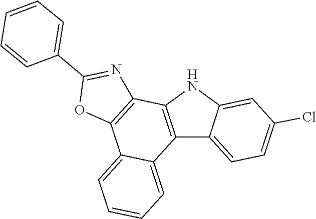 [SMILES: Clc1ccc2c(c1)[nH]c1c3nc(-c4ccccc4)oc3c3ccccc3c21] | 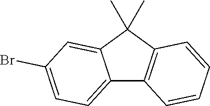 [SMILES: CC1(C)c2ccccc2-c2ccc(Br)cc21] CAS: 28320-31-2 | 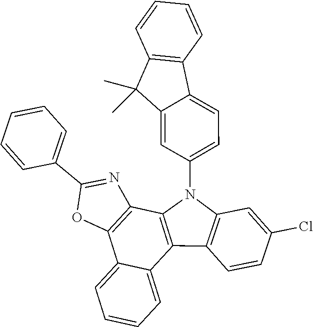 [SMILES: CC1(C)c2ccccc2-c2ccc(-n3c4cc(Cl)ccc4c4c5ccccc5c5oc(-c6ccccc6)nc5c43)cc21] | 76 |
| Sub-d15 | 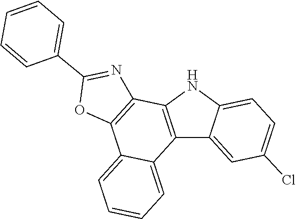 [SMILES: Clc1ccc2[nH]c3c4nc(-c5ccccc5)oc4c4ccccc4c3c2c1] | 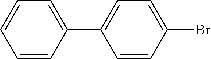 [SMILES: Brc1ccc(-c2ccccc2)cc1] CAS: 92-66-0 | 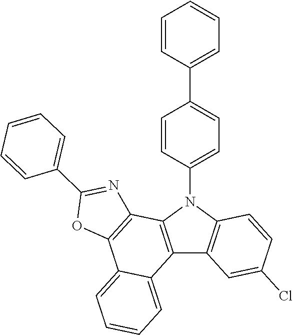 [SMILES: Clc1ccc2c(c1)c1c3ccccc3c3oc(-c4ccccc4)nc3c1n2-c1ccc(-c2ccccc2)cc1] | 84 |
| Sub-d16 | 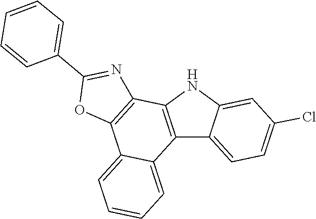 [SMILES: Clc1ccc2c(c1)[nH]c1c3nc(-c4ccccc4)oc3c3ccccc3c21] | 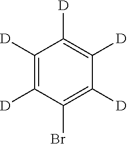 [SMILES: [2H]c1c([2H])c([2H])c(Br)c([2H])c1[2H]] CAS: 4165-57-5 | 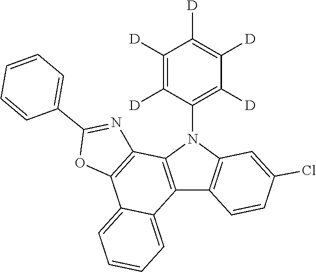 [SMILES: [2H]c1c([2H])c([2H])c(-n2c3cc(Cl)ccc3c3c4ccccc4c4oc(-c5ccccc5)nc4c32)c([2H])c1[2H]] | 82 |

### Synthesis of Compound 15

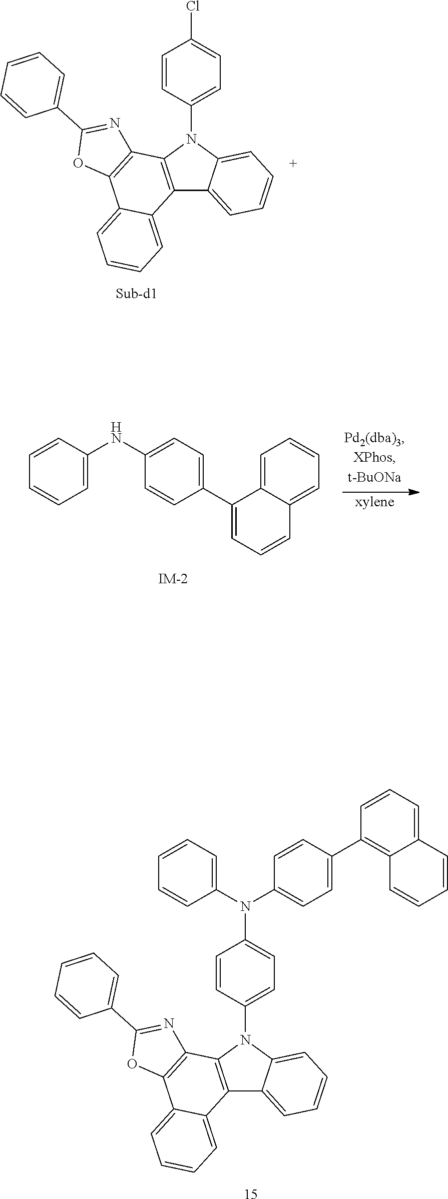

Under the protection of nitrogen, Sub-d1 (11.12 g, 25 mmol), IM-2 (7.38 g, 25 mmol), tris(dibenzylideneacetone)dipalladium (Pd₂(dba)₃, 0.46 g, 0.5 mmol), (2-dicyclohexylphosphino-2′,4′,6′-triisopropylbiphenyl) (XPhos, 0.48 g, 1 mmol), sodium tert-butoxide (9.61 g, 50 mmol) and xylene (120 mL) were sequentially added into a 250 mL three-necked flask, heating was performed to reflux, and a reaction was carried out under stirring overnight; after the system was cooled to room temperature, the resulting reaction solution was extracted with dichloromethane (100 mL×3), organic phases were mixed, dried over anhydrous magnesium sulfate, and filtered, and distillation was performed under reduced pressure to remove a solvent to obtain a crude product. The crude product was purified by silica gel column chromatography using a dichloromethane/n-heptane as a mobile phase to obtain an orange-yellow solid (15.13 g, 86%: yield, m/z=704.27 [M+H]⁺).

Referring to the synthesis method for Compound 15, compounds in Table 4 were synthesized by using Reactant E shown in Table 4 instead of Sub-d1 and Reactant F shown in Table 4 instead of IM-2 (CAS: 897671-78-2), and the yields and H-NMR data are listed in Table 4.

TABLE 4

| | | | m/z | Yield |
|---|---|---|---|---|
| Reactant E | Reactant F | Compound structure and No. | ([M + H]⁺) | % |
| 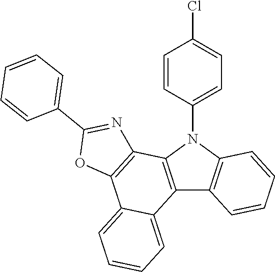 [SMILES: Clc1ccc(-n2c3ccccc3c3c4ccccc4c4oc(-c5ccccc5)nc4c32)cc1] | 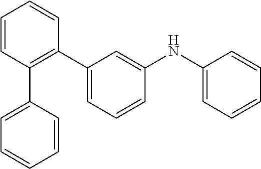 [SMILES: c1ccc(Nc2cccc(-c3ccccc3-c3ccccc3)c2)cc1] CAS: 1222634-04-9 | 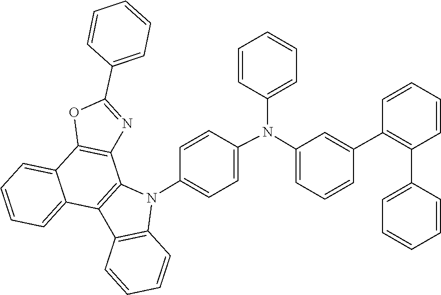 [SMILES: c1ccc(-c2nc3c(o2)c2ccccc2c2c4ccccc4n(-c4ccc(N(c5ccccc5)c5cccc(-c6ccccc6-c6ccccc6)c5)cc4)c32)cc1] 23 | 730.28 | 81 |
| 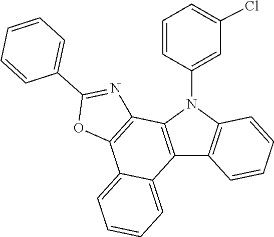 [SMILES: Clc1cccc(-n2c3ccccc3c3c4ccccc4c4oc(-c5ccccc5)nc4c32)c1] | 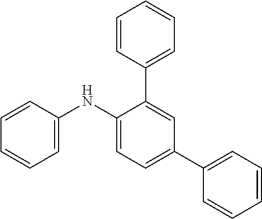 [SMILES: c1ccc(Nc2ccc(-c3ccccc3)cc2-c2ccccc2)cc1] CAS: 1417334-01-0 | 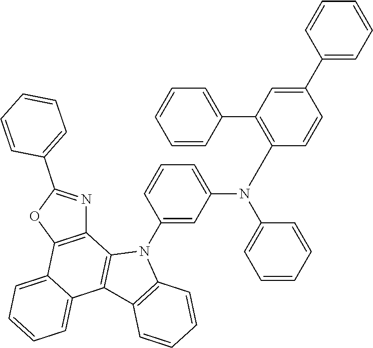 [SMILES: c1ccc(-c2ccc(N(c3ccccc3)c3cccc(-n4c5ccccc5c5c6ccccc6c6oc(-c7ccccc7)nc6c54)c3)c(-c3ccccc3)c2)cc1] 28 | 730.28 | 73 |
| 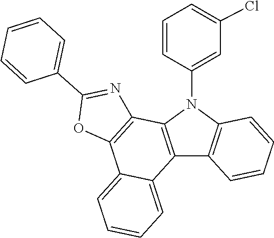 [SMILES: Clc1cccc(-n2c3ccccc3c3c4ccccc4c4oc(-c5ccccc5)nc4c32)c1] | 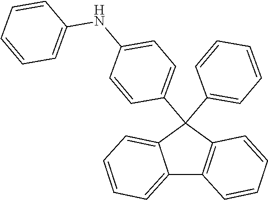 [SMILES: c1ccc(Nc2ccc(C3(c4ccccc4)c4ccccc4-c4ccccc43)cc2)cc1] CAS: 851343-53-8 | 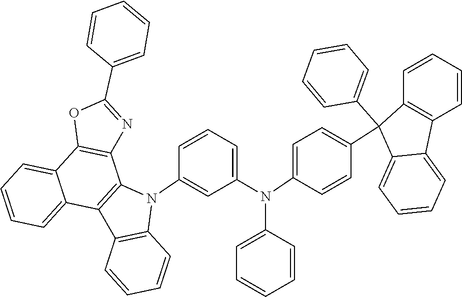 [SMILES: c1ccc(-c2nc3c(o2)c2ccccc2c2c4ccccc4n(-c4cccc(N(c5ccccc5)c5ccc(C6(c7ccccc7)c7ccccc7-c7ccccc76)cc5)c4)c32)cc1] 40 | 818.31 | 73 |
| 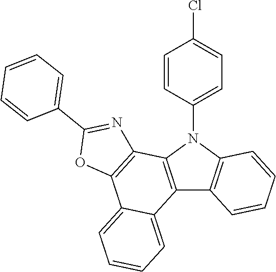 [SMILES: Clc1ccc(-n2c3ccccc3c3c4ccccc4c4oc(-c5ccccc5)nc4c32)cc1] | 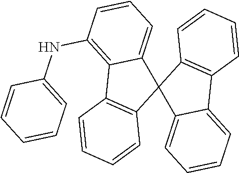 [SMILES: c1ccc(Nc2cccc3c2-c2ccccc2C32c3ccccc3-c3ccccc32)cc1] CAS: 1454679-32-3 | 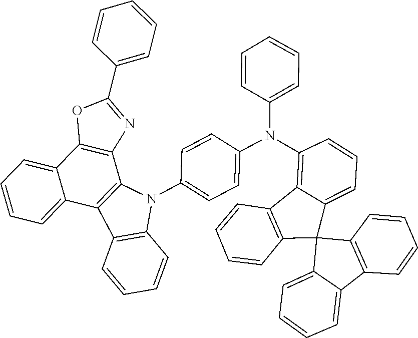 [SMILES: c1ccc(-c2nc3c(o2)c2ccccc2c2c4ccccc4n(-c4ccc(N(c5ccccc5)c5cccc6c5-c5ccccc5C65c6ccccc6-c6ccccc65)cc4)c32)cc1] 43 | 816.30 | 70 |
| 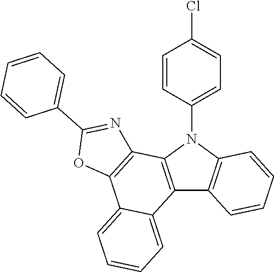 [SMILES: Clc1ccc(-n2c3ccccc3c3c4ccccc4c4oc(-c5ccccc5)nc4c32)cc1] | 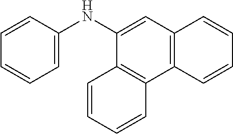 [SMILES: c1ccc(Nc2cc3ccccc3c3ccccc23)cc1] CAS: 3920-79-4 | 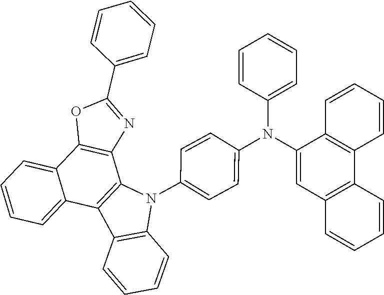 [SMILES: c1ccc(-c2nc3c(o2)c2ccccc2c2c4ccccc4n(-c4ccc(N(c5ccccc5)c5cc6ccccc6c6ccccc56)cc4)c32)cc1] 45 | 678.25 | 75 |
| 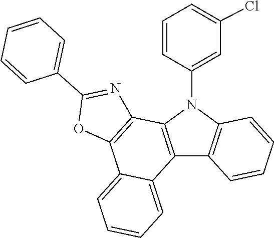 [SMILES: Clc1cccc(-n2c3ccccc3c3c4ccccc4c4oc(-c5ccccc5)nc4c32)c1] | 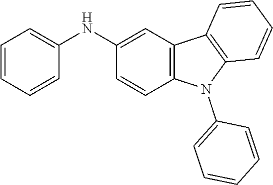 [SMILES: c1ccc(Nc2ccc3c(c2)c2ccccc2n3-c2ccccc2)cc1] CAS: 894791-43-6 | 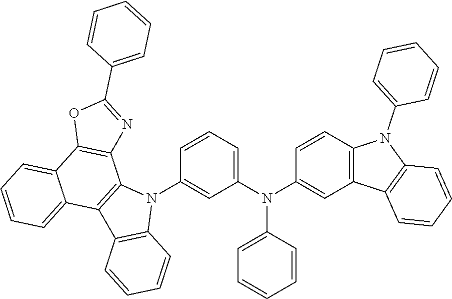 [SMILES: c1ccc(-c2nc3c(o2)c2ccccc2c2c4ccccc4n(-c4cccc(N(c5ccccc5)c5ccc6c(c5)c5ccccc5n6-c5ccccc5)c4)c32)cc1] 50 | 743.28 | 85 |
| 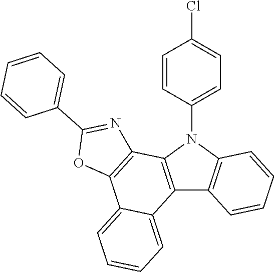 [SMILES: Clc1ccc(-n2c3ccccc3c3c4ccccc4c4oc(-c5ccccc5)nc4c32)cc1] | 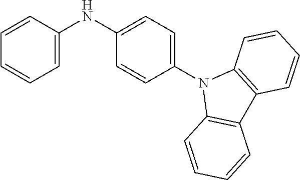 [SMILES: c1ccc(Nc2ccc(-n3c4ccccc4c4ccccc43)cc2)cc1] CAS: 858641-06-2 | 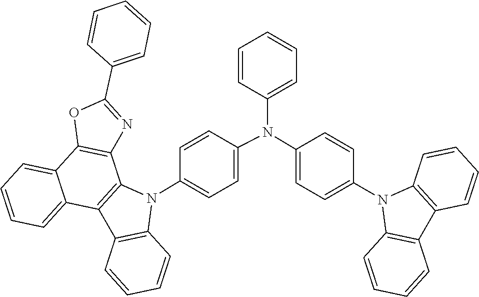 [SMILES: c1ccc(-c2nc3c(o2)c2ccccc2c2c4ccccc4n(-c4ccc(N(c5ccccc5)c5ccc(-n6c7ccccc7c7ccccc76)cc5)cc4)c32)cc1] 51 | 743.28 | 88 |
| 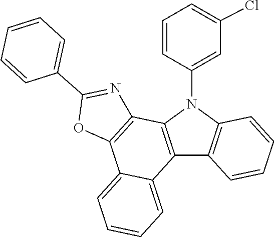 [SMILES: Clc1cccc(-n2c3ccccc3c3c4ccccc4c4oc(-c5ccccc5)nc4c32)c1] | 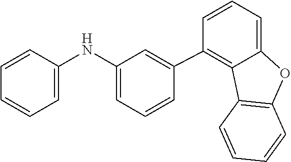 [SMILES: c1ccc(Nc2cccc(-c3cccc4oc5ccccc5c34)c2)cc1] CAS: 2271355-33-8 | 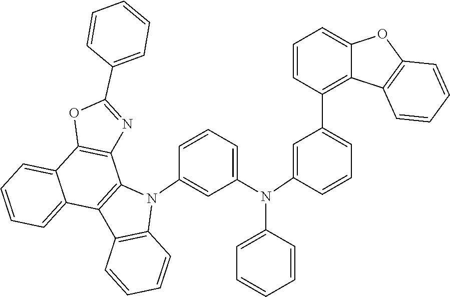 [SMILES: c1ccc(-c2nc3c(o2)c2ccccc2c2c4ccccc4n(-c4cccc(N(c5ccccc5)c5cccc(-c6cccc7oc8ccccc8c67)c5)c4)c32)cc1] 60 | 744.26 | 73 |
| 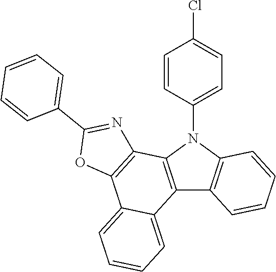 [SMILES: Clc1ccc(-n2c3ccccc3c3c4ccccc4c4oc(-c5ccccc5)nc4c32)cc1] | 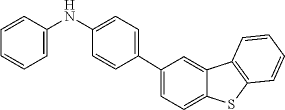 [SMILES: c1ccc(Nc2ccc(-c3ccc4sc5ccccc5c4c3)cc2)cc1] CAS: 1345614-86-9 | 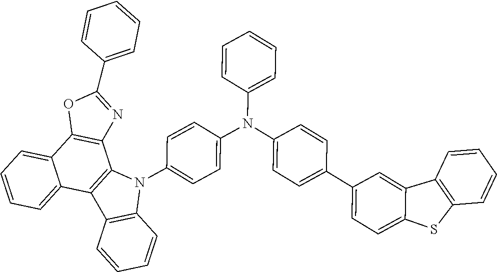 [SMILES: c1ccc(-c2nc3c(o2)c2ccccc2c2c4ccccc4n(-c4ccc(N(c5ccccc5)c5ccc(-c6ccc7sc8ccccc8c7c6)cc5)cc4)c32)cc1] 71 | 760.24 | 78 |
| 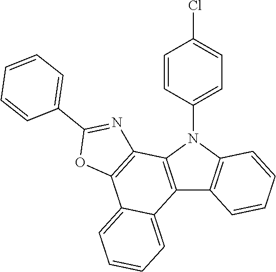 [SMILES: Clc1ccc(-n2c3ccccc3c3c4ccccc4c4oc(-c5ccccc5)nc4c32)cc1] | 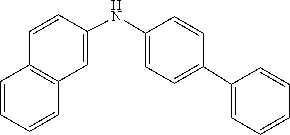 [SMILES: c1ccc(-c2ccc(Nc3ccc4ccccc4c3)cc2)cc1] CAS: 6336-92-1 | 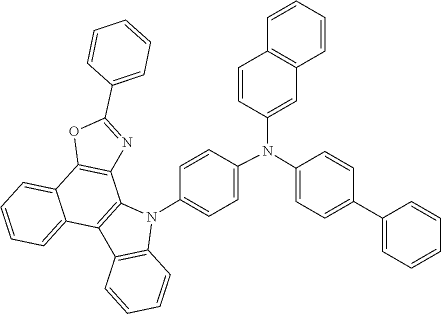 [SMILES: c1ccc(-c2ccc(N(c3ccc(-n4c5ccccc5c5c6ccccc6c6oc(-c7ccccc7)nc6c54)cc3)c3ccc4ccccc4c3)cc2)cc1] 73 | 704.27 | 78 |
| 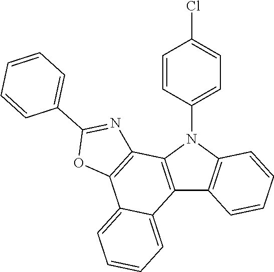 [SMILES: Clc1ccc(-n2c3ccccc3c3c4ccccc4c4oc(-c5ccccc5)nc4c32)cc1] | 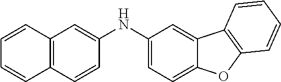 [SMILES: c1ccc2cc(Nc3ccc4oc5ccccc5c4c3)ccc2c1] CAS: 1621976-98-4 | 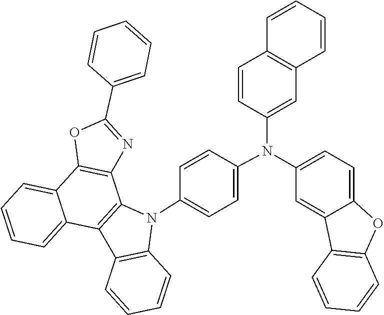 [SMILES: c1ccc(-c2nc3c(o2)c2ccccc2c2c4ccccc4n(-c4ccc(N(c5ccc6ccccc6c5)c5ccc6oc7ccccc7c6c5)cc4)c32)cc1] 77 | 718.24 | 81 |
| 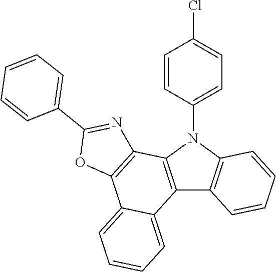 [SMILES: Clc1ccc(-n2c3ccccc3c3c4ccccc4c4oc(-c5ccccc5)nc4c32)cc1] | 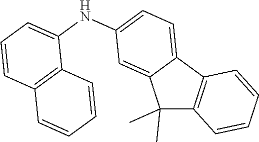 [SMILES: CC1(C)c2ccccc2-c2ccc(Nc3cccc4ccccc34)cc21] CAS: 672289-02-0 | 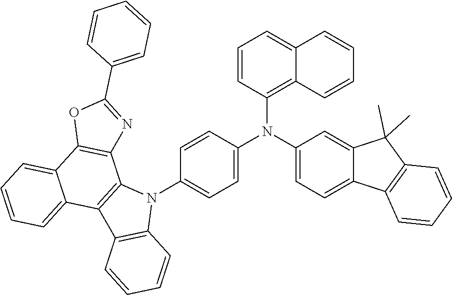 [SMILES: CC1(C)c2ccccc2-c2ccc(N(c3ccc(-n4c5ccccc5c5c6ccccc6c6oc(-c7ccccc7)nc6c54)cc3)c3cccc4ccccc34)cc21] 87 | 744.30 | 76 |
| 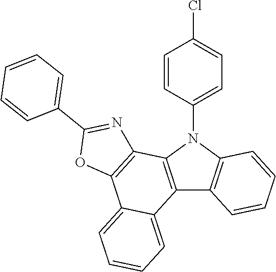 [SMILES: Clc1ccc(-n2c3ccccc3c3c4ccccc4c4oc(-c5ccccc5)nc4c32)cc1] | 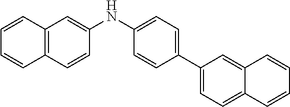 [SMILES: c1ccc2cc(Nc3ccc(-c4ccc5ccccc5c4)cc3)ccc2c1] CAS: 209804-16-0 | 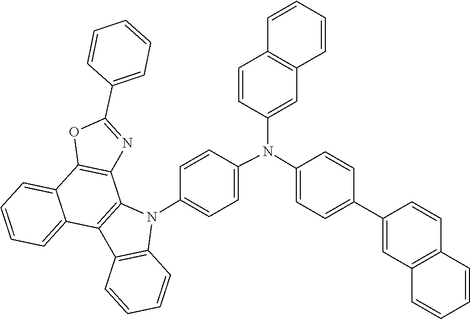 [SMILES: c1ccc(-c2nc3c(o2)c2ccccc2c2c4ccccc4n(-c4ccc(N(c5ccc(-c6ccc7ccccc7c6)cc5)c5ccc6ccccc6c5)cc4)c32)cc1] 89 | 754.28 | 79 |
| 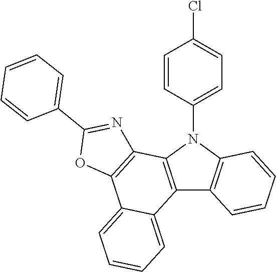 [SMILES: Clc1ccc(-n2c3ccccc3c3c4ccccc4c4oc(-c5ccccc5)nc4c32)cc1] | 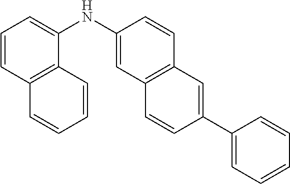 [SMILES: c1ccc(-c2ccc3cc(Nc4cccc5ccccc45)ccc3c2)cc1] CAS: 1290039-93-8 | 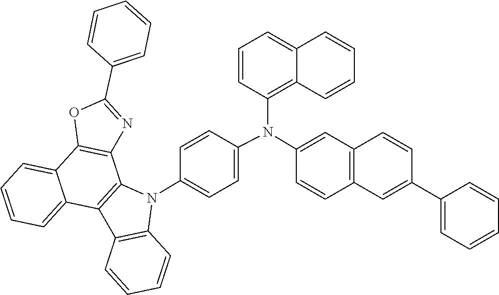 [SMILES: c1ccc(-c2ccc3cc(N(c4ccc(-n5c6ccccc6c6c7ccccc7c7oc(-c8ccccc8)nc7c65)cc4)c4cccc5ccccc45)ccc3c2)cc1] 91 | 754.28 | 77 |
| 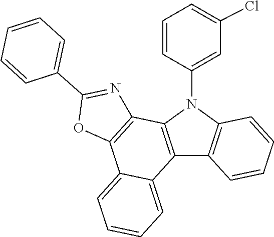 [SMILES: Clc1cccc(-n2c3ccccc3c3c4ccccc4c4oc(-c5ccccc5)nc4c32)c1] | 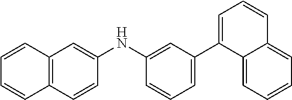 [SMILES: c1cc(Nc2ccc3ccccc3c2)cc(-c2cccc3ccccc23)c1] CAS: 2634130-32-6 | 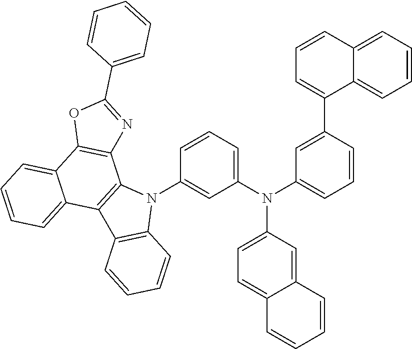 [SMILES: c1ccc(-c2nc3c(o2)c2ccccc2c2c4ccccc4n(-c4cccc(N(c5cccc(-c6cccc7ccccc67)c5)c5ccc6ccccc6c5)c4)c32)cc1] 92 | 754.28 | 79 |
| 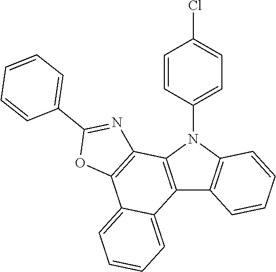 [SMILES: Clc1ccc(-n2c3ccccc3c3c4ccccc4c4oc(-c5ccccc5)nc4c32)cc1] | 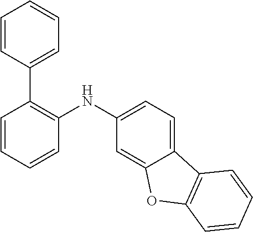 [SMILES: c1ccc(-c2ccccc2Nc2ccc3c(c2)oc2ccccc23)cc1] CAS: 1427556-44-2 | 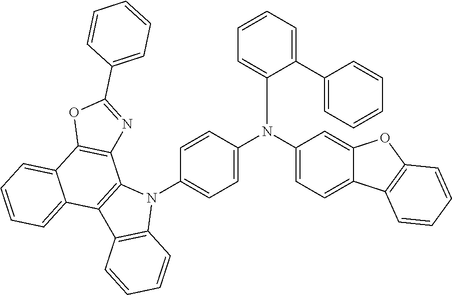 [SMILES: c1ccc(-c2nc3c(o2)c2ccccc2c2c4ccccc4n(-c4ccc(N(c5ccc6c(c5)oc5ccccc56)c5ccccc5-c5ccccc5)cc4)c32)cc1] 111 | 744.26 | 72 |
| 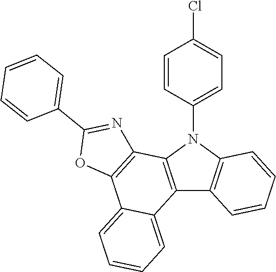 [SMILES: Clc1ccc(-n2c3ccccc3c3c4ccccc4c4oc(-c5ccccc5)nc4c32)cc1] | 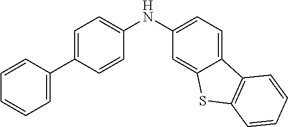 [SMILES: c1ccc(-c2ccc(Nc3ccc4c(c3)sc3ccccc34)cc2)cc1] CAS: 1290039-87-0 | 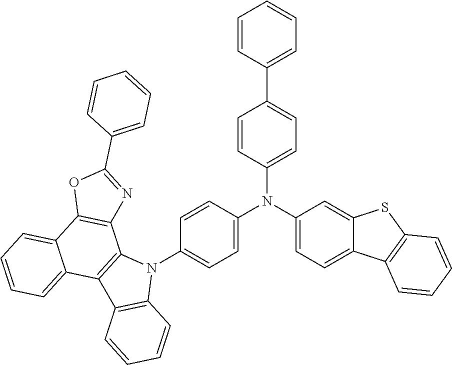 [SMILES: c1ccc(-c2ccc(N(c3ccc(-n4c5ccccc5c5c6ccccc6c6oc(-c7ccccc7)nc6c54)cc3)c3ccc4c(c3)sc3ccccc34)cc2)cc1] 115 | 760.24 | 87 |
| 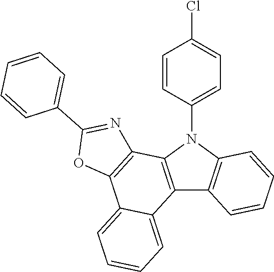 [SMILES: Clc1ccc(-n2c3ccccc3c3c4ccccc4c4oc(-c5ccccc5)nc4c32)cc1] | 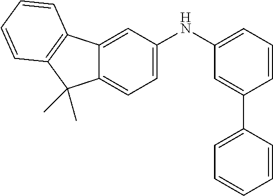 [SMILES: CC1(C)c2ccccc2-c2cc(Nc3cccc(-c4ccccc4)c3)ccc21] CAS: 1613331-97-7 | 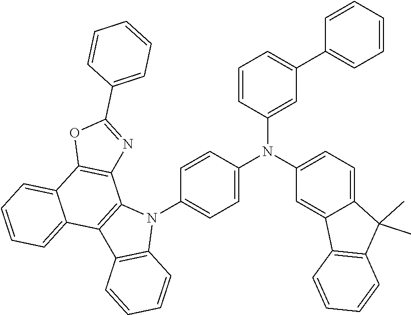 [SMILES: CC1(C)c2ccccc2-c2cc(N(c3ccc(-n4c5ccccc5c5c6ccccc6c6oc(-c7ccccc7)nc6c54)cc3)c3cccc(-c4ccccc4)c3)ccc21] 119 | 770.31 | 78 |
| 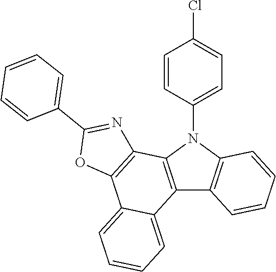 [SMILES: Clc1ccc(-n2c3ccccc3c3c4ccccc4c4oc(-c5ccccc5)nc4c32)cc1] | 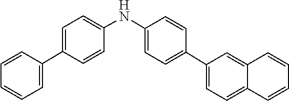 [SMILES: c1ccc(-c2ccc(Nc3ccc(-c4ccc5ccccc5c4)cc3)cc2)cc1] CAS: 897921-60-7 | 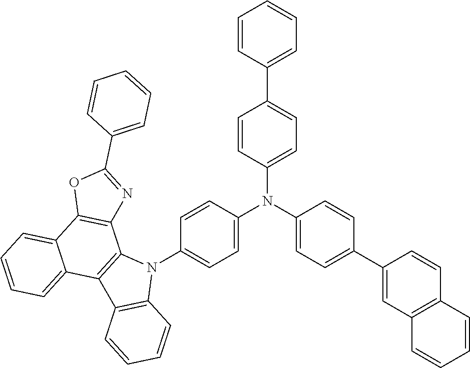 [SMILES: c1ccc(-c2ccc(N(c3ccc(-c4ccc5ccccc5c4)cc3)c3ccc(-n4c5ccccc5c5c6ccccc6c6oc(-c7ccccc7)nc6c54)cc3)cc2)cc1] 124 | 780.30 | 79 |
| 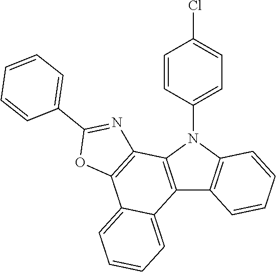 [SMILES: Clc1ccc(-n2c3ccccc3c3c4ccccc4c4oc(-c5ccccc5)nc4c32)cc1] | 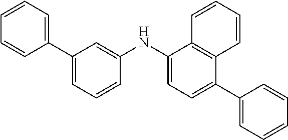 [SMILES: c1ccc(-c2cccc(Nc3ccc(-c4ccccc4)c4ccccc34)c2)cc1] CAS: 2634130-33-7 | 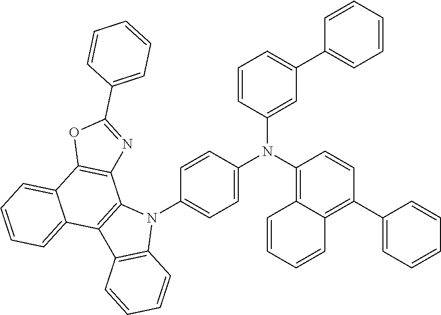 [SMILES: c1ccc(-c2cccc(N(c3ccc(-n4c5ccccc5c5c6ccccc6c6oc(-c7ccccc7)nc6c54)cc3)c3ccc(-c4ccccc4)c4ccccc34)c2)cc1] 125 | 780.30 | 75 |
| 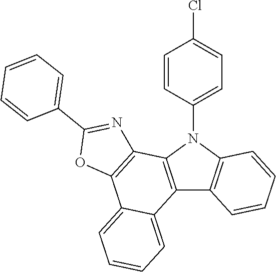 [SMILES: Clc1ccc(-n2c3ccccc3c3c4ccccc4c4oc(-c5ccccc5)nc4c32)cc1] | 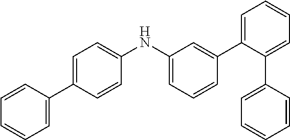 [SMILES: c1ccc(-c2ccc(Nc3cccc(-c4ccccc4-c4ccccc4)c3)cc2)cc1] CAS: 2762148-19-4 | 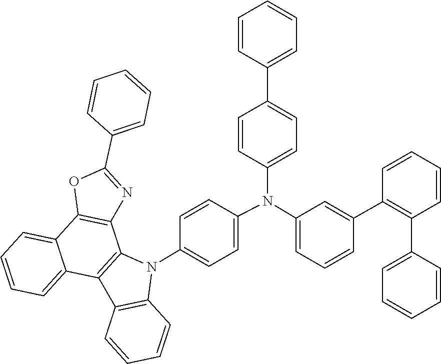 [SMILES: c1ccc(-c2ccc(N(c3ccc(-n4c5ccccc5c5c6ccccc6c6oc(-c7ccccc7)nc6c54)cc3)c3cccc(-c4ccccc4-c4ccccc4)c3)cc2)cc1] 130 | 806.31 | 81 |
| 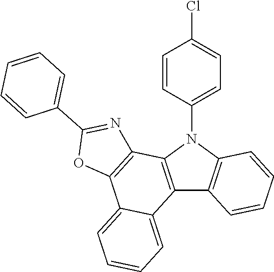 [SMILES: Clc1ccc(-n2c3ccccc3c3c4ccccc4c4oc(-c5ccccc5)nc4c32)cc1] | 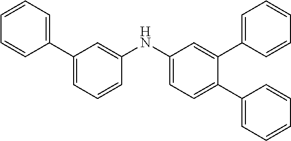 [SMILES: c1ccc(-c2cccc(Nc3ccc(-c4ccccc4)c(-c4ccccc4)c3)c2)cc1] CAS: 1372779-41-3 | 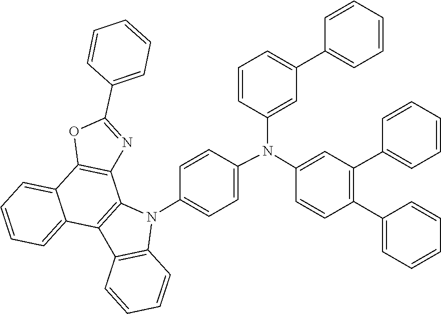 [SMILES: c1ccc(-c2cccc(N(c3ccc(-n4c5ccccc5c5c6ccccc6c6oc(-c7ccccc7)nc6c54)cc3)c3ccc(-c4ccccc4)c(-c4ccccc4)c3)c2)cc1] 134 | 806.31 | 79 |
| 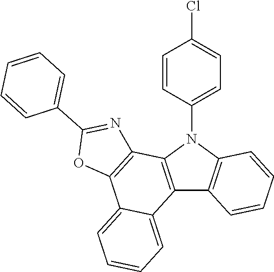 [SMILES: Clc1ccc(-n2c3ccccc3c3c4ccccc4c4oc(-c5ccccc5)nc4c32)cc1] | 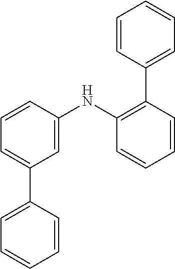 [SMILES: c1ccc(-c2cccc(Nc3ccccc3-c3ccccc3)c2)cc1] CAS: 1258515-01-3 | 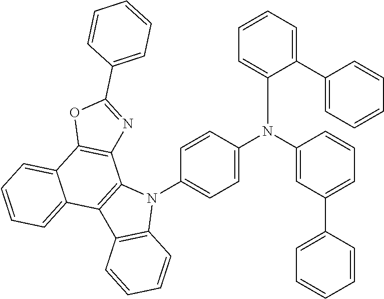 [SMILES: c1ccc(-c2cccc(N(c3ccc(-n4c5ccccc5c5c6ccccc6c6oc(-c7ccccc7)nc6c54)cc3)c3ccccc3-c3ccccc3)c2)cc1] 135 | 730.28 | 74 |
| 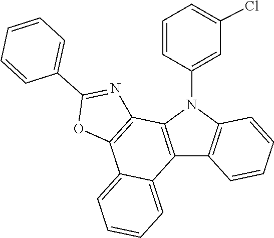 [SMILES: Clc1cccc(-n2c3ccccc3c3c4ccccc4c4oc(-c5ccccc5)nc4c32)c1] | 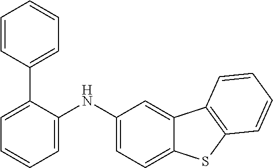 [SMILES: c1ccc(-c2ccccc2Nc2ccc3sc4ccccc4c3c2)cc1] CAS: 2170534-12-8 | 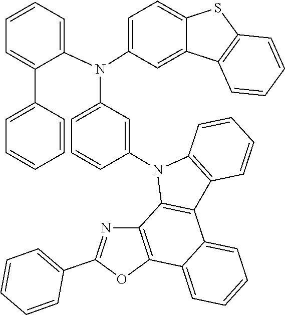 [SMILES: c1ccc(-c2nc3c(o2)c2ccccc2c2c4ccccc4n(-c4cccc(N(c5ccc6sc7ccccc7c6c5)c5ccccc5-c5ccccc5)c4)c32)cc1] 144 | 760.24 | 73 |
| 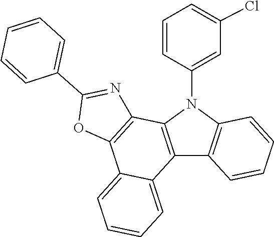 [SMILES: Clc1cccc(-n2c3ccccc3c3c4ccccc4c4oc(-c5ccccc5)nc4c32)c1] | 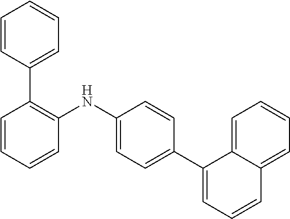 [SMILES: c1ccc(-c2ccccc2Nc2ccc(-c3cccc4ccccc34)cc2)cc1] CAS: 1848987-46-1 | 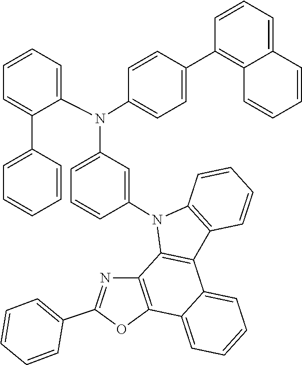 [SMILES: c1ccc(-c2nc3c(o2)c2ccccc2c2c4ccccc4n(-c4cccc(N(c5ccc(-c6cccc7ccccc67)cc5)c5ccccc5-c5ccccc5)c4)c32)cc1] 147 | 780.30 | 71 |
| 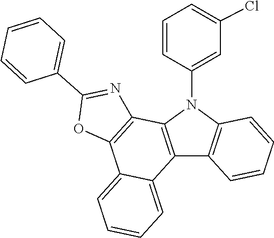 [SMILES: Clc1cccc(-n2c3ccccc3c3c4ccccc4c4oc(-c5ccccc5)nc4c32)c1] | 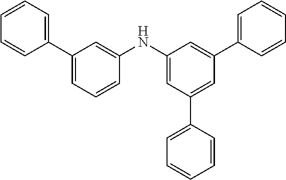 [SMILES: c1ccc(-c2cccc(Nc3cc(-c4ccccc4)cc(-c4ccccc4)c3)c2)cc1] CAS: 1372779-30-0 | 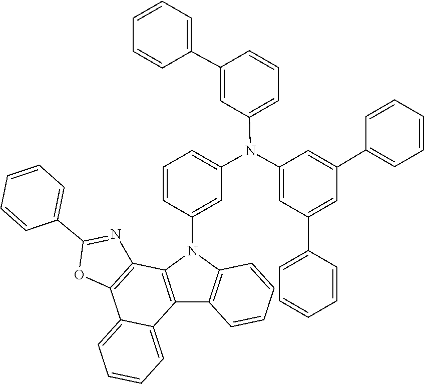 [SMILES: c1ccc(-c2cccc(N(c3cc(-c4ccccc4)cc(-c4ccccc4)c3)c3cccc(-n4c5ccccc5c5c6ccccc6c6oc(-c7ccccc7)nc6c54)c3)c2)cc1] 152 | 806.31 | 80 |
| 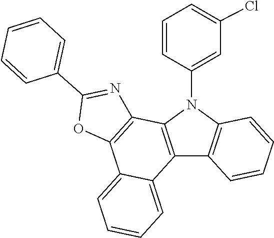 [SMILES: Clc1cccc(-n2c3ccccc3c3c4ccccc4c4oc(-c5ccccc5)nc4c32)c1] | 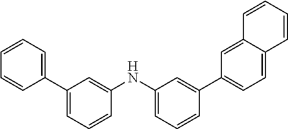 [SMILES: c1ccc(-c2cccc(Nc3cccc(-c4ccc5ccccc5c4)c3)c2)cc1] CAS: 1848987-47-2 | 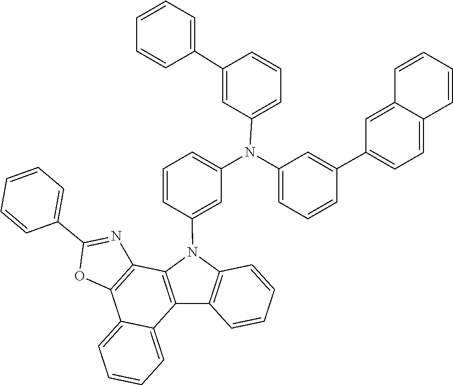 [SMILES: c1ccc(-c2cccc(N(c3cccc(-c4ccc5ccccc5c4)c3)c3cccc(-n4c5ccccc5c5c6ccccc6c6oc(-c7ccccc7)nc6c54)c3)c2)cc1] 158 | 780.30 | 78 |
| 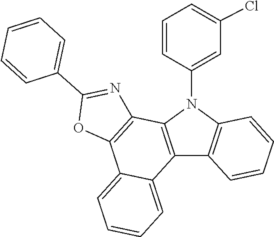 [SMILES: Clc1cccc(-n2c3ccccc3c3c4ccccc4c4oc(-c5ccccc5)nc4c32)c1] | 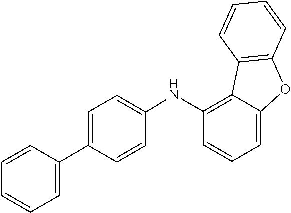 [SMILES: c1ccc(-c2ccc(Nc3cccc4oc5ccccc5c34)cc2)cc1] CAS: 1776057-10-3 | 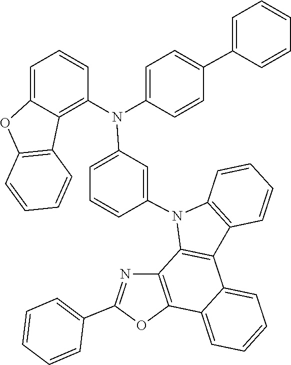 [SMILES: c1ccc(-c2ccc(N(c3cccc(-n4c5ccccc5c5c6ccccc6c6oc(-c7ccccc7)nc6c54)c3)c3cccc4oc5ccccc5c34)cc2)cc1] 163 | 744.26 | 71 |
| 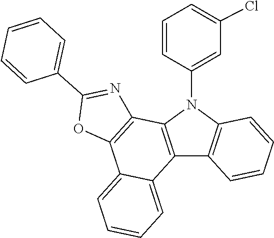 [SMILES: Clc1cccc(-n2c3ccccc3c3c4ccccc4c4oc(-c5ccccc5)nc4c32)c1] | 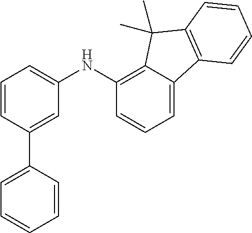 [SMILES: CC1(C)c2ccccc2-c2cccc(Nc3cccc(-c4ccccc4)c3)c21] CAS: 1644054-09-0 | 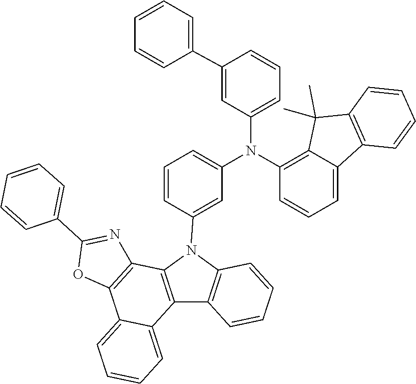 [SMILES: CC1(C)c2ccccc2-c2cccc(N(c3cccc(-c4ccccc4)c3)c3cccc(-n4c5ccccc5c5c6ccccc6c6oc(-c7ccccc7)nc6c54)c3)c21] 167 | 770.31 | 74 |
| 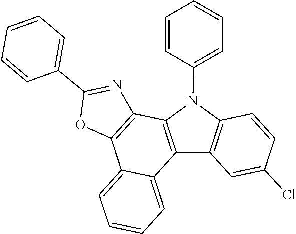 [SMILES: Clc1ccc2c(c1)c1c3ccccc3c3oc(-c4ccccc4)nc3c1n2-c1ccccc1] | 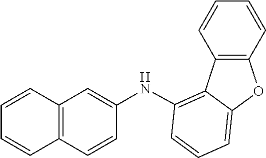 [SMILES: c1ccc2cc(Nc3cccc4oc5ccccc5c34)ccc2c1] CAS: 2230816-67-6 | 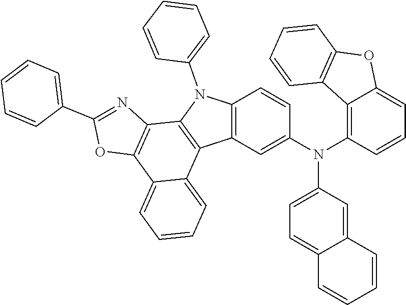 [SMILES: c1ccc(-c2nc3c(o2)c2ccccc2c2c4cc(N(c5ccc6ccccc6c5)c5cccc6oc7ccccc7c56)ccc4n(-c4ccccc4)c32)cc1] 223 | 718.24 | 75 |
| 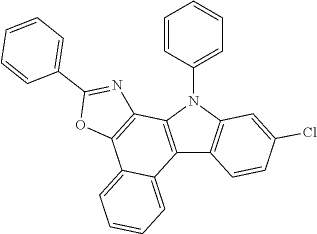 [SMILES: Clc1ccc2c3c4ccccc4c4oc(-c5ccccc5)nc4c3n(-c3ccccc3)c2c1] | 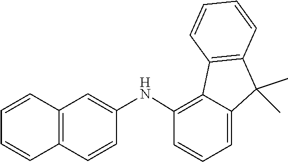 [SMILES: CC1(C)c2ccccc2-c2c(Nc3ccc4ccccc4c3)cccc21] CAS: 1613329-89-7 | 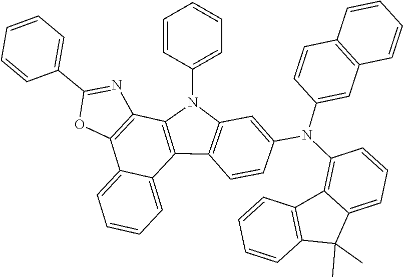 [SMILES: CC1(C)c2ccccc2-c2c(N(c3ccc4ccccc4c3)c3ccc4c5c6ccccc6c6oc(-c7ccccc7)nc6c5n(-c5ccccc5)c4c3)cccc21] 226 | 744.30 | 70 |
| 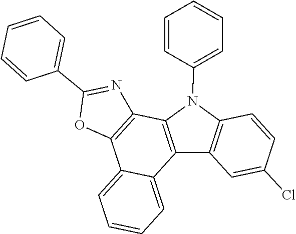 [SMILES: Clc1ccc2c(c1)c1c3ccccc3c3oc(-c4ccccc4)nc3c1n2-c1ccccc1] | 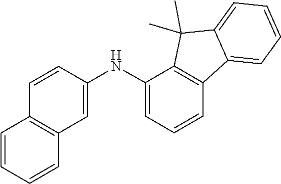 [SMILES: CC1(C)c2ccccc2-c2cccc(Nc3ccc4ccccc4c3)c21] CAS: 2089116-66-3 | 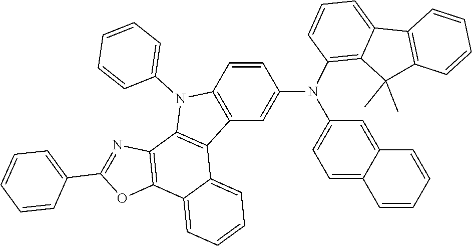 [SMILES: CC1(C)c2ccccc2-c2cccc(N(c3ccc4ccccc4c3)c3ccc4c(c3)c3c5ccccc5c5oc(-c6ccccc6)nc5c3n4-c3ccccc3)c21] 239 | 744.30 | 77 |
| 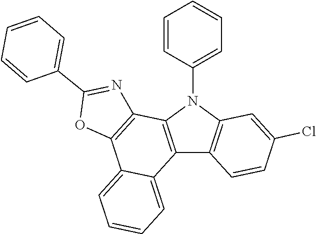 [SMILES: Clc1ccc2c3c4ccccc4c4oc(-c5ccccc5)nc4c3n(-c3ccccc3)c2c1] | 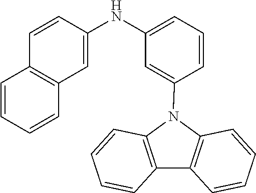 [SMILES: c1cc(Nc2ccc3ccccc3c2)cc(-n2c3ccccc3c3ccccc32)c1] CAS: 2570470-35-6 | 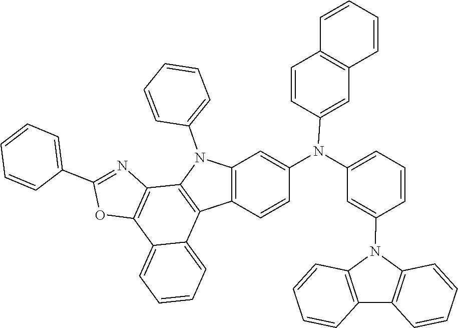 [SMILES: c1ccc(-c2nc3c(o2)c2ccccc2c2c4ccc(N(c5cccc(-n6c7ccccc7c7ccccc76)c5)c5ccc6ccccc6c5)cc4n(-c4ccccc4)c32)cc1] 242 | 793.29 | 84 |
| 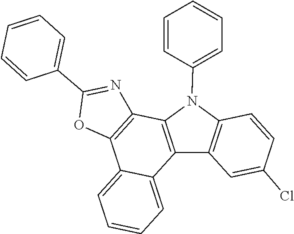 [SMILES: Clc1ccc2c(c1)c1c3ccccc3c3oc(-c4ccccc4)nc3c1n2-c1ccccc1] | 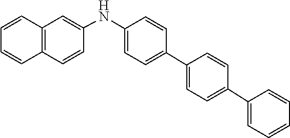 [SMILES: c1ccc(-c2ccc(-c3ccc(Nc4ccc5ccccc5c4)cc3)cc2)cc1] CAS: 897921-64-1 | 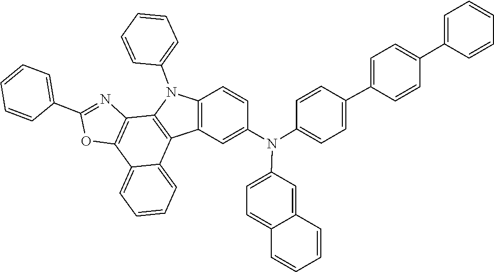 [SMILES: c1ccc(-c2ccc(-c3ccc(N(c4ccc5ccccc5c4)c4ccc5c(c4)c4c6ccccc6c6oc(-c7ccccc7)nc6c4n5-c4ccccc4)cc3)cc2)cc1] 247 | 780.30 | 85 |
| 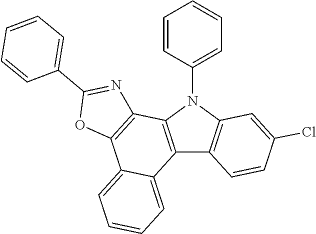 [SMILES: Clc1ccc2c3c4ccccc4c4oc(-c5ccccc5)nc4c3n(-c3ccccc3)c2c1] | 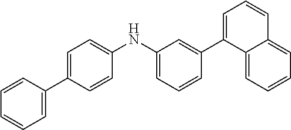 [SMILES: c1ccc(-c2ccc(Nc3cccc(-c4cccc5ccccc45)c3)cc2)cc1] CAS: 2634130-29-1 | 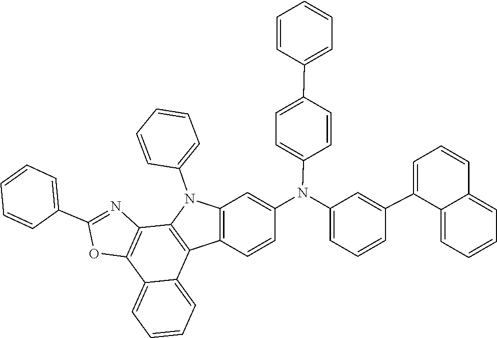 [SMILES: c1ccc(-c2ccc(N(c3cccc(-c4cccc5ccccc45)c3)c3ccc4c5c6ccccc6c6oc(-c7ccccc7)nc6c5n(-c5ccccc5)c4c3)cc2)cc1] 262 | 780.30 | 86 |
| 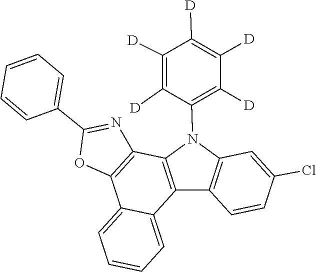 [SMILES: [2H]c1c([2H])c([2H])c(-n2c3cc(Cl)ccc3c3c4ccccc4c4oc(-c5ccccc5)nc4c32)c([2H])c1[2H]] | 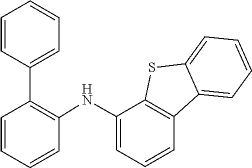 [SMILES: c1ccc(-c2ccccc2Nc2cccc3c2sc2ccccc23)cc1] CAS: 1894197-07-9 | 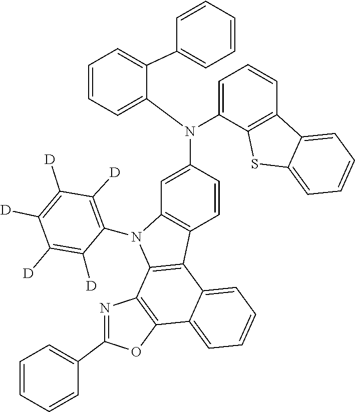 [SMILES: [2H]c1c([2H])c([2H])c(-n2c3cc(N(c4ccccc4-c4ccccc4)c4cccc5c4sc4ccccc45)ccc3c3c4ccccc4c4oc(-c5ccccc5)nc4c32)c([2H])c1[2H]] 270 | 765.27 | 76 |
| 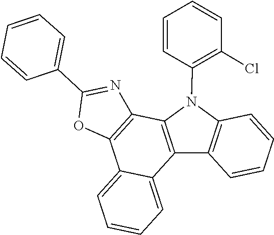 [SMILES: Clc1ccccc1-n1c2ccccc2c2c3ccccc3c3oc(-c4ccccc4)nc3c21] | 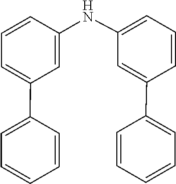 [SMILES: c1ccc(-c2cccc(Nc3cccc(-c4ccccc4)c3)c2)cc1] CAS: 169224-65-1 | 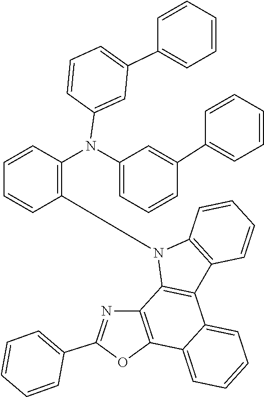 [SMILES: c1ccc(-c2cccc(N(c3cccc(-c4ccccc4)c3)c3ccccc3-n3c4ccccc4c4c5ccccc5c5oc(-c6ccccc6)nc5c43)c2)cc1] 301 | 730.28 | 65 |
| 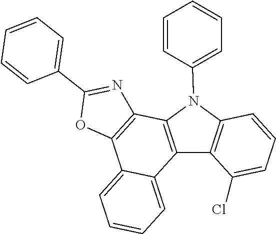 [SMILES: Clc1cccc2c1c1c3ccccc3c3oc(-c4ccccc4)nc3c1n2-c1ccccc1] | 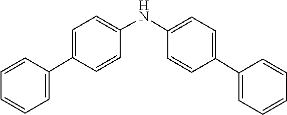 [SMILES: c1ccc(-c2ccc(Nc3ccc(-c4ccccc4)cc3)cc2)cc1] CAS: 102113-98-4 | 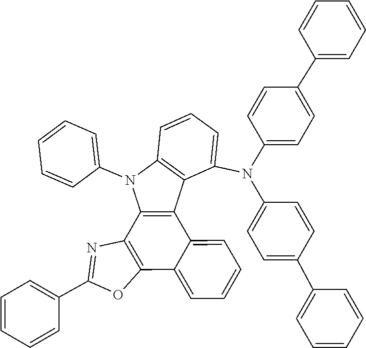 [SMILES: c1ccc(-c2ccc(N(c3ccc(-c4ccccc4)cc3)c3cccc4c3c3c5ccccc5c5oc(-c6ccccc6)nc5c3n4-c3ccccc3)cc2)cc1] 302 | 730.28 | 66 |
| 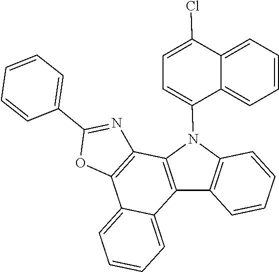 [SMILES: Clc1ccc(-n2c3ccccc3c3c4ccccc4c4oc(-c5ccccc5)nc4c32)c2ccccc12] | 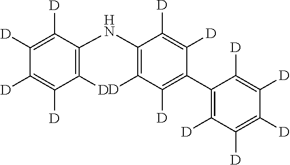 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] CAS: 1879963-18-4 | 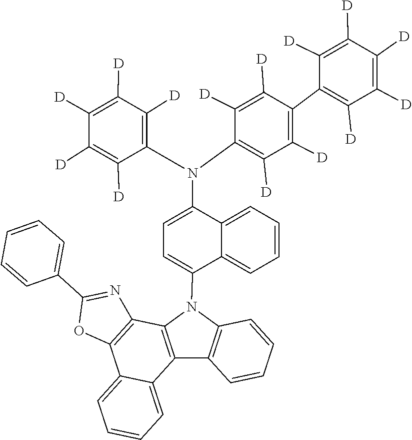 [SMILES: [2H]c1c([2H])c([2H])c(-c2c([2H])c([2H])c(N(c3c([2H])c([2H])c([2H])c([2H])c3[2H])c3ccc(-n4c5ccccc5c5c6ccccc6c6oc(-c7ccccc7)nc6c54)c4ccccc34)c([2H])c2[2H])c([2H])c1[2H]] 313 | 718.35 | 81 |
| 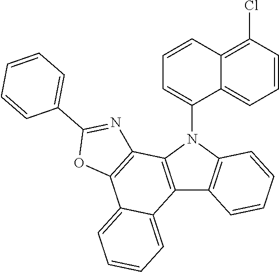 [SMILES: Clc1cccc2c(-n3c4ccccc4c4c5ccccc5c5oc(-c6ccccc6)nc5c43)cccc12] | 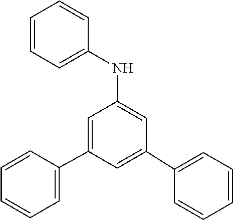 [SMILES: c1ccc(Nc2cc(-c3ccccc3)cc(-c3ccccc3)c2)cc1] CAS: 1065663-41-3 | 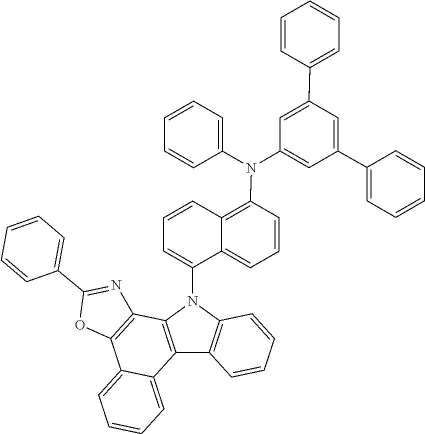 [SMILES: c1ccc(-c2cc(-c3ccccc3)cc(N(c3ccccc3)c3cccc4c(-n5c6ccccc6c6c7ccccc7c7oc(-c8ccccc8)nc7c65)cccc34)c2)cc1] 335 | 780.30 | 80 |
| 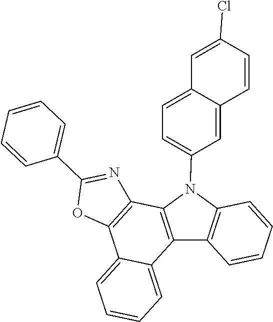 [SMILES: Clc1ccc2cc(-n3c4ccccc4c4c5ccccc5c5oc(-c6ccccc6)nc5c43)ccc2c1] | 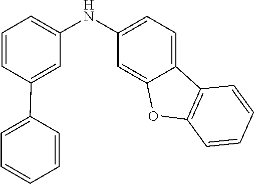 [SMILES: c1ccc(-c2cccc(Nc3ccc4c(c3)oc3ccccc34)c2)cc1] CAS: 1427556-45-3 | 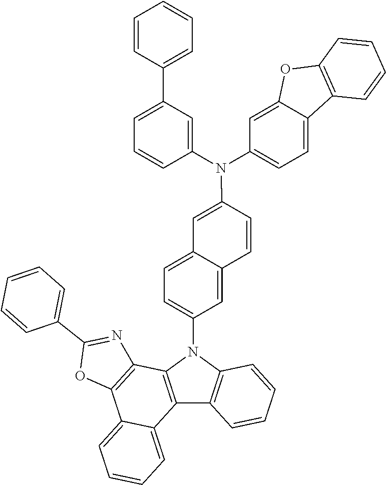 [SMILES: c1ccc(-c2cccc(N(c3ccc4cc(-n5c6ccccc6c6c7ccccc7c7oc(-c8ccccc8)nc7c65)ccc4c3)c3ccc4c(c3)oc3ccccc34)c2)cc1] 338 | 794.28 | 86 |
| 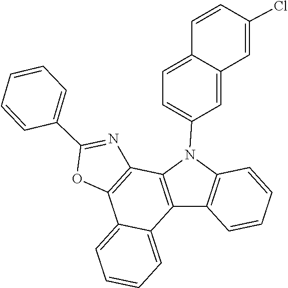 [SMILES: Clc1ccc2ccc(-n3c4ccccc4c4c5ccccc5c5oc(-c6ccccc6)nc5c43)cc2c1] | 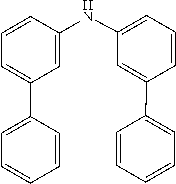 [SMILES: c1ccc(-c2cccc(Nc3cccc(-c4ccccc4)c3)c2)cc1] CAS: 169224-65-1 | 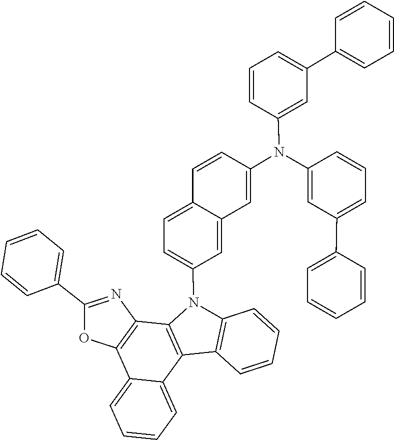 [SMILES: c1ccc(-c2cccc(N(c3cccc(-c4ccccc4)c3)c3ccc4ccc(-n5c6ccccc6c6c7ccccc7c7oc(-c8ccccc8)nc7c65)cc4c3)c2)cc1] 340 | 780.30 | 80 |
| 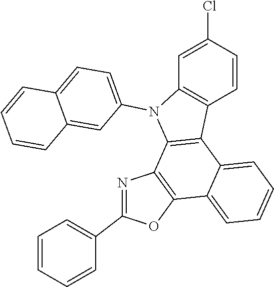 [SMILES: Clc1ccc2c3c4ccccc4c4oc(-c5ccccc5)nc4c3n(-c3ccc4ccccc4c3)c2c1] | 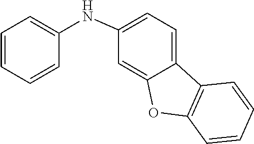 [SMILES: c1ccc(Nc2ccc3c(c2)oc2ccccc23)cc1] CAS: 406488-21-9 | 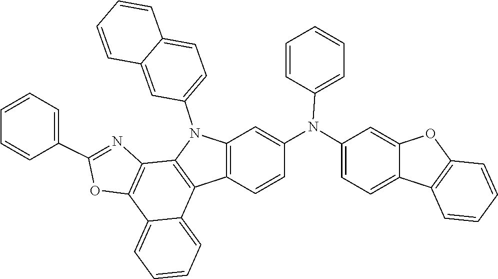 [SMILES: c1ccc(-c2nc3c(o2)c2ccccc2c2c4ccc(N(c5ccccc5)c5ccc6c(c5)oc5ccccc56)cc4n(-c4ccc5ccccc5c4)c32)cc1] 349 | 718.24 | 79 |
| 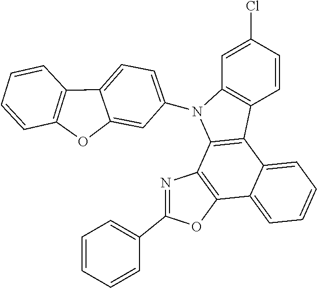 [SMILES: Clc1ccc2c3c4ccccc4c4oc(-c5ccccc5)nc4c3n(-c3ccc4c(c3)oc3ccccc34)c2c1] | 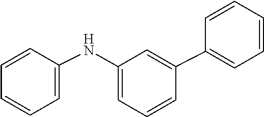 [SMILES: c1ccc(Nc2cccc(-c3ccccc3)c2)cc1] CAS: 198275-79-5 | 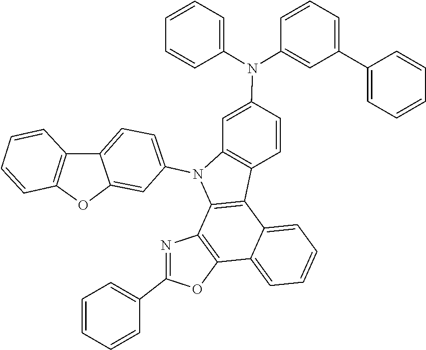 [SMILES: c1ccc(-c2cccc(N(c3ccccc3)c3ccc4c5c6ccccc6c6oc(-c7ccccc7)nc6c5n(-c5ccc6c(c5)oc5ccccc56)c4c3)c2)cc1] 351 | 744.26 | 83 |
| 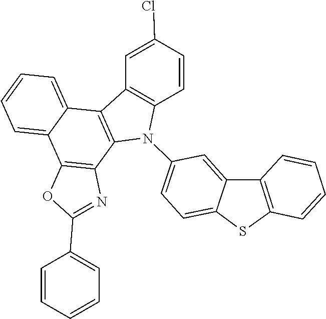 [SMILES: Clc1ccc2c(c1)c1c3ccccc3c3oc(-c4ccccc4)nc3c1n2-c1ccc2sc3ccccc3c2c1] | 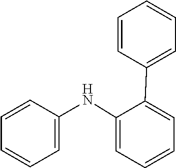 [SMILES: c1ccc(Nc2ccccc2-c2ccccc2)cc1] CAS: 35887-50-4 | 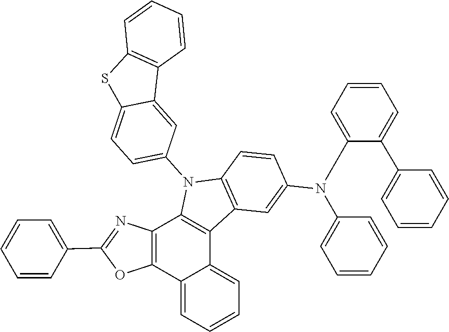 [SMILES: c1ccc(-c2nc3c(o2)c2ccccc2c2c4cc(N(c5ccccc5)c5ccccc5-c5ccccc5)ccc4n(-c4ccc5sc6ccccc6c5c4)c32)cc1] 352 | 760.24 | 67 |
| 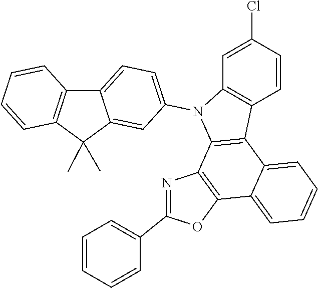 [SMILES: CC1(C)c2ccccc2-c2ccc(-n3c4cc(Cl)ccc4c4c5ccccc5c5oc(-c6ccccc6)nc5c43)cc21] | 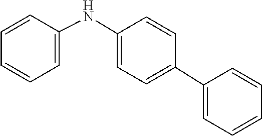 [SMILES: c1ccc(Nc2ccc(-c3ccccc3)cc2)cc1] CAS: 32228-99-2 | 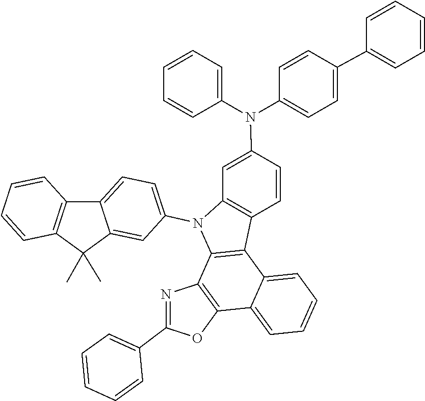 [SMILES: CC1(C)c2ccccc2-c2ccc(-n3c4cc(N(c5ccccc5)c5ccc(-c6ccccc6)cc5)ccc4c4c5ccccc5c5oc(-c6ccccc6)nc5c43)cc21] 353 | 770.31 | 73 |
| 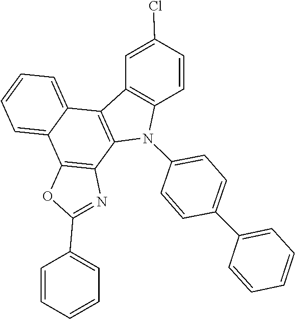 [SMILES: Clc1ccc2c(c1)c1c3ccccc3c3oc(-c4ccccc4)nc3c1n2-c1ccc(-c2ccccc2)cc1] | 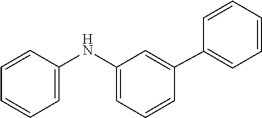 [SMILES: c1ccc(Nc2cccc(-c3ccccc3)c2)cc1] CAS: 198275-79-5 | 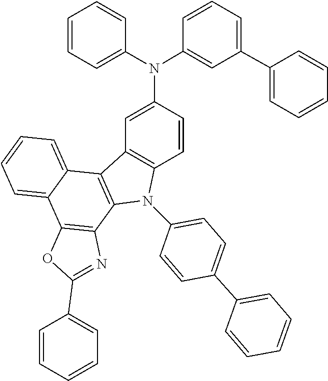 [SMILES: c1ccc(-c2ccc(-n3c4ccc(N(c5ccccc5)c5cccc(-c6ccccc6)c5)cc4c4c5ccccc5c5oc(-c6ccccc6)nc5c43)cc2)cc1] 356 | 730.28 | 82 |

The compounds synthesized above were subjected to 1H-NMR spectroscopy and the obtained data are shown in Table 5 below:

TABLE 5

| Compound | NMR data |
|---|---|
| Compound | ¹H-NMR (400 MHz, Methylene-Chloride-D2) δ ppm 8.41 (d, 1H), 8.29 (d, 2H), |
| 111 | 8.22 (d, 1H), 8.11 (d, 1H), 7.97 (d, 1H), 7.91 (d, 1H), 7.87 (t, 1H), 7.66-7.57 |
| | (m, 3H), 7.50-7.15 (m, 15H), 7.06-6.96 (m, 3H), 6.83 (d, 1H), 6.76-6.70 (m, |
| | 3H) |
| Compound | ¹H-NMR (400 MHz, Methylene-Chloride-D2) δ ppm 8.40 (d, 1H), 8.29 (d, 2H), |
| 270 | 8.16 (d, 1H), 8.11 (d, 1H), 7.99 (d, 1H), 7.96-7.84 (m, 3H), 7.66-7.54 (m, 3H), |
| | 7.50-7.25 (m, 10H), 7.18 (t, 1H), 7.11 (t, 1H), 6.95 (s, 1H), 6.80 (d, 1H), 6.74 |
| | (d, 1H), 6.69 (d, 1H) |

### Example 1: Manufacture of Red Organic Electroluminescent Device

An anode was pretreated by the following process: surface treatment was performed with UV ozone and O₂:N₂ plasma on an ITO/Ag/ITO substrate with a thickness of 100 Å, 1000 Å, and 100 Å to increase the work function of the anode, and the surface of the ITO substrate was washed with an organic solvent to remove impurities and oil stains from the surface of the ITO substrate.

PD and HT-1 was co-evaporated on an experimental substrate (the anode) at an evaporation rate ratio of 2%:98% to form a hole injection layer (HIL) with a thickness of 100 Å, and then HT-1 was vacuum-evaporated on the hole injection layer to form a hole transport layer with a thickness of 1065 Å. A Compound HT-2 was vacuum-evaporated on the hole transport layer to form a hole auxiliary layer with a thickness of 890 Å.

Next, a compound 15, RH-N and RD were co-evaporated on the hole auxiliary layer at a ratio of 49%:49%:2% to form a red organic electroluminescent layer (EML) with a thickness of 400 Å.

Compounds ET and LiQ were co-evaporated on the organic electroluminescent layer at an evaporation rate ratio of 1:1 to form an electron transport layer (ETL) with a thickness of 350 Å, Yb was evaporated on the electron transport layer to form an electron injection layer (EIL) with a thickness of 10 Å, and then magnesium (Mg) and silver (Ag) were mixed and vacuum-evaporated on the electron injection layer at an evaporation rate of 1:9 to form a cathode having a thickness of 130 Å.

In addition, a Compound CP-1 was vacuum-evaporated on the above cathode to form an organic capping layer (CPL) with a thickness of 800 Å, thus completing the manufacture of a red organic electroluminescent device.

### Examples 2 to 48

An organic electroluminescent device was manufactured by the same method as in that Example 1, except that a Compound X in Table 7 was used instead of Compound 15 in Example 1 when the organic electroluminescent layer was manufactured.

### Comparative Examples 1 to 3

An organic electroluminescent device was manufactured by the same method as that in Example 1, except that a Compound A, a Compound B, and a Compound C were respectively used instead of the Compound 15 in Example 1 when the organic electroluminescent layer was manufactured.

The structures of materials used in the above Examples and Comparative Examples are shown in Table 6 below:

TABLE 6 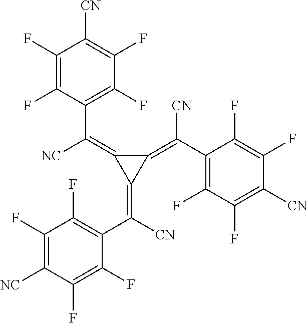 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]
PD 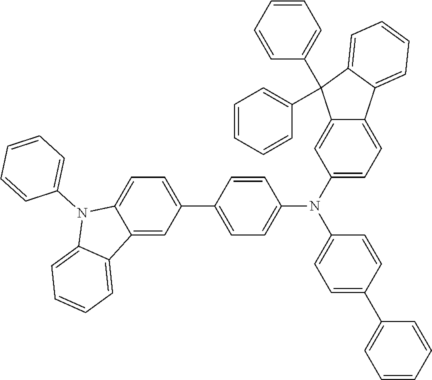 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]
HT-1 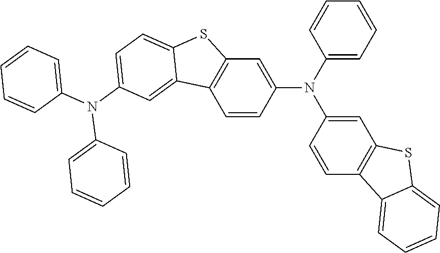 [SMILES: c1ccc(N(c2ccc3c(c2)sc2ccccc23)c2ccc3c(c2)sc2ccc(N(c4ccccc4)c4ccccc4)cc23)cc1]
HT-2 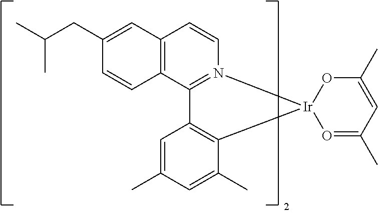
RD 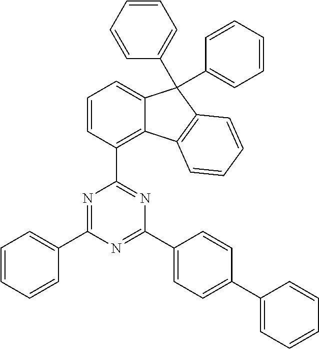 [SMILES: c1ccc(-c2ccc(-c3nc(-c4ccccc4)nc(-c4cccc5c4-c4ccccc4C5(c4ccccc4)c4ccccc4)n3)cc2)cc1]
ET 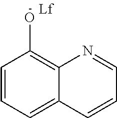 [SMILES: Oc1cccc2cccnc12]
LiQ 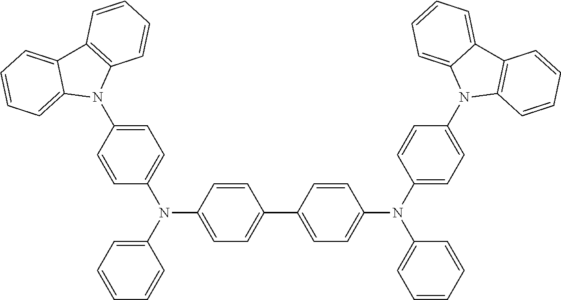 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1]
CP-1 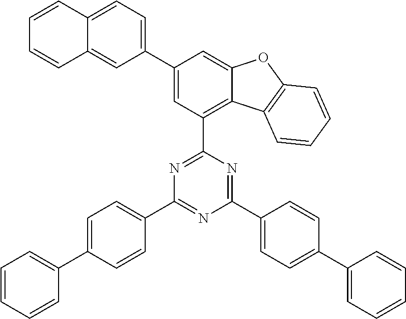 [SMILES: c1ccc(-c2ccc(-c3nc(-c4ccc(-c5ccccc5)cc4)nc(-c4cc(-c5ccc6ccccc6c5)cc5oc6ccccc6c45)n3)cc2)cc1]
RH-N 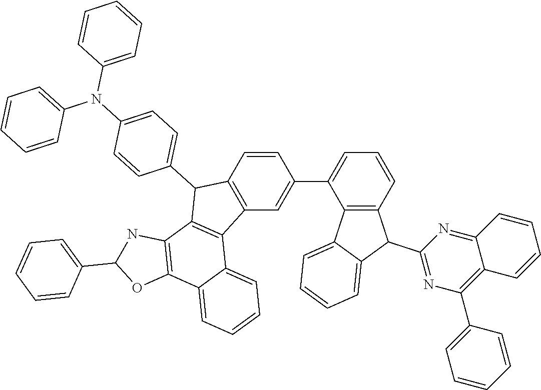 [SMILES: c1ccc(-c2nc(C3c4ccccc4-c4c(-c5ccc6c(c5)-c5c(c7c(c8ccccc58)OC(c5ccccc5)N7)C6c5ccc(N(c6ccccc6)c6ccccc6)cc5)cccc43)nc3ccccc23)cc1]
Compound A 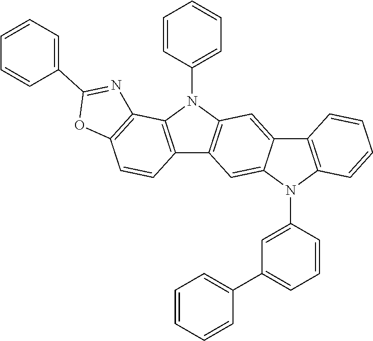 [SMILES: c1ccc(-c2cccc(-n3c4ccccc4c4cc5c(cc43)c3ccc4oc(-c6ccccc6)nc4c3n5-c3ccccc3)c2)cc1]
Compound B 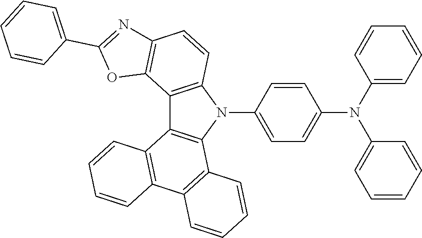 [SMILES: c1ccc(-c2nc3ccc4c(c3o2)c2c3ccccc3c3ccccc3c2n4-c2ccc(N(c3ccccc3)c3ccccc3)cc2)cc1]
Compound C

The red organic electroluminescent devices manufactured in Examples 1 to 48 and Comparative Examples 1 to 3 were subjected to performance tests. Specifically, the current-voltage-luminance (IVL) performance of the devices was tested under the condition of 10 mA/cm², and the T95 device service life was tested under the condition of 20 mA/cm². Test results are shown in Table 7 below.

TABLE 7

| | | Operating | | | | T95 (hrs) |
|---|---|---|---|---|---|---|
| | Compound | voltage | | | | @20 |
| No. | X | Volt (V) | Cd/A | CIEx | CIEy | mA/cm² |
| Example 1 | Compound 15 | 3.45 | 63.51 | 0.68 | 0.32 | 476 |
| Example 2 | Compound 23 | 3.43 | 64.00 | 0.68 | 0.32 | 481 |
| Example 3 | Compound 28 | 3.44 | 63.31 | 0.68 | 0.32 | 489 |
| Example 4 | Compound 40 | 3.45 | 63.90 | 0.68 | 0.32 | 479 |
| Example 5 | Compound 43 | 3.43 | 63.16 | 0.68 | 0.32 | 494 |
| Example 6 | Compound 45 | 3.43 | 63.20 | 0.68 | 0.32 | 484 |
| Example 7 | Compound 50 | 3.45 | 63.50 | 0.68 | 0.32 | 488 |
| Example 8 | Compound 51 | 3.44 | 64.07 | 0.68 | 0.32 | 496 |
| Example 9 | Compound 60 | 3.46 | 63.79 | 0.68 | 0.32 | 483 |
| Example 10 | Compound 71 | 3.44 | 64.90 | 0.68 | 0.32 | 491 |
| Example 11 | Compound 73 | 3.47 | 64.20 | 0.68 | 0.32 | 452 |
| Example 12 | Compound 77 | 3.43 | 64.10 | 0.68 | 0.32 | 492 |
| Example 13 | Compound 87 | 3.45 | 64.92 | 0.68 | 0.32 | 487 |
| Example 14 | Compound 89 | 3.46 | 64.25 | 0.68 | 0.32 | 493 |
| Example 15 | Compound 91 | 3.44 | 65.00 | 0.68 | 0.32 | 482 |
| Example 16 | Compound 92 | 3.43 | 63.66 | 0.68 | 0.32 | 493 |
| Example 17 | Compound 111 | 3.46 | 64.41 | 0.68 | 0.32 | 496 |
| Example 18 | Compound 115 | 3.46 | 63.43 | 0.68 | 0.32 | 477 |
| Example 19 | Compound 119 | 3.42 | 64.72 | 0.68 | 0.32 | 480 |
| Example 20 | Compound 124 | 3.46 | 64.30 | 0.68 | 0.32 | 482 |
| Example 21 | Compound 125 | 3.45 | 63.81 | 0.68 | 0.32 | 452 |
| Example 22 | Compound 130 | 3.43 | 64.70 | 0.68 | 0.32 | 478 |
| Example 23 | Compound 134 | 3.44 | 64.16 | 0.68 | 0.32 | 452 |
| Example 24 | Compound 135 | 3.47 | 63.80 | 0.68 | 0.32 | 481 |
| Example 25 | Compound 144 | 3.47 | 63.70 | 0.68 | 0.32 | 479 |
| Example 26 | Compound 147 | 3.42 | 63.25 | 0.68 | 0.32 | 492 |
| Example 27 | Compound 152 | 3.45 | 63.96 | 0.68 | 0.32 | 452 |
| Example 28 | Compound 158 | 3.45 | 63.41 | 0.68 | 0.32 | 484 |
| Example 29 | Compound 163 | 3.45 | 63.30 | 0.68 | 0.32 | 485 |
| Example 30 | Compound 167 | 3.46 | 64.28 | 0.68 | 0.32 | 495 |
| Example 31 | Compound 223 | 3.27 | 61.00 | 0.68 | 0.32 | 445 |
| Example 32 | Compound 226 | 3.28 | 60.72 | 0.68 | 0.32 | 452 |
| Example 33 | Compound 239 | 3.26 | 60.73 | 0.68 | 0.32 | 442 |
| Example 34 | Compound 242 | 3.28 | 61.27 | 0.68 | 0.32 | 453 |
| Example 35 | Compound 247 | 3.25 | 61.30 | 0.68 | 0.32 | 440 |
| Example 36 | Compound 262 | 3.27 | 60.60 | 0.68 | 0.32 | 441 |
| Example 37 | Compound 270 | 3.25 | 60.85 | 0.68 | 0.32 | 449 |
| Example 38 | Compound 301 | 3.45 | 63.78 | 0.68 | 0.32 | 486 |
| Example 39 | Compound 302 | 3.28 | 60.80 | 0.68 | 0.32 | 450 |
| Example 40 | Compound 313 | 3.47 | 64.40 | 0.68 | 0.32 | 494 |
| Example 41 | Compound 335 | 3.46 | 63.40 | 0.68 | 0.32 | 488 |
| Example 42 | Compound 338 | 3.42 | 64.12 | 0.68 | 0.32 | 490 |
| Example 43 | Compound 340 | 3.45 | 64.50 | 0.68 | 0.32 | 485 |
| Example 44 | Compound 349 | 3.25 | 61.70 | 0.68 | 0.32 | 444 |
| Example 45 | Compound 351 | 3.27 | 60.68 | 0.68 | 0.32 | 452 |
| Example 46 | Compound 352 | 3.25 | 60.60 | 0.68 | 0.32 | 447 |
| Example 47 | Compound 353 | 3.26 | 60.70 | 0.68 | 0.32 | 451 |
| Example 48 | Compound 356 | 3.25 | 61.20 | 0.68 | 0.32 | 446 |
| Comparative | Compound A | 3.65 | 51.10 | 0.68 | 0.32 | 392 |
| Example 1 | | | | | | |
| Comparative | Compound B | 3.33 | 53.30 | 0.68 | 0.32 | 357 |
| Example 2 | | | | | | |
| Comparative | Compound C | 3.45 | 54.20 | 0.68 | 0.32 | 376 |
| Example 3 | | | | | | |

As can be seen from the above Table 7, when the compounds of the present disclosure are used as host materials of red organic electroluminescent devices, the current efficiency is improved by at least 11.8% and the service life is improved by at least 12.2%. The reason is that the structure of the organic compound of the present disclosure includes a parent core structure of oxazolo/thiazolobenzo[C]carbazole, and the parent core is linked to an arylamine compound, and thus the organic compound is used as a host material of an organic electroluminescent layer. On one hand, benzo[C]carbazole itself has a large conjugated system and a suitable first excited triplet energy level, and fusing benzo[C]carbazole with an oxazole/thiazole ring can further increase the conjugated system, after linking this parent core structure to an aromatic amine, the intermolecular force can be enhanced and the carrier mobility of the compound can be increased; on the other hand, after oxazole/thiazole is fused with benzo[C]carbazole, radical cations formed from the arylamine compound during hole transport can be stabilized and the electrochemical stability of the compound can be improved. When the organic compound of the present disclosure is used as the host material of the organic electroluminescent layer, the carrier balance in a light-emitting layer can be improved, the carrier recombination region can be broadened, the exciton generation and utilization efficiency can be increased, and the luminous efficiency and service life of the device can be increased.

1. An organic compound, having a structure shown in a Formula 1: 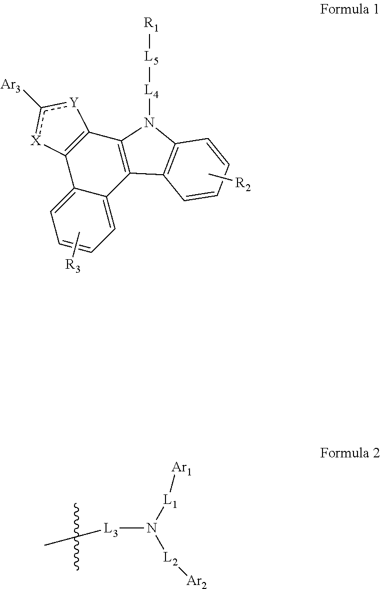
and wherein X and Y are each independently selected from 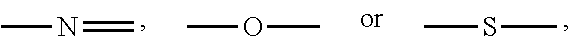
one and only one of X and Y is 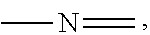
and the other is 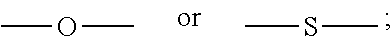
R₁ is selected from a hydrogen, a deuterium, or a structure represented by a Formula 2;
R₂ and R₃ are the same or different, and are each independently selected from a hydrogen, a deuterium, a cyano, a halogen group, an alkyl with 1 to 10 carbon atoms, a haloalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, a cycloalkyl with 3 to 10 carbon atoms, or the structure represented by the Formula 2;
and at least one of R₁, R₂, and R₃ is the group represented by the Formula 2;
L₁, L₂, L₃ and La are the same or different, and are each independently selected from a single bond, a substituted or unsubstituted arylene with 6 to 30 carbon atoms, or a substituted or unsubstituted heteroarylene with 3 to 30 carbon atoms;
L₅ is selected from a substituted or unsubstituted arylene with 6 to 30 carbon atoms, or a substituted or unsubstituted heteroarylene with 3 to 30 carbon atoms;
Ar₁, Ar₂, and Ar₃ are the same or different, and are each independently selected from a substituted or unsubstituted aryl with 6 to 30 carbon atoms, or a substituted or unsubstituted heteroaryl with 3 to 30 carbon atoms;
substituent(s) in L₁, L₂, L₃, L₄, L₅, Ar₁, Ar₂, and Ar₃ are the same or different, and are each independently selected from a deuterium, a cyano, a halogen group, an alkyl with 1 to 10 carbon atoms, a haloalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, or a cycloalkyl with 3 to 10 carbon atoms; and
optionally, in Ar₁ and Ar₂, any two adjacent substituents form a ring.

2. The organic compound according to claim 1, having a structure as shown in a Formula AA or a Formula BB below: 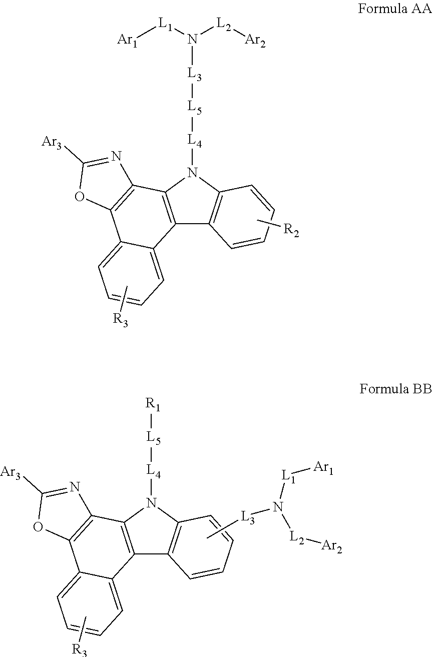
wherein in the formula BB, R₁ is a hydrogen or a deuterium.

3. The organic compound according to claim 1, wherein L₁, L₂, L₃ and L₄ are the same or different, and are each independently selected from a single bond, a substituted or unsubstituted phenylene, a substituted or unsubstituted biphenylene, a substituted or unsubstituted naphthylene, a substituted or unsubstituted fluorenylidene, a substituted or unsubstituted phenanthrylene, a substituted or unsubstituted carbazolylene, a substituted or unsubstituted dibenzofurylene, or a substituted or unsubstituted dibenzothenylene; and
optionally, substituent(s) in L₁, L₂, L₃ and L₄ are the same or different, and are each independently selected from a deuterium, a fluorine, a cyano, a methyl, an ethyl, a n-propyl, an isopropyl, a tert-butyl or a phenyl.

4. The organic compound according to claim 1, wherein Ar₁ and Ar₂ are the same or different, and are each independently selected from a substituted or unsubstituted aryl with 6 to 25 carbon atoms, or a substituted or unsubstituted heteroaryl with 12 to 24 carbon atoms;
optionally, substituent(s) in Ar₁ and Ar₂ are the same or different, and are each independently selected from a deuterium, a halogen group, a cyano, an alkyl with 1 to 5 carbon atoms, an aryl with 6 to 12 carbon atoms, or a pentadeuterophenyl;
optionally, in Ar₁ and Ar₂, any two adjacent substituents form a fluorene ring.

5. The organic compound according to claim 1, wherein Ar₁ and Ar₂ are the same or different, and are each independently selected from a substituted or unsubstituted phenyl, a substituted or unsubstituted naphthyl, a substituted or unsubstituted phenanthryl, a substituted or unsubstituted biphenyl, a substituted or unsubstituted terphenyl, a substituted or unsubstituted fluorenyl, a substituted or unsubstituted triphenylene, a substituted or unsubstituted 9,9′-spirobifluorenyl, a substituted or unsubstituted dibenzofuranyl, a substituted or unsubstituted dibenzothienyl, or a substituted or unsubstituted carbazolyl; and
optionally, substituent(s) in Ar₁ and Ar₂ are the same or different, and are each independently selected from a deuterium, a fluorine, a cyano, a methyl, an ethyl, a n-propyl, an isopropyl, a tert-butyl, a phenyl, a naphthyl or a pentadeuterophenyl.

6. The organic compound according to claim 1, wherein 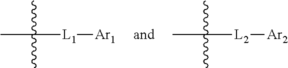
are the same or different, and are each independently selected from the group consisting of the following groups:

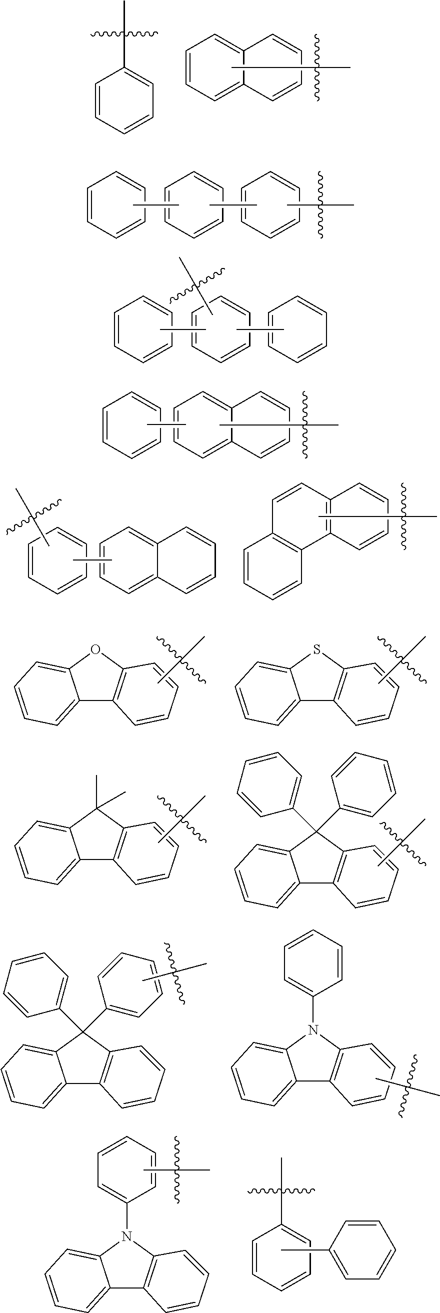

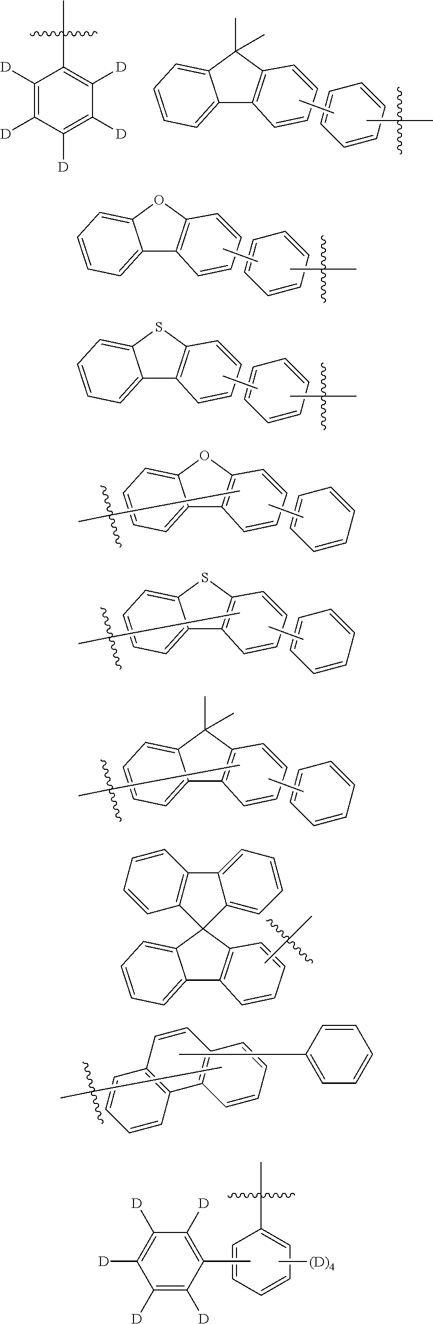

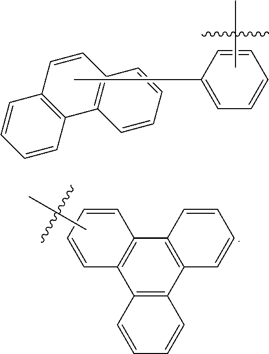

7. The organic compound according to claim 1, wherein L₅ is selected from a substituted or unsubstituted phenylene, a substituted or unsubstituted naphthylene, a substituted or unsubstituted biphenylene, a substituted or unsubstituted dibenzofurylene, a substituted or unsubstituted dibenzothenylene, or a substituted or unsubstituted fluorenylidene; and
optionally, substituent(s) in L₅ are the same or different, and are each independently selected from a deuterium, a fluorine, a cyano, a methyl, an ethyl, a n-propyl, an isopropyl, a tert-butyl or a phenyl.

8. The organic compound according to claim 1, wherein 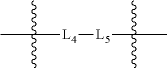
is selected from the group consisting of the following groups: 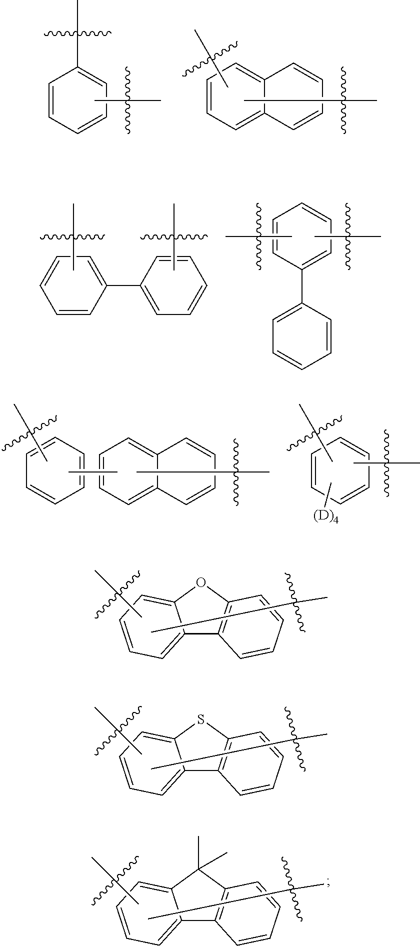
and
L₃ is selected from a single bond or the group consisting of the following groups:

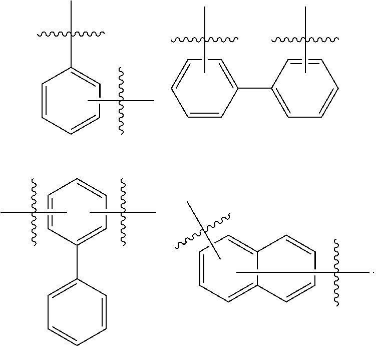

9. The organic compound according to claim 1, wherein Ar₃ is selected from a substituted or unsubstituted phenyl, a substituted or unsubstituted naphthyl, a substituted or unsubstituted biphenyl, a substituted or unsubstituted fluorenyl, a substituted or unsubstituted dibenzofuranyl, a substituted or unsubstituted dibenzothienyl, or a substituted or unsubstituted carbazolyl; and
optionally, substituent(s) in Ar₃ are the same or different, and are each independently selected from a deuterium, a fluorine, a cyano, a methyl, an ethyl, a n-propyl, an isopropyl, a tert-butyl or a phenyl.

10. The organic compound according to claim 1, wherein Ar₃ is selected from the group consisting of the following groups:

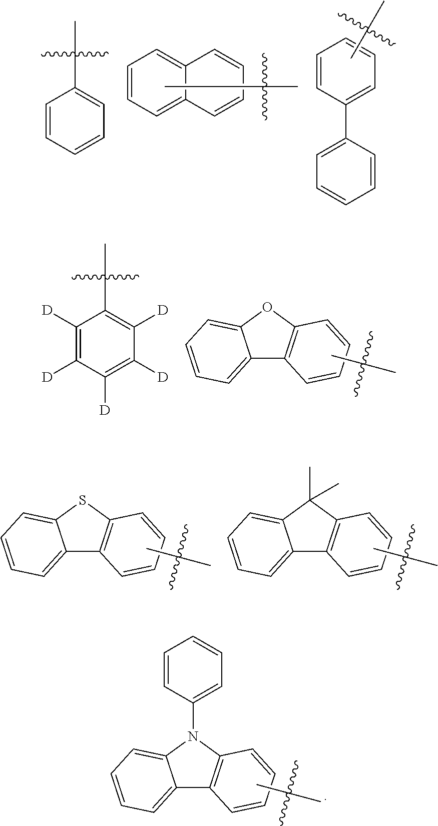

11. The organic compound according to claim 1, wherein R₁ is selected from a hydrogen, a deuterium, or the structure represented by the Formula 2;
R₂ and R₃ are the same or different, and are each independently selected from a hydrogen or the structure represented by the Formula 2;
and one and only one of R₁, R₂, and R₃ is the group structure represented by the Formula 2.

12. The organic compound according to claim 1, wherein the organic compound is selected from the group consisting of the following compounds:

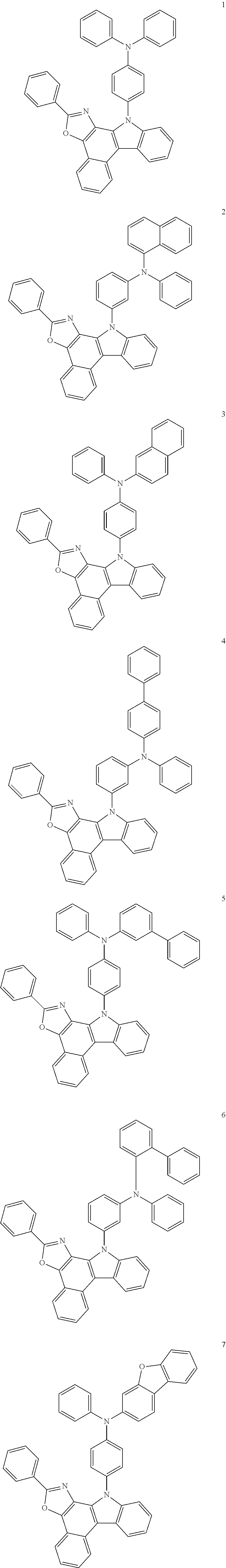

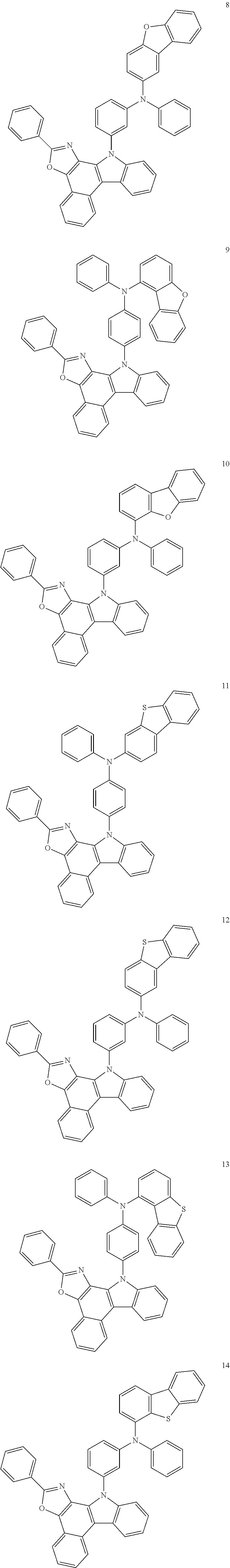

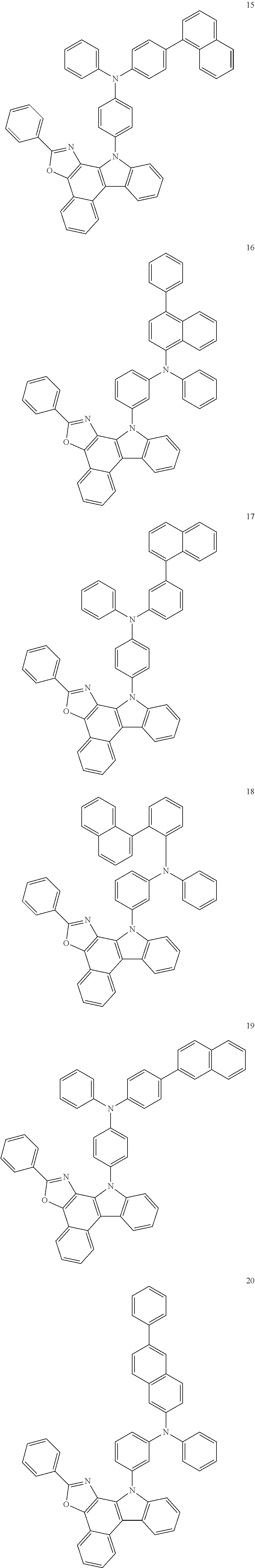

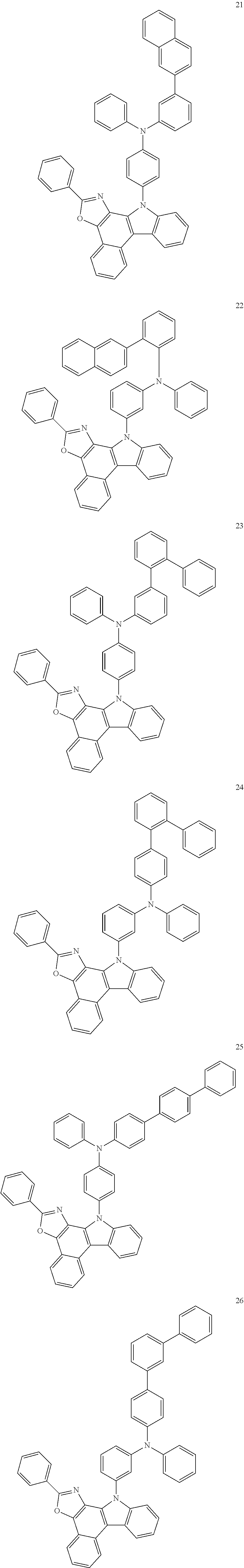

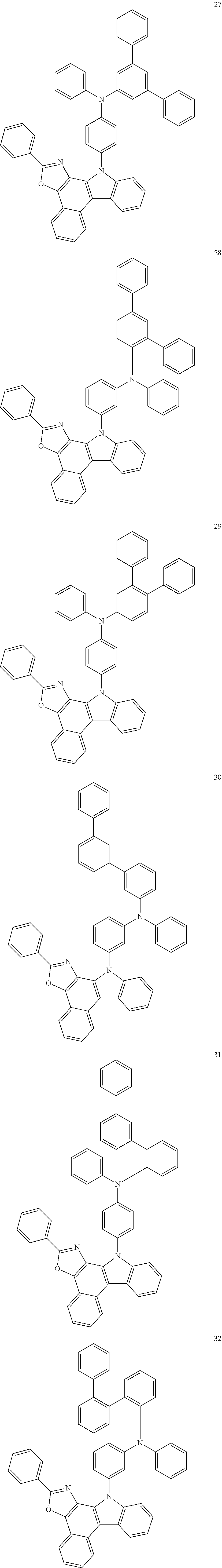

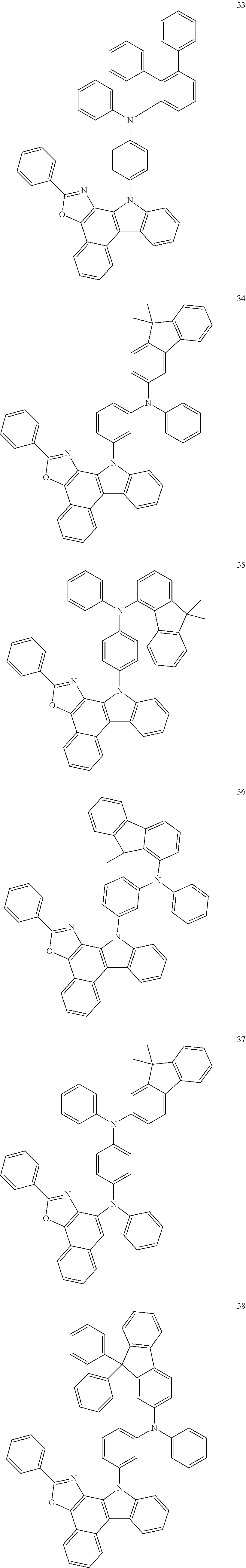

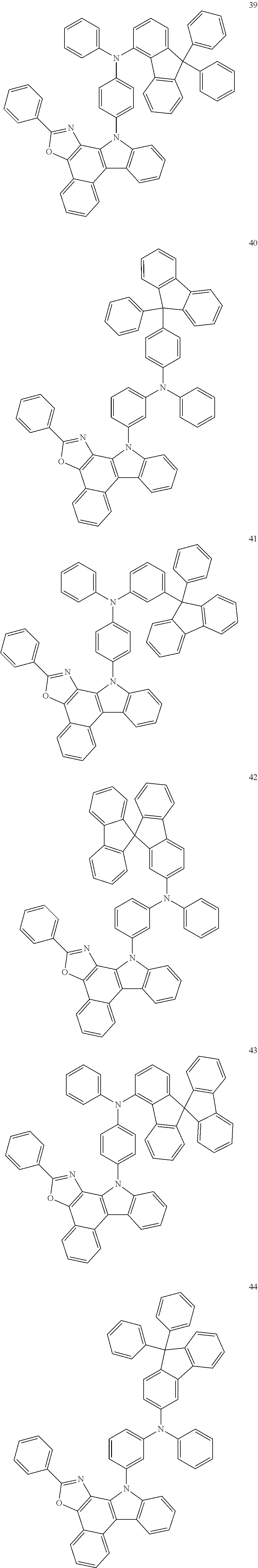

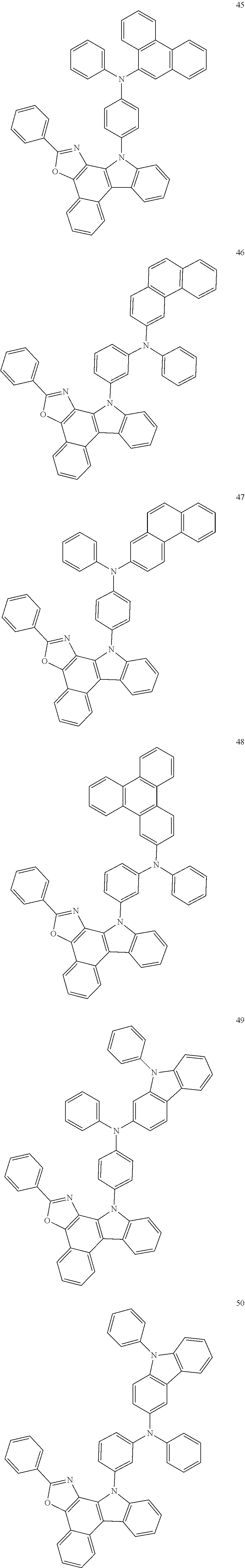

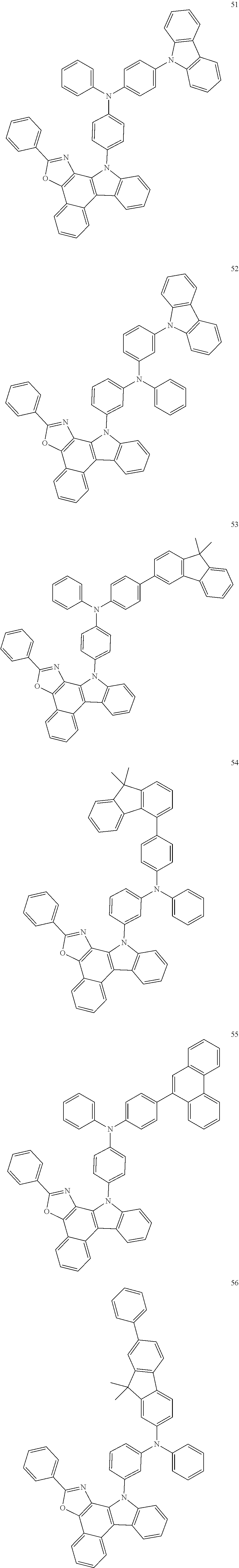

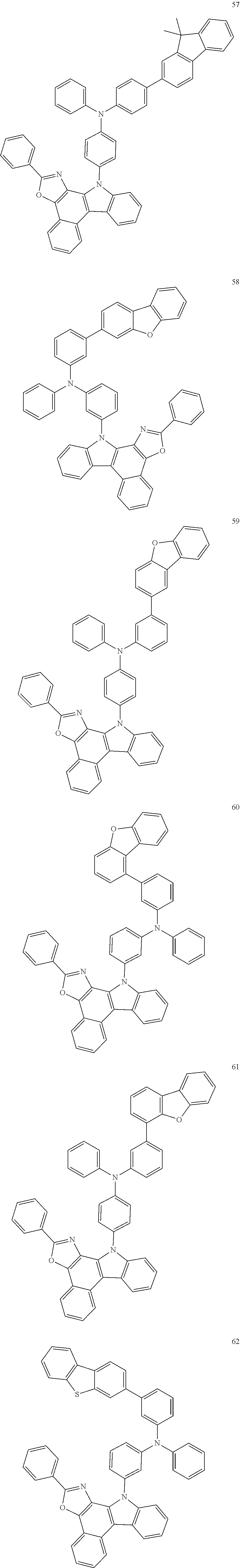

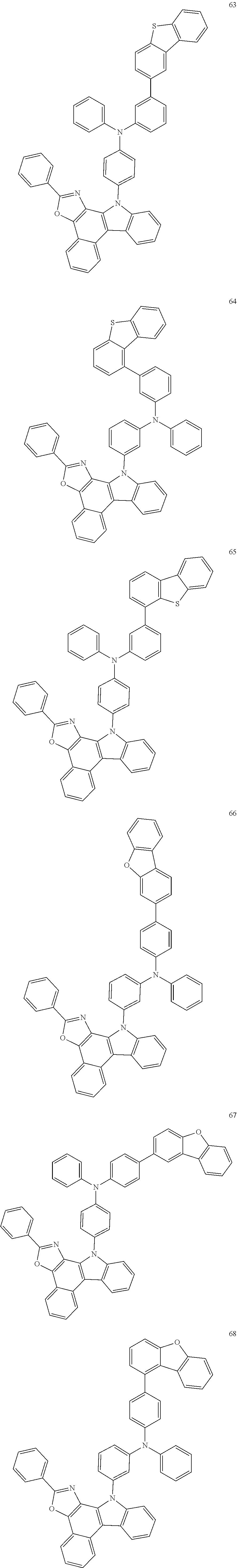

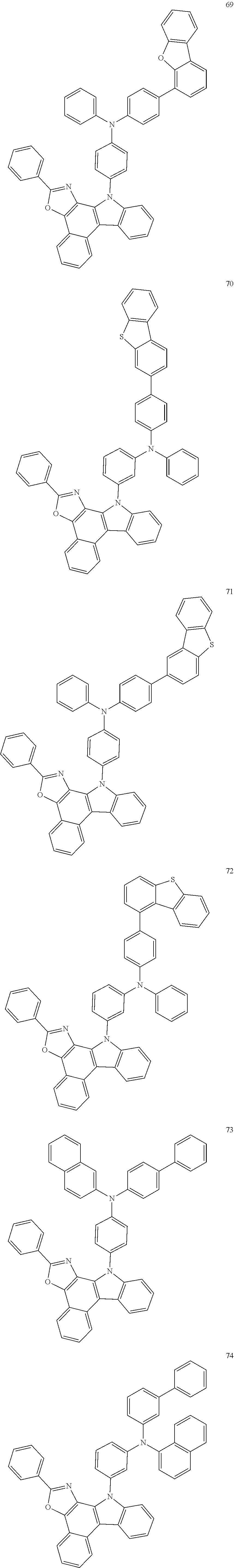

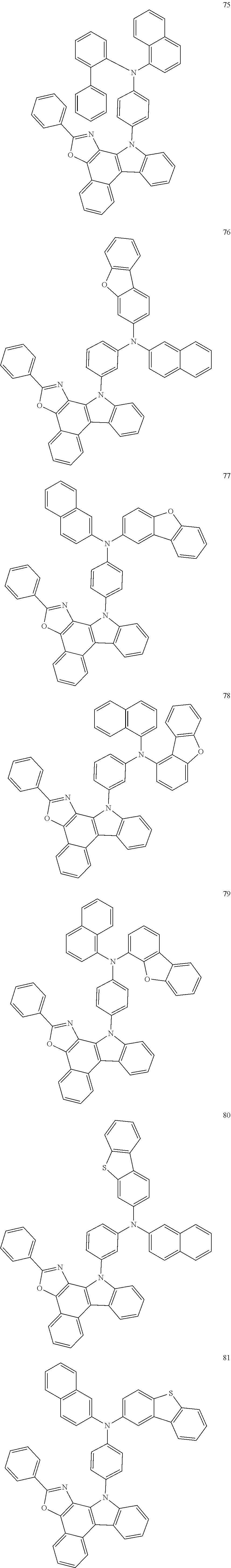

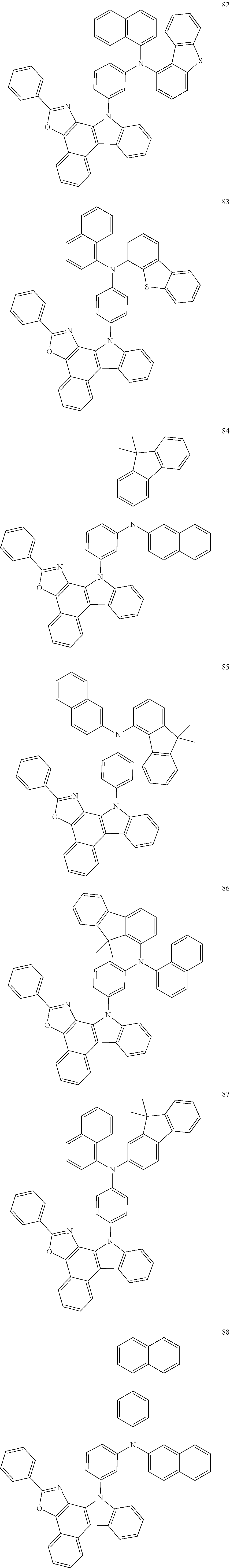

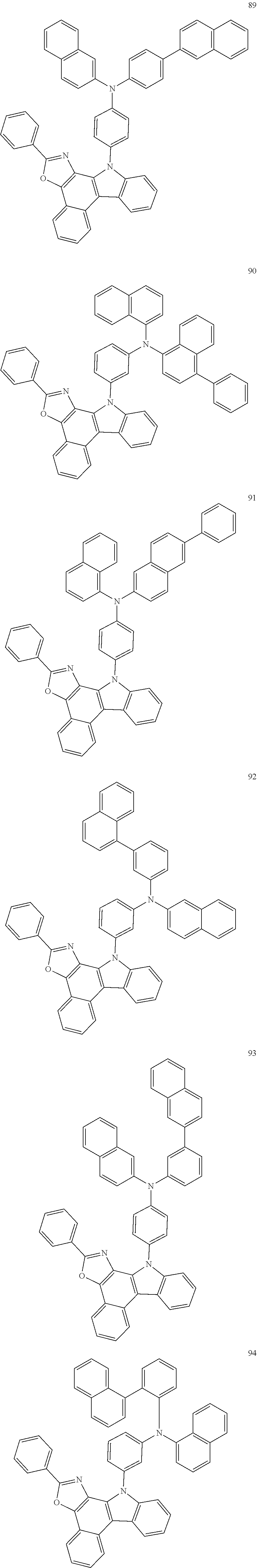

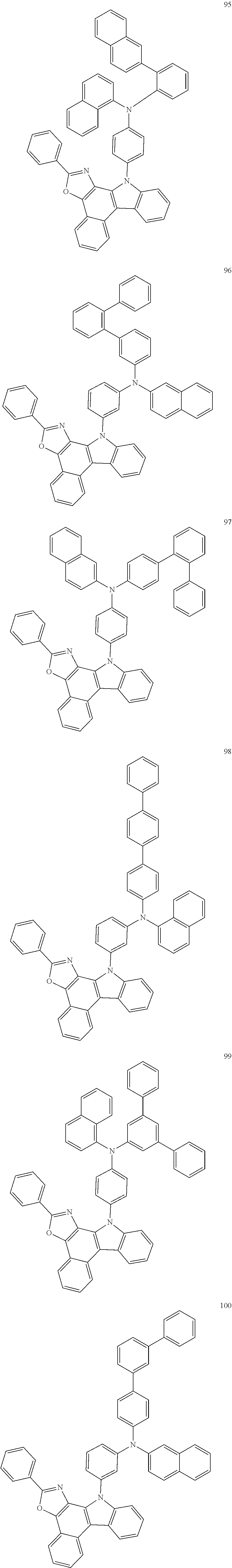

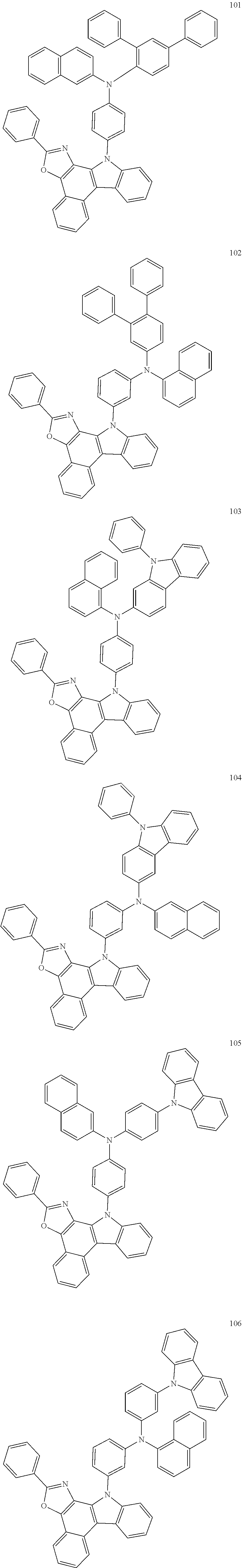

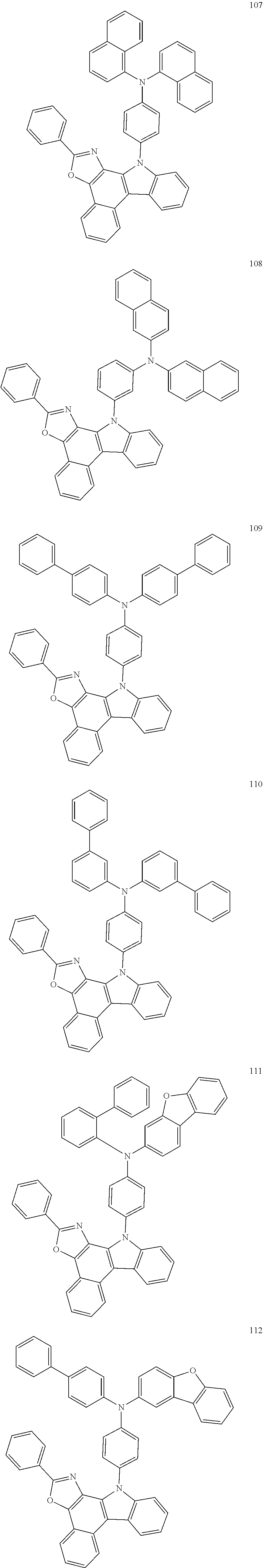

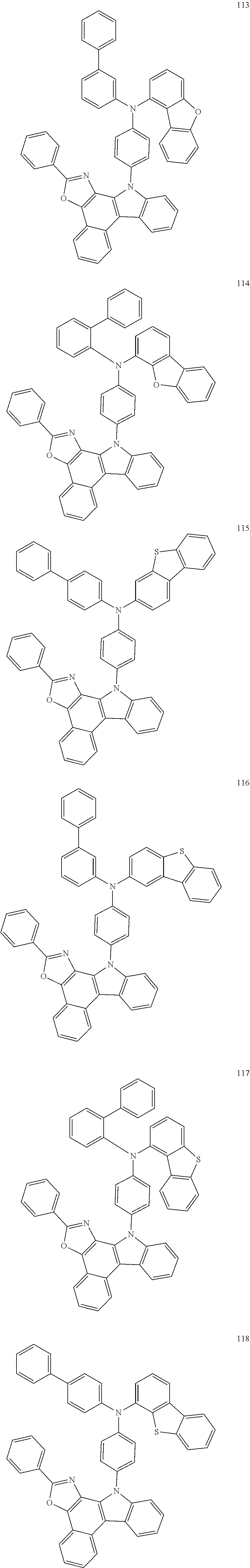

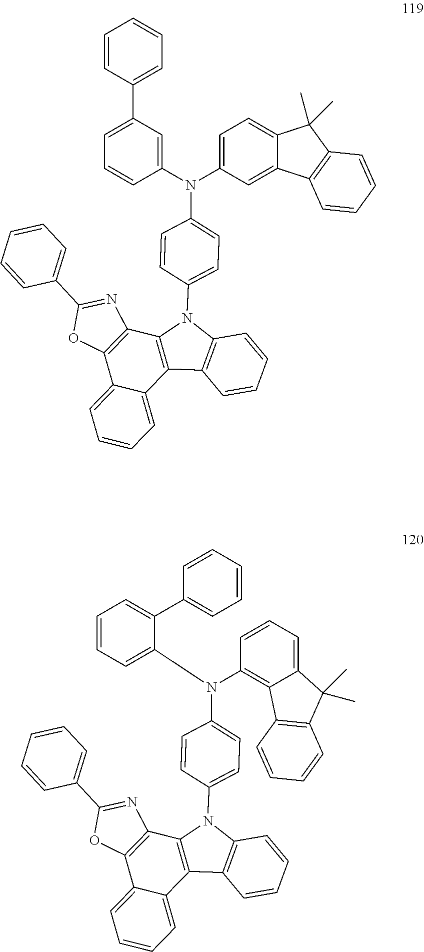 [SMILES: CC1(C)c2ccccc2-c2cc(N(c3ccc(-n4c5ccccc5c5c6ccccc6c6oc(-c7ccccc7)nc6c54)cc3)c3cccc(-c4ccccc4)c3)ccc21 ; CC1(C)c2ccccc2-c2c(N(c3ccc(-n4c5ccccc5c5c6ccccc6c6oc(-c7ccccc7)nc6c54)cc3)c3ccccc3-c3ccccc3)cccc21]

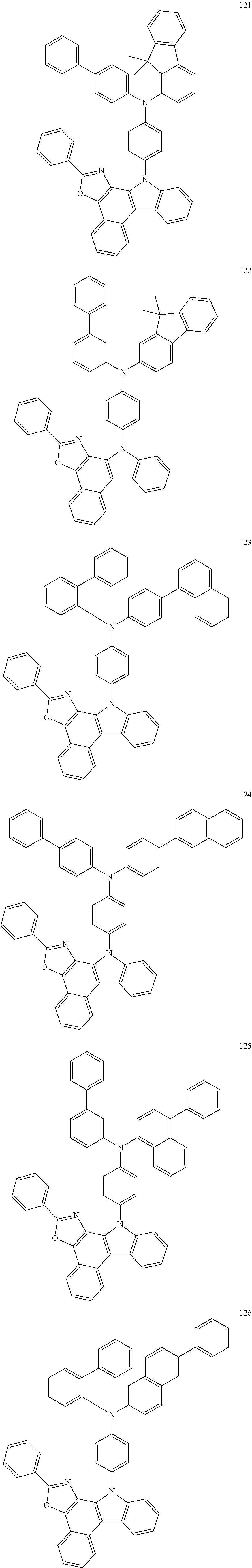

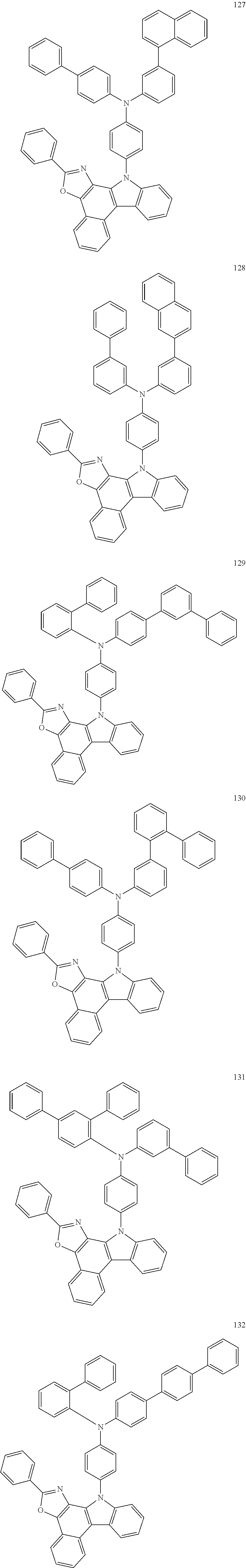

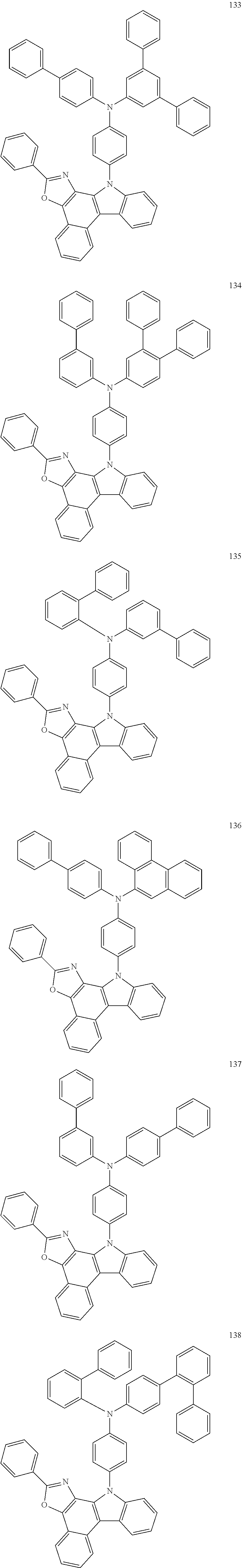

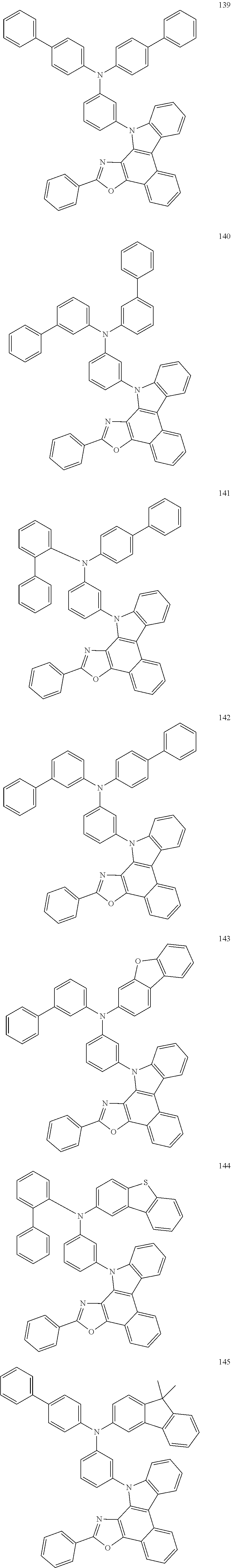

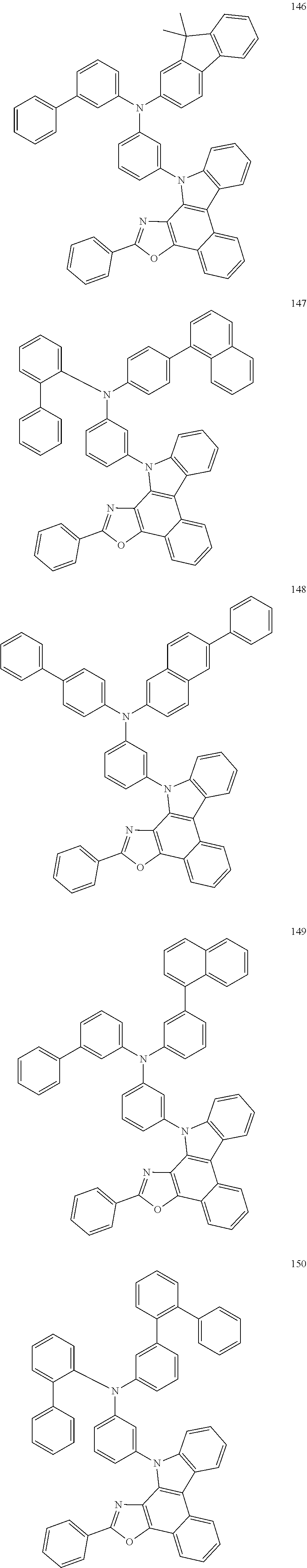

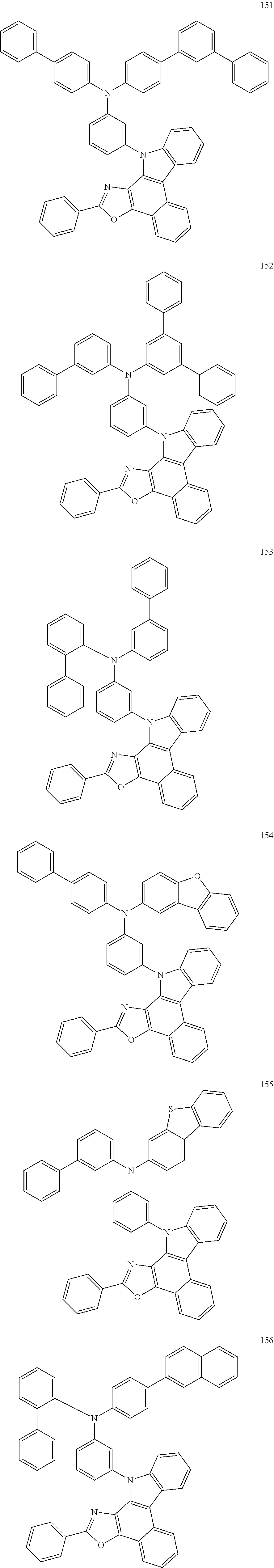

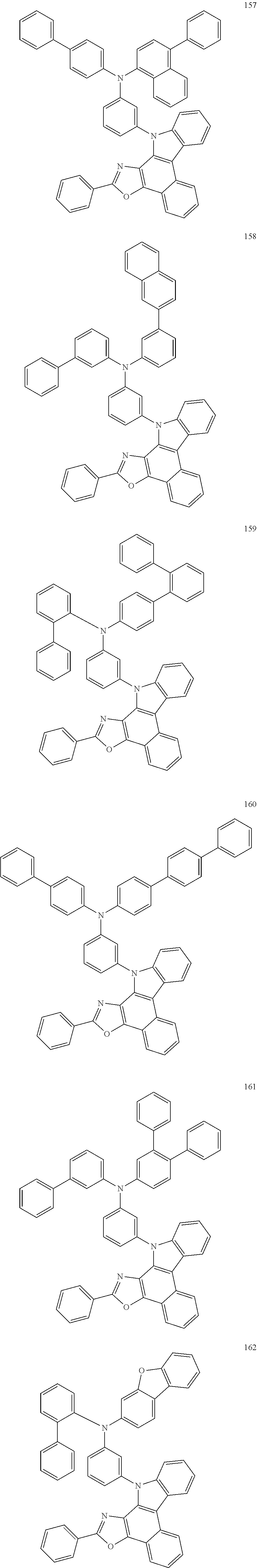

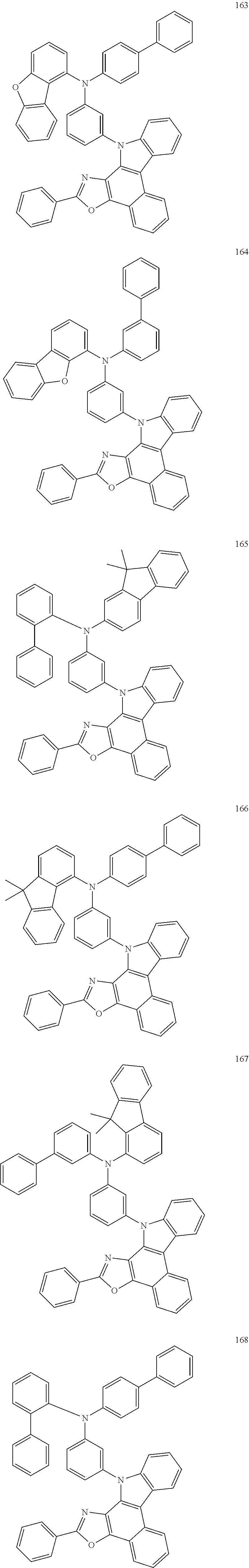

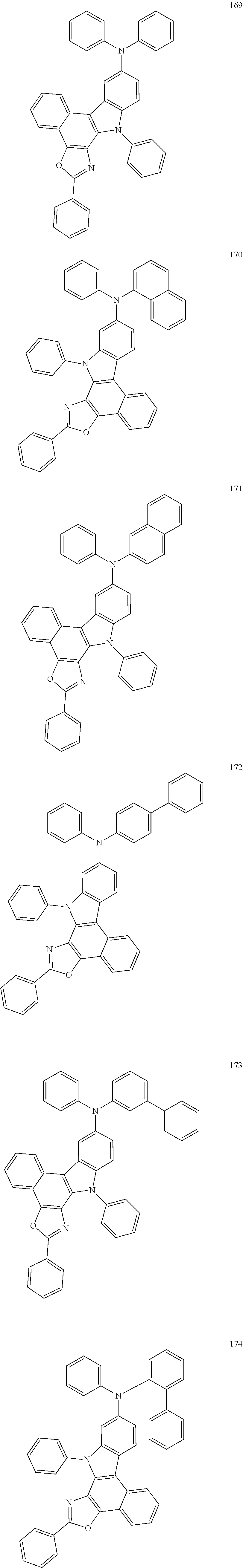

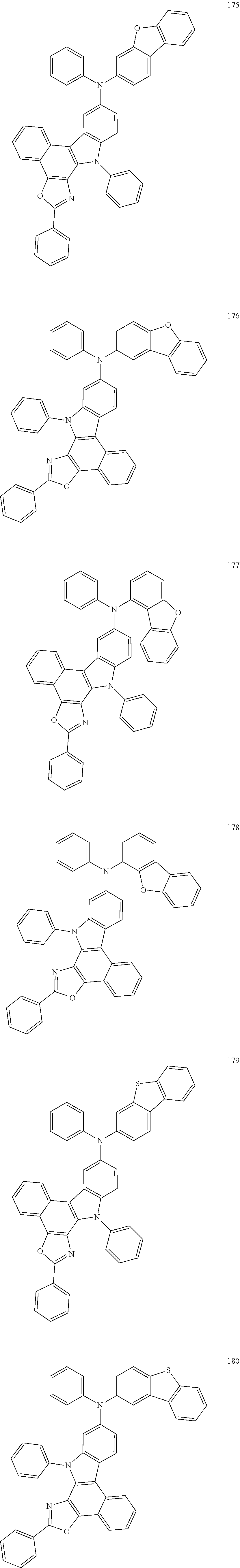

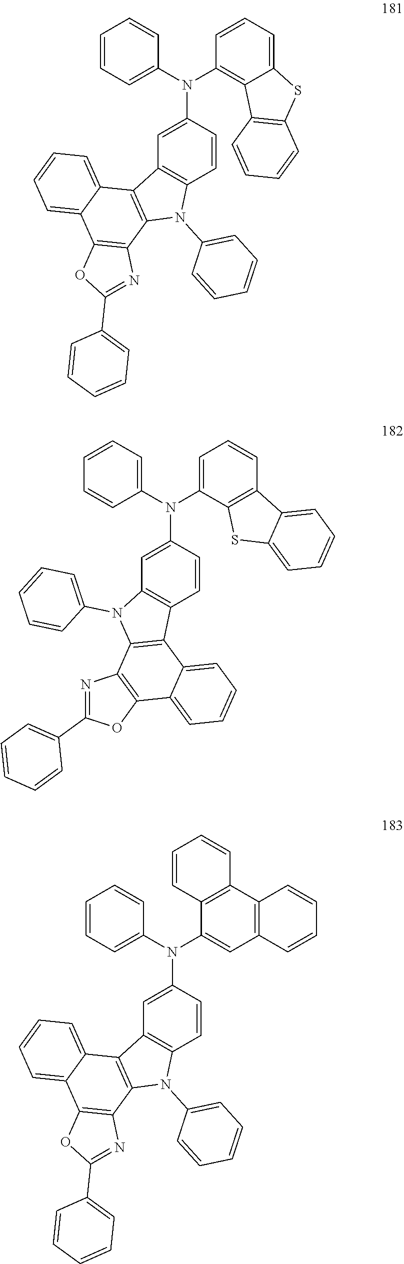 [SMILES: c1ccc(-c2nc3c(o2)c2ccccc2c2c4cc(N(c5ccccc5)c5cccc6sc7ccccc7c56)ccc4n(-c4ccccc4)c32)cc1 ; c1ccc(-c2nc3c(o2)c2ccccc2c2c4ccc(N(c5ccccc5)c5cccc6c5sc5ccccc56)cc4n(-c4ccccc4)c32)cc1 ; c1ccc(-c2nc3c(o2)c2ccccc2c2c4cc(N(c5ccccc5)c5cc6ccccc6c6ccccc56)ccc4n(-c4ccccc4)c32)cc1]

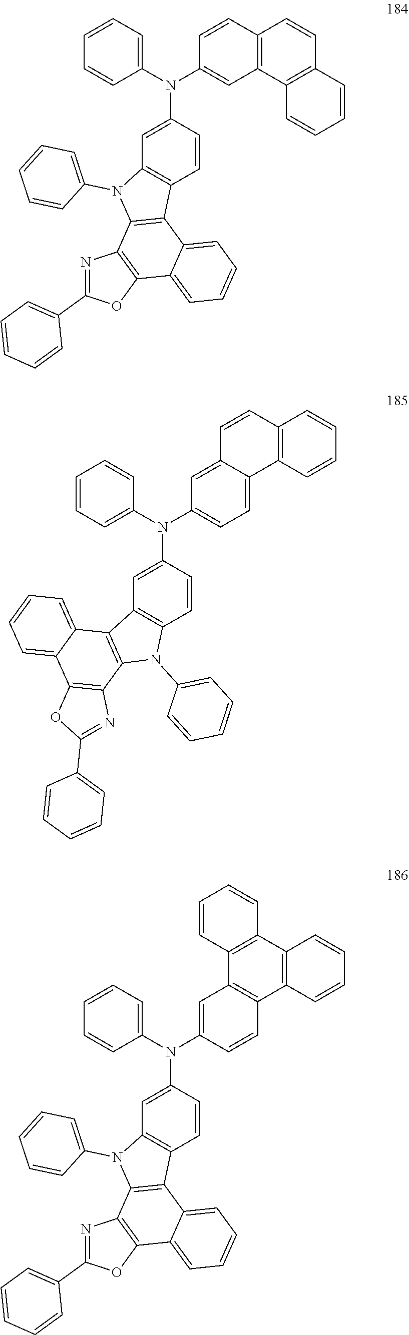 [SMILES: c1ccc(-c2nc3c(o2)c2ccccc2c2c4ccc(N(c5ccccc5)c5ccc6ccc7ccccc7c6c5)cc4n(-c4ccccc4)c32)cc1 ; c1ccc(-c2nc3c(o2)c2ccccc2c2c4cc(N(c5ccccc5)c5ccc6c(ccc7ccccc76)c5)ccc4n(-c4ccccc4)c32)cc1 ; c1ccc(-c2nc3c(o2)c2ccccc2c2c4ccc(N(c5ccccc5)c5ccc6c7ccccc7c7ccccc7c6c5)cc4n(-c4ccccc4)c32)cc1]

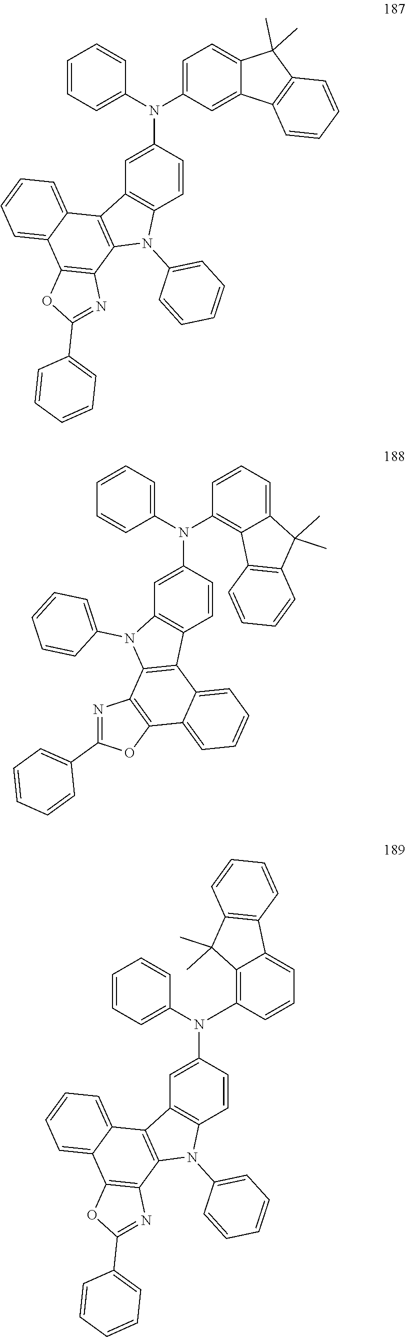 [SMILES: CC1(C)c2ccccc2-c2cc(N(c3ccccc3)c3ccc4c(c3)c3c5ccccc5c5oc(-c6ccccc6)nc5c3n4-c3ccccc3)ccc21 ; CC1(C)c2ccccc2-c2c(N(c3ccccc3)c3ccc4c5c6ccccc6c6oc(-c7ccccc7)nc6c5n(-c5ccccc5)c4c3)cccc21 ; CC1(C)c2ccccc2-c2cccc(N(c3ccccc3)c3ccc4c(c3)c3c5ccccc5c5oc(-c6ccccc6)nc5c3n4-c3ccccc3)c21]

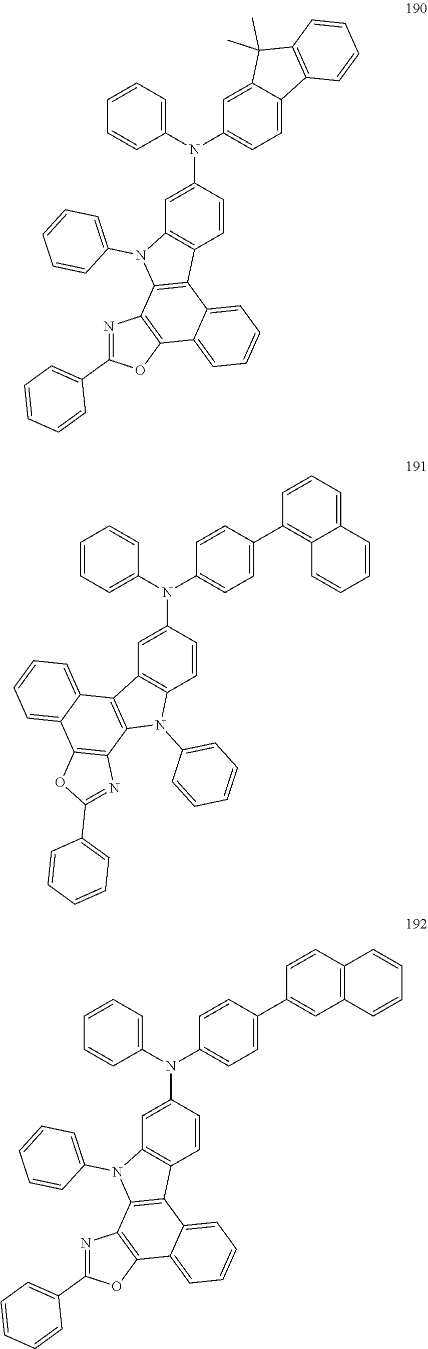 [SMILES: CC1(C)c2ccccc2-c2ccc(N(c3ccccc3)c3ccc4c5c6ccccc6c6oc(-c7ccccc7)nc6c5n(-c5ccccc5)c4c3)cc21 ; c1ccc(-c2nc3c(o2)c2ccccc2c2c4cc(N(c5ccccc5)c5ccc(-c6cccc7ccccc67)cc5)ccc4n(-c4ccccc4)c32)cc1 ; c1ccc(-c2nc3c(o2)c2ccccc2c2c4ccc(N(c5ccccc5)c5ccc(-c6ccc7ccccc7c6)cc5)cc4n(-c4ccccc4)c32)cc1]

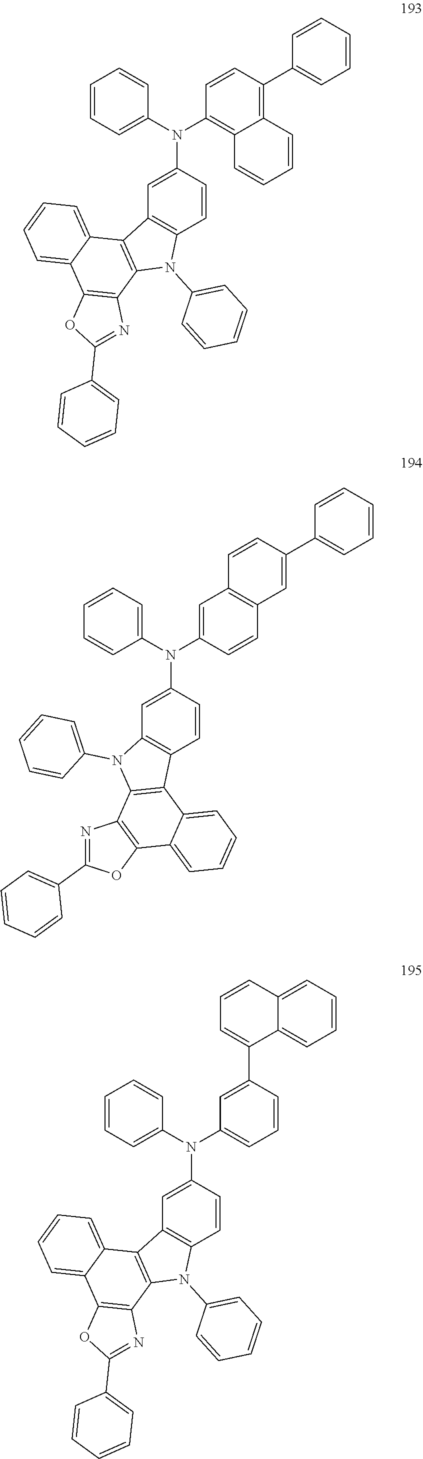 [SMILES: c1ccc(-c2nc3c(o2)c2ccccc2c2c4cc(N(c5ccccc5)c5ccc(-c6ccccc6)c6ccccc56)ccc4n(-c4ccccc4)c32)cc1 ; c1ccc(-c2ccc3cc(N(c4ccccc4)c4ccc5c6c7ccccc7c7oc(-c8ccccc8)nc7c6n(-c6ccccc6)c5c4)ccc3c2)cc1 ; c1ccc(-c2nc3c(o2)c2ccccc2c2c4cc(N(c5ccccc5)c5cccc(-c6cccc7ccccc67)c5)ccc4n(-c4ccccc4)c32)cc1]

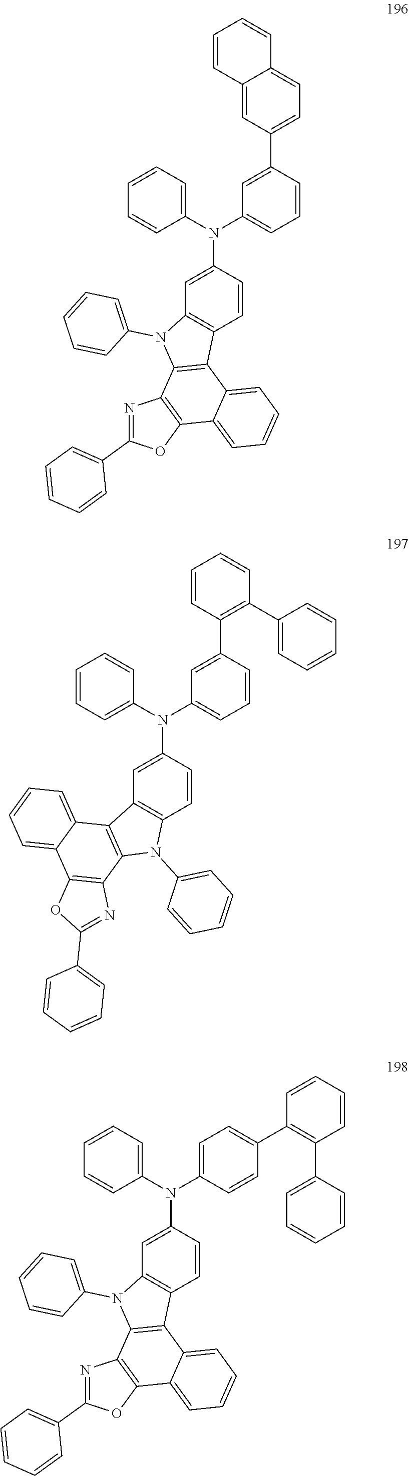 [SMILES: c1ccc(-c2nc3c(o2)c2ccccc2c2c4ccc(N(c5ccccc5)c5cccc(-c6ccc7ccccc7c6)c5)cc4n(-c4ccccc4)c32)cc1 ; c1ccc(-c2nc3c(o2)c2ccccc2c2c4cc(N(c5ccccc5)c5cccc(-c6ccccc6-c6ccccc6)c5)ccc4n(-c4ccccc4)c32)cc1 ; c1ccc(-c2nc3c(o2)c2ccccc2c2c4ccc(N(c5ccccc5)c5ccc(-c6ccccc6-c6ccccc6)cc5)cc4n(-c4ccccc4)c32)cc1]

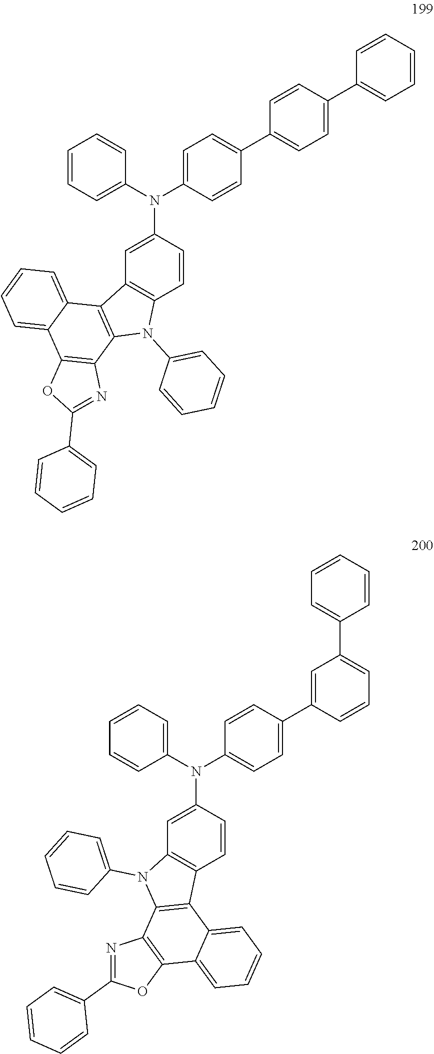 [SMILES: c1ccc(-c2ccc(-c3ccc(N(c4ccccc4)c4ccc5c(c4)c4c6ccccc6c6oc(-c7ccccc7)nc6c4n5-c4ccccc4)cc3)cc2)cc1 ; c1ccc(-c2cccc(-c3ccc(N(c4ccccc4)c4ccc5c6c7ccccc7c7oc(-c8ccccc8)nc7c6n(-c6ccccc6)c5c4)cc3)c2)cc1]

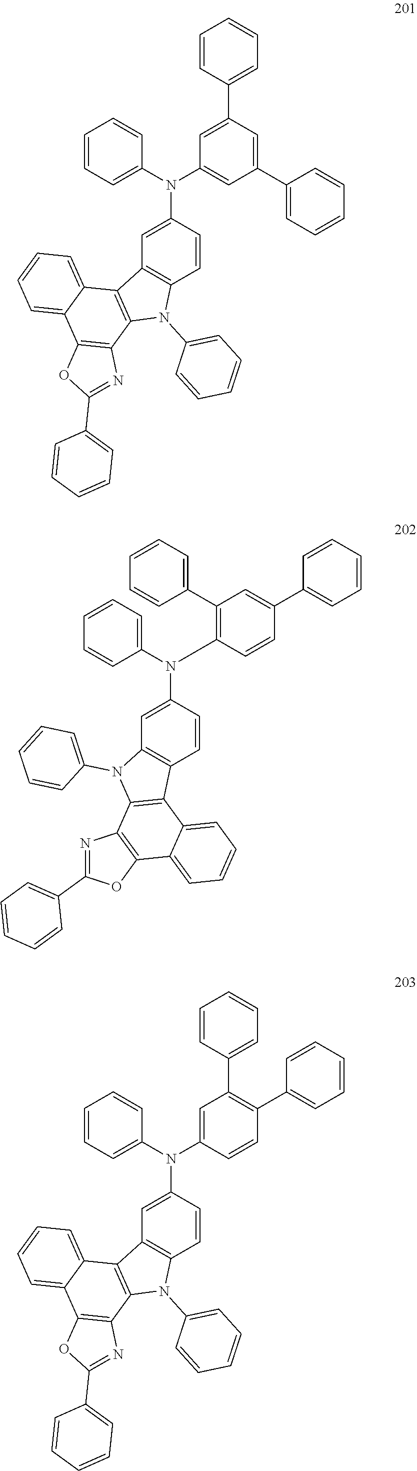 [SMILES: c1ccc(-c2cc(-c3ccccc3)cc(N(c3ccccc3)c3ccc4c(c3)c3c5ccccc5c5oc(-c6ccccc6)nc5c3n4-c3ccccc3)c2)cc1 ; c1ccc(-c2ccc(N(c3ccccc3)c3ccc4c5c6ccccc6c6oc(-c7ccccc7)nc6c5n(-c5ccccc5)c4c3)c(-c3ccccc3)c2)cc1 ; c1ccc(-c2nc3c(o2)c2ccccc2c2c4cc(N(c5ccccc5)c5ccc(-c6ccccc6)c(-c6ccccc6)c5)ccc4n(-c4ccccc4)c32)cc1]

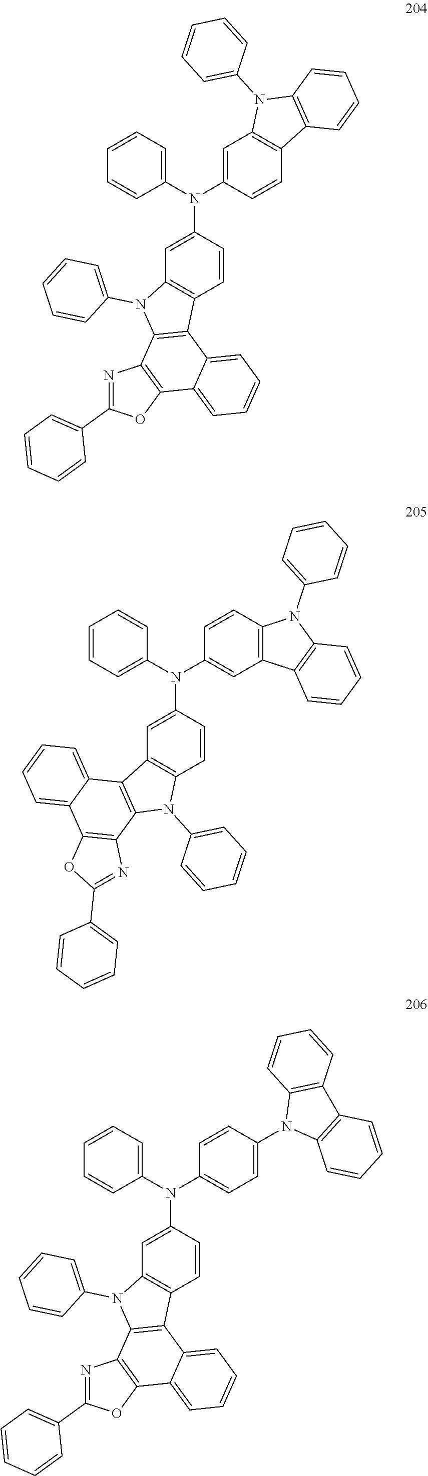 [SMILES: c1ccc(-c2nc3c(o2)c2ccccc2c2c4ccc(N(c5ccccc5)c5ccc6c7ccccc7n(-c7ccccc7)c6c5)cc4n(-c4ccccc4)c32)cc1 ; c1ccc(-c2nc3c(o2)c2ccccc2c2c4cc(N(c5ccccc5)c5ccc6c(c5)c5ccccc5n6-c5ccccc5)ccc4n(-c4ccccc4)c32)cc1 ; c1ccc(-c2nc3c(o2)c2ccccc2c2c4ccc(N(c5ccccc5)c5ccc(-n6c7ccccc7c7ccccc76)cc5)cc4n(-c4ccccc4)c32)cc1]

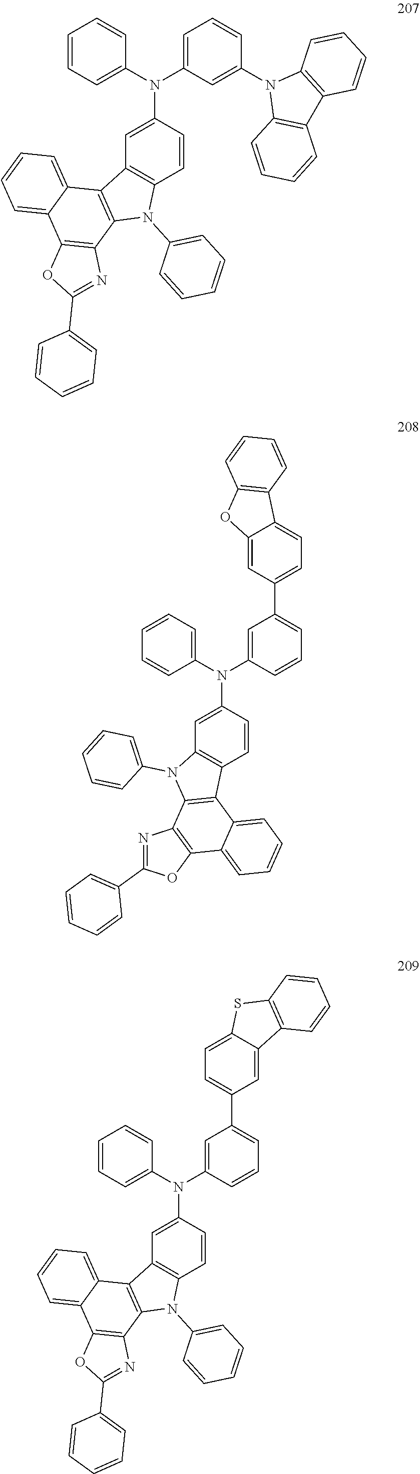 [SMILES: c1ccc(-c2nc3c(o2)c2ccccc2c2c4cc(N(c5ccccc5)c5cccc(-n6c7ccccc7c7ccccc76)c5)ccc4n(-c4ccccc4)c32)cc1 ; c1ccc(-c2nc3c(o2)c2ccccc2c2c4ccc(N(c5ccccc5)c5cccc(-c6ccc7c(c6)oc6ccccc67)c5)cc4n(-c4ccccc4)c32)cc1 ; c1ccc(-c2nc3c(o2)c2ccccc2c2c4cc(N(c5ccccc5)c5cccc(-c6ccc7sc8ccccc8c7c6)c5)ccc4n(-c4ccccc4)c32)cc1]

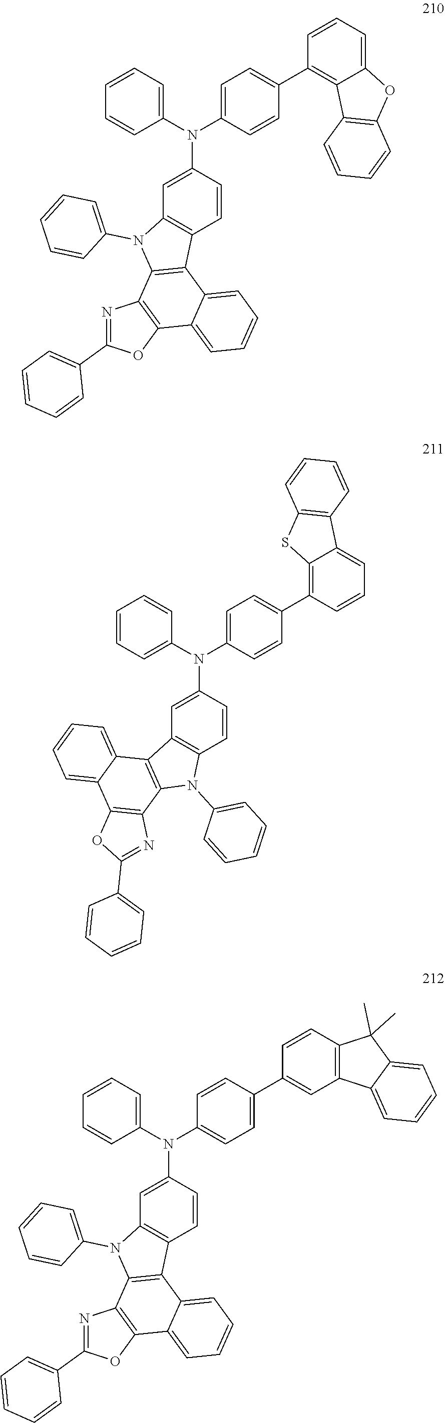 [SMILES: c1ccc(-c2nc3c(o2)c2ccccc2c2c4ccc(N(c5ccccc5)c5ccc(-c6cccc7oc8ccccc8c67)cc5)cc4n(-c4ccccc4)c32)cc1 ; c1ccc(-c2nc3c(o2)c2ccccc2c2c4cc(N(c5ccccc5)c5ccc(-c6cccc7c6sc6ccccc67)cc5)ccc4n(-c4ccccc4)c32)cc1 ; CC1(C)c2ccccc2-c2cc(-c3ccc(N(c4ccccc4)c4ccc5c6c7ccccc7c7oc(-c8ccccc8)nc7c6n(-c6ccccc6)c5c4)cc3)ccc21]

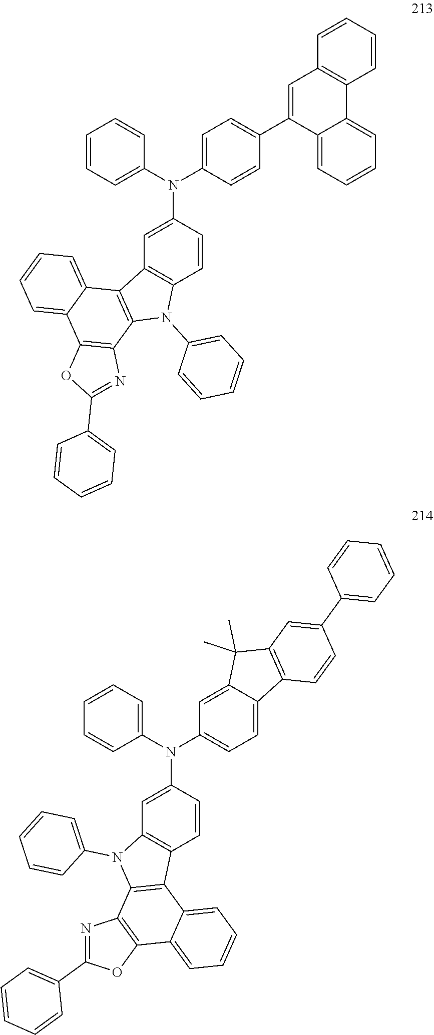 [SMILES: c1ccc(-c2nc3c(o2)c2ccccc2c2c4cc(N(c5ccccc5)c5ccc(-c6cc7ccccc7c7ccccc67)cc5)ccc4n(-c4ccccc4)c32)cc1 ; CC1(C)c2cc(-c3ccccc3)ccc2-c2ccc(N(c3ccccc3)c3ccc4c5c6ccccc6c6oc(-c7ccccc7)nc6c5n(-c5ccccc5)c4c3)cc21]

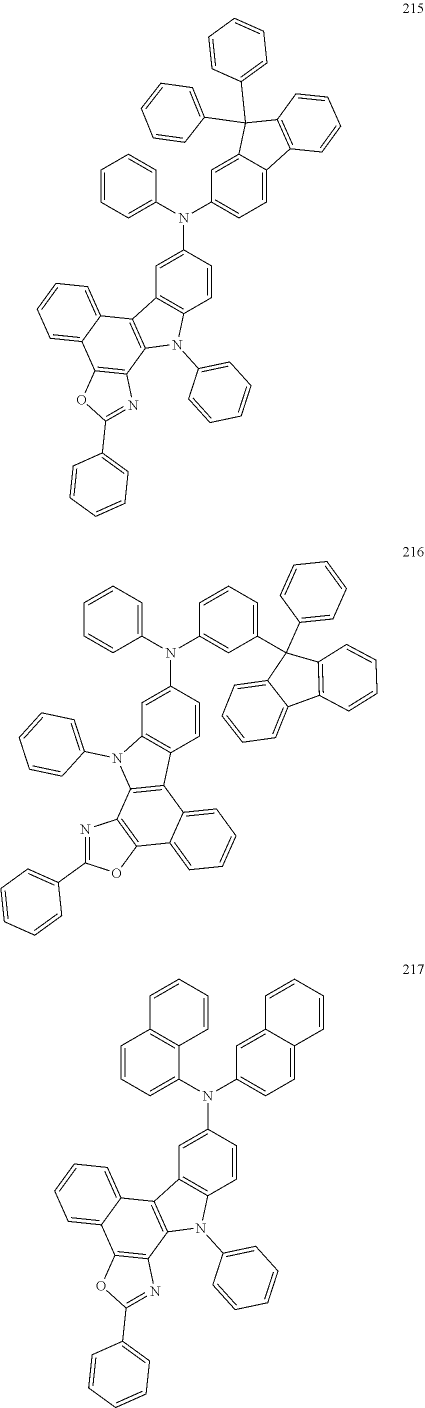 [SMILES: c1ccc(-c2nc3c(o2)c2ccccc2c2c4cc(N(c5ccccc5)c5ccc6c(c5)C(c5ccccc5)(c5ccccc5)c5ccccc5-6)ccc4n(-c4ccccc4)c32)cc1 ; c1ccc(-c2nc3c(o2)c2ccccc2c2c4ccc(N(c5ccccc5)c5cccc(C6(c7ccccc7)c7ccccc7-c7ccccc76)c5)cc4n(-c4ccccc4)c32)cc1 ; c1ccc(-c2nc3c(o2)c2ccccc2c2c4cc(N(c5ccc6ccccc6c5)c5cccc6ccccc56)ccc4n(-c4ccccc4)c32)cc1]

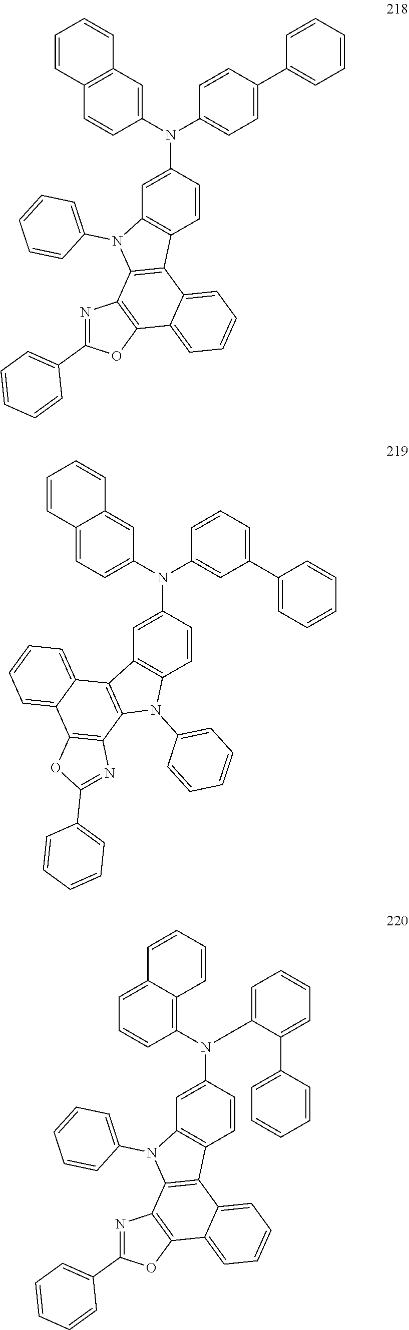 [SMILES: c1ccc(-c2ccc(N(c3ccc4ccccc4c3)c3ccc4c5c6ccccc6c6oc(-c7ccccc7)nc6c5n(-c5ccccc5)c4c3)cc2)cc1 ; c1ccc(-c2cccc(N(c3ccc4ccccc4c3)c3ccc4c(c3)c3c5ccccc5c5oc(-c6ccccc6)nc5c3n4-c3ccccc3)c2)cc1 ; c1ccc(-c2nc3c(o2)c2ccccc2c2c4ccc(N(c5ccccc5-c5ccccc5)c5cccc6ccccc56)cc4n(-c4ccccc4)c32)cc1]

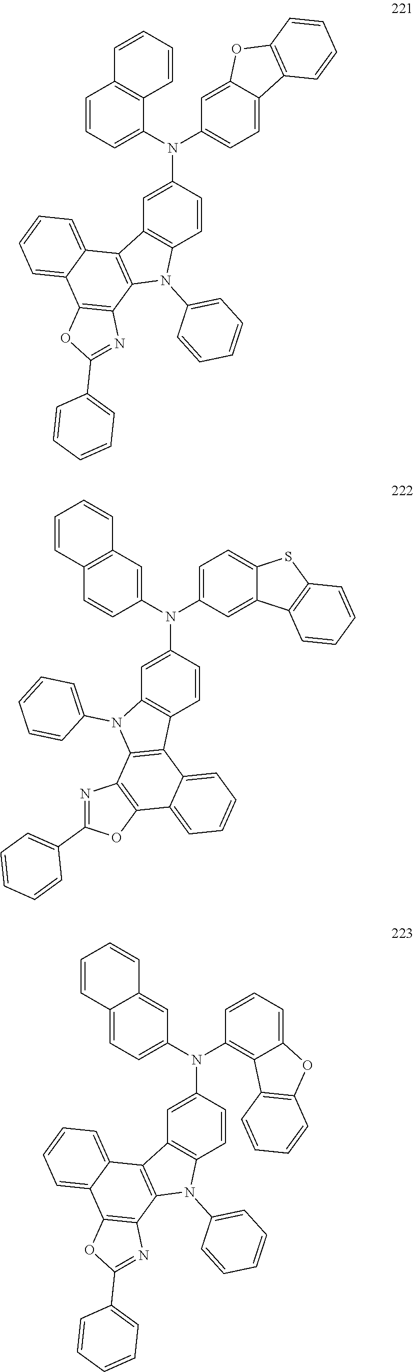

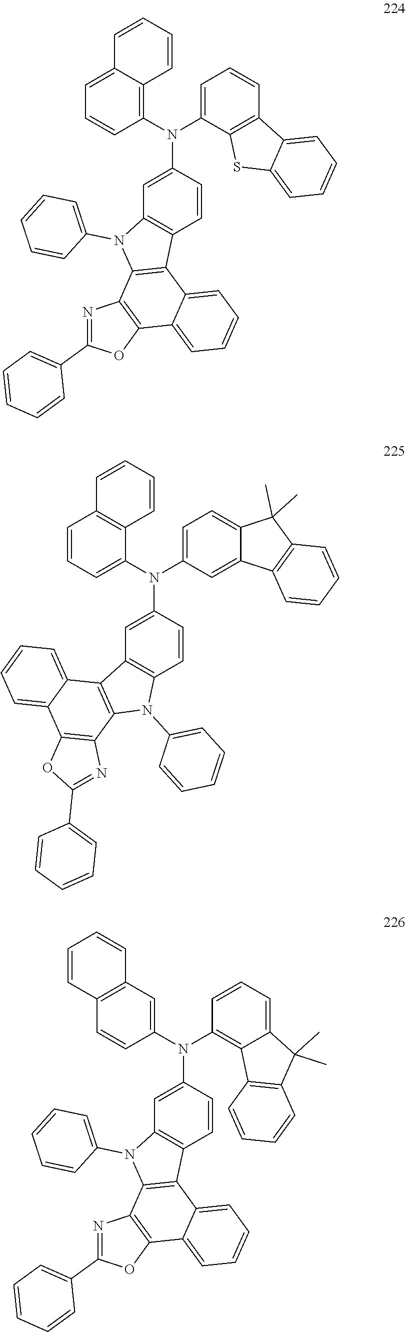 [SMILES: c1ccc(-c2nc3c(o2)c2ccccc2c2c4ccc(N(c5cccc6ccccc56)c5cccc6c5sc5ccccc56)cc4n(-c4ccccc4)c32)cc1 ; CC1(C)c2ccccc2-c2cc(N(c3ccc4c(c3)c3c5ccccc5c5oc(-c6ccccc6)nc5c3n4-c3ccccc3)c3cccc4ccccc34)ccc21 ; CC1(C)c2ccccc2-c2c(N(c3ccc4ccccc4c3)c3ccc4c5c6ccccc6c6oc(-c7ccccc7)nc6c5n(-c5ccccc5)c4c3)cccc21]

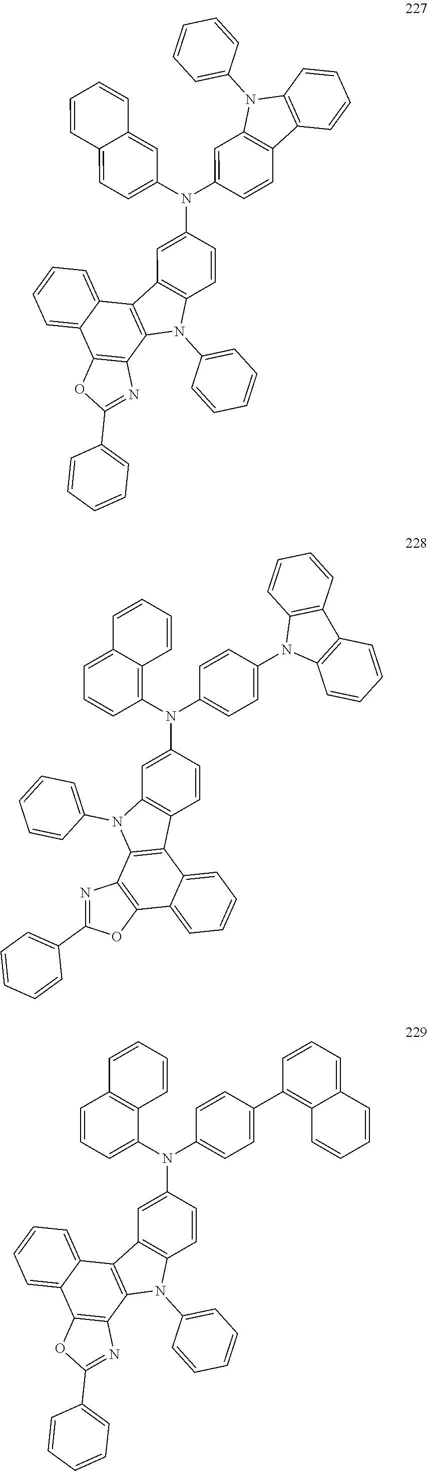 [SMILES: c1ccc(-c2nc3c(o2)c2ccccc2c2c4cc(N(c5ccc6ccccc6c5)c5ccc6c7ccccc7n(-c7ccccc7)c6c5)ccc4n(-c4ccccc4)c32)cc1 ; c1ccc(-c2nc3c(o2)c2ccccc2c2c4ccc(N(c5ccc(-n6c7ccccc7c7ccccc76)cc5)c5cccc6ccccc56)cc4n(-c4ccccc4)c32)cc1 ; c1ccc(-c2nc3c(o2)c2ccccc2c2c4cc(N(c5ccc(-c6cccc7ccccc67)cc5)c5cccc6ccccc56)ccc4n(-c4ccccc4)c32)cc1]

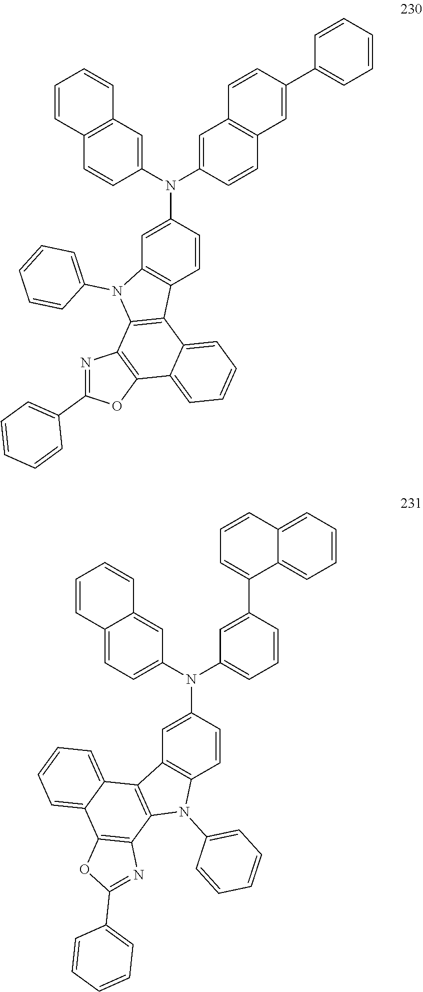 [SMILES: c1ccc(-c2ccc3cc(N(c4ccc5ccccc5c4)c4ccc5c6c7ccccc7c7oc(-c8ccccc8)nc7c6n(-c6ccccc6)c5c4)ccc3c2)cc1 ; c1ccc(-c2nc3c(o2)c2ccccc2c2c4cc(N(c5cccc(-c6cccc7ccccc67)c5)c5ccc6ccccc6c5)ccc4n(-c4ccccc4)c32)cc1]

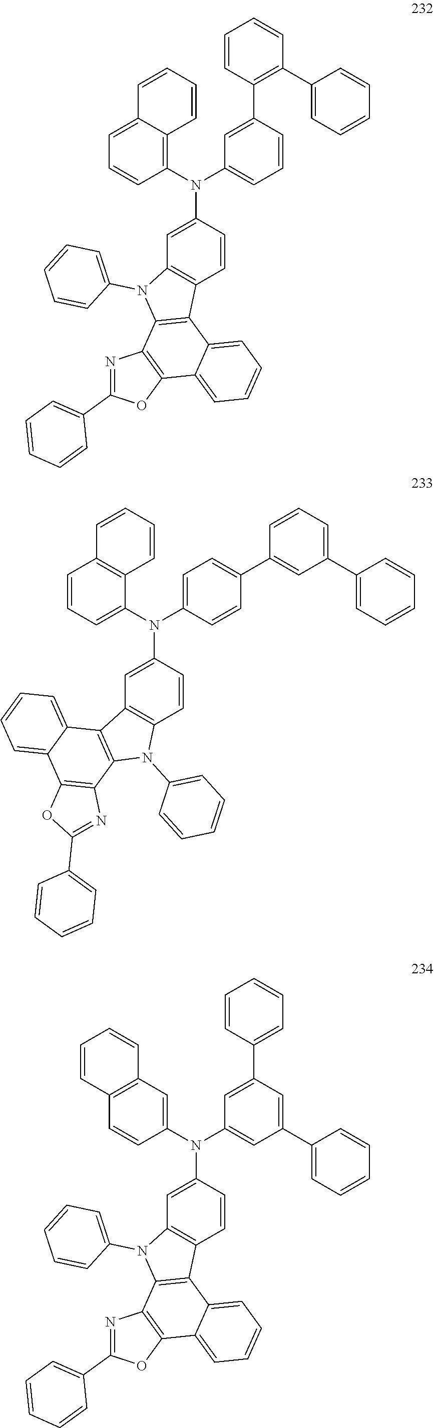 [SMILES: c1ccc(-c2nc3c(o2)c2ccccc2c2c4ccc(N(c5cccc(-c6ccccc6-c6ccccc6)c5)c5cccc6ccccc56)cc4n(-c4ccccc4)c32)cc1 ; c1ccc(-c2cccc(-c3ccc(N(c4ccc5c(c4)c4c6ccccc6c6oc(-c7ccccc7)nc6c4n5-c4ccccc4)c4cccc5ccccc45)cc3)c2)cc1 ; c1ccc(-c2cc(-c3ccccc3)cc(N(c3ccc4ccccc4c3)c3ccc4c5c6ccccc6c6oc(-c7ccccc7)nc6c5n(-c5ccccc5)c4c3)c2)cc1]

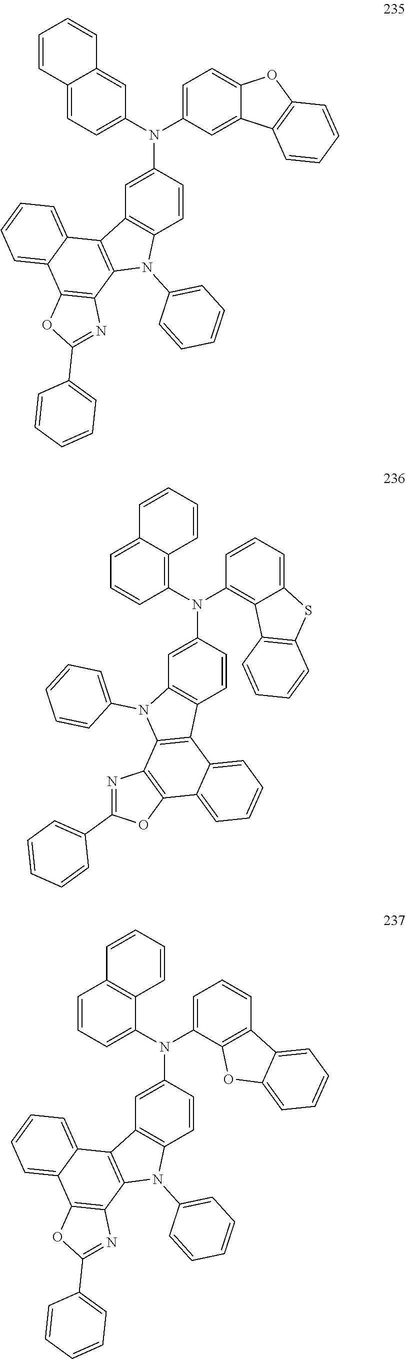 [SMILES: c1ccc(-c2nc3c(o2)c2ccccc2c2c4cc(N(c5ccc6ccccc6c5)c5ccc6oc7ccccc7c6c5)ccc4n(-c4ccccc4)c32)cc1 ; c1ccc(-c2nc3c(o2)c2ccccc2c2c4ccc(N(c5cccc6ccccc56)c5cccc6sc7ccccc7c56)cc4n(-c4ccccc4)c32)cc1 ; c1ccc(-c2nc3c(o2)c2ccccc2c2c4cc(N(c5cccc6ccccc56)c5cccc6c5oc5ccccc56)ccc4n(-c4ccccc4)c32)cc1]

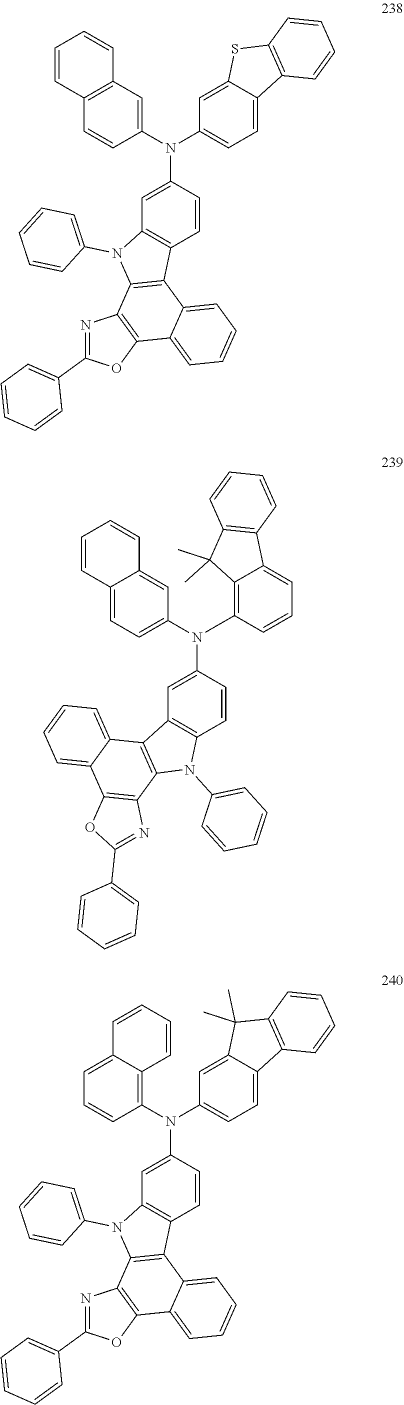 [SMILES: c1ccc(-c2nc3c(o2)c2ccccc2c2c4ccc(N(c5ccc6ccccc6c5)c5ccc6c(c5)sc5ccccc56)cc4n(-c4ccccc4)c32)cc1 ; CC1(C)c2ccccc2-c2cccc(N(c3ccc4ccccc4c3)c3ccc4c(c3)c3c5ccccc5c5oc(-c6ccccc6)nc5c3n4-c3ccccc3)c21 ; CC1(C)c2ccccc2-c2ccc(N(c3ccc4c5c6ccccc6c6oc(-c7ccccc7)nc6c5n(-c5ccccc5)c4c3)c3cccc4ccccc34)cc21]

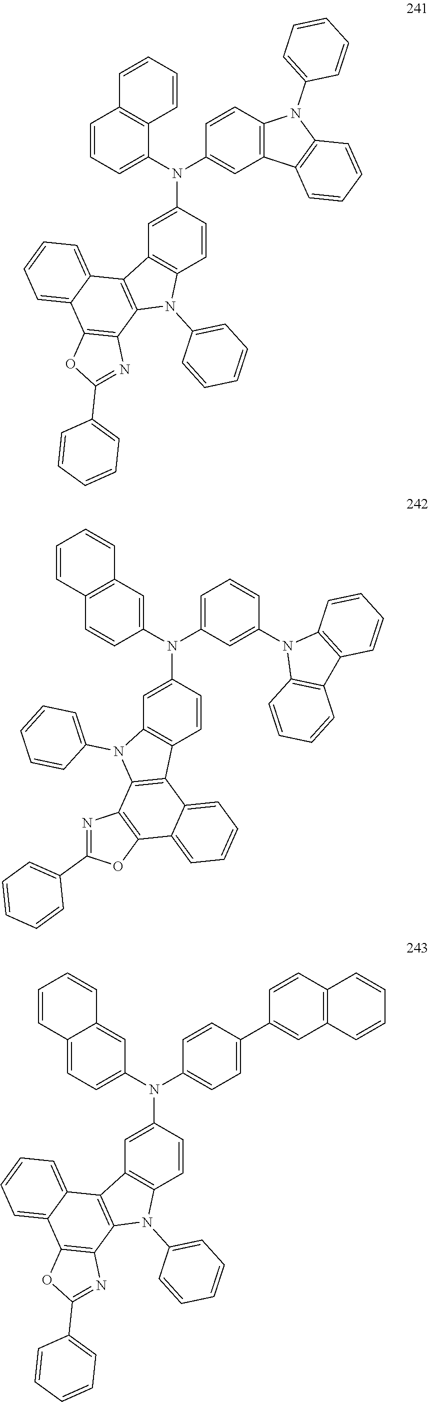

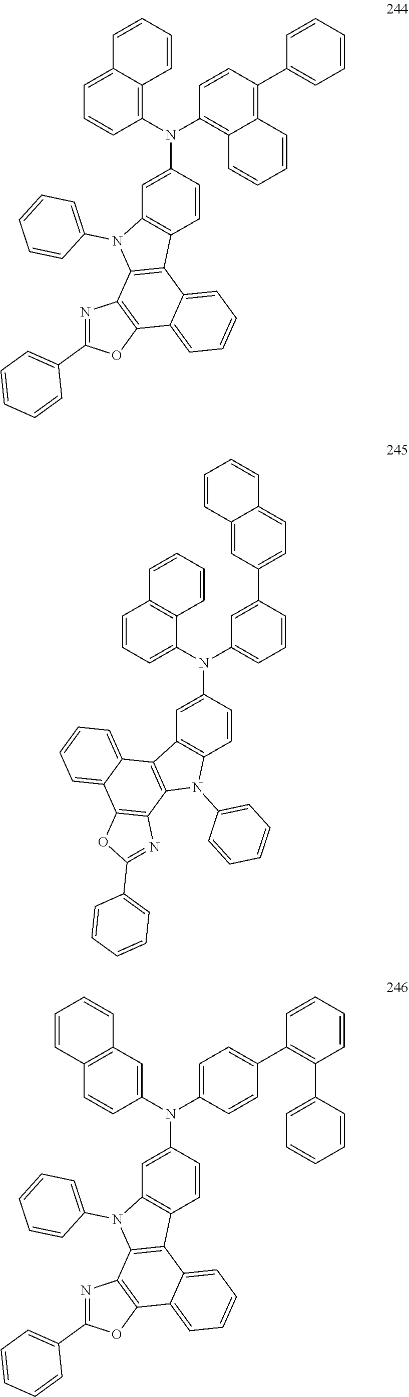 [SMILES: c1ccc(-c2nc3c(o2)c2ccccc2c2c4ccc(N(c5cccc6ccccc56)c5ccc(-c6ccccc6)c6ccccc56)cc4n(-c4ccccc4)c32)cc1 ; c1ccc(-c2nc3c(o2)c2ccccc2c2c4cc(N(c5cccc(-c6ccc7ccccc7c6)c5)c5cccc6ccccc56)ccc4n(-c4ccccc4)c32)cc1 ; c1ccc(-c2nc3c(o2)c2ccccc2c2c4ccc(N(c5ccc(-c6ccccc6-c6ccccc6)cc5)c5ccc6ccccc6c5)cc4n(-c4ccccc4)c32)cc1]

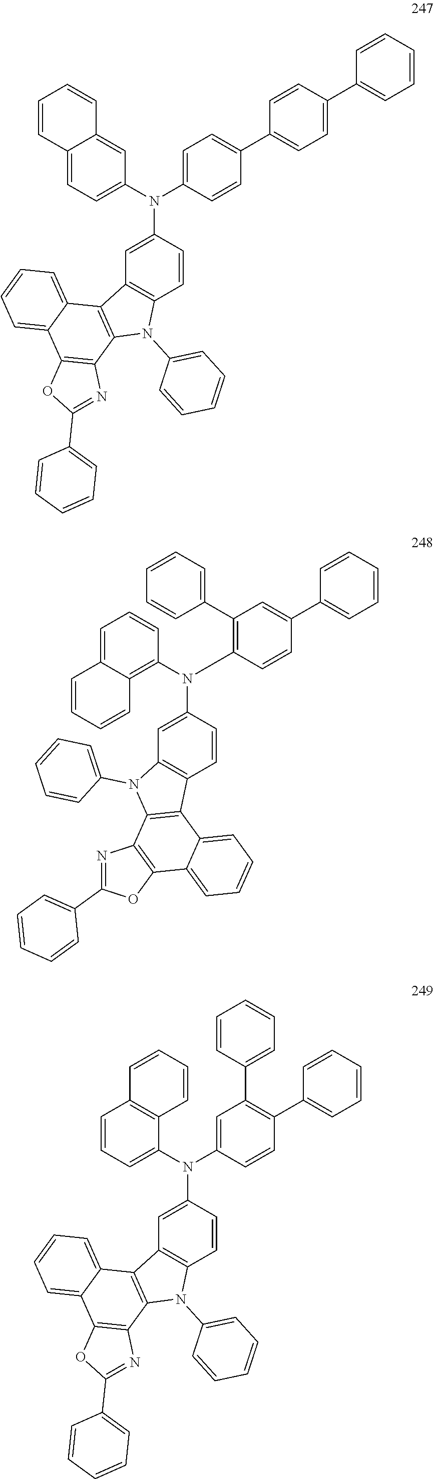 [SMILES: c1ccc(-c2ccc(-c3ccc(N(c4ccc5ccccc5c4)c4ccc5c(c4)c4c6ccccc6c6oc(-c7ccccc7)nc6c4n5-c4ccccc4)cc3)cc2)cc1 ; c1ccc(-c2ccc(N(c3ccc4c5c6ccccc6c6oc(-c7ccccc7)nc6c5n(-c5ccccc5)c4c3)c3cccc4ccccc34)c(-c3ccccc3)c2)cc1 ; c1ccc(-c2nc3c(o2)c2ccccc2c2c4cc(N(c5ccc(-c6ccccc6)c(-c6ccccc6)c5)c5cccc6ccccc56)ccc4n(-c4ccccc4)c32)cc1]

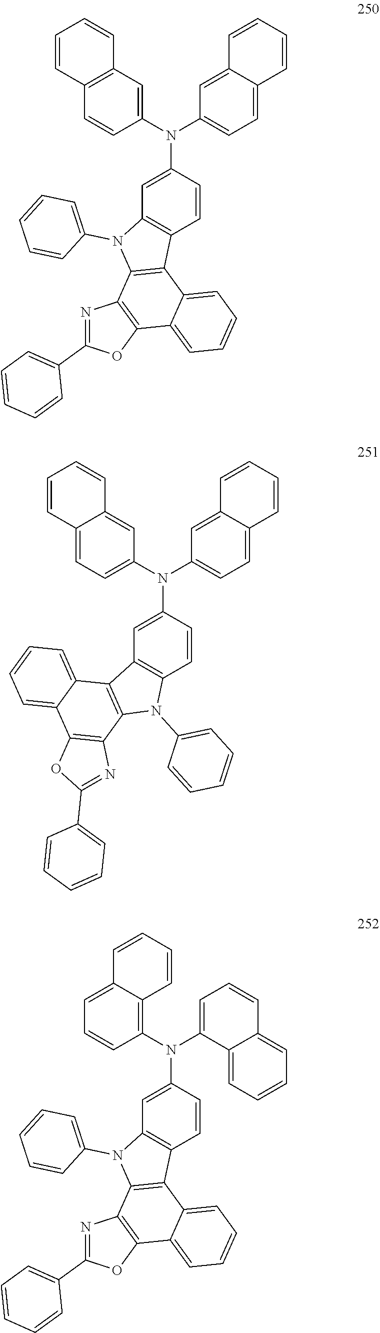 [SMILES: c1ccc(-c2nc3c(o2)c2ccccc2c2c4ccc(N(c5ccc6ccccc6c5)c5ccc6ccccc6c5)cc4n(-c4ccccc4)c32)cc1 ; c1ccc(-c2nc3c(o2)c2ccccc2c2c4cc(N(c5ccc6ccccc6c5)c5ccc6ccccc6c5)ccc4n(-c4ccccc4)c32)cc1 ; c1ccc(-c2nc3c(o2)c2ccccc2c2c4ccc(N(c5cccc6ccccc56)c5cccc6ccccc56)cc4n(-c4ccccc4)c32)cc1]

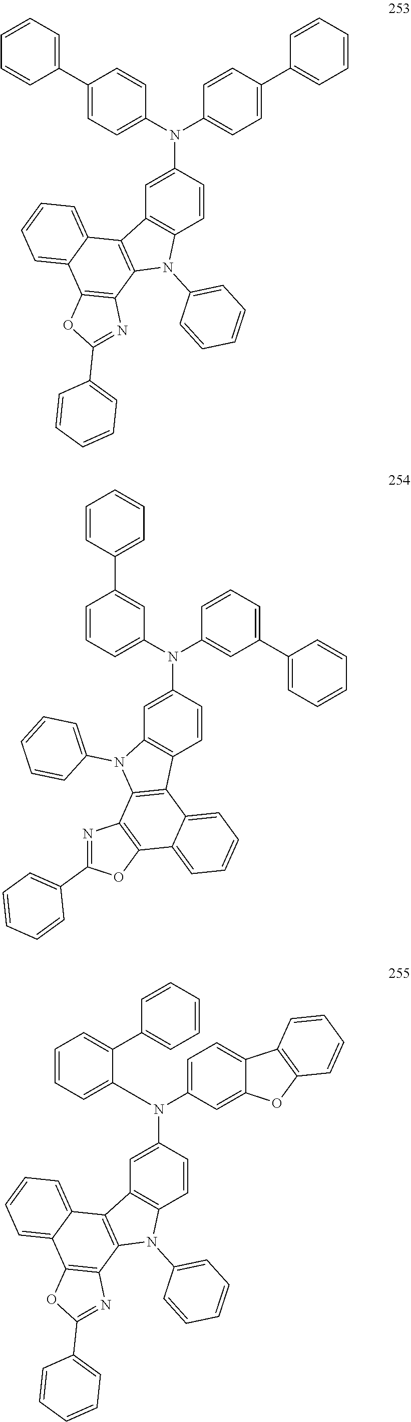 [SMILES: c1ccc(-c2ccc(N(c3ccc(-c4ccccc4)cc3)c3ccc4c(c3)c3c5ccccc5c5oc(-c6ccccc6)nc5c3n4-c3ccccc3)cc2)cc1 ; c1ccc(-c2cccc(N(c3cccc(-c4ccccc4)c3)c3ccc4c5c6ccccc6c6oc(-c7ccccc7)nc6c5n(-c5ccccc5)c4c3)c2)cc1 ; c1ccc(-c2nc3c(o2)c2ccccc2c2c4cc(N(c5ccc6c(c5)oc5ccccc56)c5ccccc5-c5ccccc5)ccc4n(-c4ccccc4)c32)cc1]

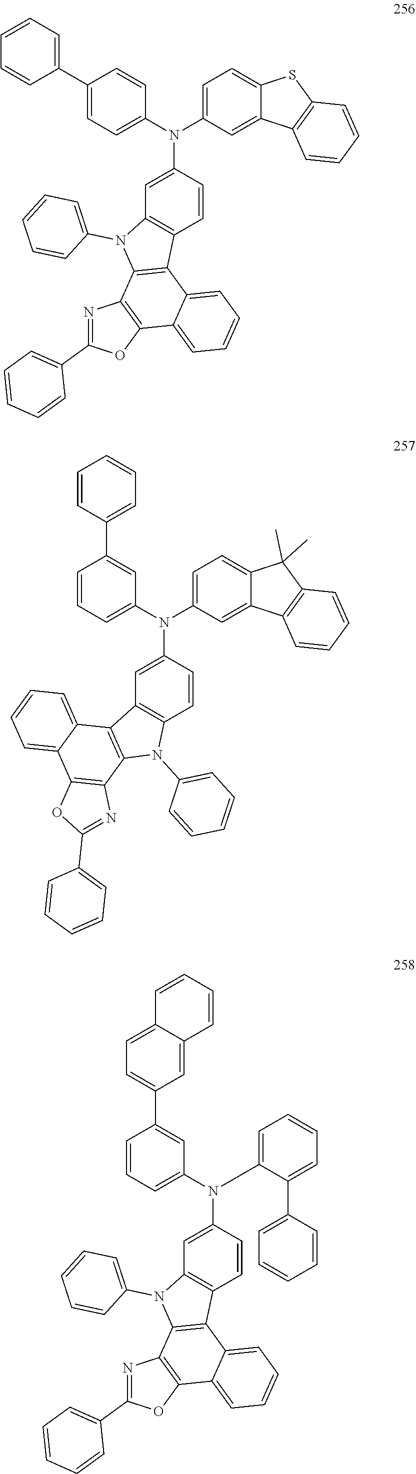 [SMILES: c1ccc(-c2ccc(N(c3ccc4sc5ccccc5c4c3)c3ccc4c5c6ccccc6c6oc(-c7ccccc7)nc6c5n(-c5ccccc5)c4c3)cc2)cc1 ; CC1(C)c2ccccc2-c2cc(N(c3cccc(-c4ccccc4)c3)c3ccc4c(c3)c3c5ccccc5c5oc(-c6ccccc6)nc5c3n4-c3ccccc3)ccc21 ; c1ccc(-c2nc3c(o2)c2ccccc2c2c4ccc(N(c5cccc(-c6ccc7ccccc7c6)c5)c5ccccc5-c5ccccc5)cc4n(-c4ccccc4)c32)cc1]

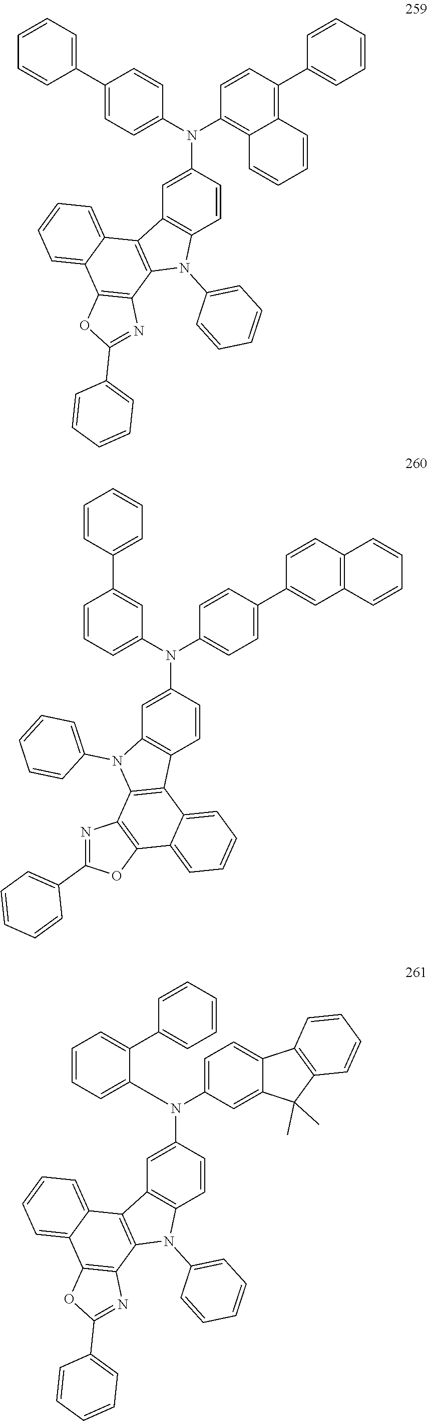

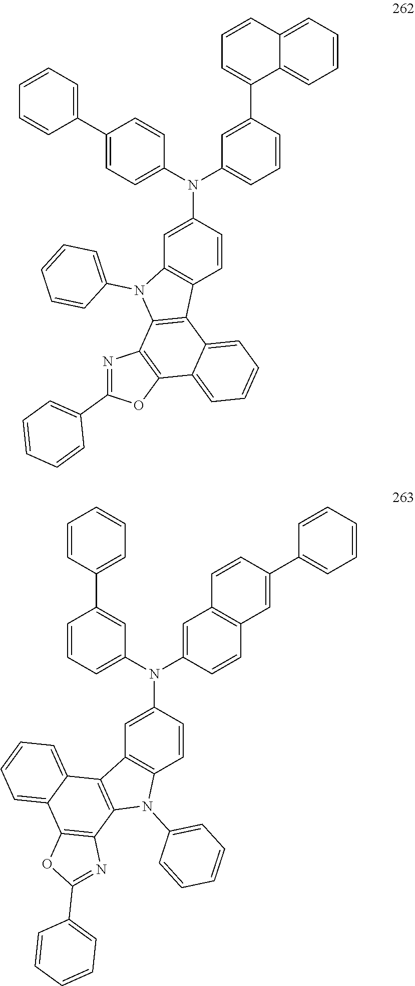 [SMILES: c1ccc(-c2ccc(N(c3cccc(-c4cccc5ccccc45)c3)c3ccc4c5c6ccccc6c6oc(-c7ccccc7)nc6c5n(-c5ccccc5)c4c3)cc2)cc1 ; c1ccc(-c2cccc(N(c3ccc4cc(-c5ccccc5)ccc4c3)c3ccc4c(c3)c3c5ccccc5c5oc(-c6ccccc6)nc5c3n4-c3ccccc3)c2)cc1]

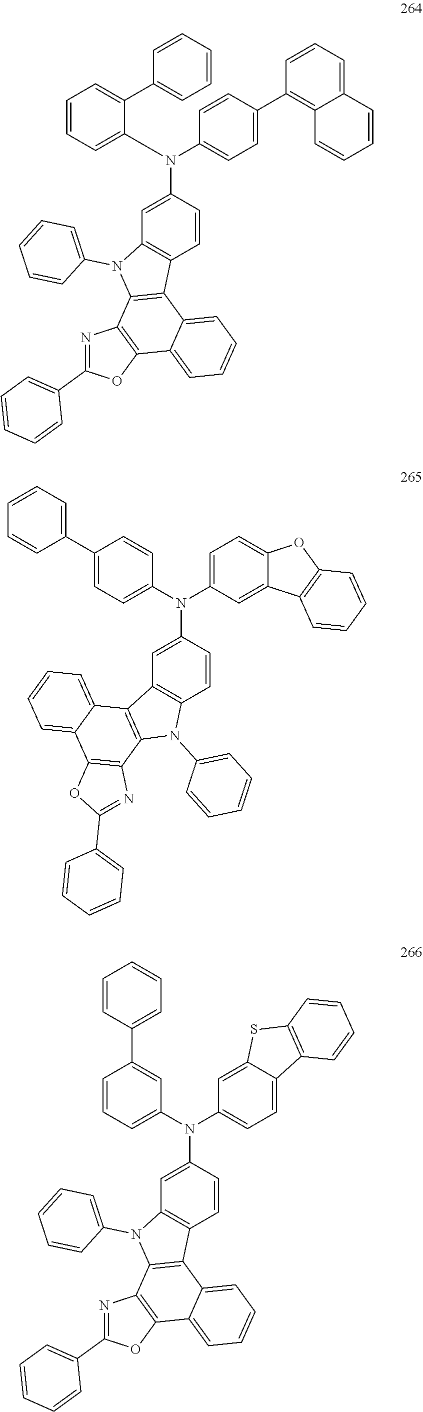

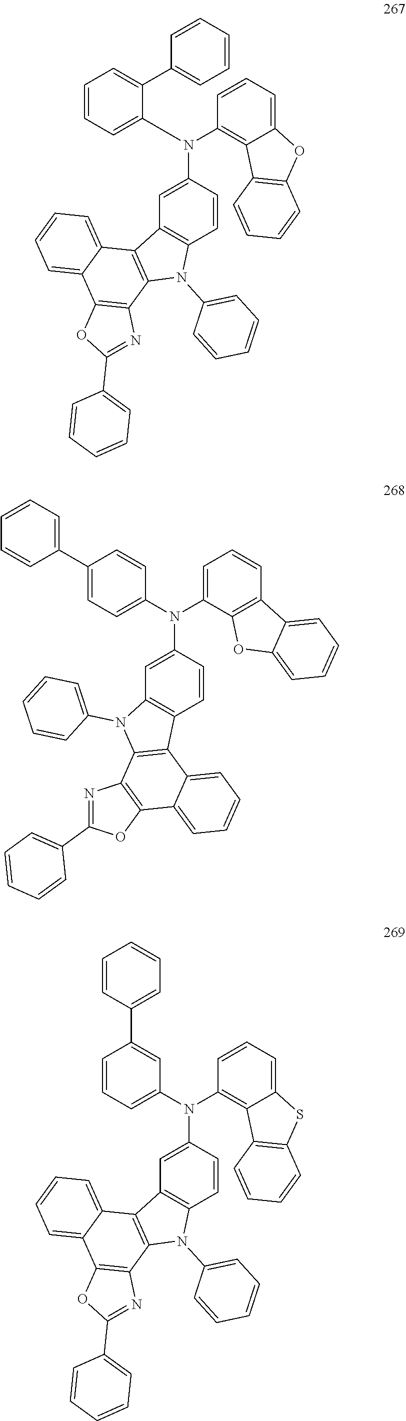 [SMILES: c1ccc(-c2nc3c(o2)c2ccccc2c2c4cc(N(c5ccccc5-c5ccccc5)c5cccc6oc7ccccc7c56)ccc4n(-c4ccccc4)c32)cc1 ; c1ccc(-c2ccc(N(c3ccc4c5c6ccccc6c6oc(-c7ccccc7)nc6c5n(-c5ccccc5)c4c3)c3cccc4c3oc3ccccc34)cc2)cc1 ; c1ccc(-c2cccc(N(c3ccc4c(c3)c3c5ccccc5c5oc(-c6ccccc6)nc5c3n4-c3ccccc3)c3cccc4sc5ccccc5c34)c2)cc1]

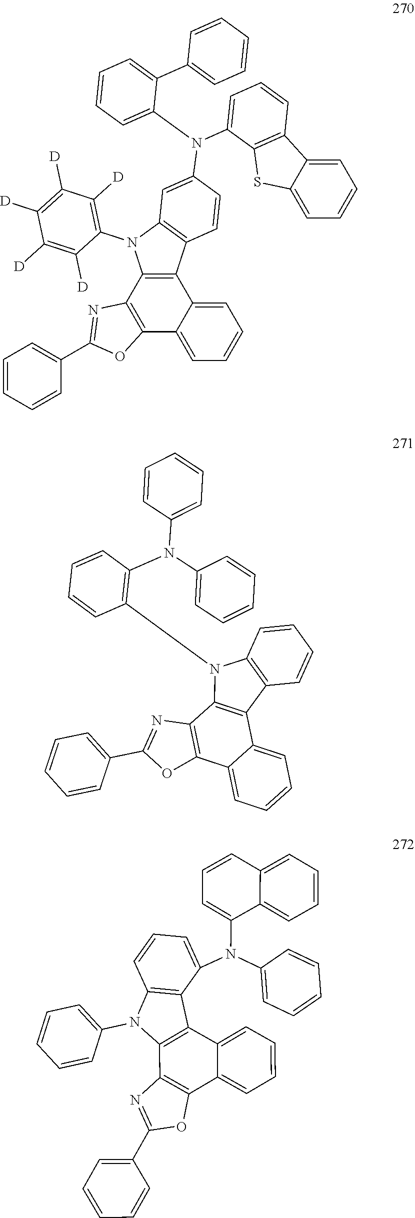 [SMILES: [2H]c1c([2H])c([2H])c(-n2c3cc(N(c4ccccc4-c4ccccc4)c4cccc5c4sc4ccccc45)ccc3c3c4ccccc4c4oc(-c5ccccc5)nc4c32)c([2H])c1[2H] ; c1ccc(-c2nc3c(o2)c2ccccc2c2c4ccccc4n(-c4ccccc4N(c4ccccc4)c4ccccc4)c32)cc1 ; c1ccc(-c2nc3c(o2)c2ccccc2c2c4c(N(c5ccccc5)c5cccc6ccccc56)cccc4n(-c4ccccc4)c32)cc1]

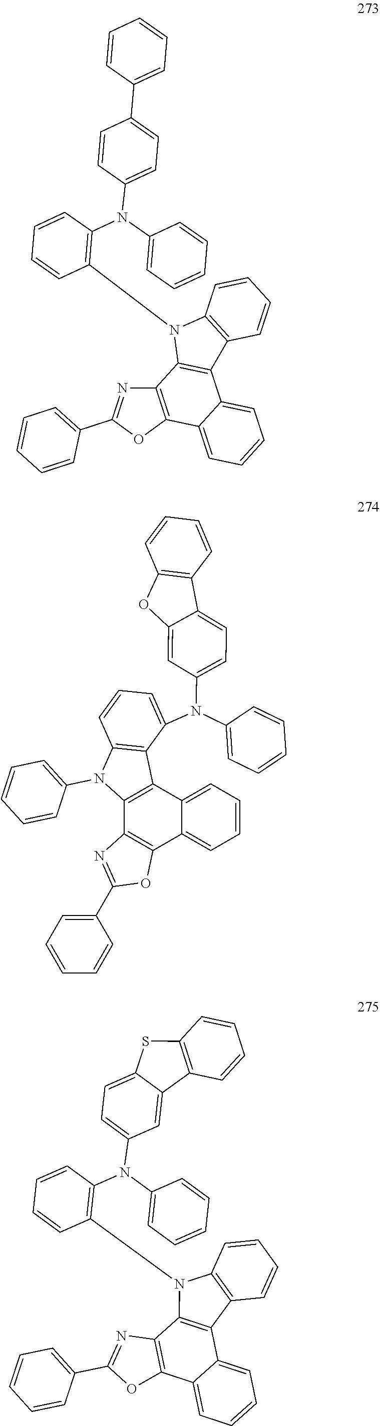 [SMILES: c1ccc(-c2ccc(N(c3ccccc3)c3ccccc3-n3c4ccccc4c4c5ccccc5c5oc(-c6ccccc6)nc5c43)cc2)cc1 ; c1ccc(-c2nc3c(o2)c2ccccc2c2c4c(N(c5ccccc5)c5ccc6c(c5)oc5ccccc56)cccc4n(-c4ccccc4)c32)cc1 ; c1ccc(-c2nc3c(o2)c2ccccc2c2c4ccccc4n(-c4ccccc4N(c4ccccc4)c4ccc5sc6ccccc6c5c4)c32)cc1]

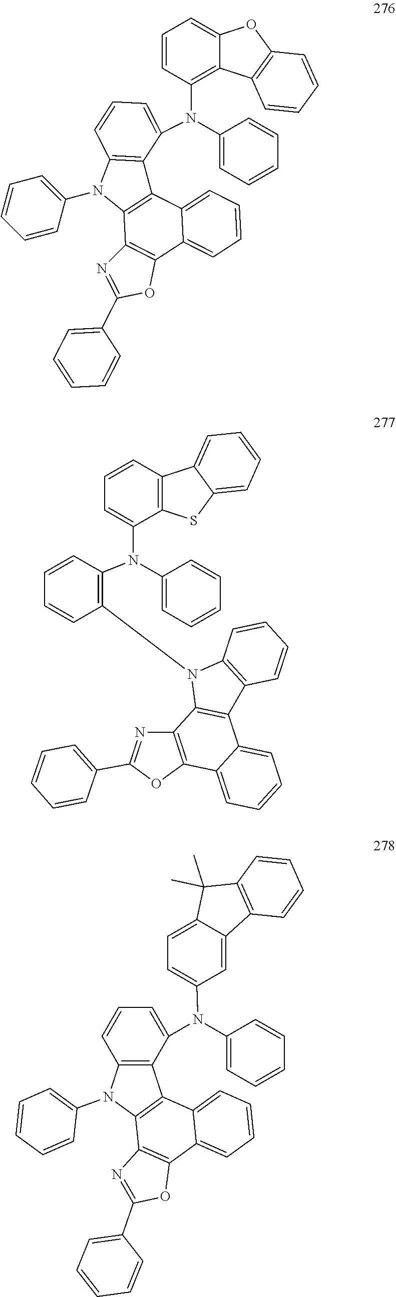 [SMILES: c1ccc(-c2nc3c(o2)c2ccccc2c2c4c(N(c5ccccc5)c5cccc6oc7ccccc7c56)cccc4n(-c4ccccc4)c32)cc1 ; c1ccc(-c2nc3c(o2)c2ccccc2c2c4ccccc4n(-c4ccccc4N(c4ccccc4)c4cccc5c4sc4ccccc45)c32)cc1 ; CC1(C)c2ccccc2-c2cc(N(c3ccccc3)c3cccc4c3c3c5ccccc5c5oc(-c6ccccc6)nc5c3n4-c3ccccc3)ccc21]

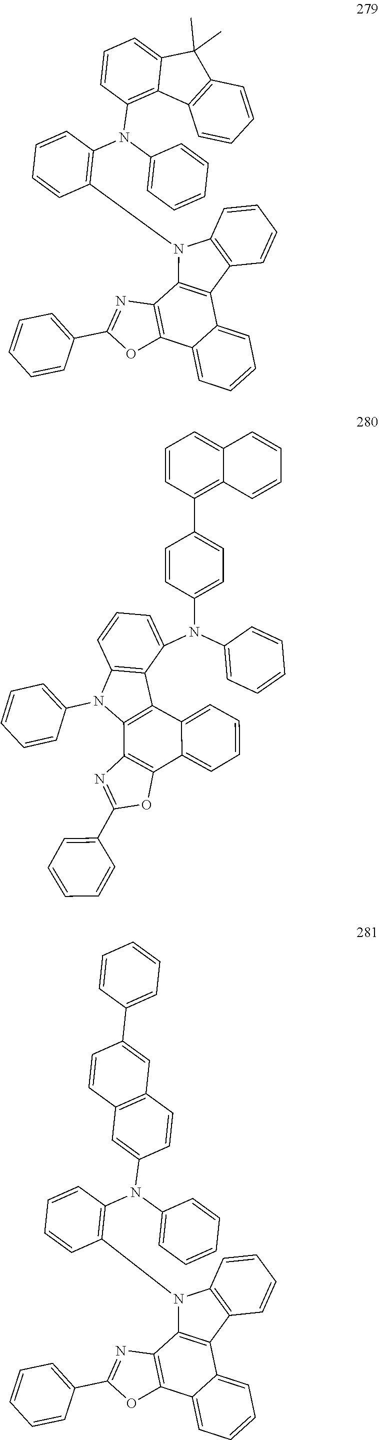

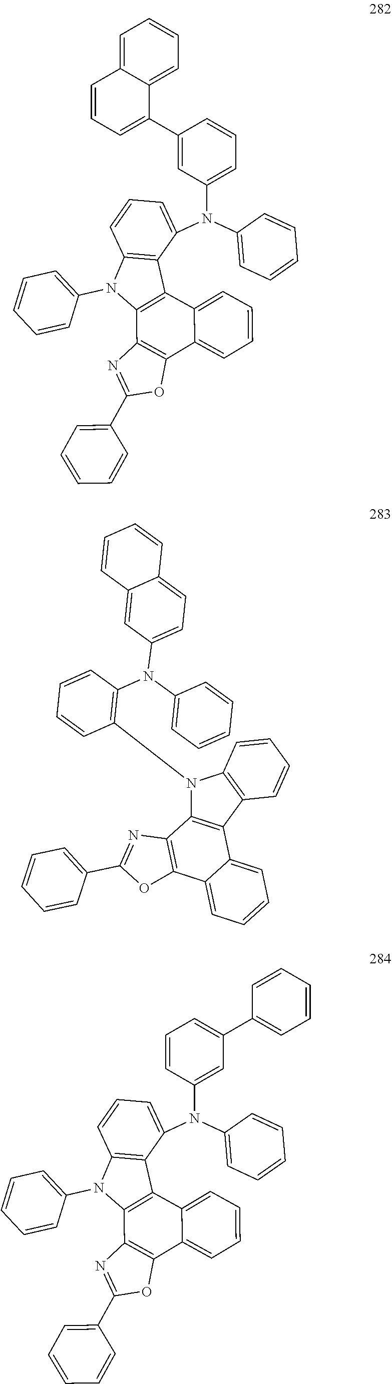 [SMILES: c1ccc(-c2nc3c(o2)c2ccccc2c2c4c(N(c5ccccc5)c5cccc(-c6cccc7ccccc67)c5)cccc4n(-c4ccccc4)c32)cc1 ; c1ccc(-c2nc3c(o2)c2ccccc2c2c4ccccc4n(-c4ccccc4N(c4ccccc4)c4ccc5ccccc5c4)c32)cc1 ; c1ccc(-c2cccc(N(c3ccccc3)c3cccc4c3c3c5ccccc5c5oc(-c6ccccc6)nc5c3n4-c3ccccc3)c2)cc1]

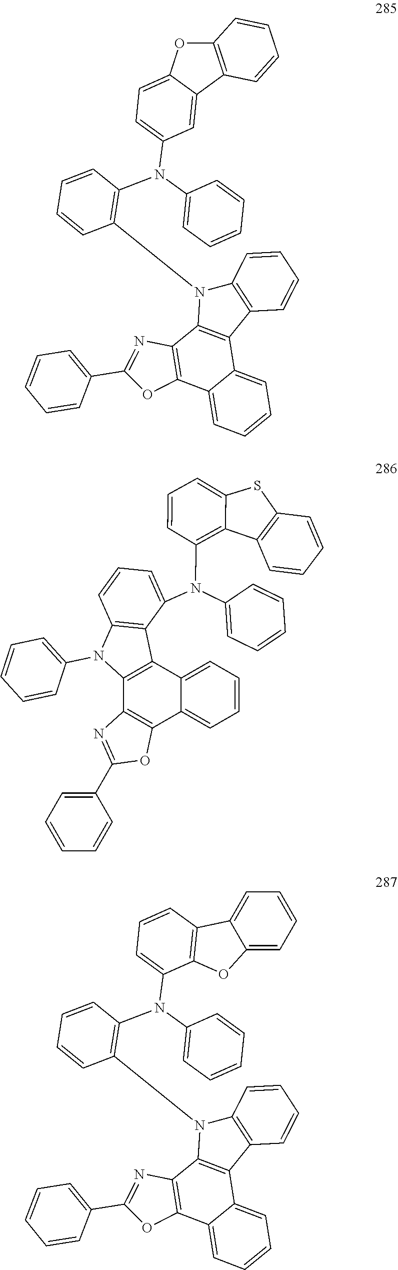 [SMILES: c1ccc(-c2nc3c(o2)c2ccccc2c2c4ccccc4n(-c4ccccc4N(c4ccccc4)c4ccc5oc6ccccc6c5c4)c32)cc1 ; c1ccc(-c2nc3c(o2)c2ccccc2c2c4c(N(c5ccccc5)c5cccc6sc7ccccc7c56)cccc4n(-c4ccccc4)c32)cc1 ; c1ccc(-c2nc3c(o2)c2ccccc2c2c4ccccc4n(-c4ccccc4N(c4ccccc4)c4cccc5c4oc4ccccc45)c32)cc1]

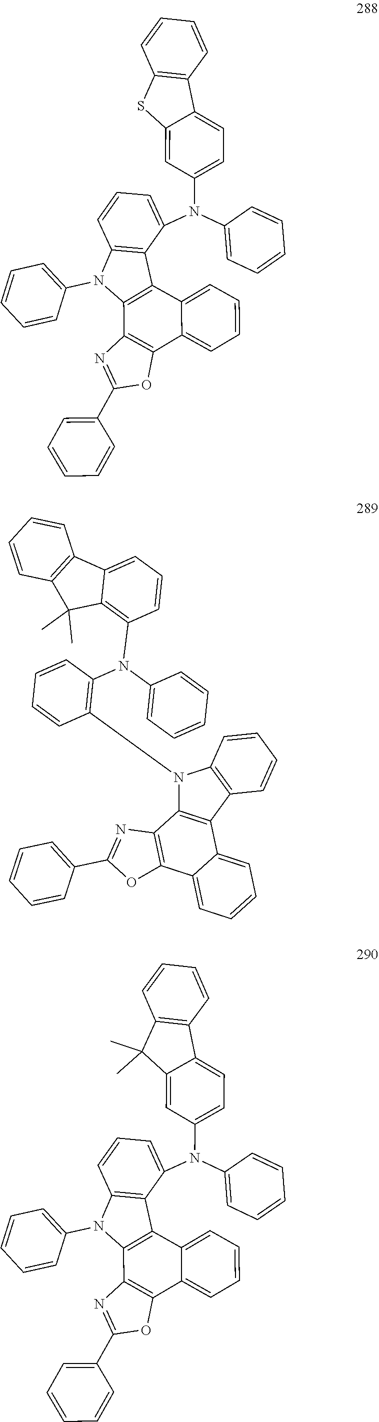 [SMILES: c1ccc(-c2nc3c(o2)c2ccccc2c2c4c(N(c5ccccc5)c5ccc6c(c5)sc5ccccc56)cccc4n(-c4ccccc4)c32)cc1 ; CC1(C)c2ccccc2-c2cccc(N(c3ccccc3)c3ccccc3-n3c4ccccc4c4c5ccccc5c5oc(-c6ccccc6)nc5c43)c21 ; CC1(C)c2ccccc2-c2ccc(N(c3ccccc3)c3cccc4c3c3c5ccccc5c5oc(-c6ccccc6)nc5c3n4-c3ccccc3)cc21]

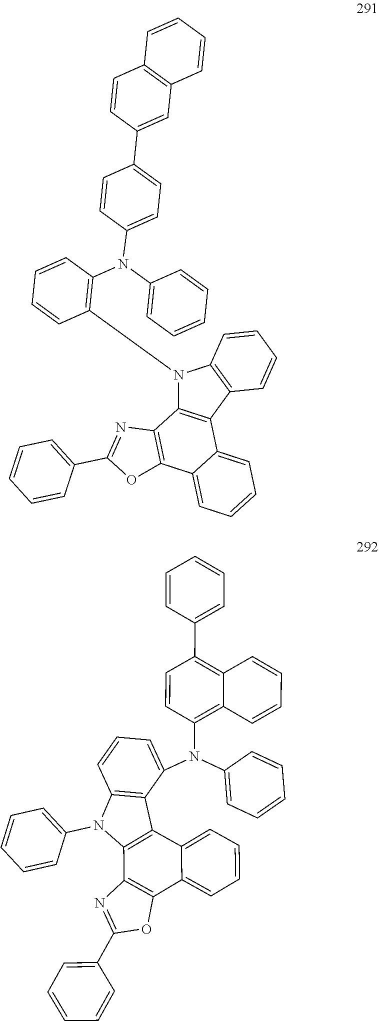 [SMILES: c1ccc(-c2nc3c(o2)c2ccccc2c2c4ccccc4n(-c4ccccc4N(c4ccccc4)c4ccc(-c5ccc6ccccc6c5)cc4)c32)cc1 ; c1ccc(-c2nc3c(o2)c2ccccc2c2c4c(N(c5ccccc5)c5ccc(-c6ccccc6)c6ccccc56)cccc4n(-c4ccccc4)c32)cc1]

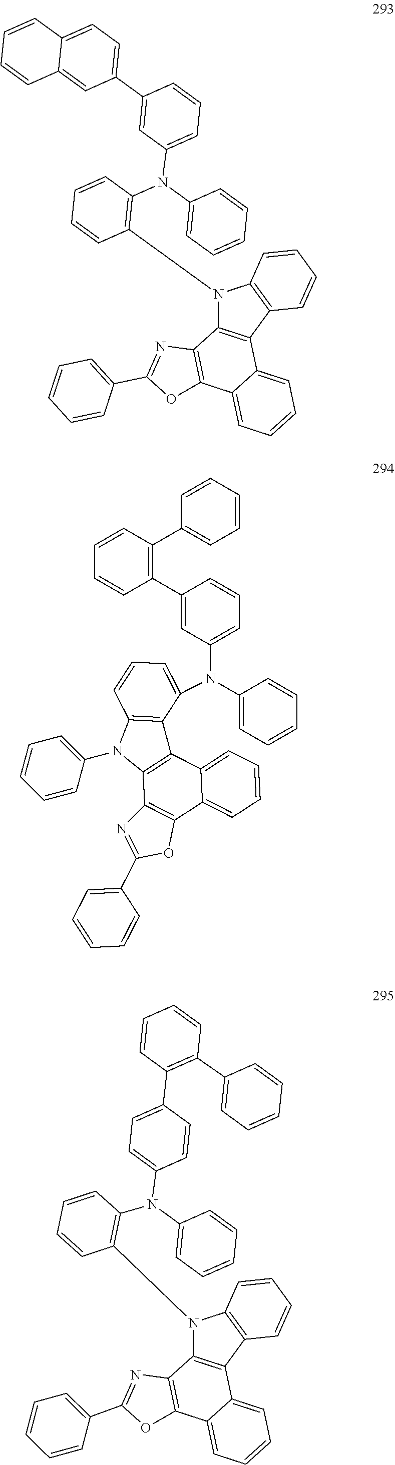 [SMILES: c1ccc(-c2nc3c(o2)c2ccccc2c2c4ccccc4n(-c4ccccc4N(c4ccccc4)c4cccc(-c5ccc6ccccc6c5)c4)c32)cc1 ; c1ccc(-c2nc3c(o2)c2ccccc2c2c4c(N(c5ccccc5)c5cccc(-c6ccccc6-c6ccccc6)c5)cccc4n(-c4ccccc4)c32)cc1 ; c1ccc(-c2nc3c(o2)c2ccccc2c2c4ccccc4n(-c4ccccc4N(c4ccccc4)c4ccc(-c5ccccc5-c5ccccc5)cc4)c32)cc1]

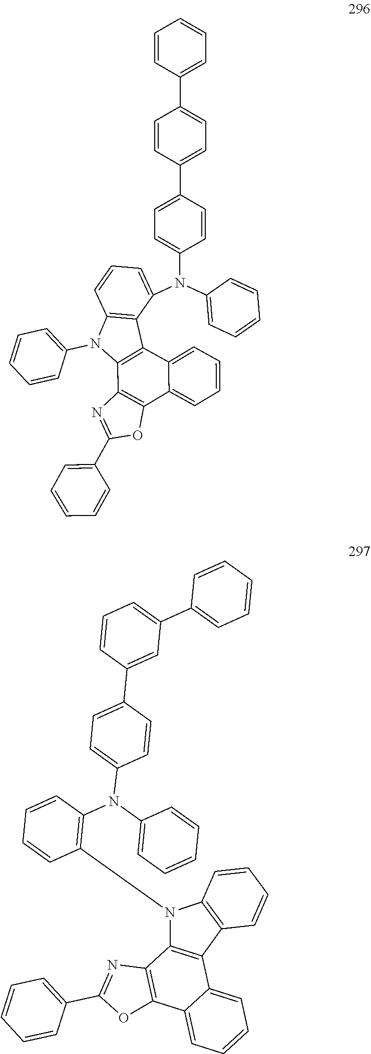 [SMILES: c1ccc(-c2ccc(-c3ccc(N(c4ccccc4)c4cccc5c4c4c6ccccc6c6oc(-c7ccccc7)nc6c4n5-c4ccccc4)cc3)cc2)cc1 ; c1ccc(-c2cccc(-c3ccc(N(c4ccccc4)c4ccccc4-n4c5ccccc5c5c6ccccc6c6oc(-c7ccccc7)nc6c54)cc3)c2)cc1]

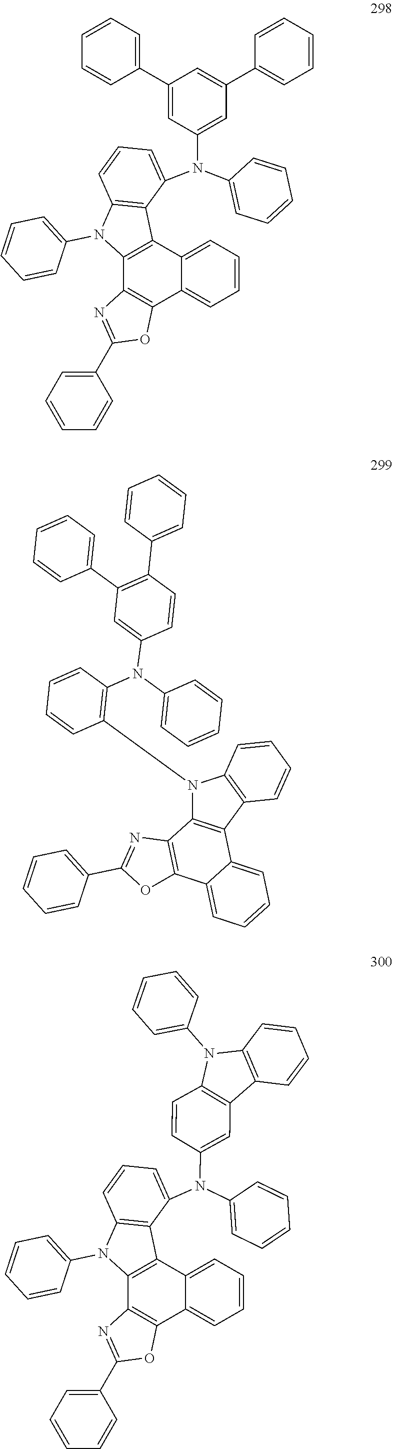 [SMILES: c1ccc(-c2cc(-c3ccccc3)cc(N(c3ccccc3)c3cccc4c3c3c5ccccc5c5oc(-c6ccccc6)nc5c3n4-c3ccccc3)c2)cc1 ; c1ccc(-c2nc3c(o2)c2ccccc2c2c4ccccc4n(-c4ccccc4N(c4ccccc4)c4ccc(-c5ccccc5)c(-c5ccccc5)c4)c32)cc1 ; c1ccc(-c2nc3c(o2)c2ccccc2c2c4c(N(c5ccccc5)c5ccc6c(c5)c5ccccc5n6-c5ccccc5)cccc4n(-c4ccccc4)c32)cc1]

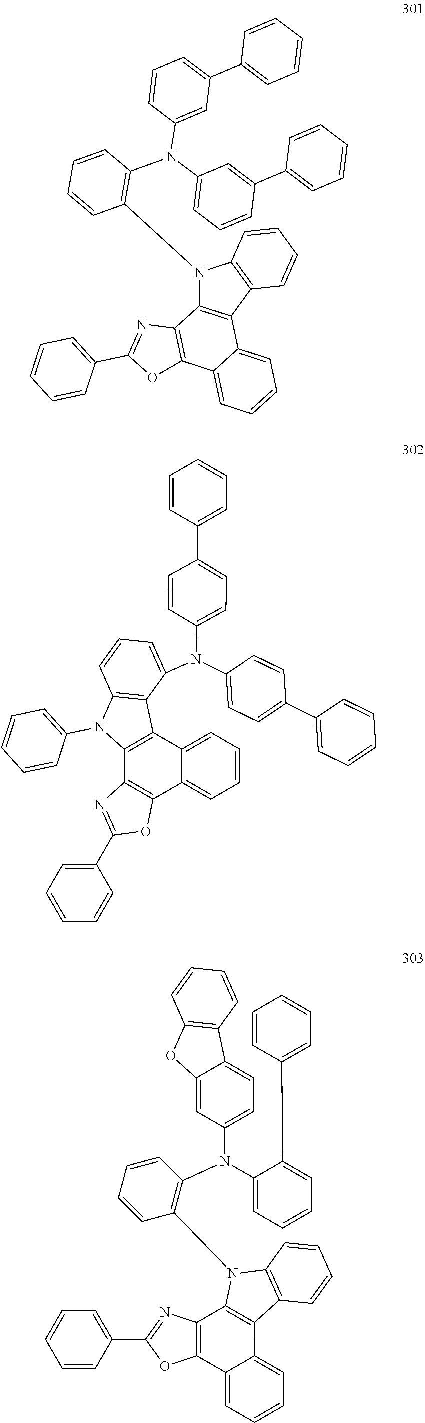 [SMILES: c1ccc(-c2cccc(N(c3cccc(-c4ccccc4)c3)c3ccccc3-n3c4ccccc4c4c5ccccc5c5oc(-c6ccccc6)nc5c43)c2)cc1 ; c1ccc(-c2ccc(N(c3ccc(-c4ccccc4)cc3)c3cccc4c3c3c5ccccc5c5oc(-c6ccccc6)nc5c3n4-c3ccccc3)cc2)cc1 ; c1ccc(-c2nc3c(o2)c2ccccc2c2c4ccccc4n(-c4ccccc4N(c4ccc5c(c4)oc4ccccc45)c4ccccc4-c4ccccc4)c32)cc1]

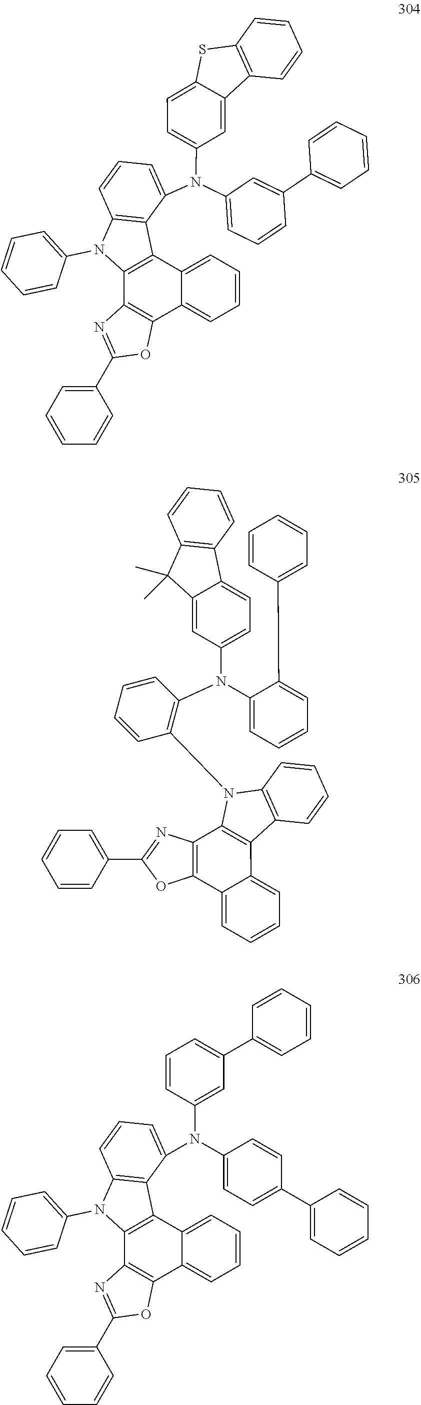 [SMILES: c1ccc(-c2cccc(N(c3ccc4sc5ccccc5c4c3)c3cccc4c3c3c5ccccc5c5oc(-c6ccccc6)nc5c3n4-c3ccccc3)c2)cc1 ; CC1(C)c2ccccc2-c2ccc(N(c3ccccc3-c3ccccc3)c3ccccc3-n3c4ccccc4c4c5ccccc5c5oc(-c6ccccc6)nc5c43)cc21 ; c1ccc(-c2ccc(N(c3cccc(-c4ccccc4)c3)c3cccc4c3c3c5ccccc5c5oc(-c6ccccc6)nc5c3n4-c3ccccc3)cc2)cc1]

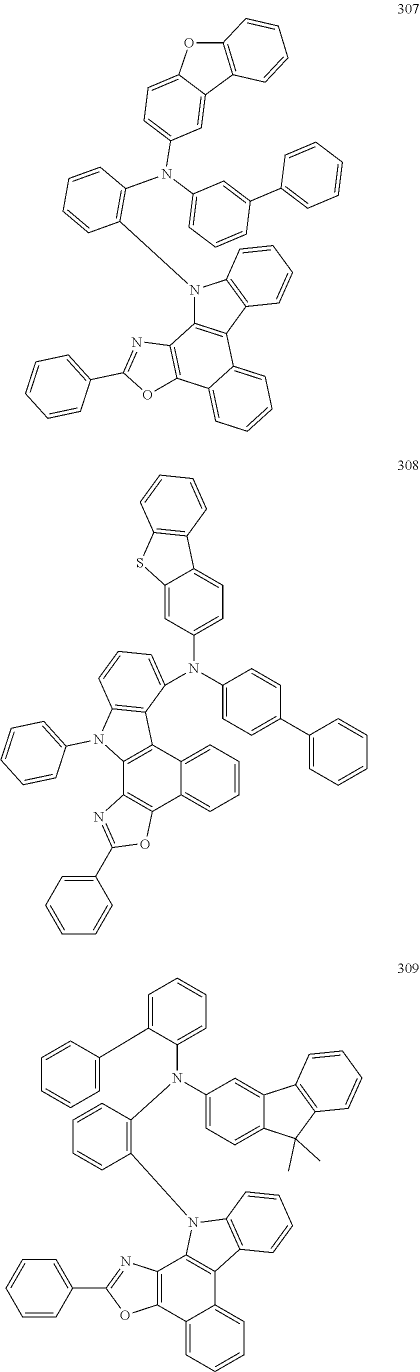 [SMILES: c1ccc(-c2cccc(N(c3ccc4oc5ccccc5c4c3)c3ccccc3-n3c4ccccc4c4c5ccccc5c5oc(-c6ccccc6)nc5c43)c2)cc1 ; c1ccc(-c2ccc(N(c3ccc4c(c3)sc3ccccc34)c3cccc4c3c3c5ccccc5c5oc(-c6ccccc6)nc5c3n4-c3ccccc3)cc2)cc1 ; CC1(C)c2ccccc2-c2cc(N(c3ccccc3-c3ccccc3)c3ccccc3-n3c4ccccc4c4c5ccccc5c5oc(-c6ccccc6)nc5c43)ccc21]

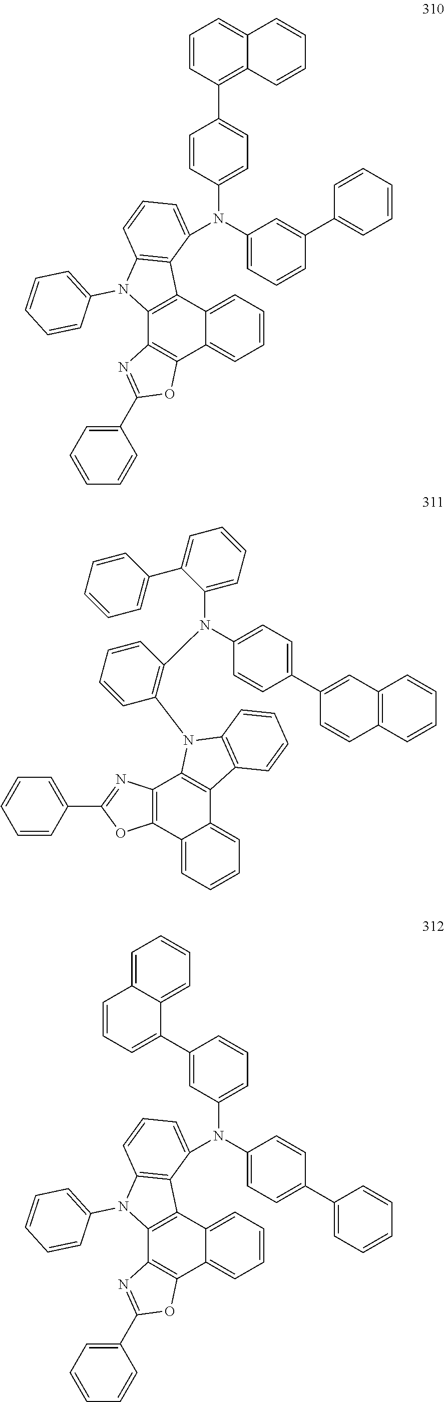 [SMILES: c1ccc(-c2cccc(N(c3ccc(-c4cccc5ccccc45)cc3)c3cccc4c3c3c5ccccc5c5oc(-c6ccccc6)nc5c3n4-c3ccccc3)c2)cc1 ; c1ccc(-c2nc3c(o2)c2ccccc2c2c4ccccc4n(-c4ccccc4N(c4ccc(-c5ccc6ccccc6c5)cc4)c4ccccc4-c4ccccc4)c32)cc1 ; c1ccc(-c2ccc(N(c3cccc(-c4cccc5ccccc45)c3)c3cccc4c3c3c5ccccc5c5oc(-c6ccccc6)nc5c3n4-c3ccccc3)cc2)cc1]

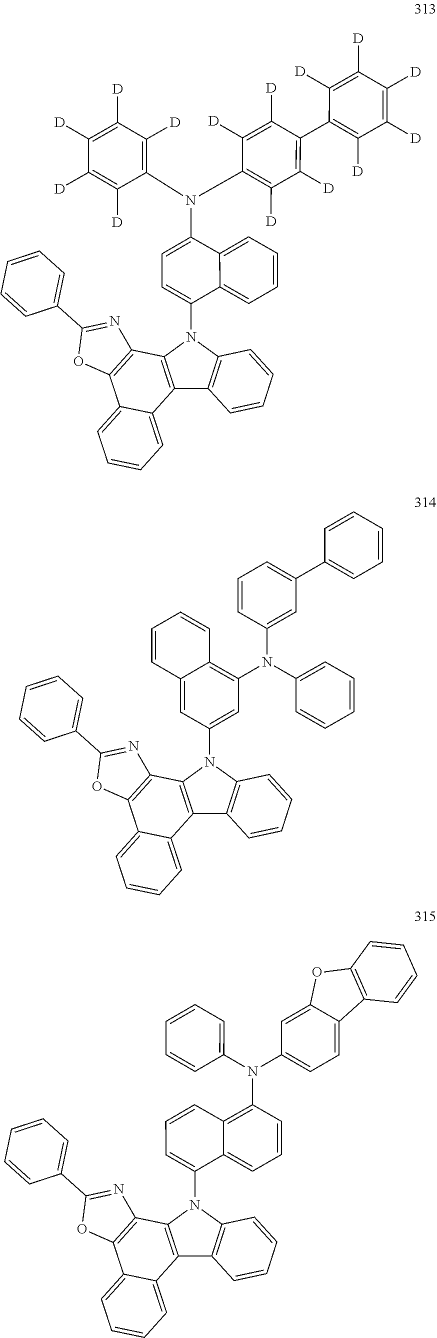 [SMILES: [2H]c1c([2H])c([2H])c(-c2c([2H])c([2H])c(N(c3c([2H])c([2H])c([2H])c([2H])c3[2H])c3ccc(-n4c5ccccc5c5c6ccccc6c6oc(-c7ccccc7)nc6c54)c4ccccc34)c([2H])c2[2H])c([2H])c1[2H] ; c1ccc(-c2cccc(N(c3ccccc3)c3cc(-n4c5ccccc5c5c6ccccc6c6oc(-c7ccccc7)nc6c54)cc4ccccc34)c2)cc1 ; c1ccc(-c2nc3c(o2)c2ccccc2c2c4ccccc4n(-c4cccc5c(N(c6ccccc6)c6ccc7c(c6)oc6ccccc67)cccc45)c32)cc1]

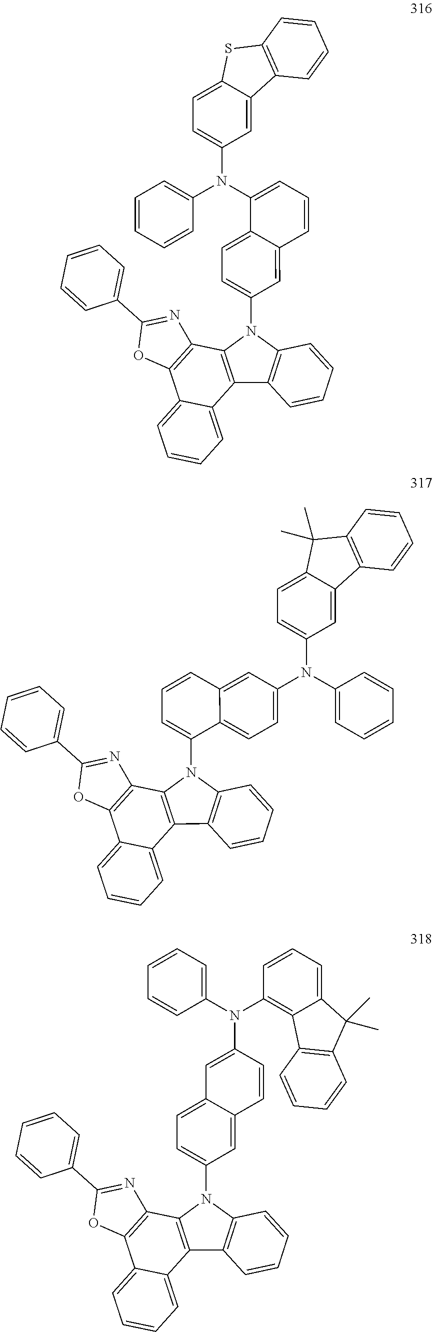 [SMILES: c1ccc(-c2nc3c(o2)c2ccccc2c2c4ccccc4n(-c4ccc5c(N(c6ccccc6)c6ccc7sc8ccccc8c7c6)cccc5c4)c32)cc1 ; CC1(C)c2ccccc2-c2cc(N(c3ccccc3)c3ccc4c(-n5c6ccccc6c6c7ccccc7c7oc(-c8ccccc8)nc7c65)cccc4c3)ccc21 ; CC1(C)c2ccccc2-c2c(N(c3ccccc3)c3ccc4cc(-n5c6ccccc6c6c7ccccc7c7oc(-c8ccccc8)nc7c65)ccc4c3)cccc21]

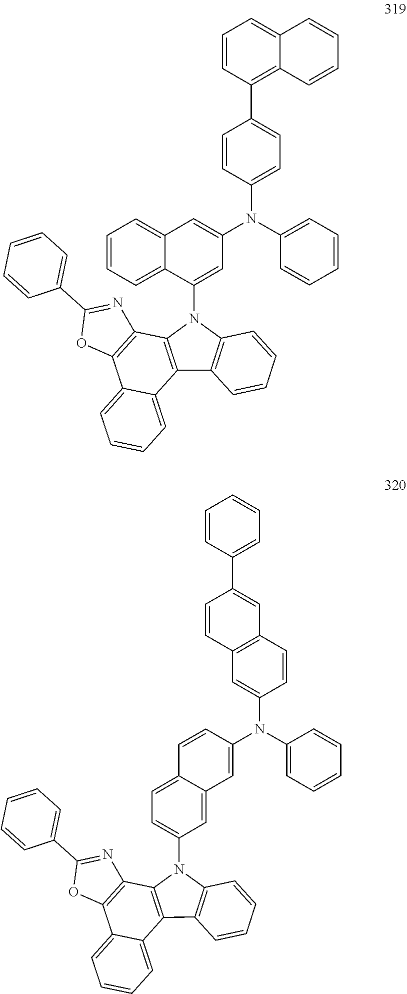 [SMILES: c1ccc(-c2nc3c(o2)c2ccccc2c2c4ccccc4n(-c4cc(N(c5ccccc5)c5ccc(-c6cccc7ccccc67)cc5)cc5ccccc45)c32)cc1 ; c1ccc(-c2ccc3cc(N(c4ccccc4)c4ccc5ccc(-n6c7ccccc7c7c8ccccc8c8oc(-c9ccccc9)nc8c76)cc5c4)ccc3c2)cc1]

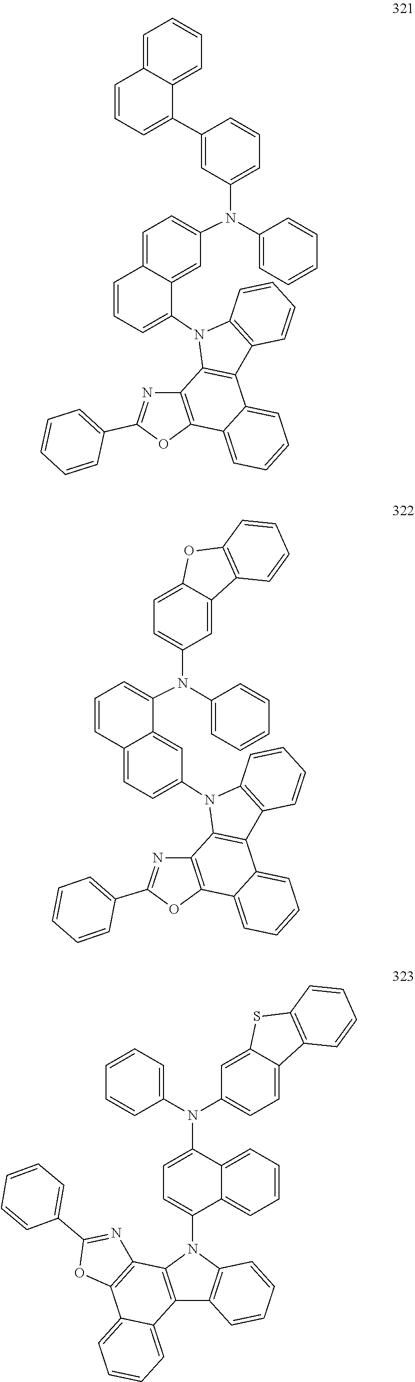 [SMILES: c1ccc(-c2nc3c(o2)c2ccccc2c2c4ccccc4n(-c4cccc5ccc(N(c6ccccc6)c6cccc(-c7cccc8ccccc78)c6)cc45)c32)cc1 ; c1ccc(-c2nc3c(o2)c2ccccc2c2c4ccccc4n(-c4ccc5cccc(N(c6ccccc6)c6ccc7oc8ccccc8c7c6)c5c4)c32)cc1 ; c1ccc(-c2nc3c(o2)c2ccccc2c2c4ccccc4n(-c4ccc(N(c5ccccc5)c5ccc6c(c5)sc5ccccc56)c5ccccc45)c32)cc1]

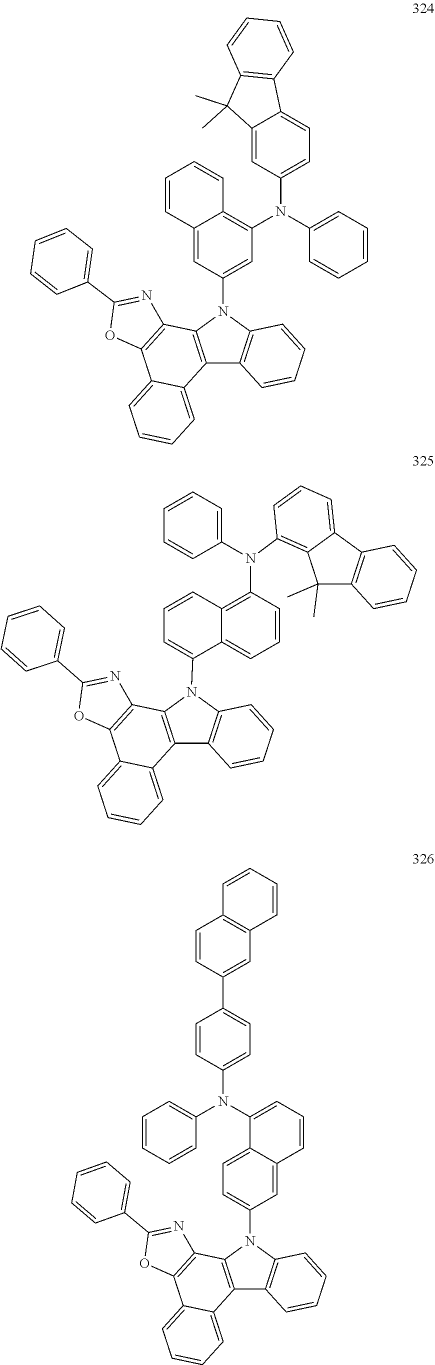 [SMILES: CC1(C)c2ccccc2-c2ccc(N(c3ccccc3)c3cc(-n4c5ccccc5c5c6ccccc6c6oc(-c7ccccc7)nc6c54)cc4ccccc34)cc21 ; CC1(C)c2ccccc2-c2cccc(N(c3ccccc3)c3cccc4c(-n5c6ccccc6c6c7ccccc7c7oc(-c8ccccc8)nc7c65)cccc34)c21 ; c1ccc(-c2nc3c(o2)c2ccccc2c2c4ccccc4n(-c4ccc5c(N(c6ccccc6)c6ccc(-c7ccc8ccccc8c7)cc6)cccc5c4)c32)cc1]

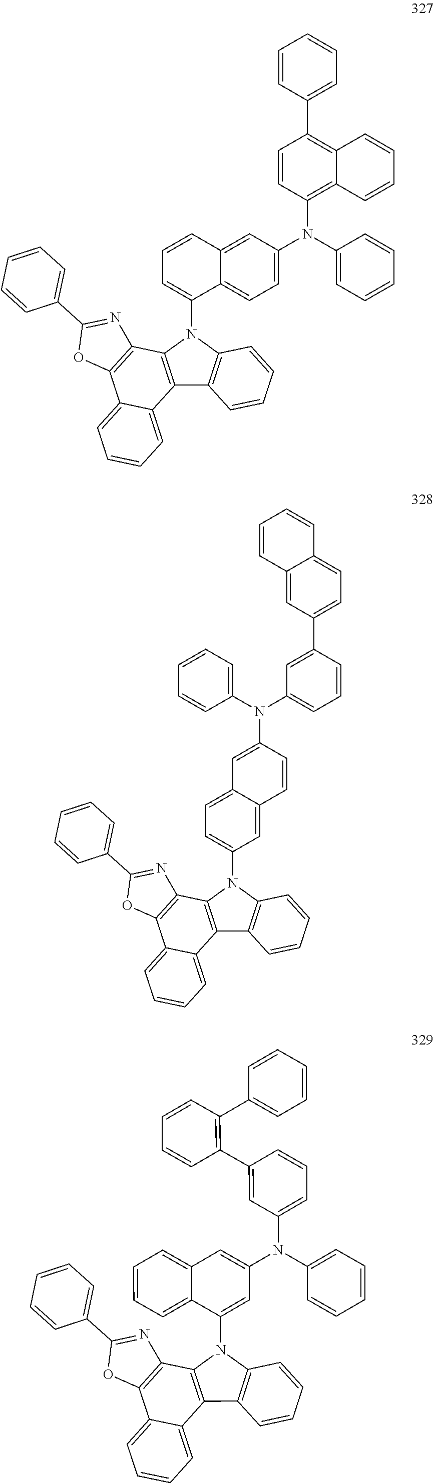 [SMILES: c1ccc(-c2nc3c(o2)c2ccccc2c2c4ccccc4n(-c4cccc5cc(N(c6ccccc6)c6ccc(-c7ccccc7)c7ccccc67)ccc45)c32)cc1 ; c1ccc(-c2nc3c(o2)c2ccccc2c2c4ccccc4n(-c4ccc5cc(N(c6ccccc6)c6cccc(-c7ccc8ccccc8c7)c6)ccc5c4)c32)cc1 ; c1ccc(-c2nc3c(o2)c2ccccc2c2c4ccccc4n(-c4cc(N(c5ccccc5)c5cccc(-c6ccccc6-c6ccccc6)c5)cc5ccccc45)c32)cc1]

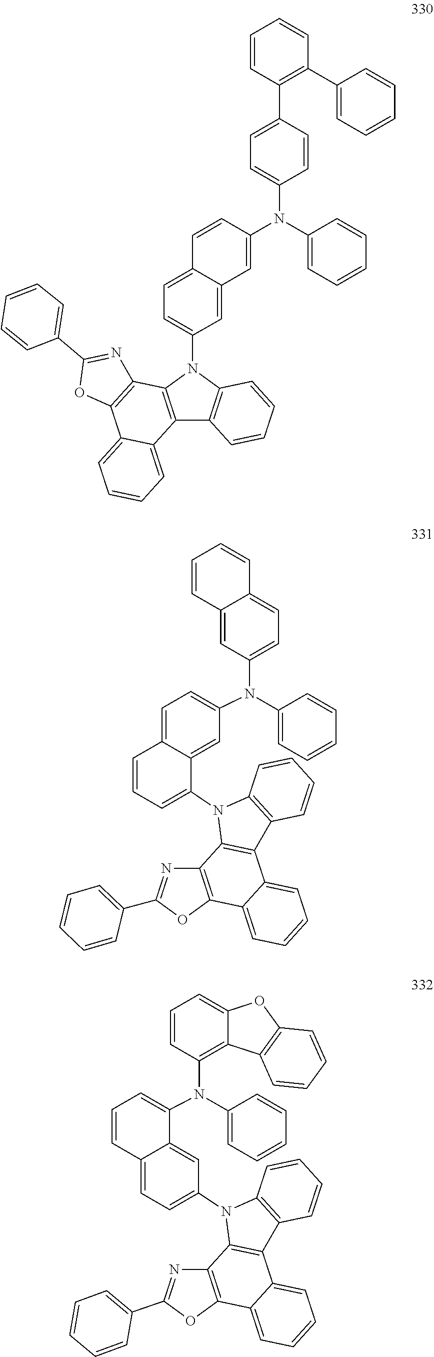 [SMILES: c1ccc(-c2nc3c(o2)c2ccccc2c2c4ccccc4n(-c4ccc5ccc(N(c6ccccc6)c6ccc(-c7ccccc7-c7ccccc7)cc6)cc5c4)c32)cc1 ; c1ccc(-c2nc3c(o2)c2ccccc2c2c4ccccc4n(-c4cccc5ccc(N(c6ccccc6)c6ccc7ccccc7c6)cc45)c32)cc1 ; c1ccc(-c2nc3c(o2)c2ccccc2c2c4ccccc4n(-c4ccc5cccc(N(c6ccccc6)c6cccc7oc8ccccc8c67)c5c4)c32)cc1]

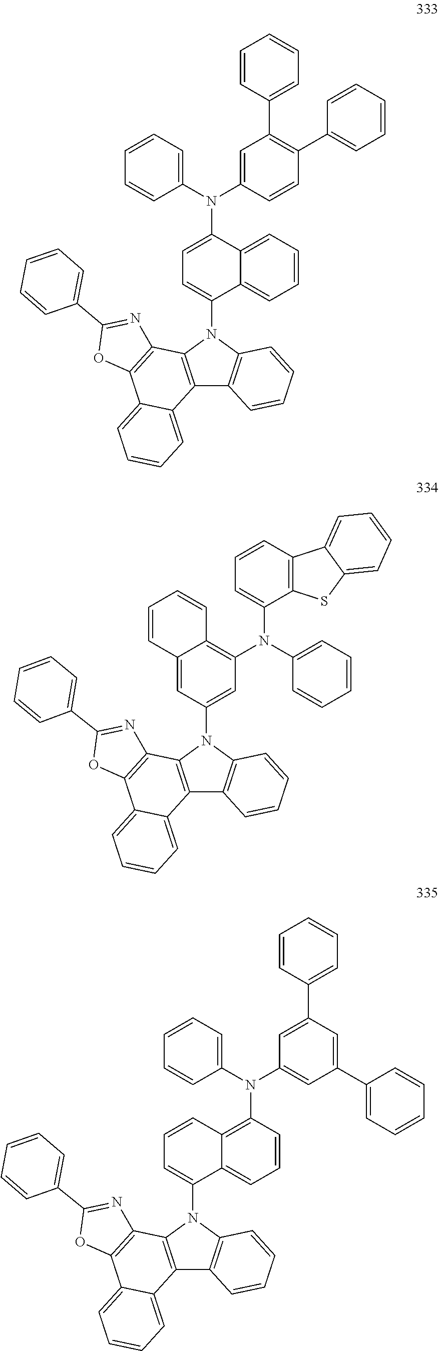 [SMILES: c1ccc(-c2nc3c(o2)c2ccccc2c2c4ccccc4n(-c4ccc(N(c5ccccc5)c5ccc(-c6ccccc6)c(-c6ccccc6)c5)c5ccccc45)c32)cc1 ; c1ccc(-c2nc3c(o2)c2ccccc2c2c4ccccc4n(-c4cc(N(c5ccccc5)c5cccc6c5sc5ccccc56)c5ccccc5c4)c32)cc1 ; c1ccc(-c2cc(-c3ccccc3)cc(N(c3ccccc3)c3cccc4c(-n5c6ccccc6c6c7ccccc7c7oc(-c8ccccc8)nc7c65)cccc34)c2)cc1]

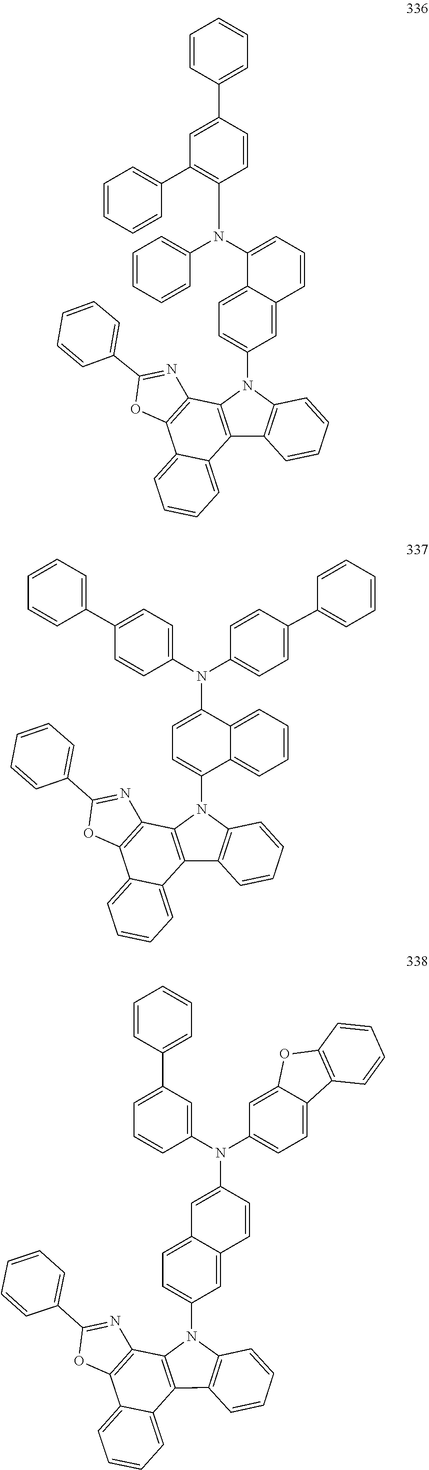

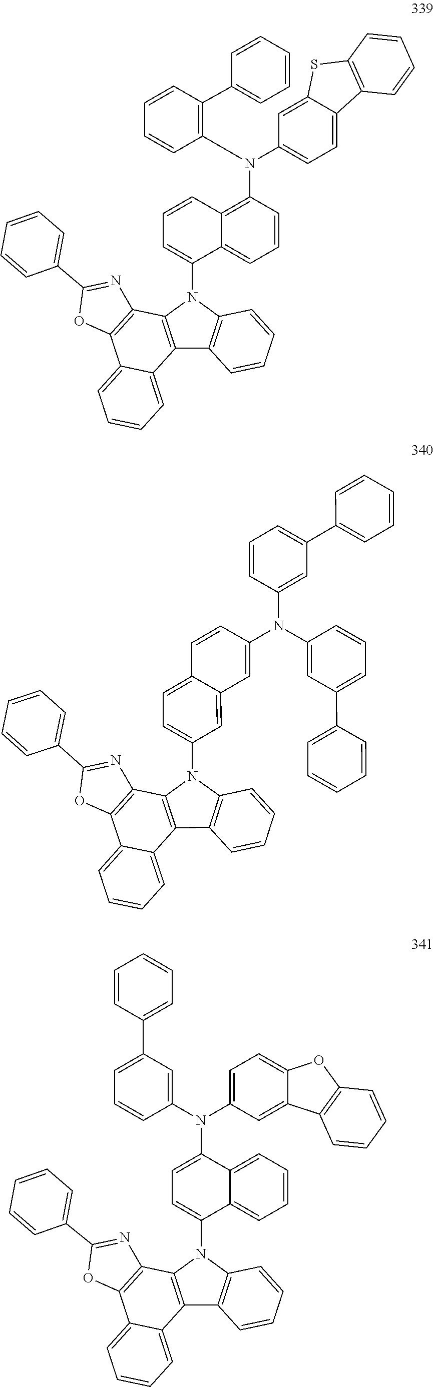

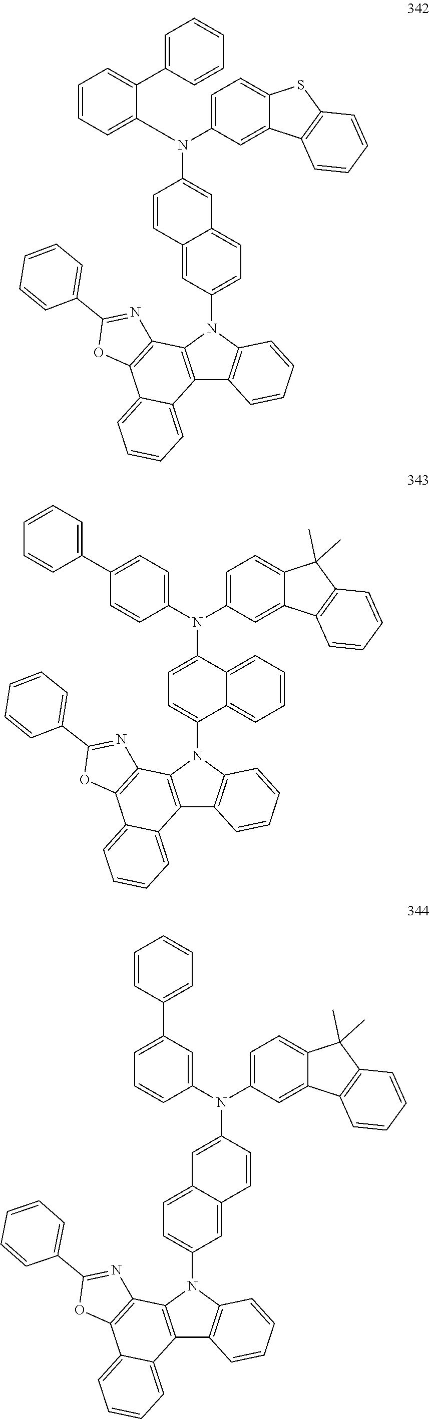 [SMILES: c1ccc(-c2nc3c(o2)c2ccccc2c2c4ccccc4n(-c4ccc5cc(N(c6ccc7sc8ccccc8c7c6)c6ccccc6-c6ccccc6)ccc5c4)c32)cc1 ; CC1(C)c2ccccc2-c2cc(N(c3ccc(-c4ccccc4)cc3)c3ccc(-n4c5ccccc5c5c6ccccc6c6oc(-c7ccccc7)nc6c54)c4ccccc34)ccc21 ; CC1(C)c2ccccc2-c2cc(N(c3cccc(-c4ccccc4)c3)c3ccc4cc(-n5c6ccccc6c6c7ccccc7c7oc(-c8ccccc8)nc7c65)ccc4c3)ccc21]

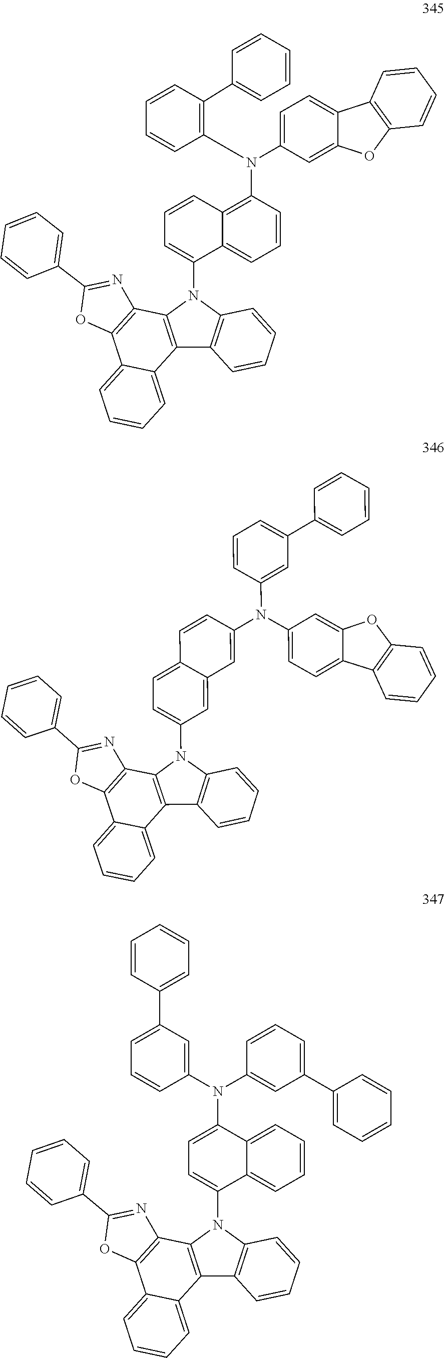 [SMILES: c1ccc(-c2nc3c(o2)c2ccccc2c2c4ccccc4n(-c4cccc5c(N(c6ccc7c(c6)oc6ccccc67)c6ccccc6-c6ccccc6)cccc45)c32)cc1 ; c1ccc(-c2cccc(N(c3ccc4ccc(-n5c6ccccc6c6c7ccccc7c7oc(-c8ccccc8)nc7c65)cc4c3)c3ccc4c(c3)oc3ccccc34)c2)cc1 ; c1ccc(-c2cccc(N(c3cccc(-c4ccccc4)c3)c3ccc(-n4c5ccccc5c5c6ccccc6c6oc(-c7ccccc7)nc6c54)c4ccccc34)c2)cc1]

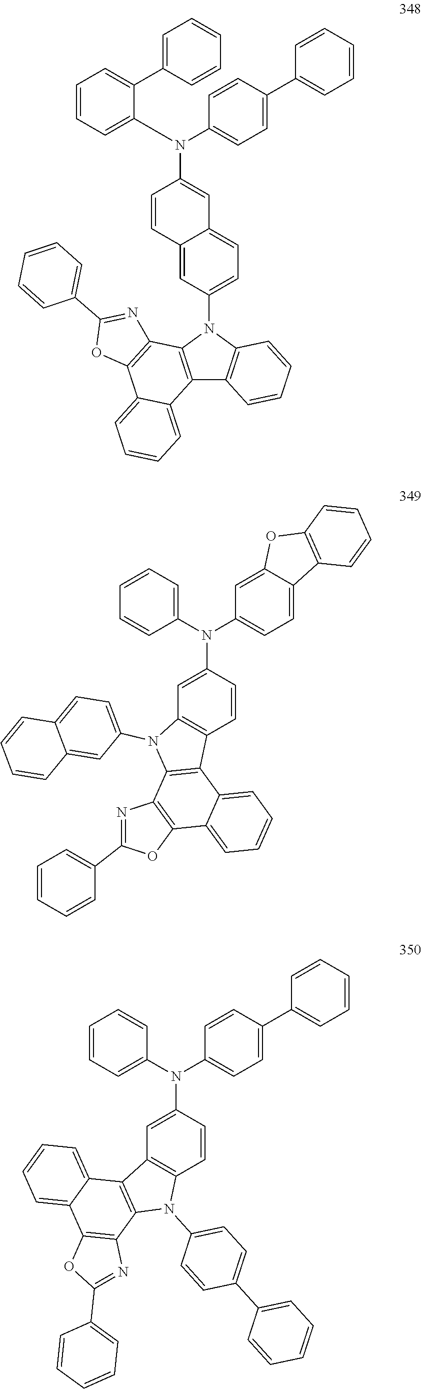 [SMILES: c1ccc(-c2ccc(N(c3ccc4cc(-n5c6ccccc6c6c7ccccc7c7oc(-c8ccccc8)nc7c65)ccc4c3)c3ccccc3-c3ccccc3)cc2)cc1 ; c1ccc(-c2nc3c(o2)c2ccccc2c2c4ccc(N(c5ccccc5)c5ccc6c(c5)oc5ccccc56)cc4n(-c4ccc5ccccc5c4)c32)cc1 ; c1ccc(-c2ccc(N(c3ccccc3)c3ccc4c(c3)c3c5ccccc5c5oc(-c6ccccc6)nc5c3n4-c3ccc(-c4ccccc4)cc3)cc2)cc1]

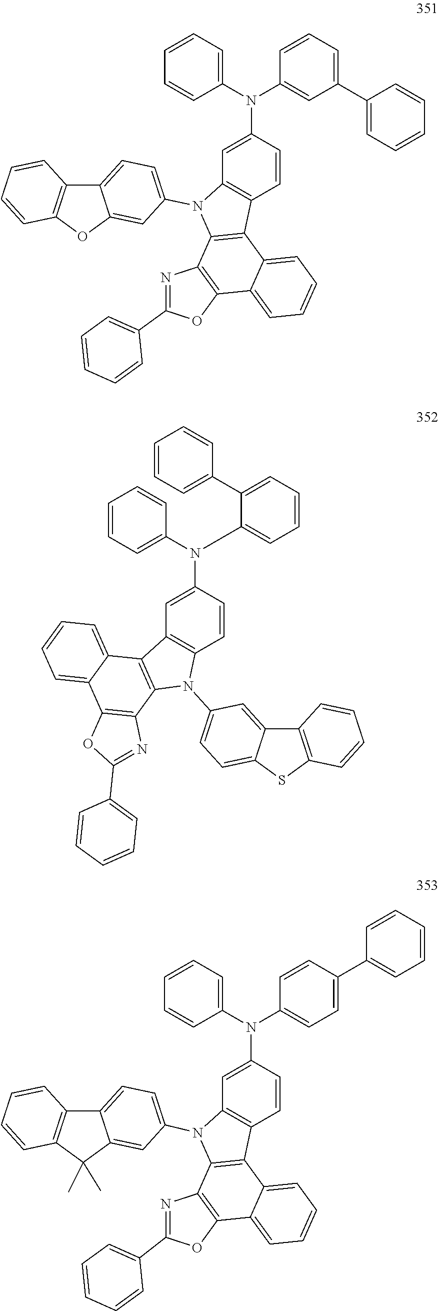 [SMILES: c1ccc(-c2cccc(N(c3ccccc3)c3ccc4c5c6ccccc6c6oc(-c7ccccc7)nc6c5n(-c5ccc6c(c5)oc5ccccc56)c4c3)c2)cc1 ; c1ccc(-c2nc3c(o2)c2ccccc2c2c4cc(N(c5ccccc5)c5ccccc5-c5ccccc5)ccc4n(-c4ccc5sc6ccccc6c5c4)c32)cc1 ; CC1(C)c2ccccc2-c2ccc(-n3c4cc(N(c5ccccc5)c5ccc(-c6ccccc6)cc5)ccc4c4c5ccccc5c5oc(-c6ccccc6)nc5c43)cc21]

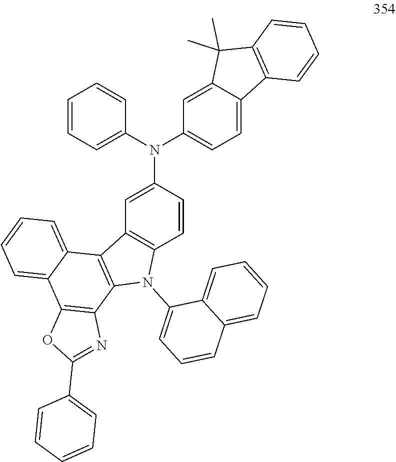 [SMILES: CC1(C)c2ccccc2-c2ccc(N(c3ccccc3)c3ccc4c(c3)c3c5ccccc5c5oc(-c6ccccc6)nc5c3n4-c3cccc4ccccc34)cc21]

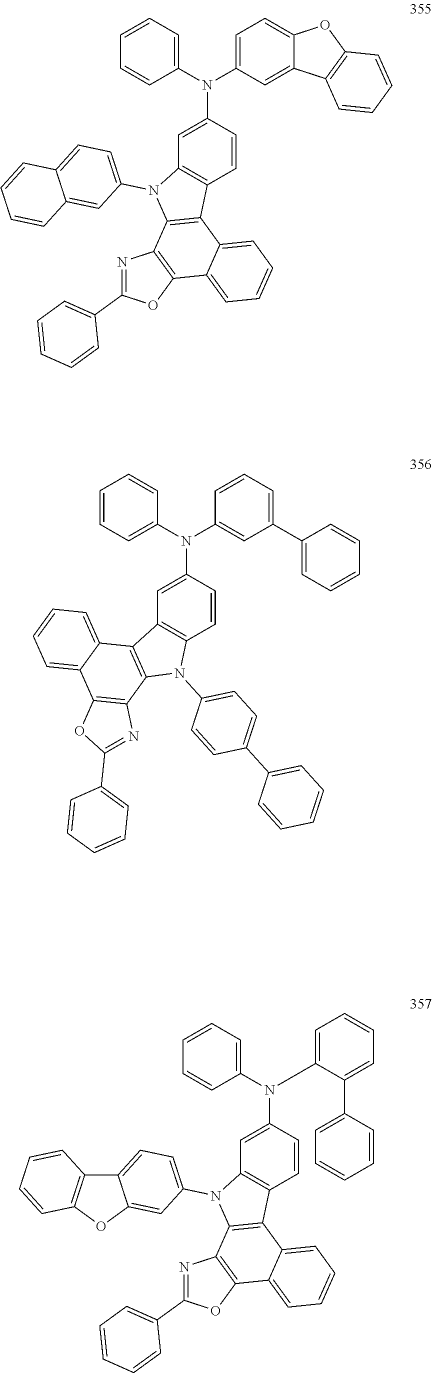 [SMILES: c1ccc(-c2nc3c(o2)c2ccccc2c2c4ccc(N(c5ccccc5)c5ccc6oc7ccccc7c6c5)cc4n(-c4ccc5ccccc5c4)c32)cc1 ; c1ccc(-c2ccc(-n3c4ccc(N(c5ccccc5)c5cccc(-c6ccccc6)c5)cc4c4c5ccccc5c5oc(-c6ccccc6)nc5c43)cc2)cc1 ; c1ccc(-c2nc3c(o2)c2ccccc2c2c4ccc(N(c5ccccc5)c5ccccc5-c5ccccc5)cc4n(-c4ccc5c(c4)oc4ccccc45)c32)cc1]

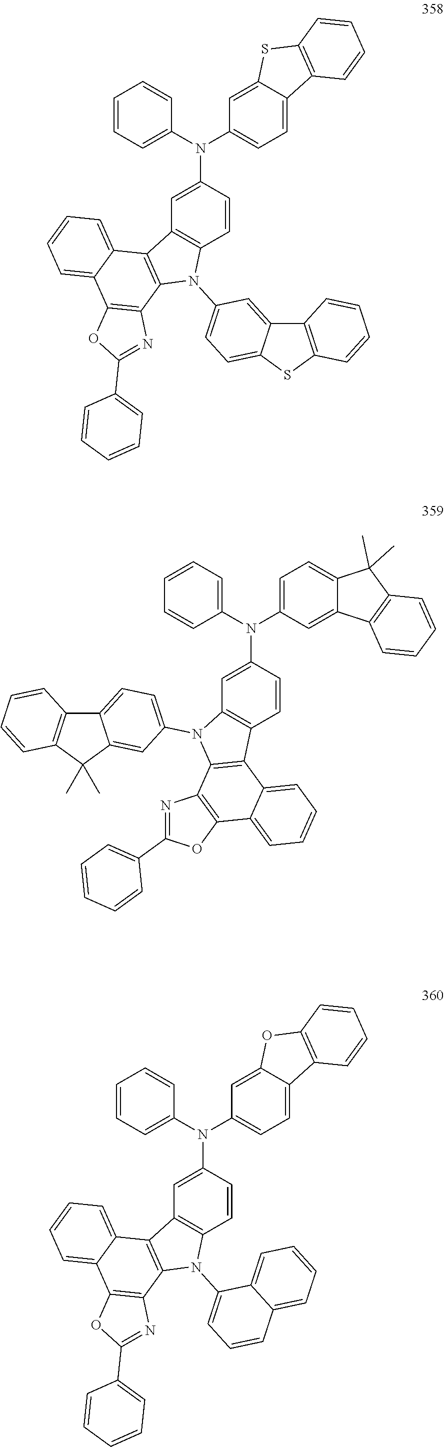 [SMILES: c1ccc(-c2nc3c(o2)c2ccccc2c2c4cc(N(c5ccccc5)c5ccc6c(c5)sc5ccccc56)ccc4n(-c4ccc5sc6ccccc6c5c4)c32)cc1 ; CC1(C)c2ccccc2-c2cc(N(c3ccccc3)c3ccc4c5c6ccccc6c6oc(-c7ccccc7)nc6c5n(-c5ccc6c(c5)C(C)(C)c5ccccc5-6)c4c3)ccc21 ; c1ccc(-c2nc3c(o2)c2ccccc2c2c4cc(N(c5ccccc5)c5ccc6c(c5)oc5ccccc56)ccc4n(-c4cccc5ccccc45)c32)cc1]

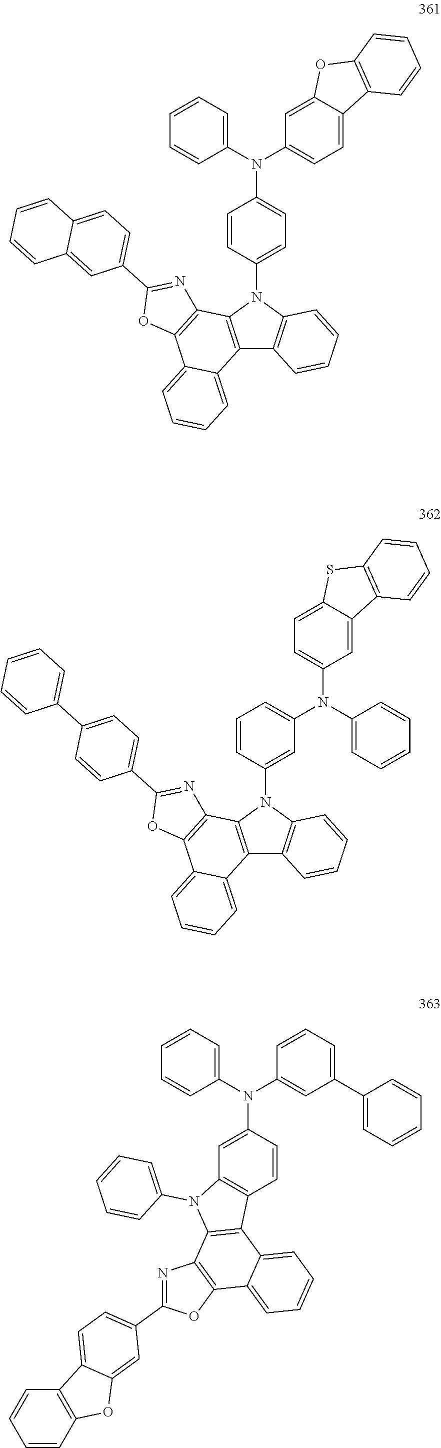 [SMILES: c1ccc(N(c2ccc(-n3c4ccccc4c4c5ccccc5c5oc(-c6ccc7ccccc7c6)nc5c43)cc2)c2ccc3c(c2)oc2ccccc23)cc1 ; c1ccc(-c2ccc(-c3nc4c(o3)c3ccccc3c3c5ccccc5n(-c5cccc(N(c6ccccc6)c6ccc7sc8ccccc8c7c6)c5)c43)cc2)cc1 ; c1ccc(-c2cccc(N(c3ccccc3)c3ccc4c5c6ccccc6c6oc(-c7ccc8c(c7)oc7ccccc78)nc6c5n(-c5ccccc5)c4c3)c2)cc1]

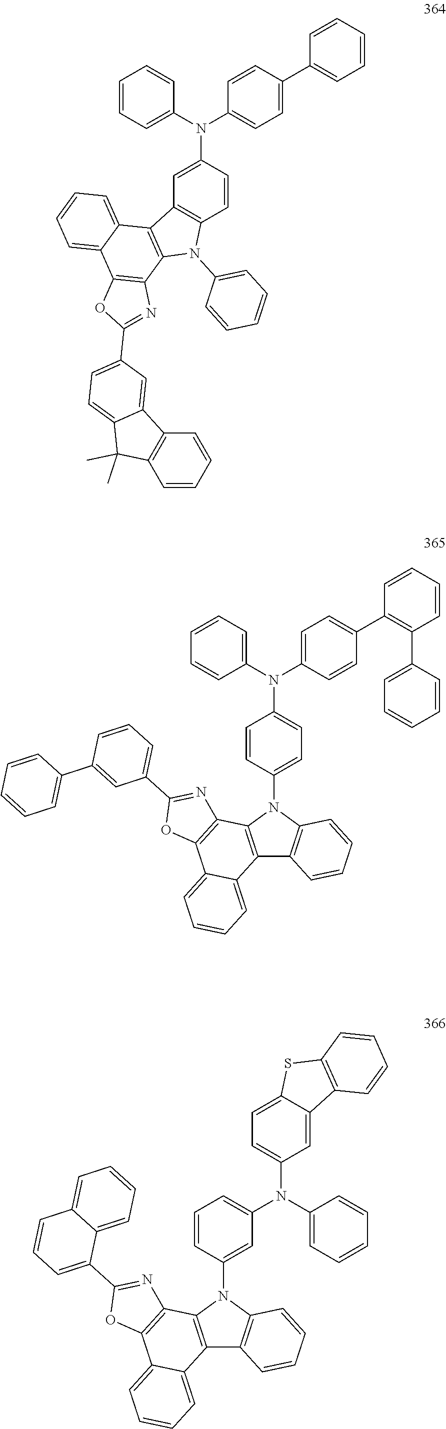 [SMILES: CC1(C)c2ccccc2-c2cc(-c3nc4c(o3)c3ccccc3c3c5cc(N(c6ccccc6)c6ccc(-c7ccccc7)cc6)ccc5n(-c5ccccc5)c43)ccc21 ; c1ccc(-c2cccc(-c3nc4c(o3)c3ccccc3c3c5ccccc5n(-c5ccc(N(c6ccccc6)c6ccc(-c7ccccc7-c7ccccc7)cc6)cc5)c43)c2)cc1 ; c1ccc(N(c2cccc(-n3c4ccccc4c4c5ccccc5c5oc(-c6cccc7ccccc67)nc5c43)c2)c2ccc3sc4ccccc4c3c2)cc1]

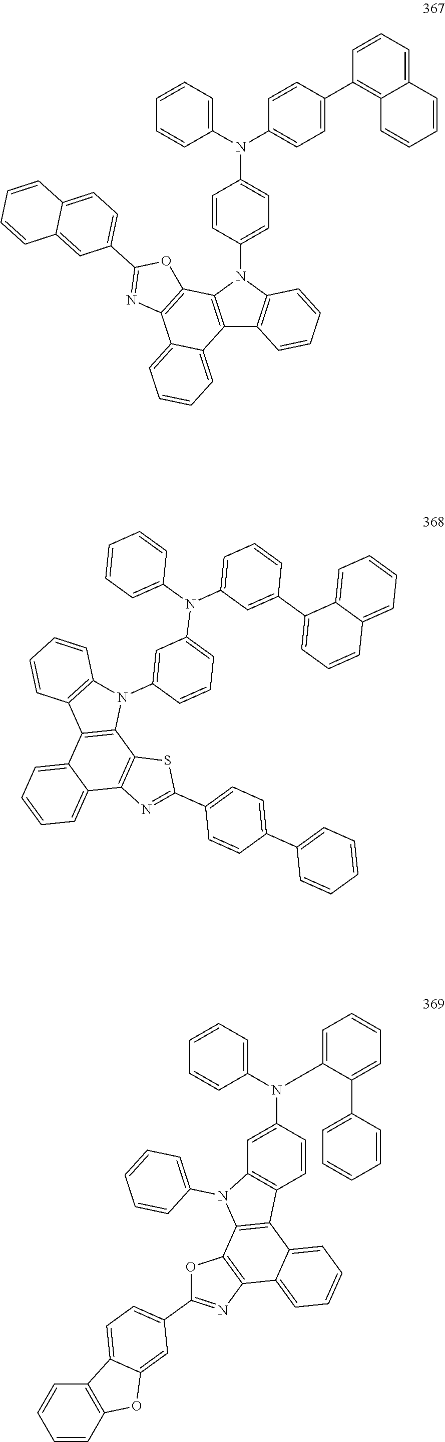 [SMILES: c1ccc(N(c2ccc(-c3cccc4ccccc34)cc2)c2ccc(-n3c4ccccc4c4c5ccccc5c5nc(-c6ccc7ccccc7c6)oc5c43)cc2)cc1 ; c1ccc(-c2ccc(-c3nc4c5ccccc5c5c6ccccc6n(-c6cccc(N(c7ccccc7)c7cccc(-c8cccc9ccccc89)c7)c6)c5c4s3)cc2)cc1 ; c1ccc(-c2ccccc2N(c2ccccc2)c2ccc3c4c5ccccc5c5nc(-c6ccc7c(c6)oc6ccccc67)oc5c4n(-c4ccccc4)c3c2)cc1]

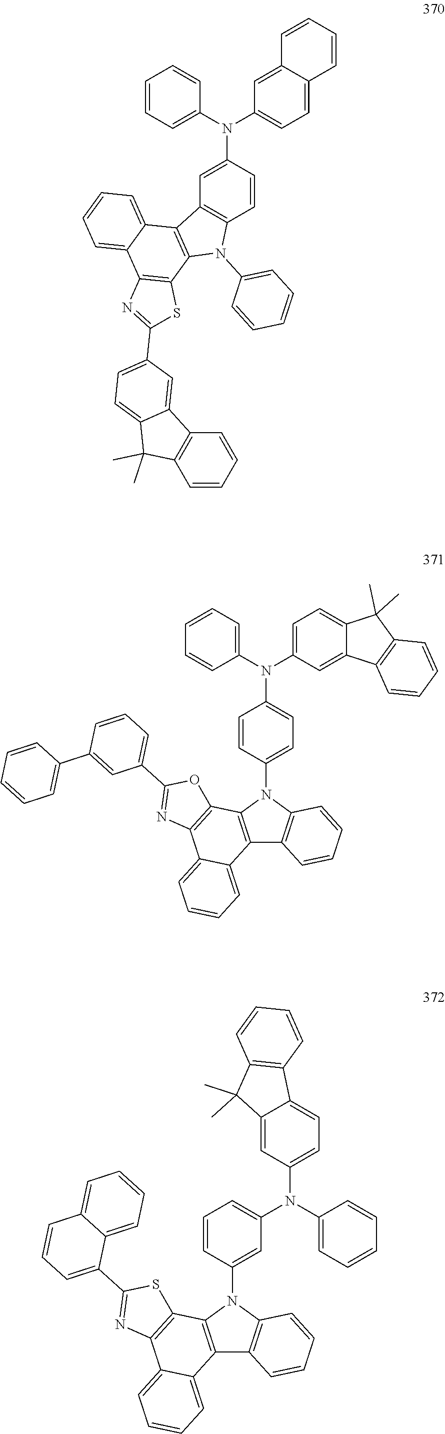 [SMILES: CC1(C)c2ccccc2-c2cc(-c3nc4c5ccccc5c5c6cc(N(c7ccccc7)c7ccc8ccccc8c7)ccc6n(-c6ccccc6)c5c4s3)ccc21 ; CC1(C)c2ccccc2-c2cc(N(c3ccccc3)c3ccc(-n4c5ccccc5c5c6ccccc6c6nc(-c7cccc(-c8ccccc8)c7)oc6c54)cc3)ccc21 ; CC1(C)c2ccccc2-c2ccc(N(c3ccccc3)c3cccc(-n4c5ccccc5c5c6ccccc6c6nc(-c7cccc8ccccc78)sc6c54)c3)cc21]

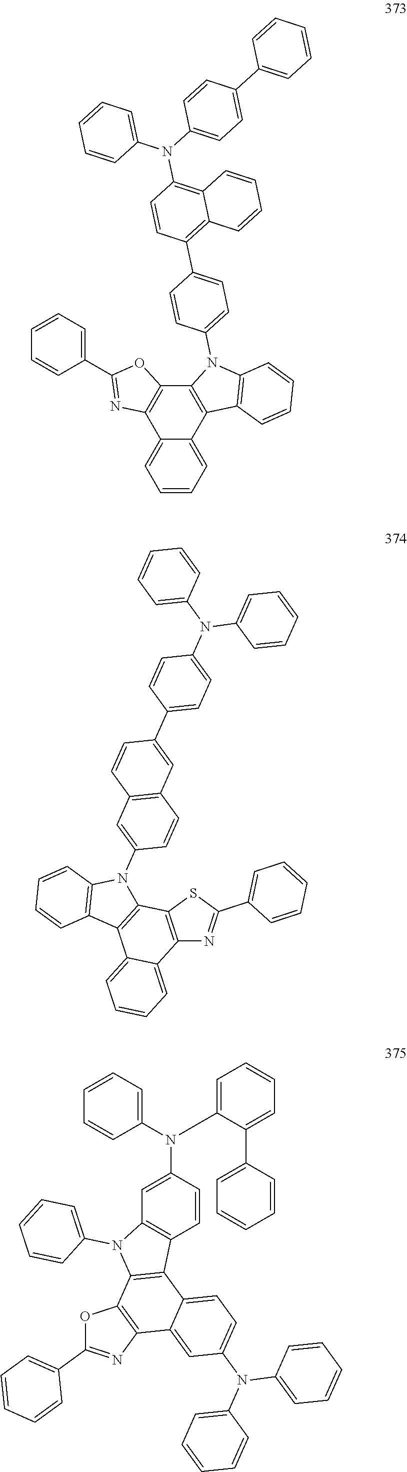 [SMILES: c1ccc(-c2ccc(N(c3ccccc3)c3ccc(-c4ccc(-n5c6ccccc6c6c7ccccc7c7nc(-c8ccccc8)oc7c65)cc4)c4ccccc34)cc2)cc1 ; c1ccc(-c2nc3c4ccccc4c4c5ccccc5n(-c5ccc6cc(-c7ccc(N(c8ccccc8)c8ccccc8)cc7)ccc6c5)c4c3s2)cc1 ; c1ccc(-c2nc3c4cc(N(c5ccccc5)c5ccccc5)ccc4c4c5ccc(N(c6ccccc6)c6ccccc6-c6ccccc6)cc5n(-c5ccccc5)c4c3o2)cc1]

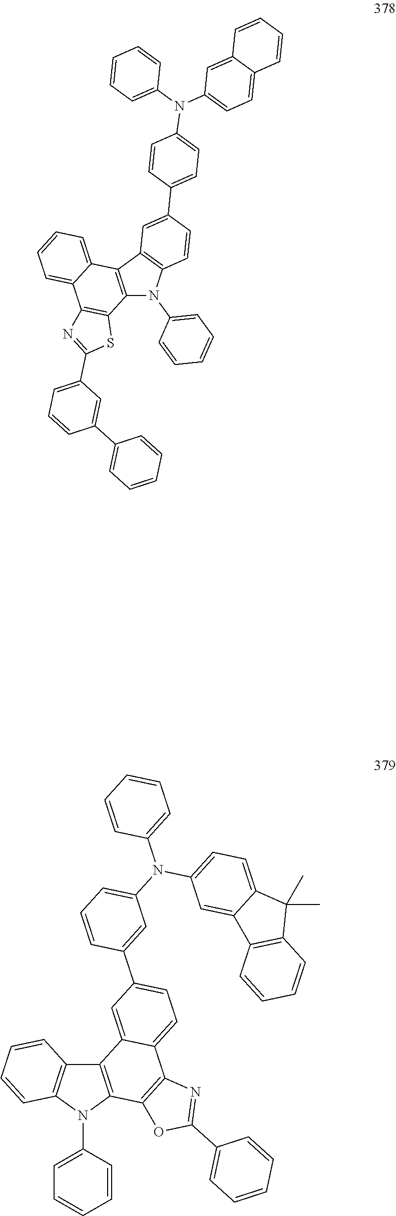 [SMILES: c1ccc(-c2cccc(-c3nc4c5ccccc5c5c6cc(-c7ccc(N(c8ccccc8)c8ccc9ccccc9c8)cc7)ccc6n(-c6ccccc6)c5c4s3)c2)cc1 ; CC1(C)c2ccccc2-c2cc(N(c3ccccc3)c3cccc(-c4ccc5c(c4)c4c6ccccc6n(-c6ccccc6)c4c4oc(-c6ccccc6)nc54)c3)ccc21]

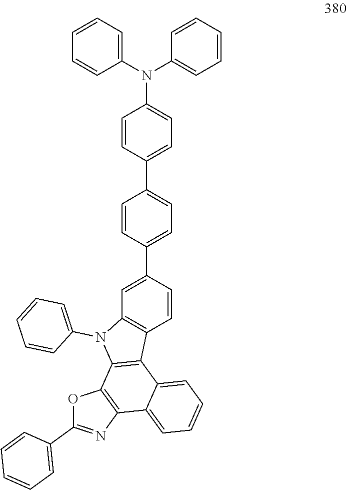 [SMILES: c1ccc(-c2nc3c4ccccc4c4c5ccc(-c6ccc(-c7ccc(N(c8ccccc8)c8ccccc8)cc7)cc6)cc5n(-c5ccccc5)c4c3o2)cc1]

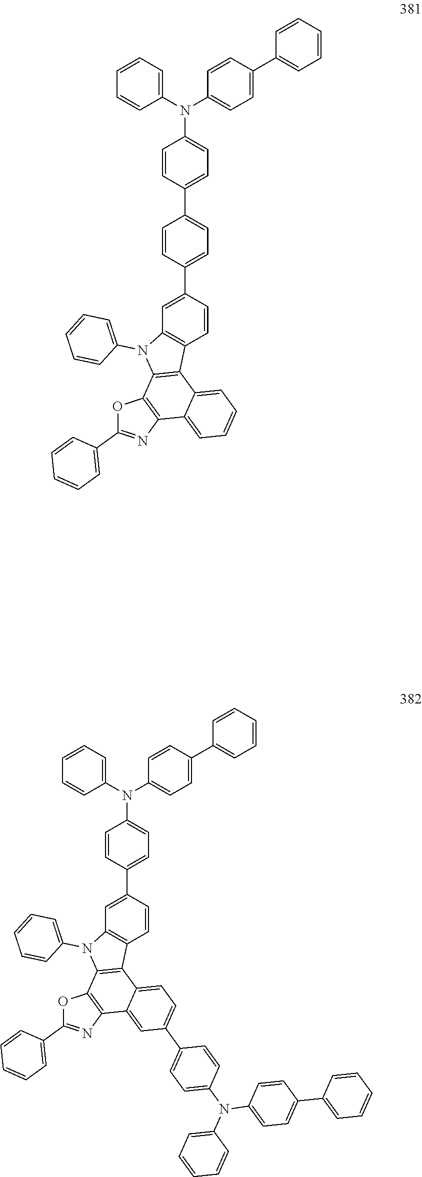 [SMILES: c1ccc(-c2ccc(N(c3ccccc3)c3ccc(-c4ccc(-c5ccc6c7c8ccccc8c8nc(-c9ccccc9)oc8c7n(-c7ccccc7)c6c5)cc4)cc3)cc2)cc1 ; c1ccc(-c2ccc(N(c3ccccc3)c3ccc(-c4ccc5c(c4)c4nc(-c6ccccc6)oc4c4c5c5ccc(-c6ccc(N(c7ccccc7)c7ccc(-c8ccccc8)cc7)cc6)cc5n4-c4ccccc4)cc3)cc2)cc1]

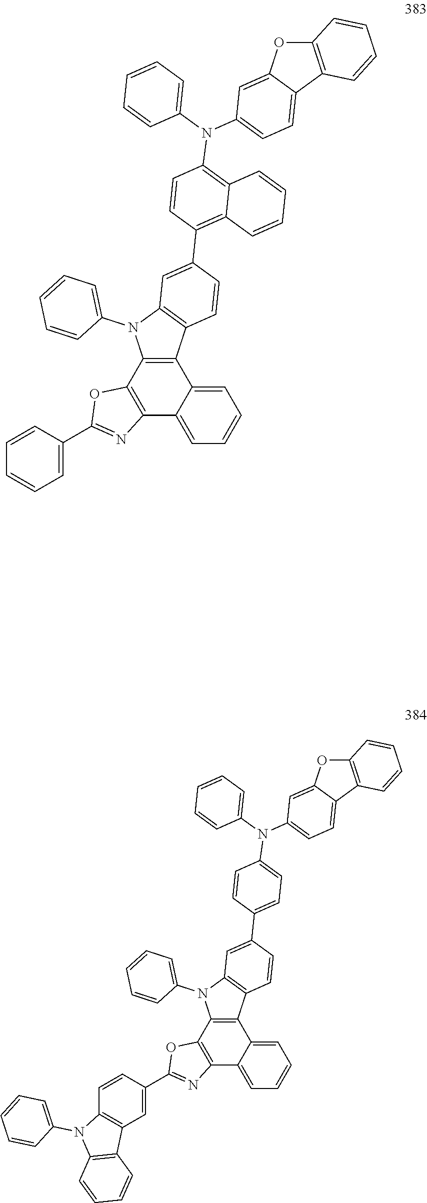 [SMILES: c1ccc(-c2nc3c4ccccc4c4c5ccc(-c6ccc(N(c7ccccc7)c7ccc8c(c7)oc7ccccc78)c7ccccc67)cc5n(-c5ccccc5)c4c3o2)cc1 ; c1ccc(N(c2ccc(-c3ccc4c5c6ccccc6c6nc(-c7ccc8c(c7)c7ccccc7n8-c7ccccc7)oc6c5n(-c5ccccc5)c4c3)cc2)c2ccc3c(c2)oc2ccccc23)cc1]

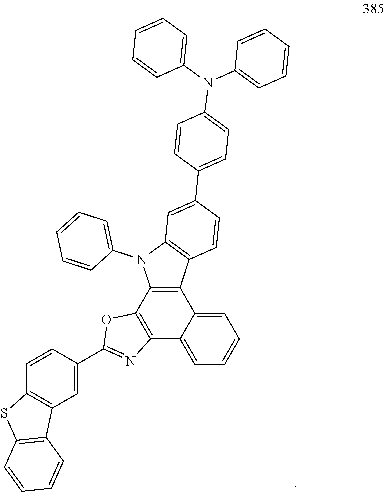 [SMILES: c1ccc(N(c2ccccc2)c2ccc(-c3ccc4c5c6ccccc6c6nc(-c7ccc8sc9ccccc9c8c7)oc6c5n(-c5ccccc5)c4c3)cc2)cc1]

13. An organic electroluminescent device, comprising an anode, a cathode, and at least one functional layer disposed between the anode and the cathode, wherein the functional layer comprises the organic compound according to claim 1.

14. The organic electroluminescent device according to claim 13, wherein the functional layer comprises an organic electroluminescent layer, and the organic electroluminescent layer comprises the organic compound.

15. An electronic apparatus, comprising the organic electroluminescent device according to claim 13.
